(19) United States
(12) Patent Application Publication
(10) Pub. No.: US 2023/0158005 A1
(43) Pub. Date: May 25, 2023

(54) Substituted Azole Dione Compounds with Antiviral Activity

(71) Applicant: Stemline Therapeutics, Inc., New York, NY (US)

(72) Inventors: Janice Chen, Downingtown, PA (US); Christopher Brooks, Brooklyn, NY (US); Ivan Bergstein, New York, NY (US)

(73) Assignee: Stemline Therapeutics, Inc., New York, NY (US)

(21) Appl. No.: 17/913,529

(22) PCT Filed: Mar. 22, 2021
(86) PCT No.: PCT/US2021/023453

§ 371 Date: Sep. 22, 2022

Related U.S. Application Data
(60) Provisional application No. 62/993,683, filed on Mar. 23, 2020.

(51) Int. Cl. A61K 31/4439; A61P 31/16

(52) U.S. Cl. CPC A61K 31/4439; A61P 31/16

(57) ABSTRACT

Provided herein are methods of using substituted azole dione compounds for treatment of viral infections.

### CROSS REFERENCE TO RELATED APPLICATIONS

This application claims priority to U.S. Provisional Application No. 62/993,683, filed on Mar. 23, 2020, the disclosure of which is hereby incorporated by reference in its entirety.

### DESCRIPTION

### Field

The present application relates to novel methods of using substituted azole dione compounds having antiviral activity.

### Background

The treatment of viral diseases faces challenges with the emergence and re-emergence of viruses that evolve faster than antiviral drugs can be developed. These events have motivated the development of broad-spectrum antiviral drugs. A method of developing such drugs is the targeting of host factors required for viral replication as they are less likely than viral proteins to mutate under drug-mediated selective pressures. Provided herein are compounds that target a broad spectrum of viruses by targeting a host factor.

Felezonexor (SL-801; Stemline Therapeutics Inc, New York, NY), the structure of which is shown below, is an oral, small molecule reversible inhibitor of exportin-1 (XPO1), also known as chromosome region maintenance 1 (CRM1).

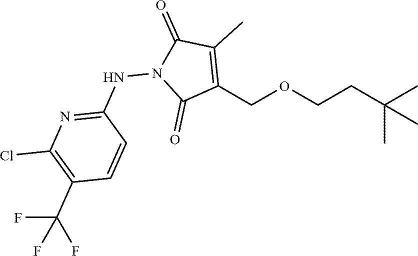 [SMILES: CC1=C(COCCC(C)(C)C)C(=O)N(Nc2ccc(C(F)(F)F)c(Cl)n2)C1=O]

XPO1 is a key protein that mediates nuclear-cytoplasmic transport of over 200 nuclear proteins, as well as mRNAs. In several pathological conditions such as cancer and viral infections, the hijacking or alteration of function of key transporter proteins, such as XPO1, is observed (Mathew C, Ghildval R, 2017).

Felezonexor is currently in clinical trials for patients with advanced solid tumors (NCT02667873). Preliminary clinical results demonstrated felezonexor’s clinical activity and a manageable safety and tolerability profile in 57 patients with relapsed/refractory, locally advanced or metastatic solid tumors. In this ongoing clinical study, felezonexor demonstrated antitumor activity with durable responses, including a partial response, in multiple patients with diverse, heavily pre-treated solid tumors. Safety and tolerability were manageable, with the most common treatment related adverse events being gastrointestinal (nausea, vomiting, and diarrhea) and constitutional (fatigue, decreased appetite) in nature (Wang et al, 2019).

XPO1 is utilized by numerous viruses to shuttle viral components from the nucleus of infected cells to the cytoplasm for replication or pathogenicity (Mathew C, Ghildval R, 2017). Inhibition of XPO1 can lead to nuclear retention of certain viral proteins of the Herpesviridae family (e.g., Herpes simplex virus 1, Human cytomegalovirus, and Epstein-Barr virus), Flaviviridae family (e.g., Dengue virus and West Nile virus), Hepadnaviridae family (e.g., Hepatitis B virus), Togaviridae family (e.g., Venezuelan equine encephalitis virus and Chikungunya virus), and Papoviridae family (e.g., Papilloma virus), as well as respiratory viruses (e.g., respiratory syncytial virus), and other viruses such as adenovirus and Nipah virus (Funk et al., 2019; Sanchez et al., 2007; Liu et al., 2012; Boyle et al., 1999; Rawlinson et al., 2009; Oh et al., 2006; Forgues et al., 2001; Atasheva et al., 2010; Thomas et al., 2013; Blachon et al., 2005; Ghildyal et al., 2009; Schmid et al., 2012; Wang et al., 2010). As such, XPO1 is an attractive target for antiviral drug discovery.

In SARS-CoV, the nuclear export of two SARS-CoV proteins, ORF 3b and ORF 9b, has been shown to be dependent on XPO1 (Freundt et al., 2009; Sharma et al., 2011). The ORF3b protein encoded by SARS-CoV contributes to viral pathogenicity through inhibition of induction type I interferons. ORF 3b is actively transported out of the nucleus via CRM1/XPO1. A homologous ORF 3 protein is encoded by the SARS-CoV2 virus. Interestingly, blocking nuclear export of ORF 9b was shown to induce caspase-3 activation and apoptosis in Vero E6 cells infected with SARS-CoV (Sharma et al., 2011). Importantly, SARS-CoV2 shares the highest nucleotide sequence identity (79.7%) with SARS-CoV (Zhou et al., 2020). In addition, recent work identified XPO1 as a hub protein with one of the highest number of connections within a 119-protein coronavirus-host interactome network generated by assembling known coronavirus-associated host proteins from six coronavirus strains (Zhou et al, 2020). Another study used an affinity-purification mass spectrometry approach to identify SARS-CoV2-human protein-protein interactions and found that Orf6 from SARS-CoV2 interacts with an mRNA nuclear export complex that is targetable by an XPO1 inhibitor (Gordon et al., 2020). Thus, inhibition of XPO1 may be a useful strategy for inhibiting SARS-CoV2 production. As such, targeting XPO1 with felezonexor to act as a host-directed inhibitor of SARS-CoV2 could offer a novel therapeutic option. Additionally, felezonexor’s targeting of a host protein potentially circumvents the emergence of drug-resistant viral strains that can arise as a consequence of new genetic mutations in viral components (Mathew C, Ghildval R, 2017).

### SUMMARY

Described herein, in certain embodiments, are methods for treating or preventing a viral infection in a subject by administering substituted azole dione compounds to the subject.

The following embodiments are encompassed.

Embodiment 1 is a method of treating a viral infection in a subject comprising administering to the subject an effective amount of a compound having the formula of Structure (II):

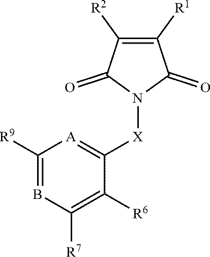

wherein:
R¹ and R² are independently selected from alkyl, substituted alkyl, and optionally substituted alkoxy, wherein at least one of R¹ and R² is methyl;
X is NR³;
R³ is H, alkyl, or acyl;
A is N or CH;
B is CR⁸;
R⁶ is selected from H, alkyl, substituted alkyl, and halogen;
R⁷ is selected from H, alkyl, substituted alkyl, halogen, optionally substituted aryl, optionally substituted heteroaryl, optionally substituted alkoxy, optionally substituted aryloxy, cyano, optionally substituted alkylthio, optionally substituted alkylsufinyl, optionally substituted alkylsulfonyl, optionally substituted arylthio, optionally substituted acyl, optionally substituted amino, carboxyl, optionally substituted alkoxycarbonyl, and optionally substituted carbamoyl, wherein R⁶ and R⁷ optionally form a fused aryl group when each of R⁶ and R⁷ is alkyl;
R⁸ is selected from H, alkyl, substituted alkyl, and halogen; and
R⁹ is selected from H, alkyl, substituted alkyl, halogen, optionally substituted aryl, and cyano, wherein R⁸ and R⁹ optionally form one or more optionally substituted fused aryl groups when each of R⁸ and R⁹ is alkyl or substituted alkyl;
wherein at least one of R⁶, R⁷, R⁸, and R⁹ is halogen selected from Br and Cl, or alkyl substituted with one or more halogen groups selected from Br, Cl, and F;
or a salt thereof.

Embodiment 2 is the method of embodiment 1, wherein the compound has the formula of Structure (IV):

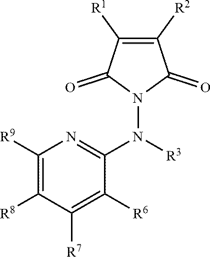

Embodiment 3 is the method of embodiment 1 or 2, wherein one of R¹ and R² is methyl, and the other of R¹ and R² is alkyl or alkyl substituted with alkoxy, hydroxy, carboxy, or alkoxycarbonyl.

Embodiment 4 is the method of any one of embodiments 1-3, wherein R³ is H or alkyl.

Embodiment 5 is the method of any one of embodiments 1-4, wherein R³ is H or methyl.

Embodiment 6 is the method of any one of embodiments 1-5, wherein R⁶ is H, R⁷ is H, R⁸ is halogen or alkyl substituted with one or more halogen groups, and R⁹ is halogen.

Embodiment 7 is the method of any one of embodiments 1-6, wherein one of R¹ and R² is methyl, and the other of R¹ and R² is alkyl or alkyl substituted with alkoxy, hydroxy, carboxy, or alkoxycarbonyl, R⁶ is H, R⁷ is H, R⁸ is CF₃, and R⁹ is Cl.

Embodiment 8 is the method of any one of embodiments 1-7, wherein the compound is 3-[(3,3-dimethylbutoxy)methyl]-1-{[6-chloro-5-(tritluoromethyl)(2-pyridyl)]amino}-4-methylazoline-2,5-dione (S03747), or a salt thereof, having the following structure:

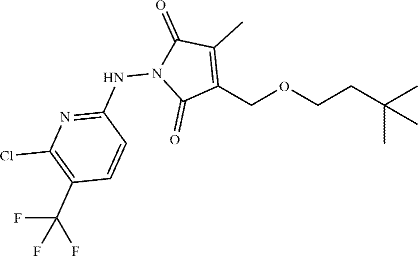 [SMILES: CC1=C(COCCC(C)(C)C)C(=O)N(Nc2ccc(C(F)(F)F)c(Cl)n2)C1=O]

Embodiment 9 is the method of any one of embodiments 1-8, wherein the compound has a selectivity index of greater than 1 in animal cells.

Embodiment 10 is the method of any one of embodiments 1-8, wherein the compound has a selectivity index of between 1 and 1000 in animal cells.

Embodiment 11 is the method of any one of embodiments 1-10, wherein the compound is administered as a pharmaceutical composition comprising the compound and a pharmaceutically acceptable excipient.

Embodiment 12 is the method of any one of embodiments 1-11, wherein the compound binds XPO1.

Embodiment 13 is the method of any one of embodiments 1-12, wherein the compound binds to Cys528 of XPO1.

Embodiment 14 is the method of embodiment 12 or 13, wherein the binding is reversible.

Embodiment 15 is the method of any one of embodiments 1-14, wherein contacting a cell with the compound increases nuclear retention of a viral protein.

Embodiment 16 is the method of any one of embodiments 1-15, wherein contacting a cell with the compound increases nuclear retention of a viral ribonucleoprotein (vRNP).

Embodiment 17 is the method of any one of embodiments 1-16, wherein contacting a cell with the compound blocks nuclear export of a vRNP or viral protein.

Embodiment 18 is the method of any one of embodiments 1-17, wherein the viral infection is caused by a virus belonging to the Togaviridae, Arenaviridae, Poxviridae, Toroviridae, Paramyxoviridae, Herpesviridae, Retroviridae, Coronaviridae, Flaviviridae, Bunyaviridae, Pneumoviridae, Filoviridae, Adenoviridae, Papovaviridiae, Hepadnaviridae, or Orthomyxoviridae family.

Embodiment 19 is the method of any one of embodiments 1-18, wherein the viral infection is caused by dengue virus (DENV), respiratory syncytial virus (RSV), Venezuelan equine encephalitis virus (VEEV), influenza virus, human immunodeficiency virus (HIV), herpes simplex virus (HSV), cytomegalovirus (CMV), Ebola virus, rubulavirus, Nipah virus, Hepatitis B virus, BK virus, JC virus, papillomavirus, adenovirus-5, cowpox virus, measles virus, varicella-zoster virus, Epstein-Barr virus, Kaposi’s sarcoma associated herpesvirus, West Nile virus, Chikungunya virus (CHIKV), or coronavirus.

Embodiment 20 is the method of any one of embodiments 1-19, wherein the viral infection is an influenza infection.

Embodiment 21 is the method of embodiment 20, wherein the influenza infection is an influenza A, influenza B, or influenza C infection.

Embodiment 22 is the method of embodiment 21, wherein the influenza A infection comprises infection by H1N1, H1N2, H3N2, H5N1, or H7N9 subtypes of influenza.

Embodiment 23 is the method of any one of embodiments 1-19, wherein the viral infection is a Coronavirus infection.

Embodiment 24 is the method of embodiment 23, wherein the Coronavirus infection comprises infection by SARS-CoV2.

Embodiment 25 is the method of embodiment 23 or 24, wherein the Coronavirus infection causes COVID-19.

Embodiment 26 is the method of any one of embodiments 1-19, wherein the viral infection is a human immunodeficiency virus (HIV) infection.

Embodiment 27 is the method of any one of embodiments 1-19, wherein the viral infection is a Nipah virus infection.

Embodiment 28 is the method of any one of embodiments 1-27, wherein the method of treating a viral infection comprises reducing the duration of infection.

Embodiment 29 is the method of any one of embodiments 1-28, wherein the method of treating a viral infection comprises reducing the symptoms of infection.

Embodiment 30 is the method of any one of embodiments 1-29, wherein the method of treating a viral infection comprises reducing the severity of the infection.

Embodiment 31 is the method of any one of embodiments 1-30, wherein the method of treating a viral infection comprises reducing viral infectivity.

Embodiment 32 is the method of any one of embodiments 1-31, wherein the method of treating a viral infection comprises reducing viral replication.

Embodiment 33 is the method of any one of embodiments 1-32, wherein the method of treating a viral infection comprises reducing viral shedding.

Embodiment 34 is the method of any one of embodiments 1-33, wherein the subject is a human patient.

Embodiment 35 is the method of any one of embodiments 1-33, wherein the subject is a cell and the method is an in vitro method.

Embodiment 36 is the method of any one of embodiments 1-33, wherein the subject is a human patient’s cell and the method is an ex vivo method.

Embodiment 37 is a compound as recited in any one of embodiments 1-36 for use in the manufacture of a medicament for treating a viral infection.

Embodiment 38 is the use of a compound as recited in any one of embodiments 1-36 for treating a viral infection.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows the viability of A549 human lung carcinoma cells exposed to DMSO control or 50, 100, 250, 500, or 1000 nM felezonexor for 48 hours. Viability is shown as a percentage of cells treated with DMSO only.

FIG. 2 shows viral titers in A549 human lung carcinoma cells infected in vitro with various strains of influenza A (H1N1) viruses and treated with DMSO control, oseltamivir, or 10, 100, or 500 nM felezonexor.

FIG. 3 shows viral titers in A549 human lung carcinoma cells infected in vitro with various strains of influenza A (H3N2) viruses and treated with DMSO control, oseltamivir, or 10, 100, or 500 nM felezonexor.

FIG. 4 shows viral titers in A549 human lung carcinoma cells infected in vitro with various strains of influenza A (H5N1 and H7N9) viruses and treated with DMSO control, oseltamivir, or 10, 100, or 500 nM felezonexor.

FIG. 5 shows viral titers in the lungs of mice intranasally infected with influenza A strain A/England/195/2009 (H1N1) after 5 days. Mice (n=7/group) were treated orally with various compounds and regimens: (A) 5% gum arabic vehicle control administered bid on Days 0, 2, 4 and qd on Days 1 and 3; (B) 40 mg/kg Tamiflu administered bid on Days 0-4; (C) proprietary reference inhibitor; (D) 100 mg/kg felezonexor administered bid on Days 0, 2, 4 and qd on Days 1 and 3; or (E) 100 mg/kg felezonexor administered qd on Days 0 and 4 and bid on Day 2.

FIG. 6 shows daily body weight assessment of influenza-infected mice (influenza A strain A/England/195/2009 (H1N1)) treated orally with various compounds and regimens: (A) 5% gum arabic vehicle control administered bid on Days 0, 2, 4 and qd on Days 1 and 3; (B) 40 mg/kg Tamiflu administered bid on Days 0-4; (C) proprietary reference inhibitor; (D) 100 mg/kg felezonexor administered bid on Days 0, 2, 4 and qd on Days 1 and 3; or (E) 100 mg/kg felezonexor administered qd on Days 0 and 4 and bid on Day 2. Body weights are normalized to that on Day 0.

FIG. 7 shows quantitation of viral titers from MT2 human T cell line infected in vitro with HIV-1 and treated with DMSO or 0.001, 0.01, 0.1, 1, or 10 µM felezonexor (left), as well as viability of uninfected MT2 cells exposed to DMSO or 0.001, 0.01, 0.1, 1, or 10 µM felezonexor (right).

FIG. 8 shows the % inhibition of infection (i.e., % activity) in Nipah virus-infected HeLa cells treated with 10, 3.33, 1.11, 0.37, 0.12, 0.041, 0.014, or 0.0046 µM felezonexor.

FIG. 9 shows the % cell count (i.e., signal) in Nipah virus-infected HeLa cells treated with 10, 3.33, 1.11, 0.37, 0.12, 0.041, 0.014, or 0.0046 µM felezonexor.

### DESCRIPTION OF THE EMBODIMENTS

### I. Introduction

Thus far, preclinical testing of felezonexor has been conducted in A549 pulmonary epithelial cells infected with a panel of 20 influenza A viruses comprising various subtypes, including H1N1, H1N2, H3N2, H5N1, and H7N9. Felezonexor reduced viral titers at least 3-fold compared to untreated controls in 17 out of 20 strains and at least 10-fold in 13 out of 20 strains. Importantly, viral titers of infected A549 cells were reduced at felezonexor concentrations achievable in human plasma and tolerable by human subjects following oral dosing (data on file). Furthermore, oral administration of felezonexor was found to decrease viral titers in the lungs of mice intranasally infected with A/England/195/2009 (H1N1) in a five-day study. The unfolding preclinical data also demonstrate that felezonexor inhibits HIV-1, Nipah virus, and potentially Venezuelan equine encephalitis virus replication in vitro (data on file). Ongoing research is aimed at assessing felezonexor against diverse families of viruses.

Based on felezonexor’s broad anti-viral properties, a potential use of felezonexor, which has demonstrated promising outcomes for treatment of diverse cancers, is to treat patients with COVID-19. Felezonexor is believed to have sufficient safety and tolerability data from ongoing clinical trials in patients with advanced solid tumors to support initiating an innovative clinical trial to test the role of felezonexor in accelerating patients’ recovery from COVID-19. Use of felezonexor to treat infections and/or diseases caused by other viruses as described herein is also contemplated.

### II. Felezonexor and Other Substituted Azole Dione Compounds

The disclosure provides compounds having the formula of Structure (I):

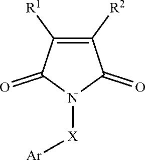

wherein R¹ and R² are independently chosen from alkyl, substituted alkyl, optionally substituted alkoxy, optionally substituted alkylthio, halogen, optionally substituted aryl, optionally substituted aryloxy, optionally substituted arylthio, and H, where R¹ and R² can also be part of a cyclic alkylene chain that form a fused ring structure, X is O, S, NR³, or CR⁴R⁵, Ar is aryl or substituted aryl, including carbocyclic aryl, heterocyclic aryl, monocyclic aryl, polycyclic aryl, and aryl fused with non-aryl (non-aromatic) rings, R³ is H, alkyl, substituted alkyl, optionally substituted acyl, or as part of a ring structure that connects the N to the Ar ring, R⁴ and R⁵ are chosen independently from H, alkyl, and substituted alkyl, or both can be part of a cyclic alkylene chain that forms a ring structure and R⁴ or R⁵ can also be part of a ring structure that connects to the Ar ring, or a salt of any of these compounds.

The disclosure provides methods for treating a viral infection comprising administering to a subject an effective amount of a compound having the formula of Structure (I):

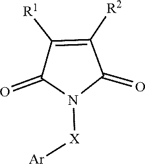

wherein R¹ and R² are independently chosen from alkyl, substituted alkyl, optionally substituted alkoxy, optionally substituted alkylthio, halogen, optionally substituted aryl, optionally substituted aryloxy, optionally substituted arylthio, and H, where R¹ and R² can also be part of a cyclic alkylene chain that form a fused ring structure, X is O, S, NR³, or CR⁴R⁵, Ar is aryl or substituted aryl, including carbocyclic aryl, heterocyclic aryl, monocyclic aryl, polycyclic aryl, and aryl fused with non-aryl (non-aromatic) rings, R³ is H, alkyl, substituted alkyl, optionally substituted acyl, or as part of a ring structure that connects the N to the Ar ring, R⁴ and R⁵ are chosen independently from H, alkyl, substituted alkyl, or both can be part of a cyclic alkylene chain that forms a ring structure and R⁴ or R⁵ can also be part of a ring structure that connects to the Ar ring, or a salt of any of these compounds. The disclosure provides methods for treating a viral infection comprising administering an effective amount of a compound having the formula of Structure (I) in vivo, ex vivo, or in vitro.

The present disclosure provides compounds with antiviral activity including:
tert-butyl 3-(1- { [6-chloro-5-(trifluoromethyl) (2-pyridyl)]amino} -4-methyl-2,5-dioxoazolin-3-yl) propanoate (S01860);
ethyl 3-(1-{[6-chloro-5-(trifluoromethyl)(2-pyridyl)]amino} -4-methyl-2,5-dioxoazolin-3-yl) propanoate (S01861);
3,4-dimethyl-1-[(4,7,8-trichloro(2-quinolyl))amino]azoline-2,5-dione (S01078);
1-[(8-bromo-4-chloro(2-quinolyl))amino]-3,4-dimethylazoline-2,5-dione (S01247);
tert-butyl 4-({2-[(3,4-dimethyl-2,5-dioxoazolinyl)amino]-7-bromo-4-quinolyl} methyl)piperazinecarboxylate (S01589);
methyl 3-(1-{[6-chloro-5-(trifluoromethyl)(2-pyridyl)]amino}-4-methyl-2,5-dioxoazolin-3-yl)propanoate (S01648);
3-(1-1[6-chloro-5-(trifluoromethyl)(2-pyridyl)]amino}-4-methyl-2,5-dioxoazolin-3-yl)-N-methoxy-N-methylpropanamide (S01796);
1-{[7-bromo-4-({4-[(2-methoxyphenyl)carbonyl]piperazinyl}methyl)(2-quinolyl)]amino} -3,4-dimethylazoline-2,5-dione (S01879);
1- { [3-bromo-6-chloro-5-(trifluoromethyl) (2-pyridyl)] amino} -3,4-dimethylazoline-2,5-dione (S01981);
1- { [6-chloro-5-(trifluoromethyl) (2-pyridyl)] amino} -3,4-dimethylazoline-2,5-dione (S00109);
1- { [6-chloro-5-(trifluoromethyl) (2-pyridyl)] methylamino} -3,4-dimethylazoline-2,5-dione (S00170);
1- { [6-bromo-5-(trifluoromethyl) (2-pyridyl)] methylamino} -3,4-dimethylazoline-2,5-dione (S01007);
1- { [6-chloro-5-(trifluoromethyl) (2-pyridyl)] amino} -4-methyl-3-(3-methylbutyl) azoline-2,5-dione (S01554);
1- { [6-chloro-5-(trifluoromethyl) (2-pyridyl)] amino} -3-(methoxymethyl)-4-methylazoline-2,5-dione (S01599);
1- { [7,8-dichloro-4-(trifluoromethyl) (2-quinolyl)] amino } -3,4-dimethylazoline-2,5-dione (S01455);
3-(1-{[6-chloro-5-(tritluoromethyl)(2-pyridyl)]amino}-4-methyl-2,5-dioxoazolin-3-yl)-N,N-diethylpropanamide (S01711);
diethyl 2-[(1- { [6-chloro-5-(trifluoromethyl) (2-pyridyl)]amino} -4-methyl-2,5-dioxoazolin-3-yl)methyl]propane-1,3-dioate (S01712);
N-(tert-butyl)-3-(1- { [6-chloro-5-(trifluoromethyl) (2-pyridyl)] amino } -4-methyl-2,5-dioxoazolin-3-yl)propanamide (S01758);
1- {[7-bromo-4-({4-[(3-methoxyphenyl)carbonyl]piperazinyl} methyl) (2-quinolyl)]amino} -3,4-dimethylazoline-2,5-dione (S01925);
1-{[6-bromo-5-(trifluoromethyl)(2-pyridyl)]amino}-3,4-dimethylazoline-2,5-dione (S00994);
1-[(4,8-dichloro(2-quinolyl))amino]-3,4-dimethylazoline-2,5-dione (S01005);
3,4-dimethyl-1- { [6-phenyl-5-(trifluoromethyl) (2-pyridyl)] amino} azoline-2,5-dione (S01266);
1- { [6-chloro-5-(trifluoromethyl) (2-pyridyl)] amino} -3-(hydroxymethyl)-4-methylazoline-2,5-dione (S01470);
N-(3,4-dimethyl-2,5-dioxoazolinyl)-N-[6-chloro-5-(trifluoromethyl) (2-pyridyl)]acetamide (S01473);
1- {[7-bromo-4-({4-[(2-chlorophenyl)carbonyl]piperazinyl} methyl) (2-quinolyl)]amino} -3,4-dimethylazoline-2,5-dione (S01878);
3-(1- { [6-chloro-5-(trifluoromethyl) (2-pyridyl)] amino } -4-methyl-2,5-dioxoazolin-3-yl)-N-methylpropanamide (S01883);
1-[(8-chloro(2-quinolyl))amino]-3,4-dimethylazoline-2,5-dione (S00585);
3,4-dimethyl-1-[(3,4,5-trichlorophenyl)amino]azoline-2,5-dione (S00832);
3,4-dimethyl-1-{[4-(trifluoromethyl)(2-quinolyl)]amino}azoline-2,5-dione (S00873);
1-[(7-bromo-4-chloro(2-quinolyl))amino]-3,4-dimethylazoline-2,5-dione (S01311);
1- { [6-(3-chloro-4-fluorophenyl)-5-(trifluoromethyl) (2-pyridyl)] amino} -(3,4-dimethyl (3,4-dimethyl methylazoline-2,5-dione (S01313);
3,4-dimethyl-1- { [6-(2-methylpropyl)-5-(trifluoromethyl) (2-pyridyl)]amino}azoline-2,5-dione (S01457);
1- { [6-chloro-4-(trifluoromethyl) (2-pyridyl)] amino} -3,4-dimethylazoline-2,5-dione (S01737);
methyl 3-(1-{[4-({4-[(tert-butyl)oxycarbonyl]piperazinyl}methyl)-7-bromo(2-quinolyl)]amino}-4-methyl-2,5-dioxoazolin-3-yl)propanoate (S01865);
1-({4-[(4-{[4-(dimethylamino)phenyl]carbonyl}piperazinyl)methyl]-7-bromo (2-quinolyl)}amino)-3,4-dimethylazoline-2,5-dione (S01880);
1-[(3-chloroisoquinolyl)amino]-3,4-dimethylazoline-2,5-dione (S01098);
1- { [6-chloro-5-(trifluoromethyl) (2-pyridyl)] amino} -3-ethyl-4-methylazoline-2,5-dione (S01553);
1-{[4-chloro-6-phenyl-5-(trifluoromethyl)(2-pyridyl)] amino}-3,4-dimethylazoline-2,5-dione (S01734);
N-[1-({2-[(3,4-dimethyl-2,5-dioxoazolinyl)amino]-7-bromo(4-quinolyl)}methyl)pyrrolidin-3-yl] (tert-butoxy) carboxamide (S01864);
1-{[7-bromo-4-({4-[(4-fluorophenyl)carbonyl]piperazinyl}methyl)(2-quinolyl)]amino} -3,4-dimethylazoline-2,5-dione (S01877);
6-[(3,4-dimethyl-2,5-dioxoazolinyl)amino]-3-(trifluoromethyl)pyridine-2-carbonitrile (S01475);
2-{[6-chloro-5-(trifluoromethyl)-2-pyridyl]amino}-4,5,6,7-tetrahydroisoindole-1,3-dione (S00186);
1- { [4-bromo-3-(trifluoromethyl)phenyl] amino} -3,4-dimethylazoline-2,5-dione (S00516);
1-[(4-chloronaphthyl)amino]-3,4-dimethylazoline-2,5-dione (S00738);
1-[(4-chloro-6-methyl(2-quinolyl))amino]-3,4-dimethylazoline-2,5-dione (S00935);
1-[(4-bromonaphthyl)amino]-3,4-dimethylazoline-2,5-dione (S00942);
1- { [7-bromo-4-(hydroxymethyl) (2-quinolyl)] amino } -3,4-dimethylazoline-2,5-dione (S01037);
{2-[(3,4-dimethyl-2,5-dioxoazolinyl)amino]-7-bromo-4-quinolyl} methylacetate (S01047);
1- { [8-chloro-4-(4-methoxyphenyl) (2-quinolyl)] amino } -3,4-dimethylazoline-2,5-dione (S01191);
1-[(4-chlorobenzo[h]quinolin-2-yl)amino]-3,4-dimethylazoline-2,5-dione (S01207);
1-[(7-bromo-4-{[4-benzylpiperazinyl]methyl}(2-quinolyl))amino]-3,4-dimethylazoline-2,5-dione (S01268);
1- { [6-(4-chlorophenyl)-5-(trifluoromethyl) (2-pyridyl)] amino} -3,4-dimethylazoline-2,5-dione (S01371);
3,4-dimethyl-1-{[6-(4-methylphenyl)-5-(trifluoromethyl)(2-pyridyl)]amino} azoline-2,5-dione (S01393);
1- { [6-(3-chlorophenyl)-5-(trifluoromethyl) (2-pyridyl)] amino} -3,4-dimethylazoline-2,5-dione (S01474);
1-{[6-chloro-5-(trifluoromethyl)(2-pyridyl)] methylamino}-3-(methoxymethyl)-4-methylazoline-2,5-dione (S01600);
phenylmethyl4-({2-[(3,4-dimethyl-2,5-dioxoazolinyl)amino]-7-bromo-4-quinolyl} methyl)piperazinecarboxylate (S01683);
1-{[6-chloro-2-phenyl-3-(tritluoromethyl)(4-pyridyl)]amino}-3,4-dimethylazoline-2,5-dione (S01688);
3,4-dimethyl-1-({6-[3-(trifluoromethyl)phenyl](2-pyridyl)}amino)azoline-2,5-dione (S01691);
1-[(7-bromo-4-{[4-(phenylcarbonyl)piperazinyl]methyl}(2-quinolyl))amino]-3,4-dimethylazoline-2,5-dione (S01699);
3-(1-1[6-chloro-5-(trifluoromethyl)(2-pyridyl)]amino-4-methyl-2,5-dioxoazolin-3-yl)-N-methyl-N-phenylpropanamide (S01759);
3,4-dimethyl-1-{[6-benzyl-5-(trifluoromethyl)(2-pyridyl)]amino}azoline-2,5-dione (S01762);
1-{[4-({4-[(2,4-dimethylphenyl)carbonyl]piperazinyl}methyl)-7-bromo(2-quinolyl)]amino} -3,4-dimethylazoline-2,5-dione (S01800);
1-{[7-bromo-4-({4-[(4-methoxyphenyl)carbonyl]piperazinyl}methyl)(2-quinolyl)]amino}-3,4-dimethylazoline-2,5-dione (S01801);
N-[6-chloro-5-(trifluoromethyl)(2-pyridyl)]-N-[4-(hydroxymethyl)-3-methyl-2,5-dioxoazolinyl]acetamide (S01820);
1-[(7-bromo-4-{[4-(phenylsulfonyl)piperazinyl]methyl}(2-quinolyl))amino]-3,4-dimethylazoline-2,5-dione (S01822);
1-[(4-chloro-8-methyl(2-quinolyl))amino]-3,4-dimethylazoline-2,5-dione (S00871);
tert-butyl 4-[({2-[(3,4-dimethyl-2,5-dioxoazolinyl)amino]-7-bromo-4-quinolyl} methyl)amino]piperidinecarboxylate (S01862);
tert-butyl 4-[4-({2-[(3,4-dimethyl-2,5-dioxoazolinyl)amino]-7-bromo-4-quinolyl} methyl)piperazinyl]piperidinecarboxylate (S01928);
1-[(4-{[4-(3,3-dimethylbutanoyl)piperazinyl]methyl}-7-bromo(2-quinolyl)) amino]-3,4-dimethylazoline-2,5-dione (S01929);
methylethyl 3-(1-1[6-chloro-5-(trifluoromethyl) (2-pyridyl)]amino}-4-methyl-2,5-dioxoazolin-3-yl) propanoate (S02022);
methylpropyl 3-(1-{[6-chloro-5-(trifluoromethyl) (2-pyridyl)] amino}-4-methyl-2,5-dioxoazolin-3-yl) propanoate (S02264);
tert-butyl 2-(1-1[6-chloro-5-(trifluoromethyl)(2-pyridyl)]amino}-4-methyl-2,5-dioxoazolin-3-yl)acetate (S02225);
1-{[6-chloro-5-(tritluoromethyl) (2-pyridyl)]amino}-3-(ethoxymethyl)-4-methylazoline-2,5-dione (S02366);
3-butyl-1-1[6-chloro-5-(trifluoromethyl) (2-pyridyl)]amino}-4-methylazoline-2,5-dione (S03448);
1-{[6-chloro-5-(trifluoromethyl) (2-pyridyl)]amino}-4-methyl-3-[2-(2-methyl (1,3-dioxolan-2-yl)) ethyl]azoline-2,5-dione (S03456);
1-{[6-chloro-5-(trifluoromethyl)(2-pyridyl)]amino}-3-[(2-methoxyethoxy)methyl]-4-methylazoline-2,5-dione (S03742);
1- { [6-chloro-5-(trifluoromethyl) (2-pyridyl)] amino} -4-(3-hydroxyhexyl)-3-methylazoline-2,5-dione (S03552);
1- { [6-chloro-5-(trifluoromethyl) (2-pyridyl)] amino} -4-(3-hydroxypentyl)-3-methylazoline-2,5-dione (S03745);
1- { [6-chloro-5-(trifluoromethyl) (2-pyridyl)] amino} -4-methyl-3-[(3-methylbutoxy)methyl]azoline-2,5-dione (S03405);
3-(butoxymethyl)-1- { [6-chloro-5-(trifluoromethyl) (2-pyridyl)] amino } -4-methylazoline-2,5-dione (S03518);
3-[(3,3-dimethylbutoxy)methyl]-1-{[6-chloro-5-(trifluoromethyl) (2-pyridyl)]amino}-4-methylazoline-2,5-dione (S03747, also known as SL-801 or felezonexor);
1- { [6-chloro-5-(trifluoromethyl) (2-pyridyl)] amino} -3-(2-ethoxyethyl)-4-methylazoline-2,5-dione (S03960);
1- { [6-chloro-5-(trifluoromethyl) (2-pyridyl)] amino} -4-methyl-3-[(2-methylpropoxy)methyl]azoline-2,5-dione (S03963);
3-[(2,2-dimethylpropoxy)methyl]-1-{[6-chloro-5-(trifluoromethyl) (2-pyridyl)]amino}-4-methylazoline-2,5-dione (S03962);
4-[(1,3-dimethylbutoxy)methyl]-1-{[6-chloro-5-(trifluoromethyl) (2-pyridyl)]amino}-3-methylazoline-2,5-dione (S03964);
4-[(tert-butoxy)methyl]-1-{[6-chloro-5-(trifluoromethyl)(2-pyridyl)]amino}-3-methylazoline-2,5-dione (S03873);
1- { [6-chloro-5-(trifluoromethyl) (2-pyridyl)]amino} -4-methyl-3-[2-(2-methylpropoxy) ethyl] azoline-2,5-dione (S03 9 5 5);
1- { [6-chloro-5-(trifluoromethyl) (2-pyridyl)]amino} -4-methyl-3-[2-(3-methylbutoxy) ethyl] azoline-2,5-dione (S03956);
1- { [6-chloro-5-(trifluoromethyl) (2-pyridyl)] amino} -3-methyl-4-(2-propoxyethyl)azoline-2,5-dione (S04034);
or a salt of any of these compounds.

Additional embodiments have been synthesized and tested, and details are set forth in the description below and in the examples, tables, and figures (drawings) presented herein. Other features, objects, and advantages will be apparent from the description, examples, tables, drawings, and from the claims.

Compositions containing compounds having any of the following structures may be excluded from the claims, depending on the methods of use.

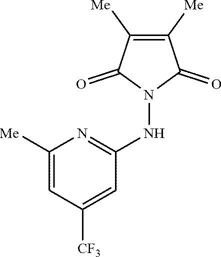 [SMILES: CC1=C(C)C(=O)N(Nc2cc(C(F)(F)F)cc(C)n2)C1=O]

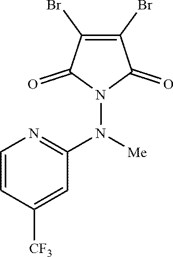 [SMILES: CN(c1cc(C(F)(F)F)ccn1)N1C(=O)C(Br)=C(Br)C1=O]

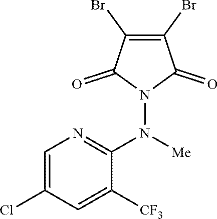 [SMILES: CN(c1ncc(Cl)cc1C(F)(F)F)N1C(=O)C(Br)=C(Br)C1=O]

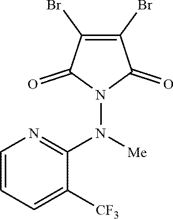 [SMILES: CN(c1ncccc1C(F)(F)F)N1C(=O)C(Br)=C(Br)C1=O]

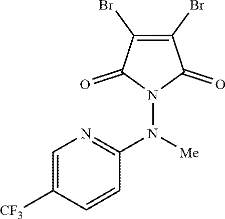 [SMILES: CN(c1ccc(C(F)(F)F)cn1)N1C(=O)C(Br)=C(Br)C1=O]

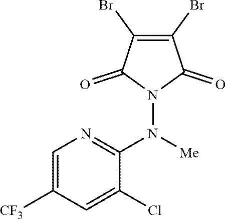 [SMILES: CN(c1ncc(C(F)(F)F)cc1Cl)N1C(=O)C(Br)=C(Br)C1=O]

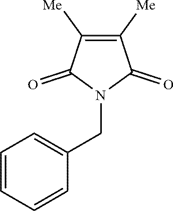 [SMILES: CC1=C(C)C(=O)N(Cc2ccccc2)C1=O]

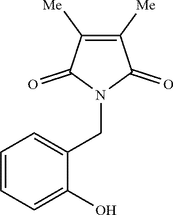 [SMILES: CC1=C(C)C(=O)N(Cc2ccccc2O)C1=O]

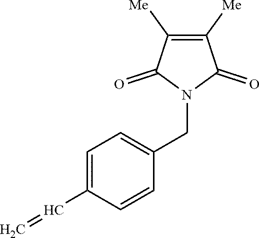 [SMILES: C=Cc1ccc(CN2C(=O)C(C)=C(C)C2=O)cc1]

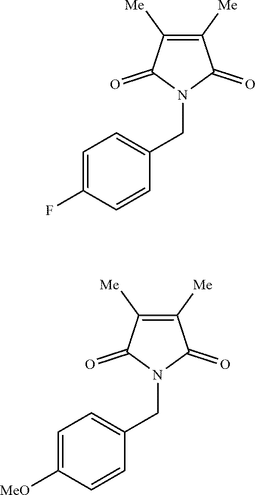 [SMILES: CC1=C(C)C(=O)N(Cc2ccc(F)cc2)C1=O ; COc1ccc(CN2C(=O)C(C)=C(C)C2=O)cc1]

### A. Definitions

Unless defined otherwise, technical and scientific terms used herein have the meaning commonly understood by a person skilled in the art. As used herein, the following terms have the meanings ascribed to them unless specified otherwise.

All publications, patents, and patent applications and ATCC deposits cited herein, are hereby expressly incorporated by reference for all purposes.

The term “compound” is intended to refer to a molecule having the structure and activity disclosed herein. A compound can be isolated, pure, substantially pure, or may be in a composition containing a mixture of other components. Purity of a composition containing a compound can be determined, for example, using analytical chemistry techniques such as high performance liquid chromatography (HPLC), liquid chromatography-mass spectrometry (LC-MS), gas chromatography-mass spectrometry (GS-MS), or other analytical techniques known to one of skill in the art. A composition as provided herein may contain one or more compounds, in a mixture with suitable vehicles, carriers, excipients, inert ingredients, and the like. If desired, a composition as provided herein may contain additional active ingredients including DNA-damaging agents and the like, as well as one or more compounds, in a mixture with suitable vehicles, carriers, excipients, inert ingredients, and the like.

The terms “pharmaceutical composition” or “medicament” refer to a composition suitable for pharmaceutical use in a subject, e.g., as an antiviral agent.

The terms “effective amount” or “sufficient amount” or any grammatical equivalent thereof, are understood to refer to an amount of a compound sufficient to produce at least one desired effect.

The term “subject” is understood to refer to animals, typically mammalian animals, such as primates (humans, apes, gibbons, chimpanzees, orangutans, macaques), domestic animals (dogs and cats), farm animals (horses, cattle, goats, sheep, pigs) and experimental animals (mouse, rat, rabbit, guinea pig). Subjects include animal disease models (e.g., tumor-prone mice, tumor-bearing mice, or mice receiving xenograft tumors). Subjects also include cells from any of the animals in either an in vitro or ex vivo context.

As used herein, the singular forms “a,” “an,” “the,” and “is” include plural referents unless the context clearly indicates otherwise. Thus, for example, reference to “a compound” includes a plurality of compounds.

### B. Chemical Terminology Definitions

“Alkyl” refers to an aliphatic hydrocarbon group. An alkyl group may be optionally substituted. Preferred alkyl groups are “C1 to C6 alkyl” such as methyl, ethyl, n-propyl, isopropyl, n-butyl, t-butyl, and like groups. “Substituted alkyl” refers to an alkyl group that is substituted by one or more substituents such as halogen (Cl, Br, F, I), C3 to C7 cycloalkyl, optionally substituted aryl, optionally substituted heteroaryl, C1 to C6 alkoxy, optionally substituted aryloxy, hydroxy, optionally substituted amino, optionally substituted cyclic amino, nitro, thio, cyano, oxo, C1 to C7 acyl, C1 to C7 acyloxy, carboxy, C1 to C6 alkoxycarbonyl, optionally substituted carbamoyl, optionally substituted cyclic aminocarbonyl, β-mercapto, C1 to C4 alkylthio, C1 to C4 alkylsulfinyl, or C1 to C4 alkylsulfonyl groups. Substituted alkyl groups may have one, two, three, four, five, or more substituents, and multiply substituted alkyl groups may be substituted with the same or with different substituents. The alkyl group may be a saturated alkyl without any alkene or alkyne moieties, or an unsaturated alkyl having at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether substituted or unsubstituted, saturated or unsaturated, may be branched, straight chain, or cyclic. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

“Alkoxy” refers to an OR group, wherein R is an alkyl or substituted alkyl. Preferred alkoxy groups are “C1 to C6 alkoxy” such as methoxy, ethoxy, n-propoxy, isopropoxy, n-butoxy, t-butoxy, and like groups.

The term “alkylthio” refers to sulfide groups such as methylthio, ethylthio, n-propylthio, isopropylthio, n-butylthio, t-butylthio, and like groups. The term “alkylsulfoxide” indicates sulfoxide groups such as methylsulfoxide, ethylsulfoxide, n-propylsulfoxide, isopropylsulfoxide, n-butylsulfoxide, sec-butylsulfoxide, and the like. The term “alkylsulfonyl” encompasses groups such as methylsulfonyl, ethylsulfonyl, n-propylsulfonyl, isopropylsulfonyl, n-butylsulfonyl, t-butylsulfonyl, and the like.

“Acyl” refers to alkyl, heteroalkyl, alkenyl, heteroalkenyl, alkynyl, heteroalkynyl, aryl, or heteroaryl groups coupled to an additional group via a carbonyl group, e.g., —C(O)—alkyl, or —C(O)—aryl. Preferred acyl groups are C1 to C7 acyl such as formyl, acetyl, propionyl, butyryl, pentanoyl, pivaloyl, hexanoyl, heptanoyl, benzoyl, and the like.

The term “amide” refers to a group with the formula —C(O)NHR or — NHC(O)R, where R is optionally substituted and is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds can be amidified.

“Aryl” or “aromatic” refers to a group with at least one ring structure having a conjugated pi electron system, i.e., having the characteristics of aromaticity in terms of electron distribution through the ring system. An aryl may be optionally substituted. Typically, the ring systems contain 5-12 ring atoms in each ring. An aryl group may be monocyclic or a fused-ring polycyclic aryl. An aryl group may be a carbocyclic aryl wherein all ring atoms are carbon, e.g., phenyl. An aryl group may be a heteroaryl or heterocyclic aryl containing at least one ring heteroatom such as oxygen, sulfur and/or nitrogen. Heterocyclic aryl groups may be monocyclic or polycyclic. Examples of heteroaryl groups include maleimidyl, imidazolyl, indolyl, pyrrolidinyl, pyridinyl, pyrimidinyl, pyrazinyl, pyridazinyl, pyrrolyl, furanyl, oxazolyl, dioxazolyl, isoxazolyl, phthalimidyl, thiazolyl, and the like. Aryl groups can be fused to other aryl groups or to non-aryl (non-aromatic) groups.

As examples of the substituents of said “optionally substituted amino” and “optionally substituted carbamoyl,” there may be mentioned phenyl, substituted phenyl, C1 to C6 alkyl, C1 to C6 substituted alkyl, C2 to C7 alkenyl, C2 to C7 substituted alkenyl, C2 to C7 alkynyl, C2 to C7 substituted alkynyl, C7 to C12 phenylalkyl, C7 to C12 substituted phenylalkyl, heteroaryl, C1 to C6 alkyl, C1 to C6 substituted alkyl, C1 to C7 acyl, C1 to C7 alkoxycarbonyl, optionally substituted carbamoyl, C1 to C4 alkylsulfonyl, and the like. The “optionally substituted amino” and “optionally substituted carbamoyl” are may be mono-substituted or disubstituted, with the same or with different substituents.

“Alkoxycarbonyl” refers to an “alkoxy” group attached to a carbonyl group.

“Cycloalkyl” refers to a monocyclic or polycyclic radical which contains only carbon and hydrogen, and may be saturated, partially unsaturated, or fully unsaturated. A cycloalkyl group may be optionally substituted. Preferred cycloalkyl groups include groups having from three to twelve ring atoms, more preferably from 5 to 10 ring atoms.

“Cyclic amino” as in “optionally substituted cyclic amino,” refers to cyclic groups containing at least one ring nitrogen including piperazino, morpholino, piperidino, pyrrolidino, and the like.

Examples of “cyclic aminocarbonyl” as in “optionally substituted cyclic aminocarbonyl,” include piperazinocarbonyl, morpholinocarbonyl, piperidinocarbonyl, pyrrolidinocarbonyl, and the like.

Substituents of “optionally substituted alkoxy,” “optionally substituted alkylthio,” “optionally substituted aryl,” “optionally substituted aryloxy,” “optionally substituted arylthio,” “optionally substituted acyl,” “optionally substituted heteroaryl,” “optionally substituted alkylthio,” “optionally substituted alkylsufinyl” “optionally substituted alkylsulfonyl,” “optionally substituted alkoxycarbonyl,” “optionally substituted cyclic amino,” and “optionally substituted cyclic aminocarbonyl” are defined in the same manner as substituents of “substituted alkyl.”

“Halogen” refers to fluorine, chlorine, bromine, or iodine atoms. One or more halogens can be present in a compound, where the halogens can be the same or different.

Embodiments of compounds may possess one or more chiral centers and each center may exist in the R or S configuration, such that it includes diastereomeric, enantiomeric, and epimeric forms as well as the appropriate mixtures thereof. Embodiments may exist as geometric isomers, such that it includes all cis, trans, syn, anti, entgegen (E), and zusammen (Z) isomers, as well as the appropriate mixtures thereof.

Compounds can exist in unsolvated as well as solvated forms with pharmaceutically acceptable solvents such as water, ethanol, and the like. In general, the solvated forms are considered equivalent to the unsolvated forms for the purposes herein.

Any salt as disclosed herein can include salts with inorganic bases, salts with organic bases, salts with inorganic acids, salts with organic acids, and salts with basic or acidic amino acids. In some embodiments, the salt is a pharmaceutically acceptable salt, suitable and appropriate for administration to a subject.

Unless otherwise indicated, when a substituent is deemed to be “optionally substituted,” it is meant that the substituent is a group that may be substituted with one or more group(s) as recited herein or as known to one of skill in the art.

Descriptions of compounds are in accordance with principles of chemical bonding known to those skill in the art. Accordingly, where a group may be substituted by one or more of a number of substituents, such substitutions are selected so as to comply with principles of chemical bonding and to give compounds which are not inherently unstable, and/or would be known to one of ordinary skill in the art as likely to be unstable, under ambient conditions such as aqueous, neutral, physiological conditions.

It is understood that compounds can be described by one of skill in the art using different terminology than the terms used here, without affecting the accuracy of the description. Compounds, structures, substituents, groups, and the like can be described using any of: IUPAC nomenclature; chemical name which is a “trivial” or “common” name; trade name; CAS registry number; SMILES notation; or other descriptors. For example, compounds described herein as “substituted azole diones” or “substituted azoline diones” could alternately be described as “substituted maleimides” or “substituted 2,5-pyrrolediones” or “substituted pyrroles” in combination with other descriptors, preferably according to standardized chemical terminology, to provide a complete description of one or more compounds.

### C. Substituted Azole Dione Compounds

Provided herein are substituted azole (azoline) dione compounds that can be used for their antiviral properties where compounds can be described by the formula of Structure (I):

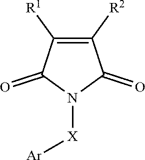

wherein:
Structure (I) contains an azoline dione heterocycle;
R¹ and R²are independently chosen from alkyl, substituted alkyl, optionally substituted alkoxy, optionally substituted alkylthio, halogen, optionally substituted aryl, optionally substituted aryloxy, optionally substituted arylthio, and H; both R¹ and R² can also be part of a cyclic alkylene chain that form a fused ring structure;
X is O, S, NR³, or CR⁴R⁵;
Ar is aryl or substituted aryl, including carbocyclic aryl, heterocyclic aryl, monocyclic aryl, polycyclic aryl, and aryl fused with non-aryl (non-aromatic) rings;
R³ is H, alkyl, substituted alkyl, or optionally substituted acyl, or is part of a ring structure that connects the N to the Ar ring;
R⁴ and R⁵ are chosen independently from H, alkyl, and substituted alkyl, or both can be part of a cyclic alkylene chain that forms a ring structure;
R⁴ or R⁵ can also be part of a ring structure that connects to the Ar ring;
or a salt thereof.

In one aspect, compounds are provided having Structure (II):

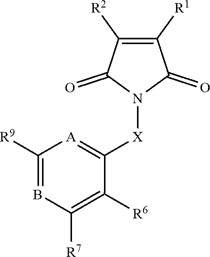

wherein:
Structure (II) contains an azoline dione heterocycle;
R¹, R², and X are defined as above for Structure (I);
A is N or CH;
B is CR⁸ or N;
R⁶, R⁷, R⁸, and R⁹ are independently chosen from H, alkyl, substituted alkyl, halogen, optionally substituted aryl, optionally substituted heteroaryl, optionally substituted alkoxy, optionally substituted aryloxy, cyano, nitro, optionally substituted alkylthio, optionally substituted alkylsufinyl, optionally substituted alkylsulfonyl, optionally substituted arylthio, optionally substituted acyl, optionally substituted amino, carboxyl, optionally substituted alkoxycarbonyl, optionally substituted carbamoyl, etc. Also included are the structures wherein two adjacent substitutions (R⁶ and R⁷, R⁷ and R⁸, R⁸ and R⁹) are part of a cyclic alkylene group that form a fused ring structure;
or a salt thereof.

In some embodiments, the variables of Structure (II) are as follows:
R¹ and R²are independently selected from alkyl, substituted alkyl, and optionally substituted alkoxy, wherein at least one of R¹ and R² is methyl;
X is NR³;
R³ is H, alkyl, or acyl;
A is N or CH;
B is CR⁸;
R⁶ is selected from H, alkyl, substituted alkyl, and halogen;
R⁷is selected from H, alkyl, substituted alkyl, halogen, optionally substituted aryl, optionally substituted heteroaryl, optionally substituted alkoxy, optionally substituted aryloxy, cyano, optionally substituted alkylthio, optionally substituted alkylsufinyl, optionally substituted alkylsulfonyl, optionally substituted arylthio, optionally substituted acyl, optionally substituted amino, carboxyl, optionally substituted alkoxycarbonyl, and optionally substituted carbamoyl, and wherein R⁶ and R⁷ can form an aryl group forming a fused ring structure when each of R⁶ and R⁷ is alkyl;
R⁸ is selected from H, alkyl, substituted alkyl, and halogen; and
R⁹ is selected from H, alkyl, substituted alkyl, halogen, optionally substituted aryl, and cyano, wherein R⁸ and R⁹ can form one or more optionally substituted aryl groups forming a fused ring structure when each of R⁸ and R⁹ is alkyl or substituted alkyl,
and wherein at least one of R⁶, R⁷, R⁸, and R⁹ is halogen selected from Br and Cl, or substituted alkyl substituted with one or more halogen groups selected from Br, Cl, and F.

In one aspect, compounds are provided having Structure (III):

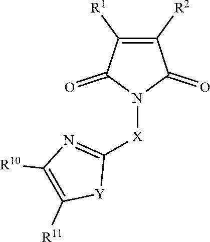

wherein:
Structure (III) contains an azoline dione heterocycle;
R¹, R², and X are defined as above for Structure (I);
Y is O, S, or NR¹²;
R¹⁰ and R¹¹ are independently chosen from H, alkyl, substituted alkyl, halogen, optionally substituted aryl, optionally substituted heteroaryl, optionally substituted alkoxy, optionally substituted aryloxy, cyano, nitro, optionally substituted alkylthio, optionally substituted alkylsufinyl, optionally substituted alkylsulfonyl, optionally substituted arylthio, optionally substituted acyl, optionally substituted amino, carboxyl, optionally substituted alkoxycarbonyl, optionally substituted carbamoyl, etc; R¹⁰ and R¹¹ could also be an alkylene group that form a “fused” ring with the heterocycle structure;
R¹² is H, alkyl, substituted alkyl, aryl, acyl, or sulfonyl groups;
or a salt thereof.

In certain non-limiting embodiments of compounds having Structure (II), X is NR³, A is N, and B is CR⁸ and compounds are provided having Structure (IV), wherein R¹, R², R³, R⁶, R⁷, R⁸, and R⁹ are defined above for Structure (II):

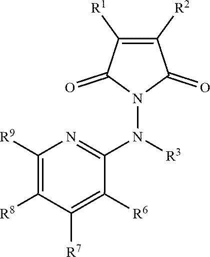

In certain non-limiting embodiments of compounds having Structure (IV), one of R¹ and R² is alkyl and the other of R¹ and R² is alkyl or substituted alkyl (such as alkyl substituted by one or more substituents selected from halogen, C1 to C6 alkoxy, hydroxy, optionally substituted amino, C1 to C7 acyl, carboxy, C1 to C6 alkoxycarbonyl, and optionally substituted carbamoyl), R³ is H, R⁶ and R⁷ are both H, R⁸ is halogen or alkyl substituted by one or more halogen groups, and R⁹ is halo, and compounds are provided having Structure (IV-A):

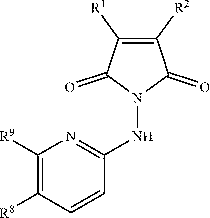

In some variations of compounds having Structure (IV-A), one of R¹ and R² is C1 to C6 alkyl and the other of R¹ and R² is C1 to C6 alkyl optionally substituted by C1 to C6 alkoxy, R⁸ is C1 to C6 alkyl substituted by one or more halogen groups, and R⁹ is halogen.

In certain non-limiting embodiments of compounds having Structure (II), X is NR³, A is N, B is CR⁸; R⁸ and R⁹ form a fused and substituted benzene ring, providing compounds having Structure (V), wherein R¹, R², R³, R⁶, and R⁷ are defined above for Structure (II); R¹³, R¹⁴, R¹⁵, and R¹⁶ are defined as for R⁶-R¹¹ above for Structure (II) and Structure (III):

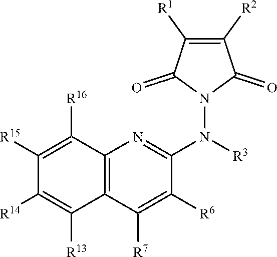

In certain non-limiting embodiments of compounds having Structure (II), X is CR⁴R⁵, A is N or CH, and B is CR⁸, providing compounds having Structure (VI), wherein R¹, R², R⁴, R⁶, R⁷, R⁸, and R⁹ are as defined above for Structure (II) and Structure (III):

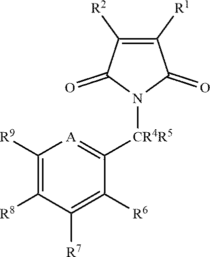

In certain non-limiting embodiments of compounds having Structure (III), X is NR³, and Y is S; R¹⁰ and R¹¹ form a fused substituted benzene ring, providing compounds having Structure (VII), wherein R¹, R², and R³ are as defined above for Structure (II); R¹⁷, R¹⁸, R¹⁹, and R²⁰ are defined as for R⁶-R¹¹ above for Structure (II) and Structure (III):

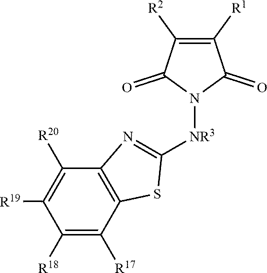

In certain non-limiting embodiments of compounds having Structure (II), X is NR³, A is N or CH, and B is CR⁸; R⁶ and R⁷ form a fused and substituted benzene ring, providing compounds having Structure (VIII), wherein R¹, R², R³, R⁸, and R⁹ are as defined above for Structure (II); R²¹, R²², R²³, and R²⁴ are defined as for R⁶-R¹¹ above for Structure (II) and Structure (III):

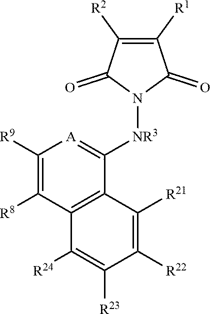

In certain non-limiting embodiments of compounds having Structure (II), X is NR³, A is N, and B is CR⁸; R⁹ is a substituted benzene ring, providing compounds having Structure (IX), wherein R¹, R², R³, R⁶, R⁷, and R⁸ are defined as above for Structure (II); R²⁵, R²⁶, R²⁷, R²⁸, and R²⁹ are defined as for R⁶-R¹¹ above for Structure (II) and Structure (III):

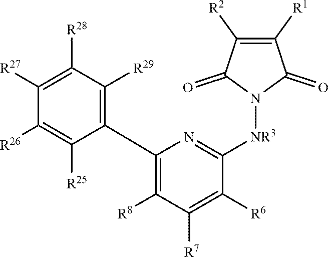

In certain non-limiting embodiments of compounds having Structure (III), X is NR³, and Y is S; R¹⁰ is a substituted benzene ring, providing compounds having Structure (X), wherein R¹, R², R³, and R¹¹ are as defined above for Structure (II) and Structure (III); R³⁰, R³¹, R³², R³³, and R³⁴ are defined as for R⁶-R¹¹ above for Structure (II) and Structure (III):

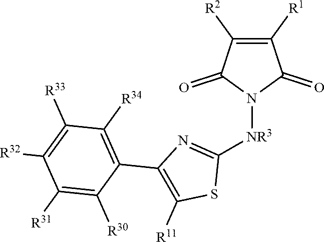

In certain non-limiting embodiments of compounds having Structure (II), X is NR³, A is N or CH, and B is N, providing compounds having Structure (XI), wherein R¹, R², R³, R⁶, R⁷, and R⁹ are as defined above for Structure (II):

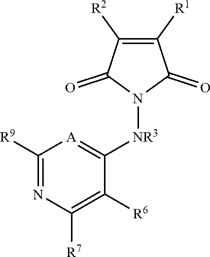

In any of the embodiments of Structures (I)-(XI), one of R¹ and R² is alkyl and the other of R¹ and R² is alkyl or substituted alkyl. In some variations, R¹ and R²are independently selected from alkyl, substituted alkyl, and optionally substituted alkoxy, wherein at least one of R¹ and R² is methyl. In some embodiments, one of R¹ and R² is methyl, and the other of R¹ and R² is alkyl or alkyl substituted with alkoxy, hydroxy, carboxy, or alkoxycarbonyl. In certain embodiments of Structures (I)-(XI), where applicable, at least one of R⁶, R⁷, R⁸, and R⁹ is halogen selected from Br and Cl, or alkyl substituted with one or more halogen groups selected from Br, Cl, and F. In some variations, R⁸ is halogen or alkyl substituted by one or more halogen groups.

Representative compounds are shown in Tables 1, 2, and 3 herein. It is generally understood that the compounds disclosed in Tables 1, 2, and 3 are for the purpose of illustration only, and by no means restrict the scope.

### D. Representative Synthesis Schemes

Representative schemes for synthesis of compounds having any of Structures (I) to (XI) are presented below. The representative schemes presented here do not restrict the scope in any way. It is understood that one of skill in the art can adapt methods presented herein, and/or different methods known in the art, to synthesize additional compounds within the scope, including analogs having different substitutions or substitution patterns. It is further understood that, although certain substitutions have been observed to produce structures with higher activity than other structures, provided are compounds with all substitutions having all levels of activity.

In Method 1 (Scheme 1), an anhydride (1) is reacted with a substituted hydrazine (2) or a benzylamine (3) to form the compounds having the general structure shown as (4) below,

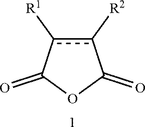

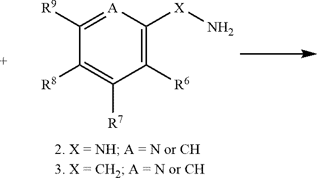

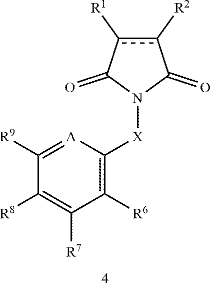

The reaction can be carried out in common organic solvents such as THF, chloroform, DMF, acetic acid, etc., at temperatures ranging from ambient to elevated, for times ranging from several hours to a few days. Usually, no other additives are needed. The required anhydrides and hydrazines/benzylamines are either purchased from commercial sources or synthesized according to procedures known in the literature. In cases where the starting materials are unknown in the literature, synthetic methods are developed, as illustrated by certain syntheses described in the Examples.

In Method 2 (Scheme 2), an imide (5) is reacted with a benzylalcohol (6) under typical Mitsunobu conditions to form compounds having the general structure shown as (7) below:

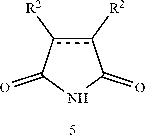

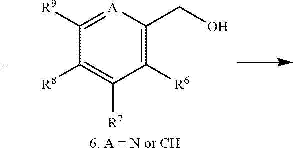

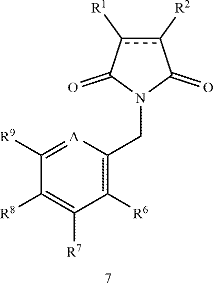

Typical Mitsunobu conditions include the use of a phosphine (triphenylphosphine, tributylphosphine etc.) and an azo compound (diethyl azo-dicarboxalate, diisopropyl azo-dicarboxylate, etc.). The reaction can be carried out with an added base, usually triethylamine, or without an added base, in solvents such as THF, at ambient or elevated temperature for several hours. The required imides and benzylalcohols are either purchased from commercial sources or synthesized according to procedures known in the literature. In cases where the starting materials are unknown in the literature, synthetic methods are developed, as illustrated by certain syntheses described in the Examples.

In Method 3 (Scheme 3), an imide (5) is reacted with a benzylbromide (8) under typical nucleophilic replacement reaction condition to form the compounds having the general structure shown as (9) below:

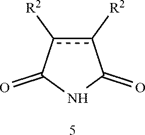

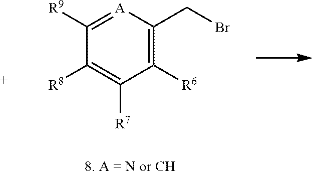

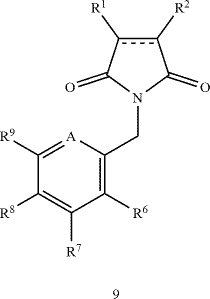

The typical reaction condition is: refluxing in a suitable solvent (acetone, DMF, etc.) in the presence of an added base (potassium carbonate, cesium carbonate, etc.) for several hours to a few days. The required imides and benzylbromides are either purchased from commercial sources or synthesized according to procedures known in the literature. In cases where the starting materials are unknown in the literature, synthetic methods are developed, as illustrated by certain syntheses described in the Examples.

In Method 4 (Scheme 4), an aryl boronic acid (10) is reacted with an N-hydroxyimide (11) under Cu(I) mediated coupling condition to form compounds having the general structure shown as (12) below:

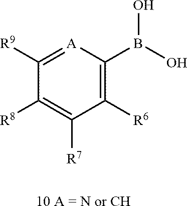

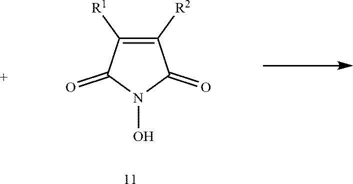

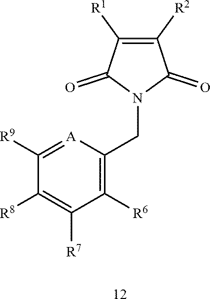

The typical reaction condition is: stirring at room temperature in suitable solvents (DCE, THF, DMF, etc.) in the presence of an added base (pyridine, triethylamine, etc.), with added Cu(I) species, such as CuCl, for several hours to overnight. The required aryl boronic acids and N-hydroxyimides are either purchased from commercial sources or synthesized according to procedures known in the literature. In cases where the starting materials are unknown in the literature, synthetic methods are developed, as illustrated by certain syntheses described in the Examples.

Syntheses of particular embodiments are disclosed in the Examples. Representative synthesized compounds presented as embodiments in Tables 1, 2, and 3 are presented solely for illustration and by no means restricts the scope disclosed herein.

### III. Biological Activities of the Substituted Azole Dione Compounds

### A. Biological Activity

The compounds used in the methods described herein, i.e., the compounds identified in Section II above, can be used to achieve one, two, or more of the following results: (i) inhibition of viral assembly; (ii) inhibition of viral replication; (iii) inhibition of viral growth; (iv) killing of host cells comprising a virus targeted by the methods; (v) treating viral infection; (vi) preventing viral infection; (vii) treating disease caused by or associated with a virus; (viii) preventing disease caused by or associated with a virus. In a specific embodiment, the compounds used in the methods described herein are effective against one, two, or more of the virus families or specific viruses described herein.

While not being bound by any mechanisms, the compounds herein are believed to inhibit viral replication and bind to XPO1, specifically at Cys528 of XPO1. This binding is reversible for felezonexor and is believed to be reversible for other compounds herein. In some embodiments, contacting a cell with a compound herein blocks nuclear export of a viral ribonucleoprotein (vRNP) or viral protein. Thus, contacting a cell with the compound can increase nuclear retention of a viral protein or vRNP.

In some embodiments, a compound described herein inhibits replication. In some embodiments, the compound decreases the infectivity of the virus. In some embodiments, the compound decreases the viral titer of the virus. In some embodiments, the compound reduces the duration of infection, symptoms of infection, severity of infection, viral infectivity, viral replication, and/or viral shedding (including from one cell to another cell, from one part of the body to another part of the body, or from one subject to another subject).

In certain embodiments, a compound described herein is effective as an antiviral agent against the Orthomyxoviridae family, i.e., can be used in a method of treating or preventing a viral infection or disease caused by a member of the Orthomyxoviridae family. In a specific embodiment, a compound described herein is effective as an antiviral agent against influenza A virus infection, e.g., an H3N2 subtype influenza A infection or an H1N1 subtype influenza A infection.

In certain embodiments, a compound described herein is effective as an antiviral agent against the Togaviridae family, i.e., can be used in a method of treating or preventing a viral infection or disease caused by a member of the Togaviridae family.

In certain embodiments, a compound described herein is effective as an antiviral agent against Venezuelan equine encephalitis virus (VEEV), i.e., can be used in a method of treating or preventing a viral infection or disease caused by Venezuelan equine encephalitis virus (VEEV).

In certain embodiments, a compound described herein is effective as an antiviral agent against the Pneumoviridae family, i.e., can be used in a method of treating or preventing a viral infection or disease caused by a member of the Pneumoviridae family.

In certain embodiments, a compound described herein is effective as an antiviral agent against respiratory syncytial virus (RSV), i.e., can be used in a method of treating or preventing a viral infection or disease caused by RSV. In a specific embodiment, a compound described herein is effective as an antiviral agent against an A2 strain of RSV.

In certain embodiments, a compound described herein is effective as an antiviral agent against the Flaviviridae family, i.e., can be used in a method of treating or preventing a viral infection or disease caused by a member of the Flaviviridae family.

In certain embodiments, a compound described herein is effective as an antiviral agent against dengue virus (DENV) infection, i.e., can be used in a method of treating or preventing a viral infection or disease caused by DENV. In a specific embodiment, a compound described herein is effective as an antiviral agent against a DV2 Thai strain DENV infection.

In certain embodiments, a compound described herein is effective as an antiviral agent against the Paramyxoviridae family, i.e., can be used in a method of treating or preventing a viral infection or disease caused by a member of the Paramyxoviridae family. In a specific embodiment, a compound described herein is effective as an antiviral agent against Nipah virus.

In certain embodiments, the compounds used in the methods described herein act via inhibition of viral component export from the nucleus, wherein viral component assembly and replication requires export to the cytoplasm. Examples of such viral components include viral nucleic acids, viral proteins, and viral ribonucleoprotein (vRNPs). In a specific embodiment, the methods described herein result in inhibition of the export of vRNP(s). In another specific embodiment, inhibition of vRNP nuclear export causes nuclear retention of vRNP(s), which prevents viral assembly and replication from occurring in the cytoplasm. In specific embodiments, prevention of viral assembly and replication inhibits viral release from the host cell.

In a specific embodiment, a composition described herein comprises the compound (such as a compound of Structures (I)-(XI)) in an amount effective to increase nuclear retention of vRNPs. In a specific embodiment, a composition described herein comprises the compound in an amount effective to increase nuclear retention of vRNPs and a carrier. In a specific embodiment, a composition described herein comprises the compound in an amount effective to increase nuclear retention of vRNPs by at least 2-fold, 3-fold, 4-fold, 5-fold, 6-fold, 7-fold, 8-fold, 9-fold, 10-fold, 20-fold, 50-fold, 75-fold, 100-fold, or 1,000-fold as compared to nuclear retention in the absence of the composition. In a specific embodiment, a composition described herein comprises the compound in an amount effective to increase nuclear retention of vRNPs by between 2-fold and 5-fold, between 2-fold and 10-fold, between 2-fold and 20-fold, between 5-fold and 100-fold, between 10-fold and 50-fold, between 10-fold and 100-fold, or between 100-fold and 1,000-fold as compared to nuclear retention in the absence of the composition. In a specific embodiment, a composition described herein comprises a compound in an amount effective to increase nuclear retention of vRNPs by at least 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs as compared to nuclear retention in the absence of the composition. In a specific embodiment, a composition described herein comprises the compound in an amount effective to increase nuclear retention of vRNPs by between 0.5 log and 2.5 logs, 1 log and 3 logs, 2 logs and 4 logs,3 logs and 5 logs, 4 logs and 6 logs, 5 logs and 7 logs, 6 logs and 8 logs, 7 logs and 9 logs, or 8 logs and 10 logs as compared to viral replication in the absence of the composition.

In one aspect, provided are methods for treating a viral infection in a subject or treating or preventing a disease associated with or caused by a virus in a subject, comprising administering to the subject a compound that targets Exportin 1 (XPO1; Genbank Gene ID: 7514; also known as CRM1) or a pharmaceutical composition comprising such a compound, wherein said compound is one identified in Section II. In another specific embodiment, said compound binds XPO1so as to prevent binding of XPO1 to viral proteins or vRNPs. In a specific embodiment, said viral protein is nuclear export protein of influenza (NEP or NS2). In another specific embodiment, said compound binds XPO1 reversibly.

In one aspect, provided are methods for treating a viral infection in a subject or treating or preventing a disease associated with or caused by a virus in a subject, comprising administering to the subject a compound that targets Exportin 1 (XPO1; Genbank Gene ID: 7514; also known as CRM1) or a pharmaceutical composition comprising such a compound, wherein said compound is one identified in Section II. In another specific embodiment, said compound binds XPO1 so as to prevent binding of XPO1 to viral proteins and vRNPs. In a specific embodiment, said viral protein is NEP (NS2). In another specific embodiment, said compound binds XPO1 reversibly.

In a specific embodiment, provided herein is a method for treating a viral infection in a subject, comprising administering to the subject a compound that targets Exportin 1 (XPO1; Genbank Gene ID: 7514; also known as CRM1) or a pharmaceutical composition comprising such a compound, wherein said compound is one identified in Section II. In another specific embodiment, said compound binds XPO1 so as to prevent binding of XPO1 to viral proteins or vRNPs. In a specific embodiment, said viral protein is NEP (NS2). In another specific embodiment, said compound binds XPO1 reversibly.

In another embodiment, provided herein is a method for preventing a disease in a subject caused by or associated with a viral infection, comprising administering to the subject a compound that targets Exportin 1 (XPO1; Genbank Gene ID: 7514; also known as CRM1) or a pharmaceutical composition comprising such a compound, wherein said compound is one identified in Section II. In another specific embodiment, said compound binds XPO1 so as to prevent binding of XPO1 to viral proteins or vRNPs. In a specific embodiment, said viral protein is NEP (NS2). In another specific embodiment, said compound binds XPO1 reversibly.

Viral infections and/or diseases that can be treated using the methods described herein include, without limitation, those caused by viruses belonging to the Togaviridae, Arenaviridae, Poxviridae, Toroviridae, Paramyxoviridae, Herpesviridae, Retroviridae, Coronaviridae, Flaviviridae, Bunyaviridae, Pneumoviridae, Filoviridae, Adenoviridae, or Orthomyxoviridae families. In a specific embodiment, the methods provided herein are used to treat a viral infection and/or disease caused by dengue virus (DENV), respiratory syncytial virus (RSV), Venezuelan equine encephalitis virus (VEEV), influenza virus, human immunodeficiency virus (HIV), herpes simplex virus (HSV), cytomegalovirus (CMV), Ebola virus, rubulavirus, Nipah virus, or coronavirus. In a specific embodiment, the virus is a Venezuelan equine encephalitis virus (VEEV). In another specific embodiment, the virus is Nipah virus. In another specific embodiment, the virus is an influenza virus, e.g., influenza A, B, or C. In another embodiment, the virus is H1N1, H1N2, H3N2, H5N1, or H7N9 subtype of influenza A. In some embodiments, the virus is Coronavirus. In some embodiments, the virus is SARS-CoV2. In some embodiments, the disease caused by the viral infection is COVID-19. In a further specific embodiment, the virus is any virus, strain, or subtype of the foregoing viruses.

In certain embodiments, treatment of a viral infection or disease caused by or associated therewith with a compound described herein or composition thereof, or a combination of a compound described herein and another therapy, results in a beneficial or therapeutic effect. In specific embodiments, the treatment of a viral infection or disease caused by or associated therewith with a compound described herein or composition thereof, or a combination of a compound described herein and another therapy results in one, two, three, four, five, or more, or all of the following effects: (i) the reduction or amelioration of the severity of a viral infection or a disease or a symptom caused by or associated therewith; (ii) the reduction in the duration of a viral infection or a disease or a symptom caused by or associated therewith; (iii) the regression of a viral infection or a disease or a symptom caused by or associated therewith; (iv) the reduction of the particles/titer of a virus; (v) the reduction in organ failure associated with a viral infection or a disease caused by or associated therewith; (vi) the reduction in hospitalization of a subject; (vii) the reduction in hospitalization length; (viii) the increase in the survival of a subject; (ix) the elimination of a viral infection or a disease or a symptom caused by or associated therewith; (x) the inhibition of the progression of a viral infection or a disease or a symptom caused by or associated therewith; (xi) the prevention of the spread of a virus from a cell, tissue, organ or subject to another cell, tissue, organ or subject; and/or (xii) the enhancement or improvement the therapeutic effect of another therapy.

In certain embodiments, the administration of a compound described herein or composition thereof, or a combination of a compound described herein and another therapy to a subject to prevent a disease caused by or associated with a viral infection results in one or more of the prophylactic/beneficial effects. In a specific embodiment, the administration of a compound described herein or composition thereof, or a combination of compound described herein and another therapy to a subject to prevent a disease caused by or associated with a viral infection results in one, two or more, or all of the following effects: (i) the inhibition of the development or onset of a disease caused by or associated with a viral infection or a symptom thereof; (ii) the inhibition of the recurrence of a disease caused by or associated with a viral infection or a symptom associated therewith; and (iii) the reduction or inhibition in viral infection and/or replication.

The amount of a compound described herein or composition which will be effective in the treatment and/or prevention of an infection or disease described herein will depend on a number of factors, including, e.g., the nature of the disease. The precise dose to be employed will also depend on the route of administration, and the seriousness of the infection or disease caused by or associated therewith. For example, effective doses may also vary depending upon means of administration, target site, physiological state of the patient (including age, body weight, and health), whether the patient is human or an animal, other medications administered, and whether treatment is prophylactic or therapeutic.

In specific embodiments, “effective amount” of a compound or composition thereof administered to a subject refers to the amount of the compound or composition which is sufficient to achieve one, two, three, four, or more of the following effects: (i) reduce or ameliorate the severity of a viral infection, disease or symptom caused by or associated therewith; (ii) reduce the duration of a viral infection, disease or symptom caused by or associated therewith; (iii) prevent the progression of a viral infection, disease or symptom caused by or associated therewith; (iv) cause regression of a viral infection, disease or symptom caused by or associated therewith; (v) prevent the development or onset of a viral infection, disease or symptom caused by or associated therewith; (vi) prevent the recurrence of a viral infection, disease or symptom caused by or associated therewith; (vii) reduce or prevent the spread of a virus from one cell to another cell, one tissue to another tissue, or one organ to another organ; (viii) prevent or reduce the spread of a virus from one subject to another subject; (ix) reduce organ failure associated with a viral infection; (x) reduce hospitalization of a subject; (xi) reduce hospitalization length; (xii) increase the survival of a subject with a viral infection or disease caused by or associated therewith; (xiii) eliminate a viral infection or disease caused by or associated therewith; (xiv) inhibit or reduce viral replication; (xv) reduce viral numbers/titer; and/or (xvi) enhance or improve the prophylactic or therapeutic effect(s) of another therapy.

In certain embodiments, the effective amount does not result in complete protection from a disease caused by or associated with a viral infection, but results in a lower titer or reduced number of viruses compared to an untreated subject with a viral infection. In certain embodiments, the effective amount results in a 0.5 fold, 1 fold, 1.5 fold, 2 fold, 3 fold, 4 fold, 5 fold, 6 fold, 7 fold, 8 fold, 9 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titer/number of viruses relative to an untreated subject with a viral infection. In some embodiments, the effective amount results in a reduction in titer/number of viruses relative to an untreated subject with a viral infection of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. Benefits of a reduction in the titer, number or total burden of viruses include, but are not limited to, less severe symptoms of the infection, fewer symptoms of the infection and a reduction in the length of the disease caused by or associated with the infection.

In another aspect, provided herein is a method for inhibiting assembly, replication, release, growth, or infectivity of a virus, comprising administering to the subject a compound that targets XPO1 or a pharmaceutical composition comprising such a compound, wherein said compound is one identified in Section II. In another specific embodiment, said compound binds XPO1 so as to prevent binding of XPO1 to viral proteins or vRNPs. In a specific embodiment, said viral protein is NEP (NS2). In another specific embodiment, said compound binds XPO1 reversibly.

In a specific embodiment, provided herein is a method for inhibiting viral assembly in a cell, comprising contacting a cell infected with a virus with an effective amount of a compound that targets Exportin 1 (XPO1; Genbank Gene ID: 7514; also known as CRM1) or a pharmaceutical composition comprising such a compound, wherein said compound is one identified in Section II. In another specific embodiment, said compound binds XPO1 so as to prevent binding of XPO1 to viral proteins or vRNPs. In a specific embodiment, said viral protein is NEP (NS2). In another specific embodiment, said compound binds XPO1 reversibly.

In another specific embodiment, provided herein is a method for inhibiting viral replication in a cell, comprising contacting a cell infected with a virus with an effective amount of a compound that targets Exportin 1 (XPO1; Genbank Gene ID: 7514; also known as CRM1) or a pharmaceutical composition comprising such a compound, wherein said compound is one identified in Section II. In another specific embodiment, said compound binds XPO1 so as to prevent binding of XPO1 to viral proteins or vRNPs. In a specific embodiment, said viral protein is NEP (NS2). In another specific embodiment, said compound binds XPO1 reversibly.

In another specific embodiment, provided herein is a method for inhibiting viral assembly and replication in a cell, comprising contacting a cell infected with a virus with an effective amount of a compound that targets Exportin 1 (XPO1; Genbank Gene ID: 7514; also known as CRM1) or a pharmaceutical composition comprising such a compound, wherein said compound is one identified in Section II. In another specific embodiment, said compound binds XPO1so as to prevent binding of XPO1 to viral proteins or vRNPs. In a specific embodiment, said viral protein is NEP (NS2). In another specific embodiment, said compound binds XPO1 reversibly.

“EC50” refers to the concentration of an active compound required to yield a half-maximal response. In a specific embodiment, a compound described herein has an EC50 of between 0.1 nM and 1000 nM. In another specific embodiment, the EC50 is between 0.1 nM and 2.5 nM, 0.1 nM and5 nM, 0.1 nM and 10 nM, 10 nM and 20 nM, 10 nM and 30 nM, 10 nM and 40 nM, 10 nM and 50 nM, 50 nM and 75 nM, 50 nM and 100 nM, 100 nM and 250 nM, 100 nM and 500 nM, 100 nM and 750 nM, and 100 nM and 1000 nM. In another specific embodiment, the EC50 is less than 100 nM, less than 50 nM, less than 25 nM, less than 10 nM, less than 5 nM, less than 1 nM, or less than 0.5 nM.

“CC50” refers to the concentration of an active compound required to reduce cell viability by 50% when compared to untreated controls. In a specific embodiment, a compound described herein has a CC50 of between 0.5 µM and 10 µM in animal cells (e.g., human cells, such as human cells from a cell line). In another specific embodiment, the CC50 is between 0.5 µM and 0.75 µM, 0.5 µM and 1.0 µM, 1 µM and 2.5 µM, 1 µM and 5 µM, 1 µM and 7.5 µM, 1 µM and 10 µM in animal cells (e.g., human cells, such as human cells from a cell line). In another specific embodiment, a compound described herein has a CC50 greater than 0.5 µM, greater than 0.75 µM, greater than 1.0 µM, greater than 2.5 µM, greater than 5 µM, greater than 7.5 µM, or greater than 10 µM in animal cells (e.g., human cells, such as human cells from a cell line).

“SI” refers to the selectivity index and is the ratio of CC50 to EC50 (i.e., CC50/EC50). It is understood that the higher the SI value, the more effective and safe the treatment is likely to be (i.e., administration of a compound of Structure (I) or any variation thereof). In a specific embodiment, a compound described herein has an SI of greater than 1 in animal cells (e.g., human cells, such as human cells from a cell line). In another specific embodiment, a compound described herein has an SI greater than 1, greater than 5, greater than 10, greater than 25, greater than 50, greater than 75, greater than 100, or greater than 1000 in animal cells (e.g., human cells, such as human cells from a cell line). In another specific embodiment, the SI is between 1 and 100, 1 and 75, 1 and 50, 1 and 25, 1 and 10, 1 and 5, 5 and 100, 10 and 100, 25 and 100, 50 and 100, or 75 and 100 in animal cells (e.g., human cells, such as human cells from a cell line). In further embodiments, the SI is between 1 and 1000, 1 and 750, 1 and 500, 1 and 250, 10 and 1000, 25 and 1000, 50 and 1000, 75 and 1000, 100 and 1000, 250 and 1000, 500 and 1000, or 750 and 1000 in animal cells (e.g., human cells, such as human cells from a cell line).

### B. Patient Population

As used herein, the terms “subject” and “patient” when used in the context of administration of a compound described herein or a composition thereof are used interchangeably to refer to an animal. In a specific embodiment, a subject is a human.

In certain embodiments, a compound described or composition described herein may be administered to a naïve subject, i.e., a subject that does not have a disease caused by a virus described herein, or has not been and is not currently infected with a virus described herein. In one embodiment, a compound or composition described herein is administered to a naïve subject that is at risk of acquiring an infection with a virus described herein. A compound or composition described herein may also be administered to a subject that is and/or has been infected with a virus described in herein. Thus, the compound or composition may be used to treat a patient prophylactically and that concept is included in all embodiments of treatments and uses described herein.

In certain embodiments, a compound or composition described herein is administered to a patient who has been diagnosed with an infection with a virus. In some embodiments, a compound or composition described herein is administered to a patient who has become infected with a virus but has not been positively diagnosed with the virus infection. In some embodiments, a compound or composition described herein is administered to a patient infected with a virus before symptoms manifest or symptoms become severe (e.g., before the patient requires hospitalization). In some embodiments, a compound or composition described herein is administered to a patient infected with a virus after symptoms become severe (e.g., after the patient requires hospitalization).

### C. Viruses

Viral infections and/or diseases that can be treated using the methods described herein include, without limitation, those caused by viruses belonging to the Togaviridae, Arenaviridae, Poxviridae, Poroviridae, Paramyxoviridae, Herpesviridae, Retroviridae, Coronaviridae, Flaviviridae, Bunyaviridae, Pneumoviridae, Filoviridae, Adenoviridae, Papovaviridae, Hepadnaviridae, or Orthomyxoviridae families.

In a specific embodiment, the methods provided herein are used to treat a viral infection and/or disease caused by dengue virus (DENV), respiratory syncytial virus (RSV), Venezuelan equine encephalitis virus (VEEV), influenza virus, human immunodeficiency virus (HIV), herpes simplex virus (HSV), cytomegalovirus (CMV), Ebola virus, Nipah virus, or rubulavirus. In a specific embodiment, the virus is a Venezuelan equine encephalitis virus (VEEV). In a specific embodiment, the virus is Nipah virus. In another specific embodiment, the virus is an influenza virus, e.g., influenza A, B, or C.

In a specific embodiment, the virus is a dengue virus (DENV), respiratory syncytial virus (RSV), Venezuelan equine encephalitis virus (VEEV), influenza virus, human immunodeficiency virus (HIV), herpes simplex virus (HSV), cytomegalovirus (CMV), Ebola virus, rubulavirus, Nipah virus, Hepatitis B virus, BK virus, JC virus, papillomavirus, adenovirus-5, cowpox virus, measles virus, varicella-zoster virus, Epstein-Barr virus, Kaposi’s sarcoma associated herpesvirus, West Nile virus, Chikungunya virus (CHIKV), or coronavirus.

In a specific embodiment, the virus belonging to the Togaviridae family is Venezuelan equine encephalitis virus (VEEV). In a specific embodiment, the VEEV is VEEV TC-83 (see, e.g., GenBank Accession No. L01443).

In a specific embodiment, the virus belonging to the Paramyxoviridae family is Nipah virus. In a specific embodiment, the Nipah virus is the Malaysian strain.

In a specific embodiment, the virus belonging to the Arenaviridae family is Junin virus. In a specific embodiment, the JUNV is JUNV Candid #1. See, e.g., Emonet, Sebastien F., et al. “Rescue from cloned cDNAs and in vivo characterization of recombinant pathogenic Romero and live-attenuated Candid# 1 strains of Junin virus, the causative agent of Argentine hemorrhagic fever disease.” Journal of virology 85.4 (2011): 1473-1483, for a description of Junin virus.

In a specific embodiment, the virus belonging to the Herpesviridae family is herpes simplex virus-1 (HSV-1). In a specific embodiment, the virus belonging to the Herpesviridae family is herpes simplex virus-2 (HSV-2). In a specific embodiment, the virus belonging to the Herpesviridae family is varicella zoster virus (VZV). In a specific embodiment, the virus belonging to the Herpesviridae family is Epstein-Barr virus (EBV), lymphocryptovirus. In a specific embodiment, the virus belonging to the Herpesviridae family is cytomegalovirus (CMV). In a specific embodiment, the virus belonging to the Herpesviridae family is herpes lymphotropic virus (roseolovirus). In a specific embodiment, the virus belonging to the Herpesviridae is Kaposi’s sarcoma-associated herpesvirus.

In a specific embodiment, the virus belonging to the Retroviridae family is HIV-1. In a specific embodiment, the HIV-1 is HIV-1BaL (see, e.g., GenBank Accession Number: DQ318211). In a specific embodiment, the HIV-1 is HIV-1 RF (see, e.g., Otto MJ, Garber S, Winslow D, Reid CD, Aldrich P, Jadhav PK, Patterson CE, Hoge CN, Cheng YS. In vitro isolation and identification of HIV variants with reduced sensitivity to C-2 symmetrical inhibitors of HIV-1 protease. Proc Natl Acad Sci USA 90:7543-7547, 1993).

In a specific embodiment, the virus belonging to the Flaviviridae family is DENV. In a specific embodiment, the DENV is serotype 1 (DENV-1). In a specific embodiment, the DENV-1 is DENV-1 PRS41393 (see, e.g., Warfield, Kelly L., et al. “Inhibition of endoplasmic reticulum glucosidases is required for in vitro and in vivo dengue antiviral activity by the iminosugar uv-4.” Antiviral research 129 (2016): 93-98, for a description of the virus.) In a specific embodiment, the DENV is serotype 2 (DENV-2). In a specific embodiment, the DENV-2 is DENV-2 New Guinea C (see, e.g., ATCC No. VR-1584). In a specific embodiment, the DENV is serotype 3 (DENV-3). In a specific embodiment, the DENV-3 is DENV-3 H37 (see, e.g., GenBank Accession No. M93130). In a specific embodiment, the DENV is serotype 4 (DENV-4). In a specific embodiment, the DENV-4 is DENV-4 H24 (see, e.g., GenBank Accession No. AY947539).

In a specific embodiment, the virus belonging to the Flaviviridae family is yellow fever virus (YFV). In a specific embodiment, the YFV is YFV 17D (see, e.g., GenBank Accession No. X03700 and Rice, Charles M., et al. “Nucleotide sequence of yellow fever virus: implications for flavivirus gene expression and evolution.” Science 229.4715 (1985): 726-733). In a specific embodiment, the virus belonging to the Flaviviridae family is Japanese encephalitis virus (JEV). In a specific embodiment, the JEV is JEV 14-14-2 (see, e.g., GenBank Accession No. JN604986).

In a specific embodiment, the virus belonging to the Flaviviridae family is zika virus (ZIKV). In a specific embodiment, the ZIKV is ZIKV FSS 13025 (see, e.g., GenBank accession No. JN860885 and Haddow, Andrew D., et al. “Genetic characterization of Zika virus strains: geographic expansion of the Asian lineage.” PLoS Negl Trop Dis 6.2 (2012): e1477).

In a specific embodiment, the virus belonging to the Flaviviridae family is West Nile virus. In a specific embodiment, the West Nile virus is WNV Cy2016 (see, e.g., GenBank Accession No. MF797870).

In a specific embodiment, the virus belonging to the Togaviridae family is Chikungunya virus (CHIKV). In a specific embodiment, CHIKV is CHIKV 181/25 (see, e.g., GenBank Accession No. L37661).

In a specific embodiment, the virus belonging to the Bunyaviridae family is Rift Valley fever virus (RVFV). In a specific embodiment, the RVFV is RVFV MP12 (see, e.g., GenBank Accession No. DQ380208.1). In a specific embodiment, the virus belonging to the Poxviridae family is vaccinia virus (VACV). In a specific embodiment, the VACV is VACV NYCBH (see, e.g., Monath, Thomas P., et al. “ACAM2000 clonal Vero cell culture vaccinia virus (New York City Board of Health strain)-a second-generation smallpox vaccine for biological defense.” International journal of infectious diseases 8 (2004): 31-44).

In a specific embodiment, the virus belonging to the Pneumoviridae family is respiratory syncytial virus (RSV).

In a specific embodiment, the virus belonging to the Filoviridae family is EBOV. In a specific embodiment, the virus belonging to the Filoviridae family is Marburg virus (MARV).

In a specific embodiment, the virus belonging to the Orthomyxoviridae family is an influenza virus. In a specific embodiment, the virus belonging to the Orthomyxoviridae family is an influenza B virus. In a specific embodiment, the virus belonging to the Orthomyxoviridae family is an influenza C virus. In a specific embodiment, the virus belonging to the Orthomyxoviridae family is an influenza A virus.

In a specific embodiment, the virus belonging to the Orthomyxoviridae family is an H1N2 subtype influenza A virus. In a specific embodiment, the virus belonging to the Orthomyxoviridae family is an H2N2 subtype influenza A virus. In a specific embodiment, the virus belonging to the Orthomyxoviridae family is an H3N1 subtype influenza A virus. In a specific embodiment, the virus belonging to the Orthomyxoviridae family is an H3N8 subtype influenza A virus. In a specific embodiment, the virus belonging to the Orthomyxoviridae family is an H5N1 subtype influenza A virus. In a specific embodiment, the virus belonging to the Orthomyxoviridae family is an H5N2 subtype influenza A virus. In a specific embodiment, the virus belonging to the Orthomyxoviridae family is an H5N3 subtype influenza A virus. In a specific embodiment, the virus belonging to the Orthomyxoviridae family is an H5N8 subtype influenza A virus. In a specific embodiment, the virus belonging to the Orthomyxoviridae family is an H5N9 subtype influenza A virus. In a specific embodiment, the virus belonging to the Orthomyxoviridae family is an H7N1 subtype influenza A virus. In a specific embodiment, the virus belonging to the Orthomyxoviridae family is an H7N2 subtype influenza A virus. In a specific embodiment, the virus belonging to the Orthomyxoviridae family is an H7N3 subtype influenza A virus. In a specific embodiment, the virus belonging to the Orthomyxoviridae family is an H7N4 subtype influenza A virus. In a specific embodiment, the virus belonging to the Orthomyxoviridae family is an H7N7 subtype influenza A virus. In a specific embodiment, the virus belonging to the Orthomyxoviridae family is an H7N9 subtype influenza A virus. In a specific embodiment, the virus belonging to the Orthomyxoviridae family is an H9N2 subtype influenza A virus. In a specific embodiment, the virus belonging to the Orthomyxoviridae family is an H10N7 subtype influenza A virus. In a specific embodiment, the virus belonging to the Orthomyxoviridae family is an H3N2 subtype influenza A virus. In a specific embodiment, the virus belonging to the Orthomyxoviridae family is an H1N1subtype influenza A virus.

In a specific embodiment, the virus belonging to the Orthomyxoviridae family is an H1N1 subtype, A/Puerto Rico/8/1934 strain, influenza A virus. In a specific embodiment, the virus belonging to the Orthomyxoviridae family is an H1N1 subtype, A/USSR/90/1977 strain, influenza A virus. In a specific embodiment, the virus belonging to the Orthomyxoviridae family is an H1N1 subtype, A/New Jersey/8/1976 strain, influenza A virus. In a specific embodiment, the virus belonging to the Orthomyxoviridae family is an A/Solomon Islands/03/2006 strain, influenza A virus. In a specific embodiment, the virus belonging to the Orthomyxoviridae family is an H1N1 subtype, A/New Caledonia/20/1999 strain, influenza A virus. In a specific embodiment, the virus belonging to theOrthomyxoviridae family is an H1N1 subtype, A/Hawaii/15/2001 strain, influenza A virus. In a specific embodiment, the virus belonging to the Orthomyxoviridae family is an H1N1 subtype, A/California/07/09 strain, influenza A virus. In a specific embodiment, the virus belonging to the Orthomyxoviridae family is an H1N1 subtype, A/Washington/29/2009 strain, influenza A virus.

In a specific embodiment, the virus belonging to the Orthomyxoviridae family is an H1N2 subtype, A/Minnesota/19/2011 strain, influenza A virus.

In a specific embodiment, the virus belonging to the Orthomyxoviridae family is an H3N2 subtype, A/Texas/71/2007 strain, influenza A virus. In a specific embodiment, the virus belonging to the Orthomyxoviridae family is an H3N2 subtype, A/Fujian/413/2002 strain, influenza A virus. In a specific embodiment, the virus belonging to the Orthomyxoviridae family is an H3N2 subtype, A/Perth/16/2009 strain, influenza A virus. In a specific embodiment, the virus belonging to the Orthomyxoviridae family is an H3N2 subtype, A/Indiana/08/2011 strain, influenza A virus. In a specific embodiment, the virus belonging to the Orthomyxoviridae family is an H3N2 subtype, A/California/02/2014 strain, influenza A virus. In a specific embodiment, the virus belonging to the Orthomyxoviridae family is an H3N2 subtype, A/Alaska/140/2015 strain, influenza A virus.

In a specific embodiment, the virus belonging to the Orthomyxoviridae family is an H5N1 subtype, A/Hong Kong/156/1997 strain, influenza A virus. In a specific embodiment, the virus belonging to the Orthomyxoviridae family is an H5N1 subtype, A/Vietnam/1203/2004 strain, influenza A virus. In a specific embodiment, the virus belonging to the Orthomyxoviridae family is an H5N1 subtype, A/Indonesia/05/2005 strain, influenza A virus. In a specific embodiment, the virus belonging to the Orthomyxoviridae family is an H5N1 subtype, A/Egypt/N6658/2010 strain, influenza A virus.

In a specific embodiment, the virus belonging to the Orthomyxoviridae family is an H7N9 subtype, A/Anhui/1/2013 strain, influenza A virus.

In a specific embodiment, the virus is a Venezuelan equine encephalitis virus (VEEV). In another specific embodiment, the virus is an influenza virus, e.g., influenza A, B, or C. In another embodiment, the virus is H1N1, H1N2, H3N2, H5N1, or H7N9 subtype of influenza. In a further specific embodiment, the virus is any virus, strain, or subtype of the foregoing viruses.

In some embodiments, the virus belongs to theCoronaviridae family. In some embodiments, the virus is a Coronavirus. In some embodiments, the virus is SARS-CoV2. In some embodiments, the disease caused by the viral infection is COVID-19. In a further specific embodiment, the virus is any virus, strain, or subtype of the foregoing viruses.

In some embodiments, the virus belongs to the Hepadnaviridae family. In a specific embodiment, the virus belonging to the Hepadnaviridae family is Hepatitis B virus.

In some embodiments, the virus belongs to the Papovaviridiae family. In a specific embodiment, the virus belonging to the Papovaviridiae family is BK virus, JC virus, or papillomavirus. In some embodiments, the virus is BK virus. In some embodiments, the virus is JC virus. In some embodiments, the virus is papillomavirus.

In some embodiments, the virus belongs to the Adenoviridae family. In a specific embodiment, the virus belonging to the Adenoviridae family is Human adenovirus A, Human adenovirus B, Human adenovirus C, Human adenovirus D, Human adenovirus E, Human adenovirus F, or Human adenovirus G. In some embodiments, the virus is a Human adenovirus C. In some embodiments, the virus causes respiratory disease. In some embodiments, the virus is adenovirus-5.

In some embodiments, the virus belongs to the Poxviridae family. In a specific embodiment, the virus belonging to the Poxviridae family is cowpox virus.

In some embodiments, the virus belongs to the Paramyxoviridae family. In a specific embodiment, the virus belonging to the Paramyxoviridae family is measles virus.

### Exemplary Embodiments

The present disclosure is further described by the following embodiments. The features of each of the embodiments are combinable with any of the other embodiments where appropriate and practical.

Embodiment P1. A method of treating a viral infection in a subject comprising administering to the subject a compound having the formula of Structure (II):

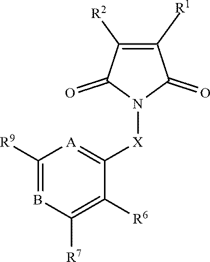

wherein
R¹ and R²are independently chosen from alkyl, substituted alkyl, and optionally substituted alkoxy, wherein at least one of R¹ and R² is methyl, and where R¹ and R² can be part of a cyclic alkylene chain that forms a fused ring structure;
X is NR³;
R³ is H, alkyl, or acyl;
A is N or CH;
B is CR⁸;
R⁶ is selected from H, alkyl, substituted alkyl, and halogen,
R⁷ is selected from H, alkyl, substituted alkyl, halogen, optionally substituted aryl, optionally substituted heteroaryl, optionally substituted alkoxy, optionally substituted aryloxy, cyano, optionally substituted alkylthio, optionally substituted alkylsufinyl, optionally substituted alkylsulfonyl, optionally substituted arylthio, optionally substituted acyl, optionally substituted amino, carboxyl, optionally substituted alkoxycarbonyl, optionally substituted carbamoyl, and wherein R⁶ and R⁷ can form an aryl group forming a fused ring structure when each of R⁶ and R⁷ is alkyl;
R⁸ is selected from H, alkyl, substituted alkyl, halogen, and
R⁹ is selected from H, alkyl, substituted alkyl, halogen, optionally substituted aryl, and cyano, wherein R⁸ and R⁹ can form one or more optionally substituted aryl groups forming a fused ring structure when each of R⁸ and R⁹ is alkyl or substituted alkyl and wherein at least one of R⁶, R⁷, R⁸, and R⁹ is halogen selected from Br or Cl, or substituted alkyl substituted with halogen selected from Br, Cl, or F;
or a salt thereof.

Embodiment P2. The method of embodiment P1, wherein one of R¹ and R² is methyl, and the other of R¹ and R² is alkyl or alkyl substituted with alkoxy, hydroxy, carboxy, or alkoxycarbonyl.

Embodiment P3. The method of embodiment P1, wherein R³ is H, methyl, or acyl.

Embodiment P4. The method of embodiment P2, wherein one of R¹ and R² is methyl, and the other of R¹ and R² is alkyl or alkyl substituted with alkoxy, hydroxy, carboxy, alkoxycarbonyl, wherein alkoxy includes cyclic alkoxy, and R⁶ is H, R⁷ is H, R⁸ is CF₃, and R⁹ is Cl.

Embodiment P5. A method of treating a viral infection in a subject comprising administering to the subject a compound having the formula of Structure (II):

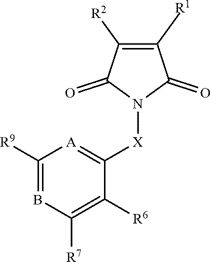

wherein
R¹ and R² are independently chosen from alkyl, substituted alkyl, and optionally substituted alkoxy, optionally substituted alkylthio, halogen, optionally substituted aryl, optionally substituted aryloxy, optionally substituted arylthio, or H, where R¹ and R² can be part of a cyclic alkylene chain that forms a fused ring structure;
X is NR³;
R³ is H, alkyl, or acyl;
A is N or CH;
B is CR⁸ or N;
R⁶, R⁷, R⁸, and R⁹ are independently chosen from H, alkyl, substituted alkyl, halogen, optionally substituted aryl, optionally substituted heteroaryl, optionally substituted alkoxy, optionally substituted aryloxy, cyano, optionally substituted alkylthio, optionally substituted alkylsufinyl, optionally substituted alkylsulfonyl, optionally substituted arylthio, optionally substituted acyl, optionally substituted amino, carboxyl, optionally substituted alkoxycarbonyl, optionally substituted carbamoyl, wherein R⁶ and R⁷, or R⁷ and R⁸, or R⁸ and R⁹ can be part of a cyclic alkylene group forming a fused ring structure;
wherein the compound is chosen from:
   tert-butyl 3-(1-{[6-chloro-5-(trifluoromethyl)(2-pyridyl)]amino}-4-methyl-2,5-dioxoazolin-3-yl)propanoate (S01860);
   ethyl 3-(1-{[6-chloro-5-(trifluoromethyl)(2-pyridyl)]amino} -4-methyl-2,5-dioxoazolin-3-yl)propanoate (S01861);
   3,4-dimethyl-1-[(4,7,8-trichloro(2-quinolyl))amino]azoline-2,5-dione (S01078),
   1-[(8-bromo-4-chloro(2-quinolyl))amino]-3,4-dimethylazoline-2,5-dione (S01247);
   tert-butyl 4-({2-[(3,4-dimethyl-2,5-dioxoazolinyl)amino]-7-bromo-4-quinolyl} methyl)piperazinecarboxylate (S01589);
   methyl 3-(1-{[6-chloro-5-(trifluoromethyl)(2-pyridyl)]amino}-4-methyl-2,5-dioxoazolin-3-yl)propanoate (S01648);
   3-(1-{[6-chloro-5-(trifluoromethyl)(2-pyridyl)]amino}-4-methyl-2,5-dioxoazolin-3-yl)-N-methoxy-N-methylpropanamide (S01796);
   1-{[7-bromo-4-({4-[(2-methoxyphenyl)carbonyl]piperazinyl}methyl) (2-quinolyl)]amino} -3,4-dimethylazoline-2,5-dione (S01879);
   1-{[3-bromo-6-chloro-5-(trifluoromethyl)(2-pyridyl)]amino}-3,4-dimethylazoline-2,5-dione (S01981);
   1-{[6-chloro-5-(trifluoromethyl)(2-pyridyl)]amino}-3,4-dimethylazoline-2,5-dione (S00109);
   1-{[6-chloro-5-(trifluoromethyl)(2-pyridyl)]methylamino}-3,4-dimethylazoline-2,5-dione (S00170);
   1-{[6-bromo-5-(trifluoromethyl)(2-pyridyl)]methylamino}-3,4-dimethylazoline-2,5-dione (S01007);
   1-{[6-chloro-5-(trifluoromethyl)(2-pyridyl)]amino}-4-methyl-3-(3-methylbutyl)azoline-2,5-dione (S01554);
   1-{[6-chloro-5-(trifluoromethyl)(2-pyridyl)]amino}-3-(methoxymethyl)-4-methylazoline-2,5-dione (S01599);
   1-{[7,8-dichloro-4-(trifluoromethyl)(2-quinolyl)]amino}-3,4-dimethylazoline-2,5-dione (S01455);
   3-(1-{[6-chloro-5-(trifluoromethyl)(2-pyridyl)]amino}-4-methyl-2,5-dioxoazolin-3-yl)-N,N-diethylpropanamide (S01711);
   diethyl 2-[(1-{[6-chloro-5-(trifluoromethyl)(2-pyridyl)]amino}-4-methyl-2,5-dioxoazolin-3-yl)methyl]propane-1,3-dioate (S01712);
   N-(tert-butyl)-3-(1-{[6-chloro-5-(trifluoromethyl)(2-pyridyl)]amino}-4-methyl-2,5-dioxoazolin-3-yl)propanamide (S01758);
   1-{[7-bromo-4-({4-[(3-methoxyphenyl)carbonyl]piperazinyl}methyl) (2-quinolyl)]amino} -3,4-dimethylazoline-2,5-dione (S01925);
   1-{[6-bromo-5-(trifluoromethyl)(2-pyridyl)]amino}-3,4-dimethylazoline-2,5-dione (S00994);
   1-[(4,8-dichloro(2-quinolyl))amino]-3,4-dimethylazoline-2,5-dione (S01005);
   3,4-dimethyl-1-{[6-phenyl-5-(trifluoromethyl)(2-pyridyl)]amino}azoline-2,5-dione (S01266);
   1-{[6-chloro-5-(trifluoromethyl)(2-pyridyl)]amino}-3-(hydroxymethyl)-4-methylazoline-2,5-dione (S01470);
   N-(3,4-dimethyl-2,5-dioxoazolinyl)-N-[6-chloro-5-(trifluoromethyl)(2-pyridyl)]acetamide (S01473);
   1-{[7-bromo-4-({4-[(2-chlorophenyl)carbonyl]piperazinyl}methyl)(2-quinolyl)]amino} -3,4-dimethylazoline-2,5-dione (S01878);
   3-(1-{[6-chloro-5-(trifluoromethyl)(2-pyridyl)]amino}-4-methyl-2,5-dioxoazolin-3-yl)-N-methylpropanamide (S01883);
   1-[(8-chloro(2-quinolyl))amino]-3,4-dimethylazoline-2,5-dione (S00585);
   3,4-dimethyl-1-[(3,4,5-trichlorophenyl)amino]azoline-2,5-dione (S00832);
   3,4-dimethyl-1-{[4-(trifluoromethyl)(2-quinolyl)]amino]azoline-2,5-dione (S00873);
   1-[(7-bromo-4-chloro(2-quinolyl))amino]-3,4-dimethylazoline-2,5-dione (S01311);
   1-{[6-(3-chloro-4-fluorophenyl)-5-(trifluoromethyl)(2-pyridyl)]amino} -(3,4-dimethylmethylazoline-2,5-dione (S01313);
   3,4-dimethyl-1-{[6-(2-methylpropyl)-5-(trifluoromethyl)(2-pyridyl)]amino}azoline-2,5-dione (S01457);
   1-{[6-chloro-4-(trifluoromethyl)(2-pyridyl)]amino} -3,4-dimethylazoline-2,5-dione (S01737);
   methyl 3-(1-{[4-({4-[(tert-butyl)oxycarbonyl]piperazinyl}methyl)-7-bromo (2-quinolyl)]amino}-4-methyl-2,5-dioxoazolin-3-yl)propanoate (S01865);
   1-({4-[(4-{[4-(dimethylamino)phenyl]carbonyl}piperazinyl)methyl]-7-bromo (2-quinolyl)}amino)-3,4-dimethylazoline-2,5-dione (S01880);
   1-[(3-chloroisoquinolyl)amino]-3,4-dimethylazoline-2,5-dione (S01098);
   1- { [6-chloro-5-(trifluoromethyl) (2-pyridyl)]amino} -3-ethyl-4-methylazoline-2,5-dione (S01553);
   1-{[4-chloro-6-phenyl-5-(trifluoromethyl)(2-pyridyl)]amino}-3,4-dimethylazoline-2,5-dione (S01734);
   N-[1-({2-[(3,4-dimethyl-2,5-dioxoazolinyl)amino]-7-bromo(4-quinolyl)}methyl)pyrrolidin-3-yl](tert-butoxy)carboxamide (S01864);
   1-{[7-bromo-4-({4-[(4-fluorophenyl)carbonyl]piperazinyl}methyl) (2-quinolyl)]amino}-3,4-dimethylazoline-2,5-dione (S01877);
   6-[(3,4-dimethyl-2,5-dioxoazolinyl)amino]-3-(trifluoromethyl)pyridine-2-carbonitrile (S01475);
   2-{[6-chloro-5-(trifluoromethyl)-2-pyridyl] amino}-4,5,6,7-tetrahydroisoindole-1,3-dione (S00186);
   1-{[4-bromo-3-(trifluoromethyl)phenyl]amino}-3,4-dimethylazoline-2,5-dione (S00516);
   1-[(4-chloronaphthyl)amino]-3,4-dimethylazoline-2,5-dione (S00738);
   1-[(4-chloro-6-methyl(2-quinolyl))amino]-3,4-dimethylazoline-2,5-dione (S00935);
   1-[(4-bromonaphthyl)amino]-3,4-dimethylazoline-2,5-dione (S00942);
   1-{[7-bromo-4-(hydroxymethyl)(2-quinolyl)]amino}-3,4-dimethylazoline-2,5-dione (S01037);
   {2-[(3,4-dimethyl-2,5-dioxoazolinyl)amino]-7-bromo-4-quinolyl} methyl acetate (S01047);
   1-{[8-chloro-4-(4-methoxyphenyl)(2-quinolyl)]amino}-3,4-dimethylazoline-2,5-dione (S01191);
   1-[(4-chlorobenzo[h]quinolin-2-yl)amino]-3,4-dimethylazoline-2,5-dione (S01207);
   1-[(7-bromo-4-{[4-benzylpiperazinyl]methyl}(2-quinolyl))amino]-3,4-dimethylazoline-2,5-dione (S01268);
   1-{[6-(4-chlorophenyl)-5-(trifluoromethyl)(2-pyridyl)]amino}-3,4-dimethylazoline-2,5-dione (S01371);
   3.4-dimethyl-1-{[6-(4-methylphenyl)-5-(trifluoromethyl)(2-pyridyl)]amino} azoline-2,5-dione (S01393);
   1-{[6-(3-chlorophenyl)-5-(trifluoromethyl)(2-pyridyl)]amino}-3,4-dimethylazoline-2,5-dione (S01474);
   1-{[6-chloro-5-(trifluoromethyl)(2-pyridyl)]methylamino}-3-(methoxymethyl)-4-methylazoline-2,5-dione (S01600);
   phenylmethyl 4-({2-[(3,4-dimethyl-2,5-dioxoazolinyl)amino]-7-bromo-4-quinolyl} methyl)piperazinecarboxylate (S01683);
   1-{[6-chloro-2-phenyl-3-(trifluoromethyl)(4-pyridyl)]amino}-3,4-dimethylazoline-2,5-dione (S01688);
   3,4-dimethyl-1-({6-[3-(trifluoromethyl)phenyl](2-pyridyl)}amino)azoline-2,5-dione (S01691);
   1-[(7-bromo-4-{[4-(phenylcarbonyl)piperazinyl]methyl}(2-quinolyl))amino]-3,4-dimethylazoline-2,5-dione (S01699);
   3-(1-{[6-chloro-5-(trifluoromethyl)(2-pyridyl)]amino}-4-methyl-2,5-dioxoazolin-3-yl)-N-methyl-N-phenylpropanamide (S01759);
   3,4-dimethyl-1-{[6-benzyl-5-(trifluoromethyl)(2-pyridyl)]amino}azoline-2,5-dione (S01762);
   1-{[4-({4-[(2,4-dimethylphenyl)carbonyl]piperazinyl}methyl)-7-bromo (2-quinolyl)]amino}-3,4-dimethylazoline-2,5-dione (S01800);
   1-{[7-bromo-4-({4-[(4-methoxyphenyl)carbonyl]piperazinyl}methyl)(2-quinolyl)]amino}-3,4-dimethylazoline-2,5-dione (S01801);
   N-[6-chloro-5-(trifluoromethyl)(2-pyridyl)]-N-[4-(hydroxymethyl)-3-methyl-2,5-dioxoazolinyl]acetamide (S01820);
   1-[(7-bromo-4-{[4-(phenylsulfonyl)piperazinyl]methyl}(2-quinolyl))amino]-3,4-dimethylazoline-2,5-dione (S01822);
   1-[(4-chloro-8-methyl(2-quinolyl))amino]-3,4-dimethylazoline-2,5-dione (S00871);
   tert-butyl 4-[({2-[(3,4-dimethyl-2,5-dioxoazolinyl)amino]-7-bromo-4-quinolyl}methyl)amino]piperidinecarboxylate (S01862);
   tert-butyl 4-[4-({2-[(3,4-dimethyl-2,5-dioxoazolinyl)amino]-7-bromo-4-quinolyl} methyl)piperazinyl]piperidinecarboxylate (S01928);
   1-[(4-{[4-(3,3-dimethylbutanoyl)piperazinyl]methyl}-7-bromo (2-quinolyl))amino]-3,4-dimethylazoline-2,5-dione (S01929);
   methylethyl 3-(1-{[6-chloro-5-(trifluoromethyl)(2-pyridyl)]amino}-4-methyl-2,5-dioxoazolin-3-yl)propanoate (S02022);
   methylpropyl 3-(1-{[6-chloro-5-(trifluoromethyl)(2-pyridyl)]amino}-4-methyl-2,5-dioxoazolin-3-yl)propanoate (S02264);
   tert-butyl 2-(1-{[6-chloro-5-(trifluoromethyl)(2-pyridyl)]amino}-4-methyl-2,5-dioxoazolin-3-yl)acetate (S02225);
   1-{[6-chloro-5-(trifluoromethyl)(2-pyridyl)]amino}-3-(ethoxymethyl)-4-methylazoline-2,5-dione (S02366);
   3-butyl-1-{[6-chloro-5-(trifluoromethyl)(2-pyridyl)]amino}-4-methylazoline-2,5-dione (S03448);
   1-{[6-chloro-5-(trifluoromethyl)(2-pyridyl)]amino}-4-methyl-3-[2-(2-methyl(1,3-dioxolan-2-yl))ethyl]azoline-2,5-dione (S03456);
   1-{[6-chloro-5-(trifluoromethyl)(2-pyridyl)]amino}-3-[(2-methoxyethoxy)methyl]-4-methylazoline-2,5-dione (S03742);
   1-{[6-chloro-5-(trifluoromethyl)(2-pyridyl)]amino}-4-(3-hydroxyhexyl)-3-methylazoline-2,5-dione (S03552);
   1-{[6-chloro-5-(trifluoromethyl)(2-pyridyl)]amino}-4-(3-hydroxypentyl)-3-methylazoline-2,5-dione (S03745);
   1-{[6-chloro-5-(trifluoromethyl)(2-pyridyl)]amino}-4-methyl-3-[(3-methylbutoxy)methyl]azoline-2,5-dione (S03405);
   3-(butoxymethyl)-1-{[6-chloro-5-(trifluoromethyl)(2-pyridyl)]amino}-4-methylazoline-2,5-dione (S03518);
   3-[(3,3-dimethylbutoxy)methyl]-1-{[6-chloro-5-(trifluoromethyl)(2-pyridyl)]amino}-4-methylazoline-2,5-dione (S03747);
   1-{[6-chloro-5-(trifluoromethyl)(2-pyridyl)]amino}-3-(2-ethoxyethyl)-4-methylazoline-2,5-dione (S03960);
   1-{[6-chloro-5-(trifluoromethyl)(2-pyridyl)]amino}-4-methyl-3-[(2-methylpropoxy)methyl]azoline-2,5-dione (S03963);
   3-[(2,2-dimethylpropoxy)methyl]-1-{[6-chloro-5-(trifluoromethyl)(2-pyridyl)]amino}-4-methylazoline-2,5-dione (S03962);
   4-[(1,3-dimethylbutoxy)methyl]-1-{[6-chloro-5-(trifluoromethyl)(2-pyridyl)]amino}-3-methylazoline-2,5-dione (S03964);
   4-[(tert-butoxy)methyl]-1-{[6-chloro-5-(trifluoromethyl)(2-pyridyl)]amino}-3-methylazoline-2,5-dione (S03873);
   1-{[6-chloro-5-(trifluoromethyl)(2-pyridyl)]amino}-4-methyl-3-[2-(2-methylpropoxy)ethyl]azoline-2,5-dione(S03955);
   1-{[6-chloro-5-(trifluoromethyl)(2-pyridyl)]amino}-4-methyl-3-[2-(3-methylbutoxy)ethyl]azoline-2,5-dione (S03956);
   1-{[6-chloro-5-(trifluoromethyl)(2-pyridyl)]amino}-3-methyl-4-(2-propoxyethyl)azoline-2,5-dione (S04034);
   and salts thereof.

Embodiment P6. The method of embodiment P5, wherein the compound is tert-butyl 3-(1-{[6-chloro-5-(trifluoromethyl)(2-pyridyl)]amino}-4-methyl-2,5-dioxoazolin-3-yl)propanoate (S01860) or a salt thereof; having the following structure:

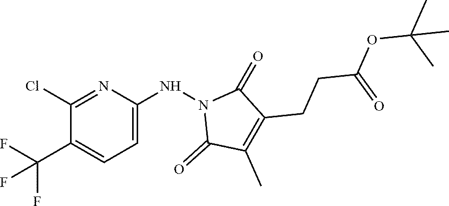 [SMILES: CC1=C(CCC(=O)OC(C)(C)C)C(=O)N(Nc2ccc(C(F)(F)F)c(Cl)n2)C1=O]

Embodiment P7. The method of embodiment P5, wherein the compound is ethyl 3-(1-{[6-chloro-5-(trifluoromethyl)(2-pyridyl)]amino}-4-methyl-2,5-dioxoazolin-3-yl)propanoate (S01861) or a salt thereof, having the following structure:

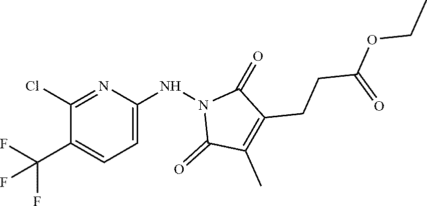 [SMILES: CCOC(=O)CCC1=C(C)C(=O)N(Nc2ccc(C(F)(F)F)c(Cl)n2)C1=O]

Embodiment P8. The method of embodiment P5, wherein the compound is 3,4-dimethyl-1-[(4,7,8-trichloro(2-quinolyl))amino]azoline-2,5-dione (S01078) or a salt thereof, having the following structure:

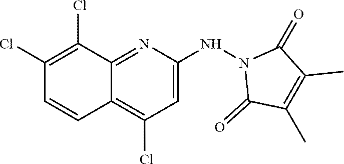 [SMILES: CC1=C(C)C(=O)N(Nc2cc(Cl)c3ccc(Cl)c(Cl)c3n2)C1=O]

Embodiment P9. The method of embodiment P5, wherein the compound is 1-[(8-bromo-4-chloro(2-quinolyl))amino]-3,4-dimethylazoline-2,5-dione (S01247) or a salt thereof, having the following structure:

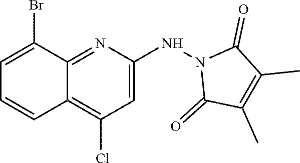 [SMILES: CC1=C(C)C(=O)N(Nc2cc(Cl)c3cccc(Br)c3n2)C1=O]

Embodiment P10. The method of embodiment P5, wherein the compound is tert-butyl 4-({2-[(3,4-dimethyl-2,5-dioxoazolinyl)amino]-7-bromo-4-quinolyl}methyl)piperazinecarboxylate (S01589) or a salt thereof, having the following structure:

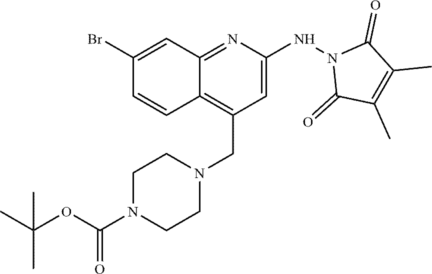 [SMILES: CC1=C(C)C(=O)N(Nc2cc(CN3CCN(C(=O)OC(C)(C)C)CC3)c3ccc(Br)cc3n2)C1=O]

Embodiment P11. The method of embodiment P5, wherein the compound is methyl 3-(1-{[6-chloro-5-(trifluoromethyl)(2-pyridyl)]amino] -4-methyl-2,5-dioxoazolin-3-yl)propanoate (S01648) or a salt thereof, having the following structure:

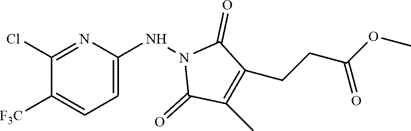 [SMILES: COC(=O)CCC1=C(C)C(=O)N(Nc2ccc(C(F)(F)F)c(Cl)n2)C1=O]

Embodiment P12. The method of embodiment P5, wherein the compound is 3-(1-{[6-chloro-5-(trifluoromethyl)(2-pyridyl)]amino}-4-methyl-2,5-dioxoazolin-3-yl)-N-methoxy-N-methylpropanamide (S01796) or a salt thereof, having the following structure:

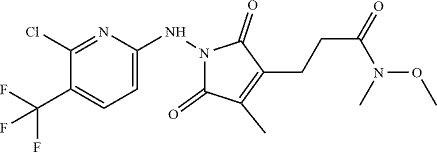 [SMILES: CON(C)C(=O)CCC1=C(C)C(=O)N(Nc2ccc(C(F)(F)F)c(Cl)n2)C1=O]

Embodiment P13. The method of embodiment P5, wherein the compound is 1-{[7-bromo-4-({4-[(2-methoxyphenyl)carbonyl]piperazinyl}methyl)(2-quinolyl)]amino}-3,4-dimethylazoline-2,5-dione (S01879) or a salt thereof, having the following structure:

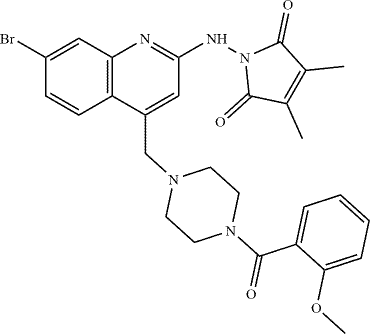 [SMILES: COc1ccccc1C(=O)N1CCN(Cc2cc(NN3C(=O)C(C)=C(C)C3=O)nc3cc(Br)ccc23)CC1]

Embodiment P14. The method of embodiment P5, wherein the compound is 1-{[3-bromo-6-chloro-5-(trifluoromethyl)(2-pyridyl)]amino}-3,4-dimethylazoline-2,5-dione (S01981) or a salt thereof, having the following structure:

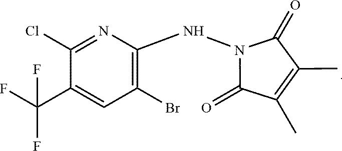

Embodiment P15. The method of embodiment P5, wherein the compound is 1-{[6-chloro-5-(trifluoromethyl)(2-pyridyl)]amino}-3,4-dimethylazoline-2,5-dione (S00109) or a salt thereof, having the following structure:

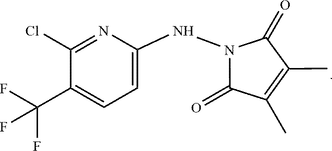

Embodiment P16. The method of embodiment P5, wherein the compound is 1-{[6-chloro-5-(trifluoromethyl)(2-pyridyl)]methylamino}-3,4-dimethylazoline-2,5-dione (S00170) or a salt thereof, having the following structure:

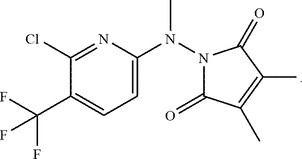

Embodiment P17. The method of embodiment P5, wherein the compound is 1- {[6-bromo-5-(trifluoromethyl) (2-pyridyl)]methylamino} -3,4-dimethylazoline-2,5-dione (S01007) or a salt thereof, having the following structure:

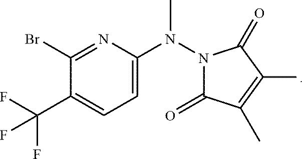

Embodiment P18. The method of embodiment P5, wherein the compound is 1- {[6-chloro-5-(trifluoromethyl) (2-pyridyl)]amino} -4-methyl-3-(3-methylbutyl)azoline-2,5-dione (S01554), or a salt thereof, having the following structure:

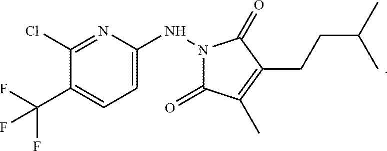

Embodiment P19. The method of embodiment P5, wherein the compound is 1-{[6-chloro-5-(trifluoromethyl)(2-pyridyl)]amino}-3-(methoxymethyl)-4-methylazoline-2,5-dione (S01599) or a salt thereof, having the following structure:

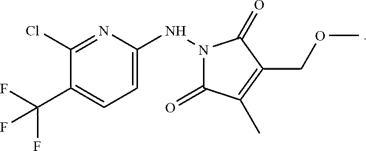

Embodiment P20. The method of embodiment P5, wherein the compound is 1- {[7,8-dichloro-4-(trifluoromethyl) (2-quinolyl)]amino} -3,4-dimethylazoline-2,5-dione (S01455) or a salt thereof, having the following structure:

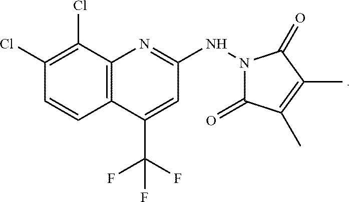

Embodiment P21. The method of embodiment P5, wherein the compound is 3-(1-{[6-chloro-5-(trifluoromethyl)(2-pyridyl)]amino}-4-methyl-2,5-dioxoazolin-3-yl)-N,N-diethylpropanamide (S01711) or a salt thereof, having the following structure:

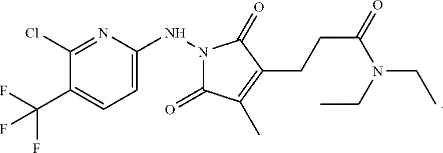

Embodiment P22. The method of embodiment P5, wherein the compound is diethyl 2-[(1-{[6-chloro-5-(trifluoromethyl)(2-pyridyl)]amino}-4-methyl-2,5-dioxoazolin-3-yl)methyl]propane-1,3-dioate (S01712) or a salt thereof, having the following structure:

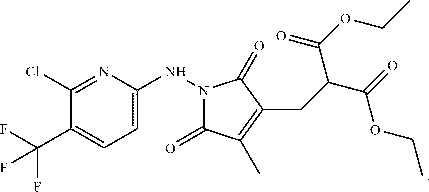

Embodiment P23. The method of embodiment P5, wherein the compound is N-(tert-butyl)-3-(1-{[6-chloro-5-(trifluoromethyl)(2-pyridyl)]amino}-4-methyl-2,5-dioxoazolin-3-yl)propanamide (S01758) or a salt thereof, having the following structure:

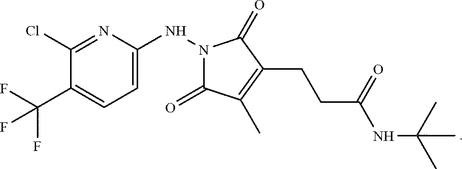

Embodiment P24. The method of embodiment P5, wherein the compound is 1-{[7-bromo-4-({4-[(3-methoxyphenyl)carbonyl]piperazinyl}methyl)(2-quinolyl)]amino]-3,4-dimethylazoline-2,5-dione (S01925) or a salt thereof, having the following structure:

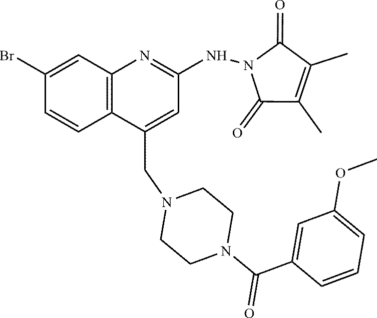 [SMILES: COc1cccc(C(=O)N2CCN(Cc3cc(NN4C(=O)C(C)=C(C)C4=O)nc4cc(Br)ccc34)CC2)c1]

Embodiment P25. The method of embodiment P5, wherein the compound is 1-{[6-bromo-5-(trifluoromethyl)(2-pyridyl)]amino} -3,4-dimethylazoline-2,5-dione (S00994) or a salt thereof, having the following structure:

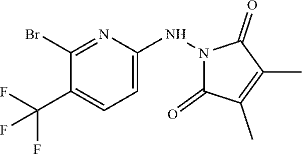 [SMILES: CC1=C(C)C(=O)N(Nc2ccc(C(F)(F)F)c(Br)n2)C1=O]

Embodiment P26. The method of embodiment P5, wherein the compound is 1-[(4,8-dichloro(2-quinolyl))amino]-3,4-dimethylazoline-2,5-dione (S01005) or a salt thereof, having the following structure:

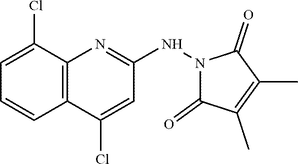 [SMILES: CC1=C(C)C(=O)N(Nc2cc(Cl)c3cccc(Cl)c3n2)C1=O]

Embodiment P27. The method of embodiment P5, wherein the compound is 3,4-dimethyl-1-{[6-phenyl-5-(trifluoromethyl)(2-pyridyl)]amino}azoline-2,5-dione (S01266) or a salt thereof, having the following structure:

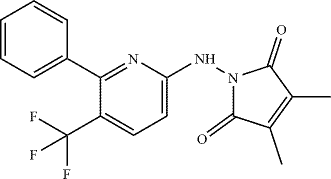 [SMILES: CC1=C(C)C(=O)N(Nc2ccc(C(F)(F)F)c(-c3ccccc3)n2)C1=O]

Embodiment P28. The method of embodiment P5, wherein the compound is 1- {[6-chloro-5-(trifluoromethyl) (2-pyridyl)]amino}-3-(hydroxymethyl)-4-methylazoline-2,5-dione (S01470) or a salt thereof, having the following structure:

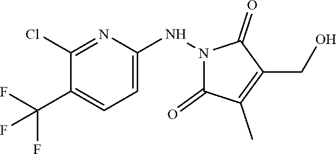 [SMILES: CC1=C(CO)C(=O)N(Nc2ccc(C(F)(F)F)c(Cl)n2)C1=O]

Embodiment P29. The method of embodiment P5, wherein the compound is N-(3,4-dimethyl-2,5-dioxoazolinyl)-N-[6-chloro-5-(trifluoromethyl)(2-pyridyl)]acetamide (S01473) or a salt thereof, having the following structure:

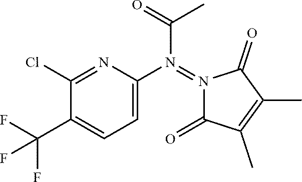

Embodiment P30. The method of embodiment P5, wherein the compound is 1-{[7-bromo-4-({4-[(2-chlorophenyl)carbonyl]piperazinyl}methyl)(2-quinolyl)]amino}-3,4-dimethylazoline-2,5-dione (S01878) or a salt thereof, having the following structure:

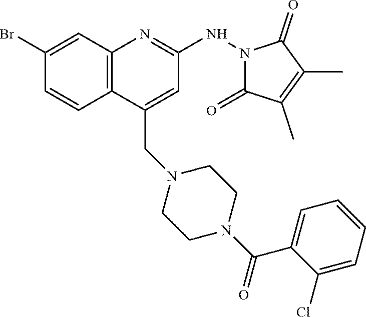 [SMILES: CC1=C(C)C(=O)N(Nc2cc(CN3CCN(C(=O)c4ccccc4Cl)CC3)c3ccc(Br)cc3n2)C1=O]

Embodiment P31. The method of embodiment P5, wherein the compound is 3-(1-{[6-chloro-5-(trifluoromethyl) (2-pyridyl)]amino}-4-methyl-2,5-dioxoazolin-3-yl)-N-methylpropanamide (S01883) or a salt thereof, having the following structure:

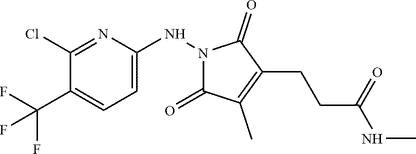 [SMILES: CNC(=O)CCC1=C(C)C(=O)N(Nc2ccc(C(F)(F)F)c(Cl)n2)C1=O]

Embodiment P32. The method of embodiment P5, wherein the compound is 1-[(8-chloro(2-quinolyl))amino]-3,4-dimethylazoline-2,5-dione (S00585) or a salt thereof, having the following structure:

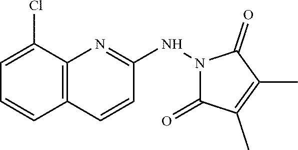 [SMILES: CC1=C(C)C(=O)N(Nc2ccc3cccc(Cl)c3n2)C1=O]

Embodiment P33. The method of embodiment P5, wherein the compound is 3,4-dimethyl-1-[(3,4,5-trichlorophenyl)amino]azoline-2,5-dione (S00832) or a salt thereof, having the following structure:

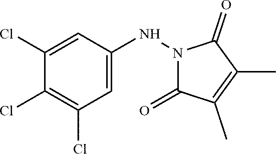 [SMILES: CC1=C(C)C(=O)N(Nc2cc(Cl)c(Cl)c(Cl)c2)C1=O]

Embodiment P34. The method of embodiment P5, wherein the compound is 3,4-dimethyl-1- {[4-(trifluoromethyl)(2-quinolyl)]amino} azoline-2,5-dione (S00873) or a salt thereof, having the following structure:

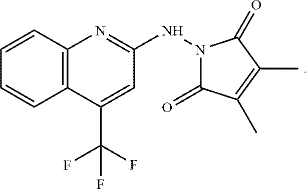

Embodiment P35. The method of embodiment P5, wherein the compound is 1-[(7-bromo-4-chloro(2-quinolyl))amino]-3,4-dimethylazoline-2,5-dione (S01311) or a salt thereof, having the following structure:

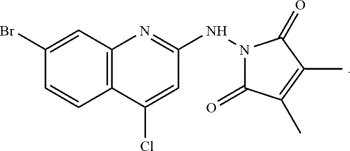

Embodiment P36. The method of embodiment P5, wherein the compound is 1-{[6-(3-chloro-4-fluorophenyl)-5-(trifluoromethyl)(2-pyridyl)]amino}-(3,4-dimethylazoline-2,5-dione (S01313) or a salt thereof, having the following structure:

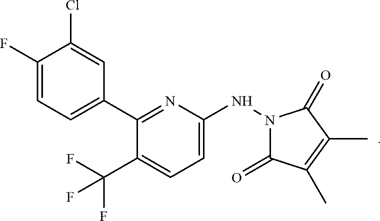

Embodiment P37. The method of embodiment P5, wherein the compound is 3,4-dimethyl-1-{[6-(2-methylpropyl)-5-(trifluoromethyl)(2-pyridyl)]amino}azoline -2,5-dione (S01457) or a salt thereof, having the following structure:

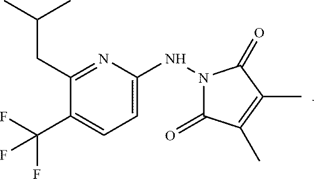

Embodiment P38. The method of embodiment P5, wherein the compound is 1-{[6-chloro-4-(trifluoromethyl)(2-pyridyl)]amino}-3,4-dimethylazoline-2,5-dione (S01737) or a salt thereof, having the following structure:

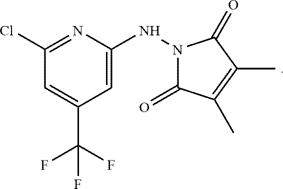

Embodiment P39. The method of embodiment P5, wherein the compound is methyl 3-(1-{[4-({4-[(tert-butyl)oxycarbonyl]piperazinyl}methyl)-7-bromo(2-quinolyl)]amino}-4-methyl-2,5-dioxoazolin-3-yl)propanoate (S01865) or a salt thereof, having the following structure:

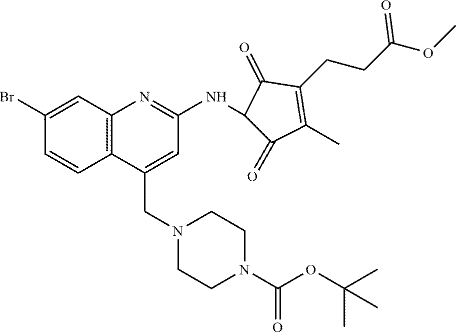 [SMILES: COC(=O)CCC1=C(C)C(=O)C(Nc2cc(CN3CCN(C(=O)OC(C)(C)C)CC3)c3ccc(Br)cc3n2)C1=O]

Embodiment P40. The method of embodiment P5, wherein the compound is 1-({4-[(4-{[4-(dimethylamino)phenyl]carbonyl}piperazinyl)methyl]-7-bromo (2-quinolyl)}amino)-3,4-dimethylazoline-2,5-dione (S01880) or a salt thereof, having the following structure:

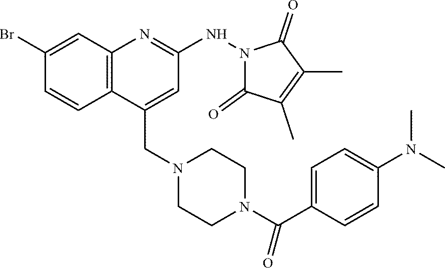 [SMILES: CC1=C(C)C(=O)N(Nc2cc(CN3CCN(C(=O)c4ccc(N(C)C)cc4)CC3)c3ccc(Br)cc3n2)C1=O]

Embodiment P41. The method of embodiment P5, wherein the compound is 1-[(3-chloroisoquinolyl)-amino]-3,4-dimethylazoline-2,5-dione (S01098) or a salt thereof, having the following structure:

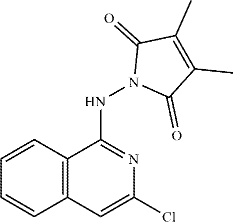 [SMILES: CC1=C(C)C(=O)N(Nc2nc(Cl)cc3ccccc23)C1=O]

Embodiment P42. The method of embodiment P5, wherein the compound is 1-{[6-chloro-5-(trifluoromethyl)(2-pyridyl)]-amino}-3-ethyl-4-methylazoline-2,5-dione (S01553) or a salt thereof, having the following structure:

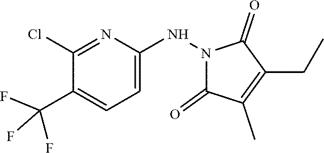 [SMILES: CCC1=C(C)C(=O)N(Nc2ccc(C(F)(F)F)c(Cl)n2)C1=O]

Embodiment P43. The method of embodiment P5, wherein the compound is 1- {[4-chloro-6-phenyl-5-(trifluoromethyl) (2-pyridyl)]amino} -3,4-dimethylazoline-2,5-dione (S01734) or a salt thereof, having the following structure:

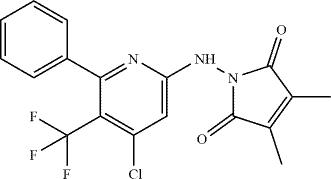 [SMILES: CC1=C(C)C(=O)N(Nc2cc(Cl)c(C(F)(F)F)c(-c3ccccc3)n2)C1=O]

Embodiment P44. The method of embodiment P5, wherein the compound is N-[1-({2-[(3,4-dimethyl-2,5-dioxoazolinyl)amino]-7-bromo(4-quinolyl)}-methyl)pyrrolidin-3-yl] (tert-butoxy)carboxamide (S01864) or a salt thereof, having the following structure:

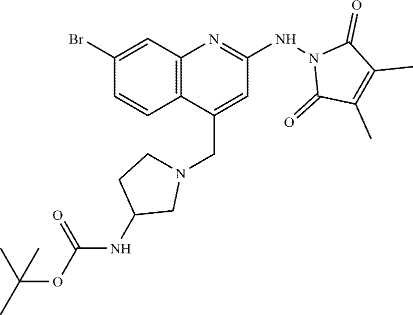 [SMILES: CC1=C(C)C(=O)N(Nc2cc(CN3CCC(NC(=O)OC(C)(C)C)C3)c3ccc(Br)cc3n2)C1=O]

Embodiment P45. The method of embodiment P5, wherein the compound is 1-{[7-bromo-4-({4-[(4-fluorophenyl)carbonyl]piperazinyl}methyl) (2-quinolyl)]amino} -3,4-dimethylazoline-2,5-dione (S01877) or a salt thereof, having the following structure:

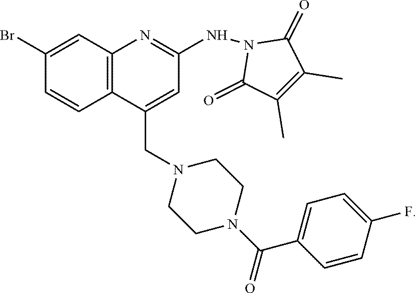 [SMILES: CC1=C(C)C(=O)N(Nc2cc(CN3CCN(C(=O)c4ccc(F)cc4)CC3)c3ccc(Br)cc3n2)C1=O]

Embodiment P46. The method of embodiment P5, wherein the compound is 6-[(3,4-dimethyl-2,5-dioxoazolinyl)amino]-3-(trifluoromethyl)pyridine-2-carbonitrile (S01475) or a salt thereof, having the following structure:

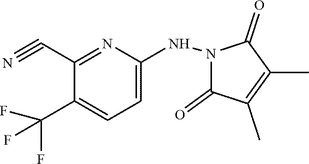 [SMILES: CC1=C(C)C(=O)N(Nc2ccc(C(F)(F)F)c(C#N)n2)C1=O]

Embodiment P47. The method of embodiment P5, wherein the compound is 2-{[6-chloro-5-(trifluoromethyl)-2-pyridyl]amino}-4,5,6,7-tetrahydroisoindole-1,3-dione (S00186) or a salt thereof, having the following structure:

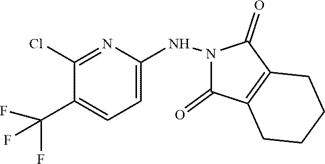 [SMILES: O=C1C2=C(CCCC2)C(=O)N1Nc1ccc(C(F)(F)F)c(Cl)n1]

Embodiment P48. The method of embodiment P5, wherein the compound is 1-{[4-bromo-3-(trifluoromethyl)phenyl]amino}-3,4-dimethylazoline-2,5-dione (S00516) or a salt thereof, having the following structure:

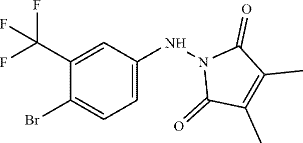 [SMILES: CC1=C(C)C(=O)N(Nc2ccc(Br)c(C(F)(F)F)c2)C1=O]

Embodiment P49. The method of embodiment P5, wherein the compound is 1-[(4-chloronaphthyl)amino]-3,4-dimethylazoline-2,5-dione (S00738) or a salt thereof, having the following structure:

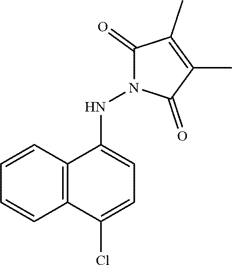 [SMILES: CC1=C(C)C(=O)N(Nc2ccc(Cl)c3ccccc23)C1=O]

Embodiment P50. The method of embodiment P5, wherein the compound is 1-[(4-chloro-6-methyl(2-quinolyl))amino]-3,4-dimethylazoline-2,5-dione (S00935) or a salt thereof, having the following structure:

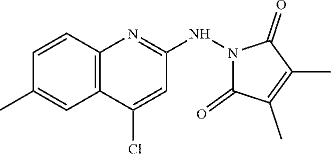

Embodiment P51. The method of embodiment P5, wherein the compound is 1-[(4-bromonaphthyl)amino]-3,4-dimethylazoline-2,5-dione (S00942) or a salt thereof, having the following structure:

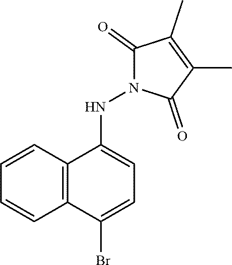 [SMILES: CC1=C(C)C(=O)N(Nc2ccc(Br)c3ccccc23)C1=O]

Embodiment P52. The method of embodiment P5, wherein the compound is 1-{[7-bromo-4-(hydroxymethyl)(2-quinolyl)]amino} -3,4-dimethylazoline-2,5-dione (S01037) or a salt thereof, having the following structure:

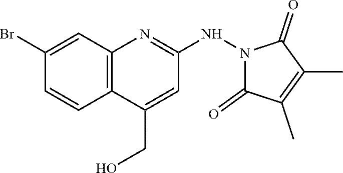 [SMILES: CC1=C(C)C(=O)N(Nc2cc(CO)c3ccc(Br)cc3n2)C1=O]

Embodiment P53. The method of embodiment P5, wherein the compound is {2-[(3,4-dimethyl-2,5-dioxoazolinyl)amino]-7-bromo-4-quinolyl} methyl acetate (S01047) or a salt thereof, having the following structure:

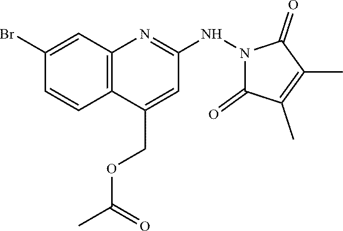 [SMILES: CC(=O)OCc1cc(NN2C(=O)C(C)=C(C)C2=O)nc2cc(Br)ccc12]

Embodiment P54. The method of embodiment P5, wherein the compound is 1-{[8-chloro-4-(4-methoxyphenyl)(2-quinolyl)]amino]-3,4-dimethylazoline-2,5-dione (S01191) or a salt thereof, having the following structure:

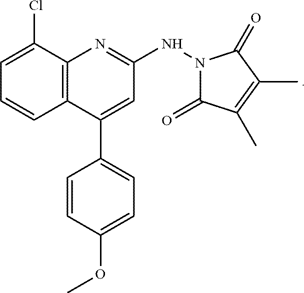

Embodiment P55. The method of embodiment P5, wherein the compound is 1-[(4-chlorobenzo[h]quinolin-2-yl)amino]-3,4-dimethylazoline-2,5-dione (S01207) or a salt thereof, having the following structure:

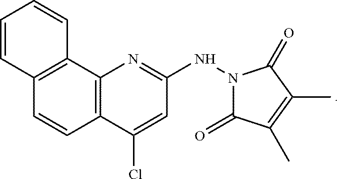

Embodiment P56. The method of embodiment P5, wherein the compound is 1-[(7-bromo-4-{[4-benzylpiperazinyl]methyl}(2-quinolyl))amino]-3,4-dimethylazoline-2,5-dione (S01268) or a salt thereof, having the following structure:

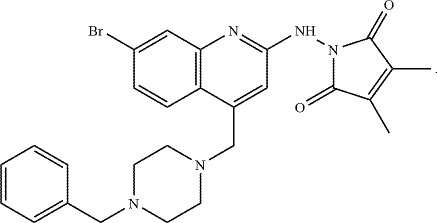

Embodiment P57. The method of embodiment P5, wherein the compound is 1- {[6-(4-chlorophenyl)-5-(trifluoromethyl) (2-pyridyl)]amino} -3,4-dimethylazoline-2,5-dione (S01371) or a salt thereof, having the following structure:

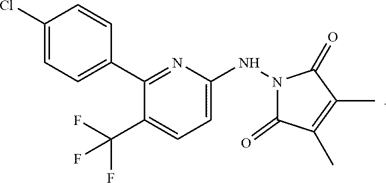

Embodiment P58. The method of embodiment P5, wherein the compound is 3,4-dimethyl-1-{[6-(4-methylphenyl)-5-(trifluoromethyl)(2-pyridyl)]amino}azoline-2,5-dione (S01393) or a salt thereof, having the following structure:

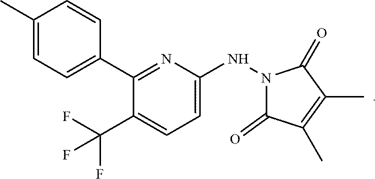

Embodiment P59. The method of embodiment P5, wherein the compound is 1- {[6-(3-chlorophenyl)-5-(trifluoromethyl) (2-pyridyl)]amino}-3,4-dimethylazoline-2,5-dione (S01474) or a salt thereof, having the following structure:

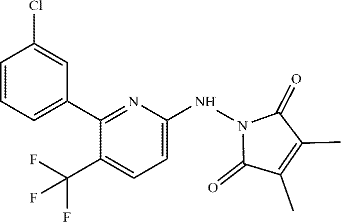 [SMILES: CC1=C(C)C(=O)N(Nc2ccc(C(F)(F)F)c(-c3cccc(Cl)c3)n2)C1=O]

Embodiment P60. The method of embodiment P5, wherein the compound is 1-{[6-chloro-5-(trifluoromethyl)(2-pyridyl)]methylamino}-3-(methoxymethyl)-4-methylazoline-2,5-dione (S01600) or a salt thereof, having the following structure:

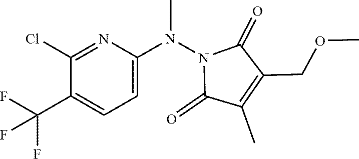 [SMILES: COCC1=C(C)C(=O)N(N(C)c2ccc(C(F)(F)F)c(Cl)n2)C1=O]

Embodiment P61. The method of embodiment P5, wherein the compound is phenylmethyl 4-({2-[3,4-dimethyl-2,5-dioxoazolinyl)amino]-7-bromo-4-quinolyl}methyl)piperazinecarboxylate (S01683) a salt thereof, having the following structure:

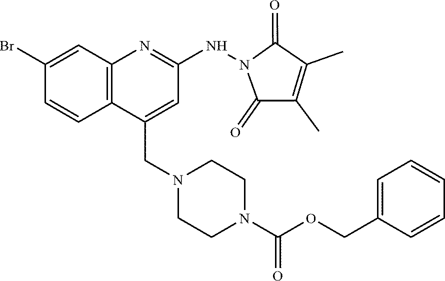 [SMILES: CC1=C(C)C(=O)N(Nc2cc(CN3CCN(C(=O)OCc4ccccc4)CC3)c3ccc(Br)cc3n2)C1=O]

Embodiment P62. The method of embodiment P5, wherein the compound is 1- {[6-chloro-2-phenyl-3-(trifluoromethyl) (4-pyridyl] amino} -3,4-dimethylazoline-2,5-dione (S01688) or a salt thereof, having the following structure:

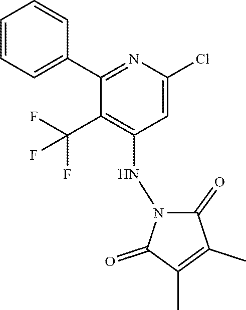 [SMILES: CC1=C(C)C(=O)N(Nc2cc(Cl)nc(-c3ccccc3)c2C(F)(F)F)C1=O]

Embodiment P63. The method of embodiment P5, wherein the compound is 3,4-dimethyl-1-({6-[3-(trifluoromethyl)phenyl](2-pyridyl)}amino)azoline-2,5-dione (S01691) or a salt thereof, having the following structure:

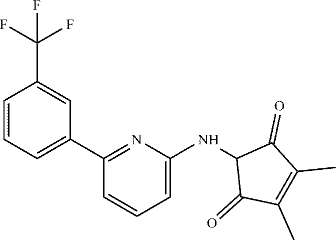 [SMILES: CC1=C(C)C(=O)C(Nc2cccc(-c3cccc(C(F)(F)F)c3)n2)C1=O]

Embodiment P64. The method of embodiment P5, wherein the compound is 1-[(7-bromo-4-{[4-(phenylcarbonyl)piperazinyl]methyl}(2-quinolyl)amino]-3,4-dimethylazoline-2,5-dione (S01699) or a salt thereof, having the following structure:

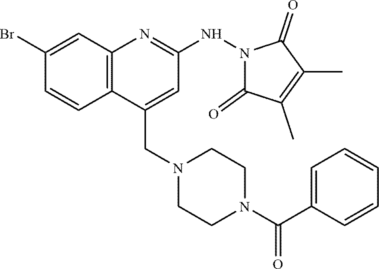 [SMILES: CC1=C(C)C(=O)N(Nc2cc(CN3CCN(C(=O)c4ccccc4)CC3)c3ccc(Br)cc3n2)C1=O]

Embodiment P65. The method of embodiment P5, wherein the compound is 3-(1-{[6-chloro-5-(trifluoromethyl)(2-pyridyl)]amino}-4-methyl-2,5-dioxoazolin-3-yl)-N-methyl-N-phenylpropanamide (S01759) or a salt thereof, having the following structure:

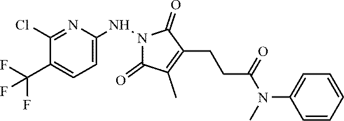 [SMILES: CC1=C(CCC(=O)N(C)c2ccccc2)C(=O)N(Nc2ccc(C(F)(F)F)c(Cl)n2)C1=O]

Embodiment P66. The method of embodiment P5, wherein the compound is 3,4-dimethyl-1-{[6-benzyl-5-(trifluoromethyl)(2-pyridyl)]amino}azoline-2,5-dione (S01762) or a salt thereof, having the following structure:

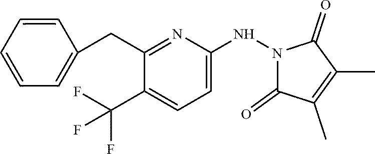 [SMILES: CC1=C(C)C(=O)N(Nc2ccc(C(F)(F)F)c(Cc3ccccc3)n2)C1=O]

Embodiment P67. The method of embodiment P5, wherein the compound is 1-{[4-({4-[(2,4-dimethylphenyl)carbonyl]piperazinyl}methyl)-7-bromo(2-quinolyl)]amino}-3,4-dimethylazoline-2,5-dione (S01800) or a salt thereof, having the following structure:

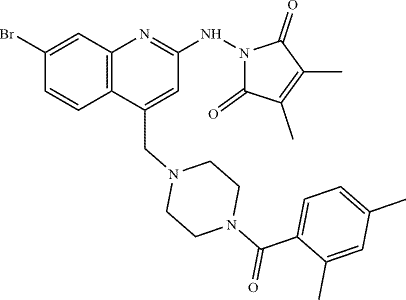 [SMILES: CC1=C(C)C(=O)N(Nc2cc(CN3CCN(C(=O)c4ccc(C)cc4C)CC3)c3ccc(Br)cc3n2)C1=O]

Embodiment P68. The method of embodiment P5, wherein the compound is 1-{[7-bromo-4-({4-[(4-methoxyphenyl)carbonyl]piperazinyl}methyl)(2-quinolyl)]amino]-3,4-dimethylazoline-2,5-dione (S01801) or a salt thereof, having the following structure:

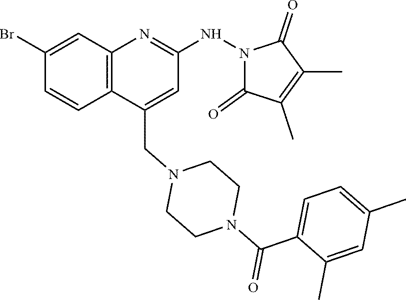 [SMILES: CC1=C(C)C(=O)N(Nc2cc(CN3CCN(C(=O)c4ccc(C)cc4C)CC3)c3ccc(Br)cc3n2)C1=O]

Embodiment P69. The method of embodiment P5, wherein the compound is N-[6-chloro-5-(trifluoromethyl)(2-pyridyl)]-N-[4-(hydroxymethyl)-3-methyl-2,5-dioxoazolinyl]acetamide (S01820) or a salt thereof, having the following structure:

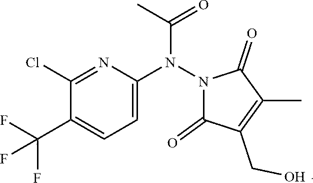

Embodiment P70. The method of embodiment P5, wherein the compound is 1-[(7-bromo-4-{[4-(phenylsulfonyl)piperazinyl]methyl}(2-quinolyl))amino]-3,4-dimethylazoline-2,5-dione (S01822) or a salt thereof, having the following structure:

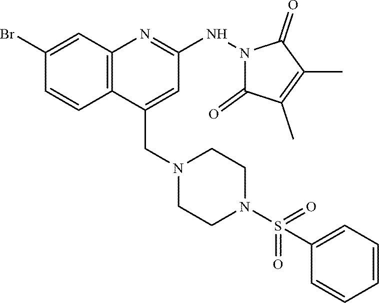 [SMILES: CC1=C(C)C(=O)N(Nc2cc(CN3CCN(S(=O)(=O)c4ccccc4)CC3)c3ccc(Br)cc3n2)C1=O]

Embodiment P71. The method of embodiment P5, wherein the compound is 1-[(4-chloro-8-methyl(2-quinolyl))amino]-3,4-dimethylazoline-2,5-dione (S00871) or a salt thereof, having the following structure:

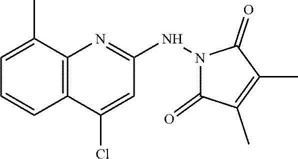 [SMILES: CC1=C(C)C(=O)N(Nc2cc(Cl)c3cccc(C)c3n2)C1=O]

Embodiment P72. The method of embodiment P5, wherein the compound is tert-butyl 4-[({2-[(3,4-dimethyl-2,5-dioxoazolinyl)amino]-7-bromo-4-quinolyl}methyl)amino]piperidinecarboxylate (S01862) or a salt thereof, having the following structure:

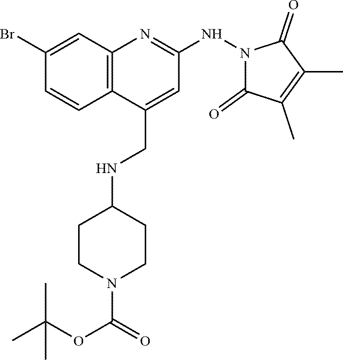 [SMILES: CC1=C(C)C(=O)N(Nc2cc(CNC3CCN(C(=O)OC(C)(C)C)CC3)c3ccc(Br)cc3n2)C1=O]

Embodiment P73. The method of embodiment P5, wherein the compound is tert-butyl 4-[4-({2-[(3,4-dimethyl-2,5-dioxoazolinyl)amino]-7-bromo-4-quinolyl}methyl)piperazinyl]piperidinecarboxylate (S01928) or a salt thereof, having the following structure:

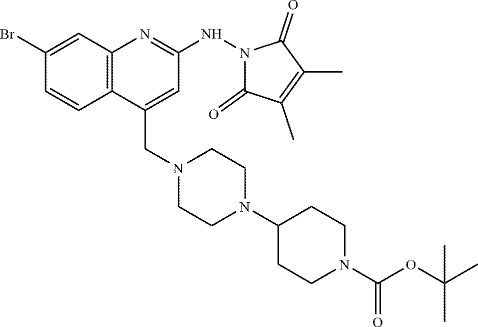 [SMILES: CC1=C(C)C(=O)N(Nc2cc(CN3CCN(C4CCN(C(=O)OC(C)(C)C)CC4)CC3)c3ccc(Br)cc3n2)C1=O]

Embodiment P74. The method of embodiment P5, wherein the compound is 1-[(4-{[4-(3,3-dimethylbutanoyl)piperazinyl]methyl}-7-bromo(2-quinolyl))amino]-3,4-dimethylazoline-2,5-dione (S01929) or a salt thereof, having the following structure:

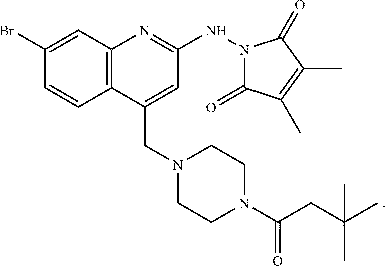

Embodiment P75. The method of embodiment P5, wherein the compound is methylethyl 3-(1-{[6-chloro-5-(trifluoromethyl) (2-pyridyl)] amino}-4-methyl-2,5-dioxoazolin-3-yl)propanoate (S02022) or a salt thereof, having the following structure:

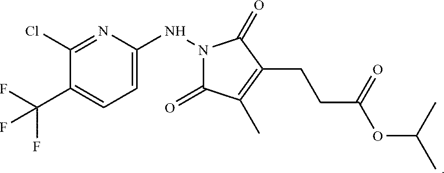

Embodiment P76. The method of embodiment P5, wherein the compound is methylpropyl 3-(1-1[6-chloro-5-(trifluoromethyl)(2-pyridyl)]amino}-4-methyl-2,5-dioxoazolin-3-yl)propanoate (S02264) or a salt thereof, having the following structure:

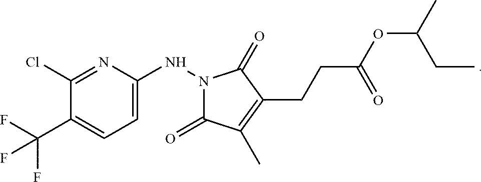

Embodiment P77. The method of embodiment P5, wherein the compound is tert-butyl 2-(1-{[6-chloro-5-(trifluoromethyl)(2-pyridyl)]amino}-4-methyl-2,5-dioxoazolin-3-yl)acetate (S02225) or a salt thereof, having the following structure:

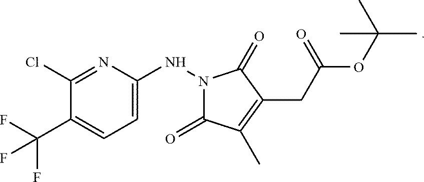

Embodiment P78. The method of embodiment P5, wherein the compound is 1- {[6-chloro-5-(trifluoromethyl) (2-pyridyl)]amino}-3-(ethoxymethyl)-4-methylazoline-2,5-dione (S02366) or a salt thereof, having the following structure:

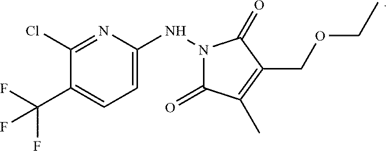

Embodiment P79. The method of embodiment P5, wherein the compound is 3-butyl-1-{[6-chloro-5-(trifluoromethyl)(2-pyridyl)]amino]-4-methylazoline-2,5-dione (S03448) or a salt thereof, having the following structure:

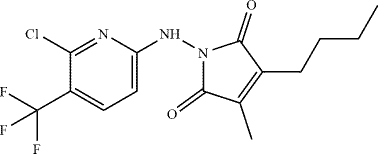 [SMILES: CCCCC1=C(C)C(=O)N(Nc2ccc(C(F)(F)F)c(Cl)n2)C1=O]

Embodiment P80. The method of embodiment P5, wherein the compound is 1-{[6-chloro-5-(trifluoromethyl)(2-pyridyl)]amino}-4-methyl-3-[2-(2-methyl(1,3-dioxolan-2-yl))ethyl]azoline-2,5-dione (S03456) or a salt thereof, having the following structure:

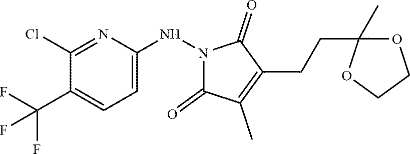 [SMILES: CC1=C(CCC2(C)OCCO2)C(=O)N(Nc2ccc(C(F)(F)F)c(Cl)n2)C1=O]

Embodiment P81. The method of embodiment P5, wherein the compound is 1-{[6-chloro-5-(trifluoromethyl)(2-pyridyl)]amino}-3-[(2-methoxyethoxy)methyl]-4-methylazoline-2,5-dione (S03742) or a salt thereof, having the following structure:

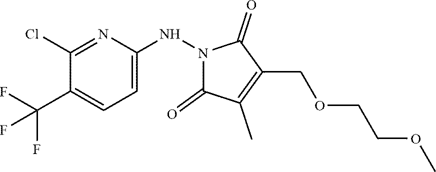 [SMILES: COCCOCC1=C(C)C(=O)N(Nc2ccc(C(F)(F)F)c(Cl)n2)C1=O]

Embodiment P82. The method of embodiment P5, wherein the compound is 1- {[6-chloro-5-(trifluoromethyl) (2-pyridyl)]-amino} -4-(3-hydroxyhexyl)-3-methylazoline-2,5-dione (S03552) or a salt thereof, having the following structure:

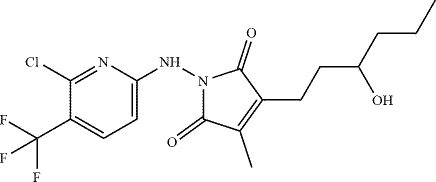 [SMILES: CCCC(O)CCC1=C(C)C(=O)N(Nc2ccc(C(F)(F)F)c(Cl)n2)C1=O]

Embodiment P83. The method of embodiment P5, wherein the compound is 1- {[6-chloro-5-(trifluoromethyl) (2-pyridyl)]amino} -4-(3-hydroxypentyl)-3-methylazoline-2,5-dione (S03745) or a salt thereof, having the following structure:

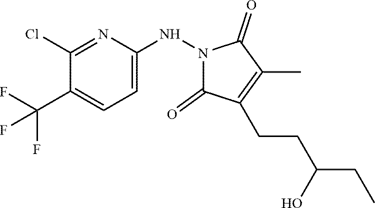 [SMILES: CCC(O)CCC1=C(C)C(=O)N(Nc2ccc(C(F)(F)F)c(Cl)n2)C1=O]

Embodiment P84. The method of embodiment P5, wherein the compound is 1- {[6-chloro-5-(trifluoromethyl) (2-pyridyl)]-amino} -4-methyl-3-[(3-methylbutoxy)methyl]azoline-2,5-dione (S03405) or a salt thereof, having the following structure:

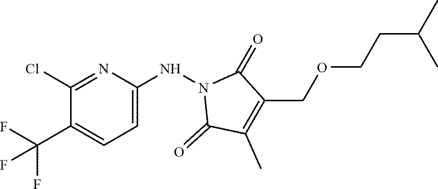 [SMILES: CC1=C(COCCC(C)C)C(=O)N(Nc2ccc(C(F)(F)F)c(Cl)n2)C1=O]

Embodiment P85. The method of embodiment P5, wherein the compound is 3-(butoxymethyl)-1-{[6-chloro-5-(trifluoromethyl)(2-pyridyl)]amino}-4-methylazoline-2,5-dione (S03518) or a salt thereof, having the following structure:

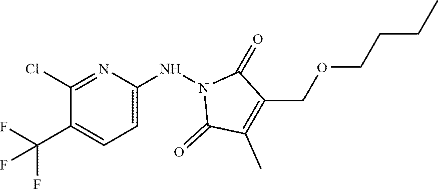 [SMILES: CCCCOCC1=C(C)C(=O)N(Nc2ccc(C(F)(F)F)c(Cl)n2)C1=O]

Embodiment P86. The method of embodiment P5, wherein the compound is 3-[(3,3-dimethylbutoxy)methyl]-1-{[6-chloro-5-(trifluoromethyl)(2-pyridyl)]amino}-4-methylazoline-2,5-dione (S03747) or a salt thereof, having the following structure:

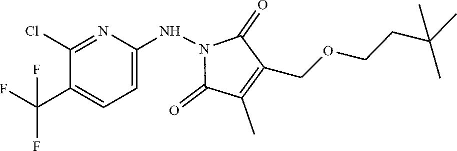 [SMILES: CC1=C(COCCC(C)(C)C)C(=O)N(Nc2ccc(C(F)(F)F)c(Cl)n2)C1=O]

Embodiment P87. The method of embodiment P5, wherein the compound is 1-{[6-chloro-5-(trifluoromethyl)(2-pyridyl)]amino}-3-(2-ethoxyethyl)-4-methylazoline-2,5-dione (S03960) or a salt thereof, having the following structure:

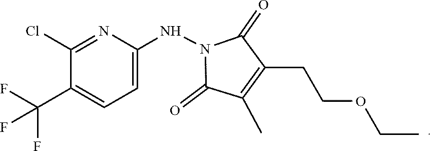

Embodiment P88. The method of embodiment P5, wherein the compound is 1- {[6-chloro-5-(trifluoromethyl) (2-pyridyl)]amino} -4-methyl-3-[(2-methylpropoxy)methyl]azoline-2,5-dione (S03963) or a salt thereof, having the following structure:

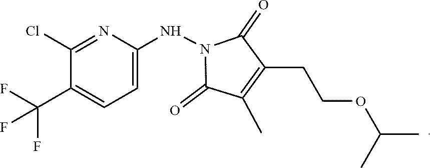

Embodiment P89. The method of embodiment P5, wherein the compound is 3-[(2,2-dimethylpropoxy)methyl]-1-{[6-chloro-5-(trifluoromethyl)(2-pyridyl)]amino}-4-methylazoline-2,5-dione (S03962) or a salt thereof, having the following structure:

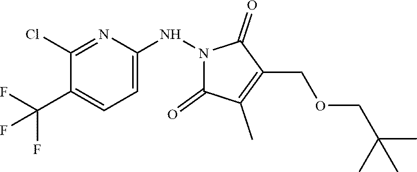 [SMILES: CC1=C(COCC(C)(C)C)C(=O)N(Nc2ccc(C(F)(F)F)c(Cl)n2)C1=O]

Embodiment P90. The method of embodiment P5, wherein the compound is 4-[(1,3-dimethylbutoxy)methyl]-1-{[6-chloro-5-(trifluoromethyl)(2-pyridyl)]amino}-3-methylazoline-2,5-dione (S03964) or a salt thereof, having the following structure:

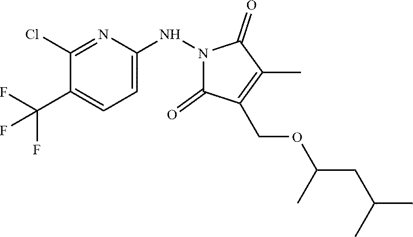 [SMILES: CC1=C(COC(C)CC(C)C)C(=O)N(Nc2ccc(C(F)(F)F)c(Cl)n2)C1=O]

Embodiment P91. The method of embodiment P5, wherein the compound is 4-[(tert-butoxy)methyl]-1-{[6-chloro-5-(trifluoromethyl)(2-pyridyl)]amino}-3-methylazoline-2,5-dione (S03873) or a salt thereof, having the following structure:

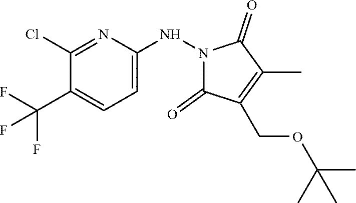 [SMILES: CC1=C(COC(C)(C)C)C(=O)N(Nc2ccc(C(F)(F)F)c(Cl)n2)C1=O]

Embodiment P92. The method of embodiment P5, wherein the compound is 1- {[6-chloro-5-(trifluoromethyl) (2-pyridyl)]amino} -4-methyl-3-[2-(2-methylpropoxy)ethyl]azoline-2,5-dione (S03955) or a salt thereof, having the following structure:

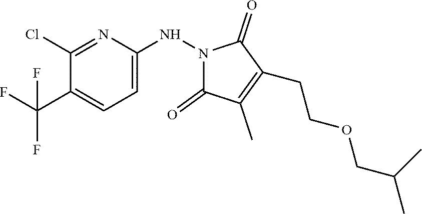 [SMILES: CC1=C(CCOCC(C)C)C(=O)N(Nc2ccc(C(F)(F)F)c(Cl)n2)C1=O]

Embodiment P93. The method of embodiment P5, wherein the compound is 1- {[6-chloro-5-(trifluoromethyl) (2-pyridyl)]amino} -4-methyl-3-[2-(3-methylbutoxy)ethyl]azoline-2,5-dione (S03956) or a salt thereof, having the following structure:

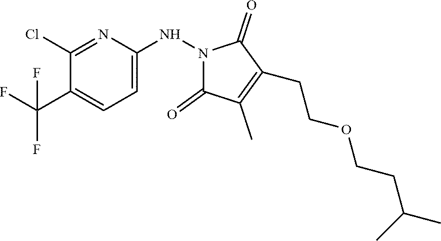 [SMILES: CC1=C(CCOCCC(C)C)C(=O)N(Nc2ccc(C(F)(F)F)c(Cl)n2)C1=O]

Embodiment P94. The method of embodiment P5, wherein the compound is 1- {[6-chloro-5-(trifluoromethyl) (2-pyridyl)]amino} -3-methyl-4-(2-propoxyethyl)azoline-2,5-dione (S04034) or a salt thereof, having the following structure:

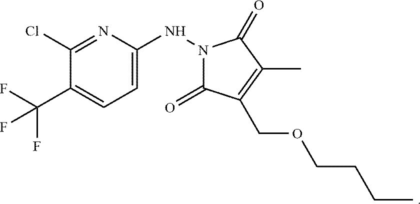

Embodiment P95. The method of any one of embodiments P1-P94, wherein the compound comprises a pharmaceutical composition comprising a pharmaceutically acceptable excipient.

Embodiment P96. The method of any one of embodiments P1-P95, comprising administering an effective amount of the compound to the subject.

Embodiment P97. A method of treating a viral infection in a subject comprising administering to the subject a compound having the formula of compound to treat cell proliferation disorders having the formula of Structure (I):

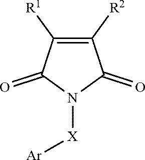

wherein
R¹ and R² are independently chosen from alkyl, substituted alkyl, optionally substituted alkoxy, optionally substituted alkylthio, halogen, optionally substituted aryl, optionally substituted aryloxy, optionally substituted arylthio, or H, where R¹ and R² can also be part of a cyclic alkylene chain that form a fused ring structure;
X is O, S, NR³, or CR⁴R⁵;
Ar is aryl or substituted aryl, including carbocyclic aryl, heterocyclic aryl, monocyclic aryl, polycyclic aryl, and aryl fused with non-aryl (non-aromatic) rings;
R³ is H, alkyl, substituted alkyl, optionally substituted acyl, or as part of a ring structure that connects the N to the Ar ring;
R⁴ and R⁵ are chosen independently from H, alkyl, substituted alkyl, or both can be part of a cyclic alkylene chain that forms a ring structure; R⁴ or R⁵ can also be part of a ring structure that connects to the Ar ring;
or a salt thereof; and
further wherein the compound does not have any one of the following structures:

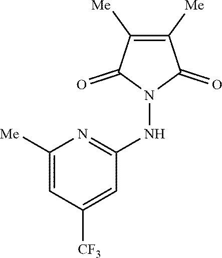 [SMILES: CC1=C(C)C(=O)N(Nc2cc(C(F)(F)F)cc(C)n2)C1=O]

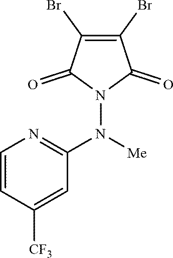 [SMILES: CN(c1cc(C(F)(F)F)ccn1)N1C(=O)C(Br)=C(Br)C1=O]

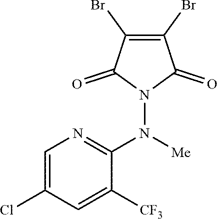 [SMILES: CN(c1ncc(Cl)cc1C(F)(F)F)N1C(=O)C(Br)=C(Br)C1=O]

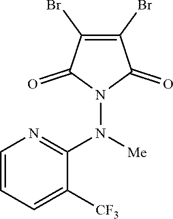 [SMILES: CN(c1ncccc1C(F)(F)F)N1C(=O)C(Br)=C(Br)C1=O]

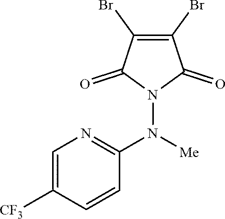 [SMILES: CN(c1ccc(C(F)(F)F)cn1)N1C(=O)C(Br)=C(Br)C1=O]

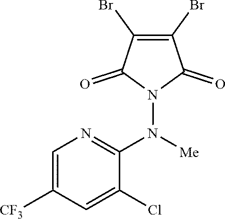 [SMILES: CN(c1ncc(C(F)(F)F)cc1Cl)N1C(=O)C(Br)=C(Br)C1=O]

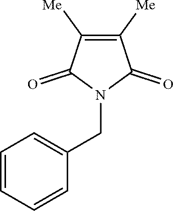 [SMILES: CC1=C(C)C(=O)N(Cc2ccccc2)C1=O]

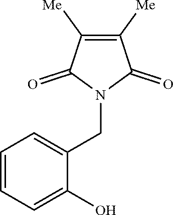 [SMILES: CC1=C(C)C(=O)N(Cc2ccccc2O)C1=O]

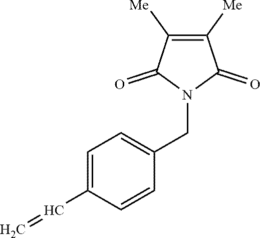 [SMILES: C=Cc1ccc(CN2C(=O)C(C)=C(C)C2=O)cc1]

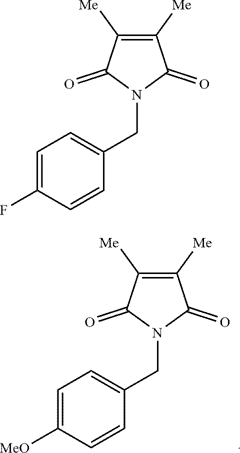

Embodiment P98. The compound of embodiment P1 having the formula of Structure (II) or Structure (III):

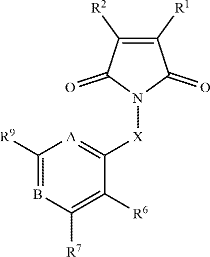

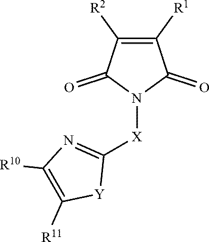

wherein
R¹ and R² are independently chosen from alkyl, substituted alkyl, optionally substituted alkoxy, optionally substituted alkylthio, halogen, optionally substituted aryl, optionally substituted aryloxy, optionally substituted arylthio, or H, where R¹ and R² can also be part of a cyclic alkylene chain that form a fused ring structure;
X is O, S, NR³, or CR⁴R⁵;
Aryl or substituted aryl includes carbocyclic aryl, heterocyclic aryl, monocyclic aryl, polycyclic aryl, and aryl fused with non-aryl (non-aromatic) rings;
R³ is H, alkyl, substituted alkyl, optionally substituted acyl, or as part of a ring structure that connects the N to the Ar ring;
R⁴ and R⁵ are chosen independently from H, alkyl, substituted alkyl, or both can be part of a cyclic alkylene chain that forms a ring structure; R⁴ or R⁵ can also be part of a ring structure that connects to the Ar ring;
A is N or CH;
B is CR⁸ or N;
R⁶, R⁷, R⁸, and R⁹ are independently chosen from H, alkyl, substituted alkyl, halogen, optionally substituted aryl, optionally substituted heteroaryl, optionally substituted alkoxy, optionally substituted aryloxy, cyano, nitro, optionally substituted alkylthio, optionally substituted alkylsufinyl, optionally substituted alkylsulfonyl, optionally substituted arylthio, optionally substituted acyl, optionally substituted amino, carboxyl, optionally substituted alkoxycarbonyl, optionally substituted carbamoyl, wherein R⁶ and R⁷, or R⁷ and R⁸, or R⁸ and R⁹ can be part of a cyclic alkylene group forming a fused ring structure;
Y is O, S, or NR¹²;
R¹⁰ and R¹¹ are independently chosen from H, alkyl, substituted alkyl, halogen, optionally substituted aryl, optionally substituted heteroaryl, optionally substituted alkoxy, optionally substituted aryloxy, cyano, nitro, optionally substituted alkylthio, optionally substituted alkylsufinyl, optionally substituted alkylsulfonyl, optionally substituted arylthio, optionally substituted acyl, optionally substituted amino, carboxyl, optionally substituted alkoxycarbonyl, optionally substituted carbamoyl, wherein R¹⁰ and R¹¹ can be part of a cyclic alkylene group forming a fused ring structure;
R¹² is H, alkyl, substituted alkyl, aryl, acyl, or sulfonyl groups;
or a salt thereof.

Embodiment P99. The compound of embodiment P2, wherein one of R¹ and R² is methyl, and the other of R¹ and R² is alkyl or alkyl substituted with alkoxy, hydroxy, carboxy, alkoxycarbonyl, optionally substituted carbamoyl, or optionally substituted cyclic aminocarbonyl.

Embodiment P100. The compound of embodiment P2, wherein both R¹ and R² is part of a cyclic alkylene chain that form a fused ring structure.

Embodiment P101. The compound of embodiment P2, wherein X is NR³ or CR⁴R⁵, wherein R³ is H, alkyl or acyl, R⁴ and R⁵ are independently chosen from H or alkyl.

Embodiment P102. The compound of embodiment P2, wherein R⁶, R⁷, R⁸, R⁹, R¹⁰, and R¹¹ are independently chosen from H, alkyl, halogen, trifluoromethyl, optionally substituted phenyl, alkoxy, cyano, or alkyl substituted with phenyl, optionally substituted amino, optionally substituted cyclic amino, or acyloxy.

Embodiment P103. The compound of embodiment P2, wherein Y is S.

Embodiment P104. The compound of embodiment P2, wherein two adjacent substitutions R⁶ and R⁷, or R⁸ and R⁹, or R¹⁰ and R¹¹ form a fused and substituted benzene ring.

Embodiment P105. The method of any one of the preceding embodiments, wherein contacting a cell with the compound inhibits viral replication.

Embodiment P106. The method of any one of the preceding embodiments, wherein the compound binds XPO1.

Embodiment P107. The method of any one of the preceding embodiments, wherein the compound binds to Cys528 of XPO1.

Embodiment P108. The method of any one of the preceding embodiments, wherein the binding is reversible.

Embodiment P109. The method of any one of the preceding embodiments, wherein contacting a cell with the compound increases nuclear retention of XPO1 in the cell.

Embodiment P110. The method of any one of the preceding embodiments, wherein contacting a cell with the compound increases nuclear retention of a viral protein.

Embodiment P111. The method of any one of the preceding embodiments, wherein contacting a cell with the compound increases nuclear retention of a viral ribonucleoprotein (vRNP).

Embodiment P112. The method of any one of the preceding embodiments, wherein contacting a cell with the compound blocks nuclear export of a vRNP or viral protein.

Embodiment P113. The method of any one of the preceding embodiments, wherein the inhibition of replication decreases the infectivity of the virus.

Embodiment P114. The method of any one of the preceding embodiments, wherein inhibition of replication decreases the viral titer of the virus.

Embodiment P115. The method of any one of the preceding embodiments, wherein the viral infection is chosen from those caused by viruses belonging to the Togaviridae, Arenaviridae, Poxviridae, Toroviridae, Paramyxoviridae, Herpesviridae, Retroviridae, Coronaviridae, Flaviviridae, Bunyaviridae, Pneumoviridae, Filoviridae, Adenoviridae, Papovaviridae, Hepadnaviridae, or Orthomyxoviridae families.

Embodiment P116. The method of any one of the preceding embodiments, wherein the viral infection is caused by dengue virus (DENV), respiratory syncytial virus (RSV), Venezuelan equine encephalitis virus (VEEV), influenza virus, human immunodeficiency virus (HIV), herpes simplex virus (HSV), cytomegalovirus (CMV), Ebola virus, rubulavirus, or coronavirus.

Embodiment P117. The method of any one of the preceding embodiments, wherein the viral infection is an influenza infection.

Embodiment P118. The method of any one of the preceding embodiments, wherein the viral infection is an influenza A, influenza B, or influenza C infection.

Embodiment P119. The method of any one of the preceding embodiments, wherein the influenza infection comprises infection by H1N1, H1N2, H3N2, H5N1, or H7N9 subtypes of influenza.

Embodiment P120. The method of any one of the preceding embodiments, wherein the viral infection is a Coronavirus infection.

Embodiment P121. The method of any one of the preceding embodiments, wherein the viral infection comprises infection by SARS-CoV2.

Embodiment P122. The method of any one of the preceding embodiments, wherein the disease caused by the viral infection is COVID-19.

Embodiment P123. The method of any one of the preceding embodiments, wherein the method of treating a viral infection comprises reducing the duration of infection.

Embodiment P124. The method of any one of the preceding embodiments, wherein the method of treating a viral infection comprises reducing the symptoms of infection.

Embodiment P125. The method of any one of the preceding embodiments, wherein the method of treating a viral infection comprises reducing the severity of the infection.

Embodiment P126. The method of any one of the preceding embodiments, wherein the method of treating a viral infection comprises reducing viral infectivity.

Embodiment P127. The method of any one of the preceding embodiments, wherein the method of treating a viral infection comprises reducing viral replication.

Embodiment P128. The method of any one of the preceding embodiments, wherein the method of treating a viral infection comprises reducing viral shedding.

Embodiment P129. The method of any one of the preceding embodiments, wherein the subject is a human patient.

Embodiment P130. The method of any one of the preceding embodiments, wherein the subject is a cell and method is an in vitro method.

Embodiment P131. The method of any one of the preceding embodiments, wherein the subject is a human patient’s cell in an ex vivo method.

Embodiment P132. A composition as recited in any of the preceding embodiments for use in the manufacture of a medicament for treating a viral infection.

Embodiment P133. The use of any compound recited in any of the preceding embodiments for treating a viral infection.

The following examples are offered to illustrate, but not to limit the claims.

### EXAMPLES

### Example 1: Felezonexor Reduces Viral Titers in A549 Human Lung Carcinoma Cells Infected with Influenza A Virus

In this example, the ability of felezonexor to inhibit influenza A viral replication in A549 human lung carcinoma cells was evaluated.

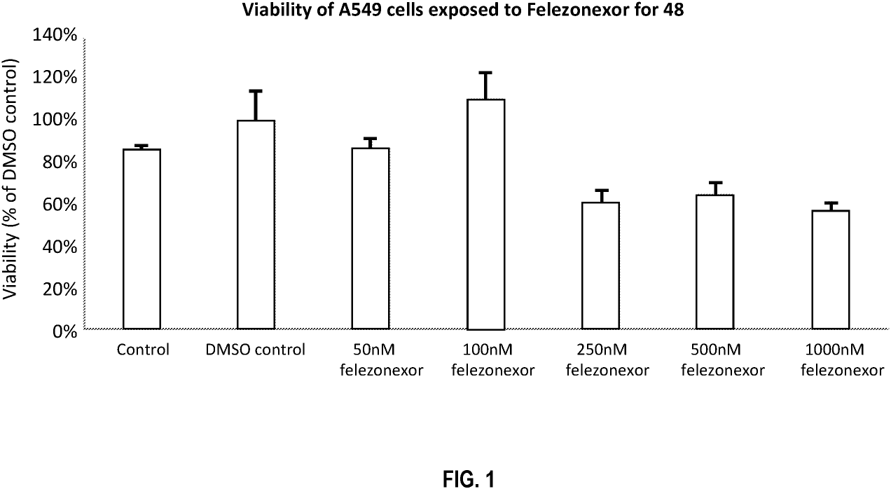

First, the cytotoxicity of felezonexor against uninfected A549 cells was determined. Cultures containing 50, 100, 250, 500, or 1000 nM felezonexor or DMSO control were evaluated in triplicate for cytotoxicity by MTT assay at the 24 or 48 hours post exposure time point. Oseltamivir carboxylate at a concentration of 10 µM was tested as an assay control. The conversion of the water soluble MTT (3-(4,5-dimethylthiazol-2-yl)-2,5-diphenyltetrazolium bromide) (Sigma) to an insoluble formazan was determined by analyzing the felezonexor treated cells. After 48 hours of incubation, 25 µL of the 12 mM MTT stock solution was added to each well. The plates were incubated at 37° C. in a humidified incubator with a 5% CO₂ atmosphere for 2.0 hours. DMSO was then added to each well, mixed and incubated for 15 minutes. Two 100 µL samples were transferred from each well to a 96-well plate and read at a wavelength of 540 nm. The absorbance was calculated as a percent relative to the vehicle control. As shown in Table A (below) and FIG. 1, A549 cells tolerated felezonexor up to 100 nM with relative viability equivalent or greater than untreated controls after 48 hours of treatment. At 500 nM a relative viability of 71.6% was observed. Based on these data, A549 cells and the 48-hour incubation time were utilized for the yield reduction assay.

TABLE A

Cytotoxicity data for felezonexor.
48 hrs post A549

| | OC | 1000 nM | 500 nM | 250 nM | 100 nM | 50 nM | Os |
|---|---|---|---|---|---|---|---|
| O.D. | 0.6065 | 0.399 | 0.455667 | 0.431333 | 0.780833 | 0.612333 | 0.516 |
| % Dif | 100 | 58.727 | 71.59893 | 66.24378 | 125.1321 | 100.9572 | 83.87528 |

Next, virus yield reduction assays were carried out. A549 cells were cultured in 24 well plates one day before treatment with felezonexor. Cells were inoculated at a MOI of 0.1 PFU/cell with influenza A virus and incubated for one hour in the presence of felezonexor. Following 1 hr virus adsorption, cells were washed; wells were then overlayed with 1.0 mL MEM containing felezonexor without serum and incubated at 37° C. under 5% CO₂ environment. Virus and cell control wells were overlayed with MEM containing 0.005% DMSO. Oseltamivir carboxylate, at a single concentration was tested as an assay control. Prior to infection, cells were pretreated with oseltamivir carboxylate for 30 minutes. Oseltamivir remained in the culture during viral inoculation and throughout the duration of the study. At 48 hours post inoculation, infected wells were scored for cytopathic effect (CPE). The supernatants collected at 48 hrs were clarified by centrifugation, aliquoted, and stored at ≤ -65° C. for analysis. Virus titers were determined by TCID50 assay in Madin-Darby Canine Kidney (MDCK) cells. Briefly, samples kept at ≤-65° C. were thawed and centrifuged to remove cellular debris. The resulting supernatant was diluted 10-fold in triplicate in 96-well microtiter plates in Modified Eagle Medium (MEM) (Gibco, Carlsbad, CA, USA) containing Penicillin/Streptomycin, 3% NaCO₃, 0.3% BSA fraction V (Sigma St. Louis, MO), 1% MEM vitamin solution (Sigma) and 1% L-glutamine (Mediatech, Manassas, VA, USA). After 10-fold serial dilutions were made, 100 µL were transferred into respective wells of a 96-well plate which contained a monolayer of MDCK cells. Plates were incubated at 37° C. ± 2° C. in 5 ± 2% CO₂ ≥ 70% humidity. After 72 hours, the wells were observed for cytopathogenic effect (CPE). Supernatant from each well (50 µl) was transferred to a 96 well plate and the hemagglutination (HA) activity determined and recorded. The HA activity of the supernatant was assessed by HA assay with 0.5% packed turkey red blood cells (tRBCs). TCID50 titers were calculated using the method of Reed-Meunch.

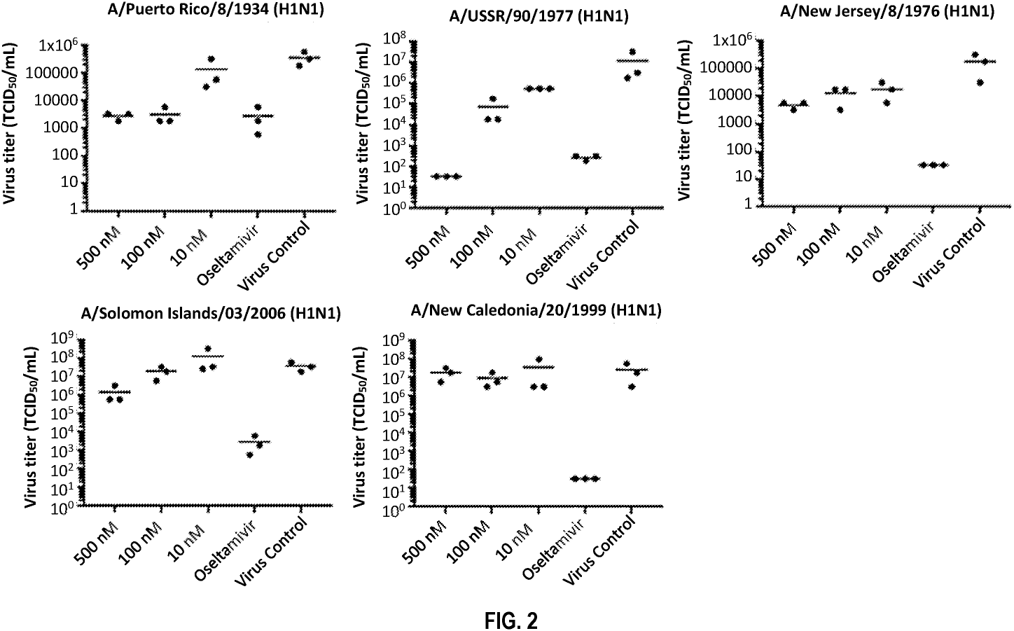

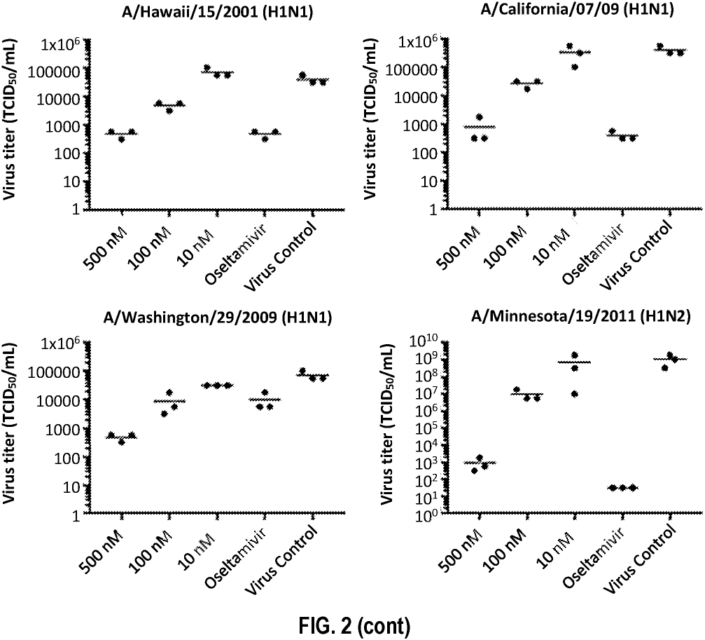

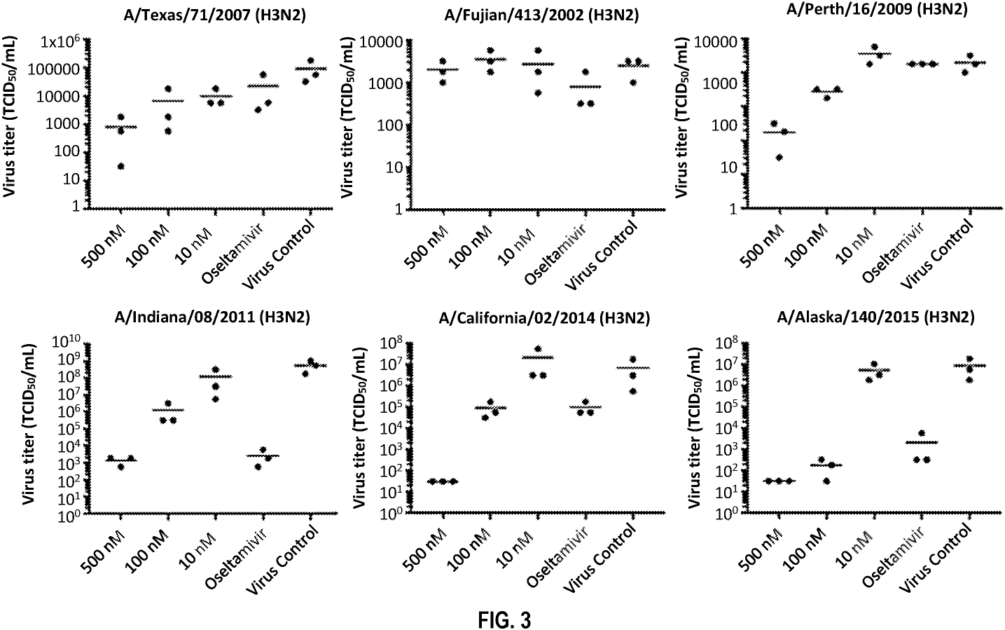

Viral titer data are presented in FIG. 2 (H1N1), FIG. 3 (H3N2) and FIG. 4 (H5N1 and H7N9). For H1N1 viruses, a reduction in viral titers was observed for all viruses except for A/New Caledonia/20/1999 (H1N1) which showed no reduction in viral titers at all concentrations tested. At 100 nM felezonexor, a reduction of greater than 1.0 log₁₀TCID50/mL was observed against A/Minnesota/19/2011 (H1N1), A/USSR/90/1977 (H1N1), A/New Jersey/8/1976 (H1N1), A/Puerto Rico/8/1934 (H1N1) A/California/07/2009 (H1N1). Only A/USSR/90/1977 (H1N1) had a reduction of greater than 1.0 log₁₀TCID50/mL at 10 nM felezonexor.

For H3N2 viruses, a reduction in viral titers was observed for all viruses except for A/Fujian/413/2002 (H3N2) which showed no reduction in viral titers at all concentrations tested. At 100 nM felezonexor, a reduction of greater than 1.0 log₁₀TCID_{50/}mL was observed against A/Indiana/08/2011 (H3N2), A/California/02/2014 (H3N2), A/Texas/71/2007 (H3N2) and A/Alaska/140/2015 (H3N2).

For avian influenza viruses of H5N1 and H7N9 subtypes, a reduction in viral titers was observed for all viruses tested. At 100 nM felezonexor, a reduction of greater than 1.0 log₁₀TCID50/mL was observed against A/Hong Kong/156/1997 (H5N1), A/Egypt/N6658/2011 (H5N1), A/Indonesia/05/2005 (H5N1), A/Anhui/1/2013 (H7N9). Against A/Vietnam/1203/2004 (H5N1), a reduction of greater than 1.0 log₁₀TCID50/mL was only observed at the 500 nM concentration. However, due to the cytotoxicity observed at the 500 nM concentration, this reduction in viral titers is likely attributed to the loss of cells and not an antiviral effect. Only A/Anhui/1/2013 (H7N9) had a reduction of greater than 1.0 log₁₀TCID50/mL at 10 nM felezonexor.

Table B provides individual virus titers (log₁₀TCID50/mL).

TABLE B

Virus titers of Influenza A strains treated with felezonexor

| Replicate Number | Felezonexor | | | Oseltamivir | Virus Control |
|---|---|---|---|---|---|
| | 500 nM | 100 nM | 10 nM | 100 µM | |
| A/Solomon Islands/03/2006 (H1N1) | | | | | |
| 1 | 6.50 | 6.75 | 7.38 | 3.75 | 7.50 |
| 2 | 5.75 | 7.50 | 8.50 | 3.25 | 7.25 |
| 3 | 5.75 | 7.25 | 7.50 | 2.75 | 7.75 |
| AVG | 6.00 | 7.17 | 7.79 | 3.25 | 7.50 |
| S.D. | 0.43 | 0.38 | 0.62 | 0.50 | 0.25 |
| DIF* | -1.50 | -0.33 | 0.29 | -4.25 | 0.00 |
| A/Hawaii/15/2001 (H1N1) | | | | | |
| 1 | 2.50 | 3.75 | 4.75 | 2.75 | 4.75 |
| 2 | 2.75 | 3.75 | 4.75 | 2.50 | 4.50 |
| 3 | 2.75 | 3.50 | 5.00 | 2.75 | 4.50 |
| AVG | 2.67 | 3.67 | 4.83 | 2.67 | 4.58 |
| S.D. | 0.14 | 0.14 | 0.14 | 0.14 | 0.14 |
| DIF* | -1.92 | -0.92 | 0.25 | -1.92 | 0.00 |
| A/California/07/2009 (H1N1) | | | | | |
| 1 | 3.25 | 4.25 | 5.50 | 2.75 | 5.50 |
| 2 | 2.50 | 4.50 | 5.75 | 2.50 | 5.75 |
| 3 | 2.50 | 4.50 | 5.00 | 2.50 | 5.50 |
| AVG | 2.75 | 4.42 | 5.42 | 2.58 | 5.58 |
| S.D. | 0.43 | 0.14 | 0.38 | 0.14 | 0.14 |
| DIF* | -2.83 | -1.17 | -0.17 | -3.00 | 0.00 |
| A/Minnesota/19/2011 (H1N1) | | | | | |
| 1 | 2.50 | 6.75 | 7.25 | 1.50 | 8.50 |
| 2 | 3.25 | 7.25 | 8.50 | 1.50 | 9.00 |
| 3 | 2.75 | 6.75 | 9.25 | 1.50 | 9.00 |
| AVG | 2.83 | 6.92 | 8.33 | 1.50 | 8.83 |
| S.D. | 0.38 | 0.29 | 1.01 | 0.00 | 0.29 |
| DIF* | -6.00 | -1.92 | -0.50 | -7.33 | 0.00 |
| A/New Jersey/8/1976 (H1N1) | | | | | |
| 1 | 3.75 | 3.50 | 4.25 | 1.50 | 5.25 |
| 2 | 3.50 | 4.25 | 4.50 | 1.50 | 5.50 |
| 3 | 3.75 | 4.25 | 3.75 | 1.50 | 4.50 |
| AVG | 3.67 | 4.00 | 4.17 | 1.50 | 5.08 |
| S.D. | 0.14 | 0.43 | 0.38 | 0.00 | 0.52 |
| DIF* | -1.42 | -1.08 | -0.92 | -3.58 | 0.00 |
| A/USSR/90/1977 (H1N1) | | | | | |
| 1 | 1.50 | 5.25 | 5.75 | 2.50 | 7.50 |
| 2 | 1.50 | 4.25 | 5.75 | 2.25 | 6.25 |
| 3 | 1.50 | 4.25 | 5.75 | 2.50 | 6.50 |
| AVG | 1.50 | 4.58 | 5.75 | 2.42 | 6.75 |
| S.D. | 0.00 | 0.58 | 0.00 | 0.14 | 0.66 |
| DIF* | -5.25 | -2.17 | -1.00 | -4.33 | 0.00 |
| * Dif = Difference from Control | | | | | |
| Replicate Number | Felezonexor | | | Oseltamivir | Virus Control |
| | 500 nM | 100 nM | 10 nM | 100 µM | |
| A/Puerto Rico/8/1934 (H1N1) | | | | | |
| 1 | 3.50 | 3.25 | 4.75 | 3.75 | 5.25 |
| 2 | 3.25 | 3.25 | 4.50 | 3.25 | 5.50 |
| 3 | 3.50 | 3.75 | 5.50 | 2.75 | 5.75 |
| AVG | 3.42 | 3.42 | 4.92 | 3.25 | 5.50 |
| S.D. | 0.14 | 0.29 | 0.52 | 0.50 | 0.25 |
| DIF* | -2.08 | -2.08 | -0.58 | -2.25 | 0.00 |
| A/Washington/29/2009 (H1N1) H275Y | | | | | |
| 1 | 2.75 | 3.50 | 4.50 | 3.75 | 5.00 |
| 2 | 2.50 | 3.75 | 4.50 | 3.75 | 4.75 |
| 3 | 2.75 | 4.25 | 4.50 | 4.25 | 4.75 |
| AVG | 2.67 | 3.83 | 4.50 | 3.92 | 4.83 |
| S.D. | 0.14 | 0.38 | 0.00 | 0.29 | 0.14 |
| DIF* | -2.17 | -1.00 | -0.33 | -0.92 | 0.00 |
| A/New Caledonia/20/1999 (H1N1) | | | | | |
| 1 | 6.75 | 7.25 | 8.00 | 1.50 | 7.25 |
| 2 | 7.25 | 6.75 | 6.50 | 1.50 | 6.50 |
| 3 | 7.50 | 6.50 | 6.50 | 1.50 | 7.75 |
| AVG | 7.17 | 6.83 | 7.00 | 1.50 | 7.17 |
| S.D. | 0.38 | 0.38 | 0.87 | 0.00 | 0.63 |
| DIF* | 0.00 | -0.33 | -0.17 | -5.67 | 0.00 |
| A/Indiana/08/2011 (H3N2) | | | | | |
| 1 | 3.25 | 5.50 | 6.75 | 2.75 | 8.25 |
| 2 | 3.25 | 6.50 | 7.50 | 3.75 | 8.50 |
| 3 | 2.75 | 5.50 | 8.50 | 3.25 | 8.25 |
| AVG | 3.08 | 5.83 | 7.58 | 3.25 | 8.33 |
| S.D. | 0.29 | 0.58 | 0.88 | 0.50 | 0.14 |
| DIF* | -5.25 | -2.50 | -0.75 | -5.08 | 0.00 |
| A/California/02/2014 (H3N2) | | | | | |
| 1 | 1.50 | 5.25 | 6.50 | 5.25 | 5.75 |
| 2 | 1.50 | 4.75 | 7.75 | 4.75 | 6.50 |
| 3 | 1.50 | 4.50 | 6.50 | 4.75 | 7.25 |
| AVG | 1.50 | 4.83 | 6.92 | 4.92 | 6.50 |
| S.D. | 0.00 | 0.38 | 0.72 | 0.29 | 0.75 |
| DIF* | -5.00 | -1.67 | 0.42 | -1.58 | 0.00 |
| A/Texas/71/2007 (H3N2) | | | | | |
| 1 | 3.25 | 4.00 | 3.75 | 3.50 | 4.50 |
| 2 | 2.75 | 3.25 | 3.75 | 4.75 | 4.75 |
| 3 | 1.50 | 2.75 | 4.25 | 3.75 | 5.25 |
| AVG | 2.50 | 3.33 | 3.92 | 4.00 | 4.83 |
| S.D. | 0.90 | 0.63 | 0.29 | 0.66 | 0.38 |
| DIF* | -2.33 | -1.50 | -0.92 | -0.83 | 0.00 |

* Dif = Difference from Control

| Replicate Number | Felezonexor | | | Oseltamivir | Virus Control |
|---|---|---|---|---|---|
| | 500 nM | 100 nM | 10 nM | 100 µM | |
| A/Perth/16/2009 (H3N2) | | | | | |
| 1 | 2.25 | 3.50 | 4.75 | 4.25 | 4.00 |
| 2 | 1.50 | 3.50 | 4.25 | 4.25 | 4.25 |
| 3 | 2.50 | 3.25 | 4.25 | 4.25 | 4.50 |
| AVG | 2.08 | 3.42 | 4.42 | 4.25 | 4.25 |
| S.D. | 0.52 | 0.14 | 0.29 | 0.00 | 0.25 |
| DIF* | -2.17 | -0.83 | 0.17 | 0.00 | 0.00 |
| A/Fujian/413/2002 (H3N2) | | | | | |
| 1 | 3.00 | 3.25 | 3.25 | 3.25 | 3.50 |
| 2 | 3.50 | 3.75 | 3.75 | 2.50 | 3.00 |
| 3 | 3.25 | 3.50 | 2.75 | 2.50 | 3.50 |
| AVG | 3.25 | 3.50 | 3.25 | 2.75 | 3.33 |
| S.D. | 0.25 | 0.25 | 0.50 | 0.43 | 0.29 |
| DIF* | -0.08 | 0.17 | -0.08 | -0.58 | 0.00 |
| A/Alaska/140/2015 (H3N2) | | | | | |
| 1 | 1.50 | 1.50 | 6.50 | 2.50 | 6.75 |
| 2 | 1.50 | 2.50 | 7.00 | 3.75 | 6.25 |
| 3 | 1.50 | 2.25 | 6.25 | 2.50 | 7.25 |
| AVG | 1.50 | 2.08 | 6.58 | 2.92 | 6.75 |
| S.D. | 0.00 | 0.52 | 0.38 | 0.72 | 0.50 |
| DIF* | -5.25 | -4.67 | -0.17 | -3.83 | 0.00 |
| A/Hong Kong/156/1997 (H5N1) | | | | | |
| 1 | 2.25 | 3.50 | 4.25 | 1.50 | 4.25 |
| 2 | 2.75 | 3.50 | 4.75 | 1.50 | 5.25 |
| 3 | 2.25 | 3.75 | 4.50 | 1.50 | 4.25 |
| AVG | 2.42 | 3.58 | 4.50 | 1.50 | 4.58 |
| S.D. | 0.29 | 0.14 | 0.25 | 0.00 | 0.58 |
| DIF* | -2.17 | -1.00 | -0.08 | -3.08 | 0.00 |
| A/Egypt/N6658/2011 (H5N1) | | | | | |
| 1 | 4.00 | 4.25 | 5.50 | 2.25 | 4.75 |
| 2 | 4.50 | 4.50 | 4.50 | 1.50 | 5.50 |
| 3 | 3.75 | 4.25 | 4.75 | 2.50 | 5.75 |
| AVG | 4.08 | 4.33 | 4.92 | 2.08 | 5.33 |
| S.D. | 0.38 | 0.14 | 0.52 | 0.52 | 0.52 |
| DIF* | -1.25 | -1.00 | -0.42 | -3.25 | 0.00 |
| A/Vietnam/1203/2004 (H5N1) | | | | | |
| 1 | 6.50 | 8.25 | 8.50 | 5.25 | 7.75 |
| 2 | 6.75 | 7.75 | 8.25 | 5.50 | 8.00 |
| 3 | 6.50 | 9.50 | 7.75 | 4.75 | 8.00 |
| AVG | 6.58 | 8.50 | 8.17 | 5.17 | 7.92 |
| S.D. | 0.14 | 0.90 | 0.38 | 0.38 | 0.14 |
| DIF* | -1.33 | 0.58 | 0.25 | -2.75 | 0.00 |
| * Dif = Difference from Control | | | | | |
| Replicate Number | Felezonexor | | | Oseltamivir | Virus Control |
| | 500 nM | 100 nM | 10 nM | 100 µM | |
| A/Indonesia/05/2005 (H5N1) | | | | | |
| 1 | 4.50 | 6.50 | 7.50 | 4.75 | 7.75 |
| 2 | 5.25 | 6.75 | 7.50 | 4.50 | 8.25 |
| 3 | 4.75 | 7.50 | 7.75 | 4.50 | 8.25 |
| AVG | 4.83 | 6.92 | 7.58 | 4.58 | 8.08 |
| S.D. | 0.38 | 0.52 | 0.14 | 0.14 | 0.29 |
| DIF* | -3.25 | -1.17 | -0.50 | -3.50 | 0.00 |
| A/Anhui/1/2013 (H7N9) | | | | | |
| 1 | 1.50 | 2.25 | 2.75 | 1.50 | 3.25 |
| 2 | 1.50 | 2.50 | 2.75 | 1.50 | 3.25 |
| 3 | 1.50 | 2.75 | 2.50 | 1.50 | 4.75 |
| AVG | 1.50 | 2.50 | 2.67 | 1.50 | 3.75 |
| S.D. | 0.00 | 0.25 | 0.14 | 0.00 | 0.87 |
| DIF* | -2.25 | -1.25 | -1.08 | -2.25 | 0.00 |

Overall, these results demonstrate that felezonexor has antiviral effects against multiple influenza subtypes.

### Example 2: Felezonexor Reduces Influenza Viral Load in Mice in Vivo

In this example, the ability of felezonexor to inhibit influenza viral load in mice in vivo was evaluated. On Day 0, mice (n=7/group) were infected intranasally with A/England/195/2009 (H1N1) at 10,000 PFU. In the vehicle control group, mice were treated orally with 5% gum arabic bid on Days 0, 2, 4 and qd on Days 1 and 3 (regimen A). In the Tamiflu control group, 40 mg/kg Tamiflu was administered bid on Days 0-4 (regimen B). In the felezonexor treatment groups, 100 mg/kg felezonexor was administered either (i) bid on Days 0, 2, 4 and qd on Days 1 and 3 (regimen D) or (ii) qd on Days 0 and 4 and bid on Day 2 (regimen E). Body weights were assessed on a daily basis. On Day 5 of the experiment, mice were sacrificed and viral load in lungs was determined.

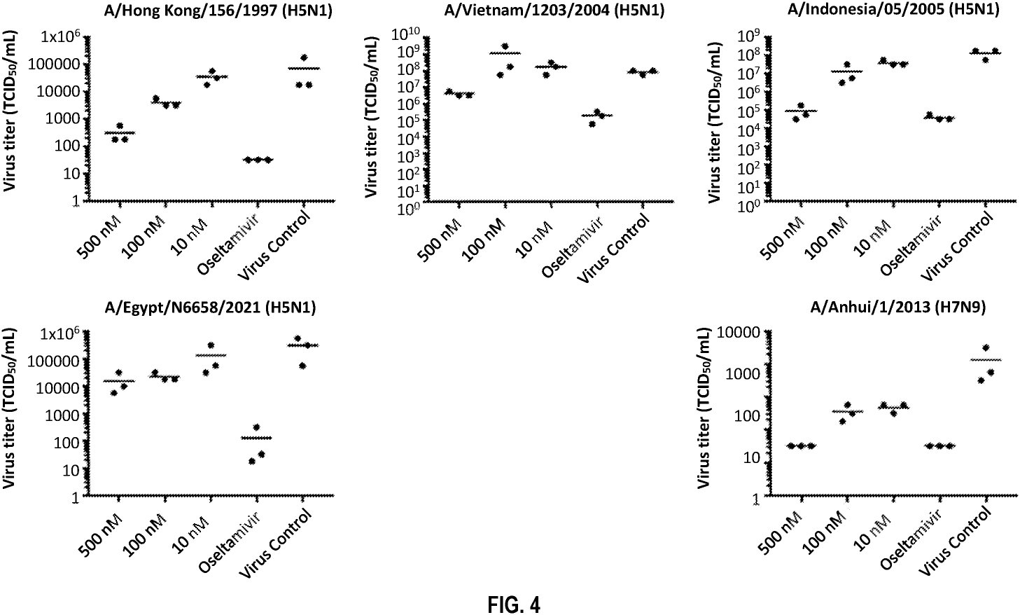

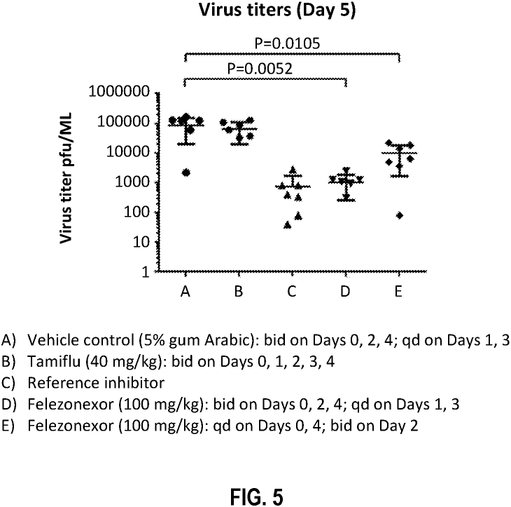

Viral titers in the lung on Day 5 are shown in FIG. 5, and body weights are shown in FIG. 6. Both regimens of felezonexor significantly reduced viral titers in vivo. Felezonexor regimen D (bid on Days 0, 2, 4; qd on Days 1, 3) resulted in a reduction in viral titers by two orders of magnitude, whereas felezonexor regimen E (qd on Days 0, 4; bid on Day 2) resulted in a reduction in viral titers by one order of magnitude. Body weight loss by Day 5 was about 10% for Regimen D and < 10% for Regimen E, indicating that Regimen E was better tolerated by these mice. Regimen C was a proprietary reference inhibitor.

Overall, these experiments demonstrate that oral administration of felezonexor is effective at reducing viral load in the lungs of influenza A-infected mice.

### Example 3: Felezonexor Reduces Viral Titers in MT-2 Human T Cell Line Infected with HIV-1

In this example, the ability of felezonexor to inhibit HIV-1 viral replication was evaluated.

First, the cytotoxicity of felezonexor against the uninfected MT-2 human T cell line was determined. MT-2 cells were incubated with 0.001 - 10 uM felezonexor for 48 hours. Next, MT2 cells were transferred to 1.5 ml Eppendorf tubes, spun down, and washed with PBS. Cells were incubated with 1X passive lysis buffer (Promega, E194) for 10 minutes with vortexing every 2-3 minutes. Next, cell lysates were transferred to a 96 well plate (Costar 3192). Luciferase reagent was added to each well, and luminescence was measured using the Veritas luminometer (Promega). For efficacy experiments, NL4-3 WT HIV-1 virus was added to MT-2 cells in the presence of DMSO control or 0.001 - 10 uM felezonexor for 3 hours to allow entry. After washing, MT-2 cells were further incubated for 48 hours in fresh media containing DMSO or felezonexor). Supernatants containing newly released HIV-1 were added to a HIV-1 reporter cell line TZM-bl, which harbors a firefly luciferase gene whose transcription depends upon HIV-1 infection. The higher luciferase reading is obtained, the more infectious HIV-1 virus is contained in the input supernatant. After 48 hours, TZM-b1 cells were lysed in 1X passive lysis buffer (Promega, E194) for 10 minutes on an orbital shaker. Cell lysates were transferred to a 96 well plate (Costar 3192). Luciferase assay reagent (Promega, E1500) was added to each well, and luminescence was measured using a luminometer.

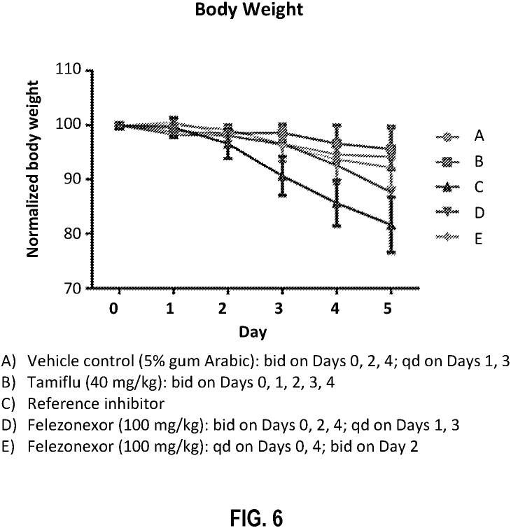

As shown in FIG. 7, felezonexor -treated MT-2 cells released much less infectious HIV-1. At 10 nM, felezonexor reduced the production of infectious virus by more than 50%. In parallel, the cytotoxicity of felezonexor on MT-2 cells was tested. At 1 µM felezonexor, about 50% of the MT-2 cells were killed. Overall, these experiments demonstrate that there is a potential therapeutic window where felezonexor displays minimal toxicity yet remains effective in reducing HIV-1 infection.

### Example 4: Felezonexor Inhibits Nipah Viral Infection in HeLa Cells

In this example, the ability of felezonexor to inhibit HIV-1 viral replication was evaluated.

First, HeLa cells were pre-treated with 0.004 - 10 µM felezonexor at threefold serial dilutions for 2 hours and then infected with Nipah virus (Malaysian strain) at a MOI of 6. Six replicates were performed for each drug concentration. After incubation for 48 hours at 37° C. and 5% CO₂, cells were fixed in 10% buffered formalin, and detection and quantification of viral infection was performed using a high-content imaging assay after immunofluorescent labeling. To measure Nipah virus antigen production, cells were immunostained with polyclonal anti-Nipah virus hyperimmune mouse ascitic fluid. Cells were also stained with Hoechst dye for nuclei detection. Images were acquired on the Opera confocal imaging instrument (Perkin Elmer) and analyzed using Perkin Elmer Acapella algorithms. Data normalization was performed using GeneData analytical software.

Percent inhibition of infection was measured by counting virus-positive syncytia and calculated according to the following formula:

$$\begin{matrix}\% I n h i b i t i o n = \\ \frac{M e d i a n \% V i r u s p o s i t i v e \left(N C\right) - \% V i r u s \text{} p o s i t i v e \left(S a m p l e\right)}{M e d i a n \% V i r u s p o s i t i v e \left(N C\right) - M e d i a n \% V i r u s p o s i t i v e \left(B C\right)} * 100 \%\end{matrix}$$

NC = neutral control with virus infection and 0.5% DMSO mock treatment
BC = blank control with no infection

Percent cell counts, an indicator of cytotoxic effects, was measured by counting the number of nuclei and calculated according to the following formula:

$$\% C e l l c o u n t s = \frac{N u c l e i n u m b e r \left(s a m p l e\right)}{M e d i a n n u c l e i n u m b e r \left(N C\right)} ∗ 100 \%$$

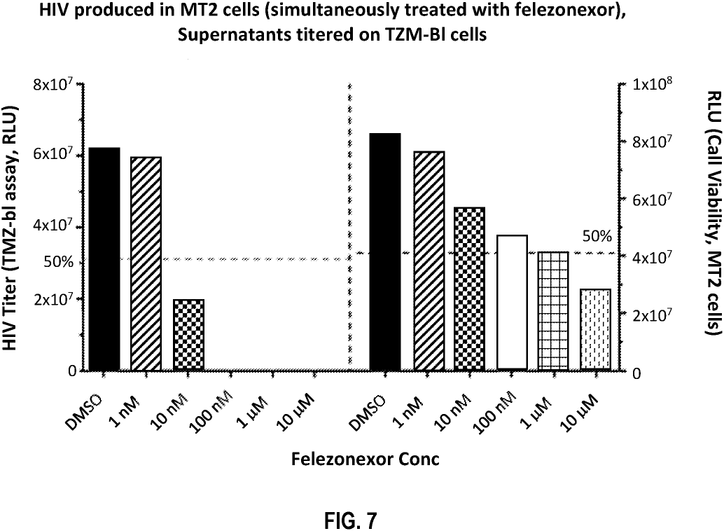

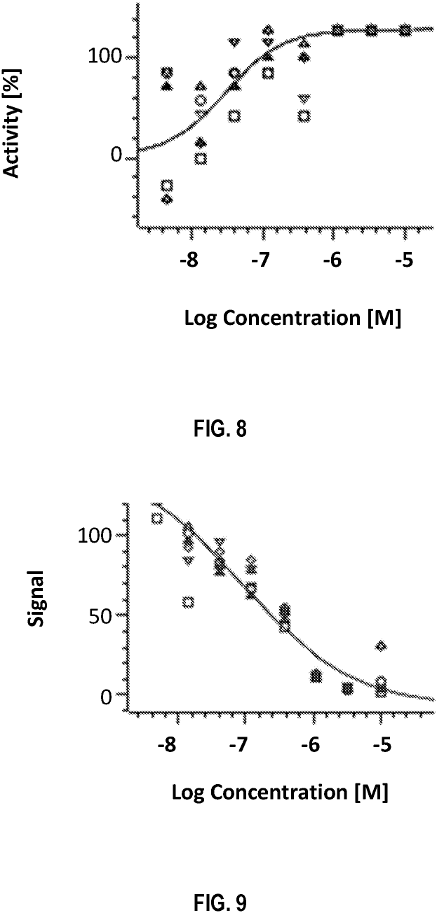

FIG. 8 shows the effect of felezonexor on Nipah virus infection in HeLa cells. The EC50 (compound concentration that reduces viral replication by 50%) of felezonexor was 0.020 µM. FIG. 9 shows the effect of felezonexor on viability of Nipah virus-infected HeLa cells. The CC50 (compound concentration that reduces cell viability by 50%) of felezonexor was 0.24, leading to a selectivity index (SI, CC50/EC50) of 12. Overall, these results demonstrate that felezonexor has antiviral effects against Nipah virus.

### Example 5: Representative Compounds of the Present Disclosure

Table 1 provides structures, ID number (“SCID”), mass, and ¹H NMR values for representative compounds of the present disclosure.

TABLE 1

Representative Compounds

| SCID | Structure | MS (m/e) | 1H NMR | |
|---|---|---|---|---|
| 1 | S00069 | 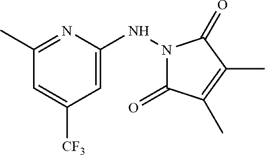 | 300.5 (M+1) | (CDC13, 400 MHz) δ: 6.89 (s, 1H), 6.55 (s, 6.52 (s, 1H),2.44 1H), 6.52 (s, 1H), 2.44 (s,3H), 2.07 (s, 6H) |
| 2 | S00073 | 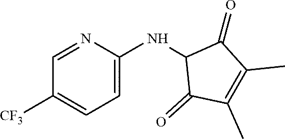 [SMILES: CC1=C(C)C(=O)C(Nc2ccc(C(F)(F)F)cn2)C1=O] | 286.4 (M + 1) | (CDCl3, 300 MHz) δ: 8.37 (s, 1H), 7.70-7.60 (dd, J = 1.8, 8.7 Hz, 1H), 6.96 (s, 1H), 6.60 (d, J = 8.4 Hz, 1H, 2.05 (s, 6H) |
| 3 | S00084 | 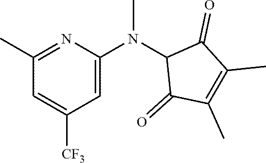 | 314.4 (M + 1) | (CDCl3, 400 MHz) δ: 6.79 (s, 1H), 6.51 (s, 1H), 3.42 (s, 3H), 2.41 (s, 3H), 2.06 (s, 6H) |
| 4 | S00200 | 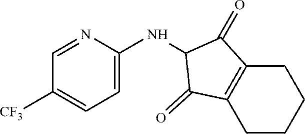 [SMILES: O=C1C2=C(CCCC2)C(=O)C1Nc1ccc(C(F)(F)F)cn1] | | (CDCl3, 300 MHz) δ: 8.37-8.39 (m, 1H), 7.64-7.67 (m, 1H), 7.26 (s, 1H), 6.61-6.64 (d, J = 8.6 Hz, 1H), 2.41 (m,4H), 1.80-1.84 (m, 4H) |
| 5 | S00109 | 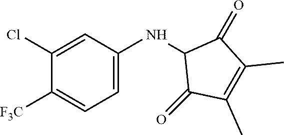 [SMILES: CC1=C(C)C(=O)C(Nc2ccc(C(F)(F)F)c(Cl)c2)C1=O] | 318.0 (M - 1) | (CDCl3, 300 MHz) δ: 7.70 (d, J = 8.7 Hz, 1H), 7.10 (s, 1H), 6.45 (d, J = 8.7 Hz, 1H), 2.07 (s, 6H) |
| 6 | S00170 | 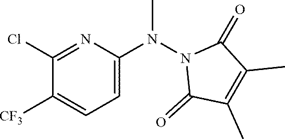 [SMILES: CC1=C(C)C(=O)N(N(C)c2ccc(C(F)(F)F)c(Cl)n2)C1=O] | | (CDCl3, 300 MHz) δ: 7.70 (d, J = 8.4 Hz, 1H), 6.40 (d, J = 8.7 Hz, 1H), 3.44 (s, 3H), 2.08 (s, 6H) |
| 7 | S00186 | 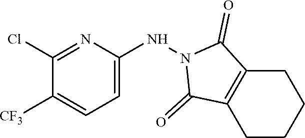 [SMILES: O=C1C2=C(CCCC2)C(=O)N1Nc1ccc(C(F)(F)F)c(Cl)n1] | | (CDCl3, 300 MHz) δ: 7.70 (d, J = 8.7 Hz, 1H), 6.45 (d, J = 8.7 Hz, 1H), 2.50-2.30 (m, 4H), 1.90-1.75 (m, 4H) |
| 8 | S00257 | 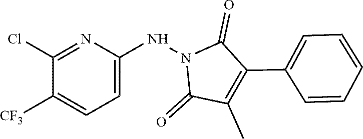 [SMILES: CC1=C(c2ccccc2)C(=O)N(Nc2ccc(C(F)(F)F)c(Cl)n2)C1=O] | | (CDCl3, 300 MHz) δ: 7.79 (d, J = 8.4 Hz, 1H), 7.65-7.62 (m,2H), 7.53-7.48 (m,3H), 7.14 (s, 1H), 6.56 (d, J = 8.4 Hz, 2.31 (s. 3H) |
| 9 | S00333 | 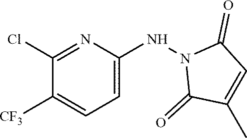 [SMILES: CC1=CC(=O)N(Nc2ccc(C(F)(F)F)c(Cl)n2)C1=O] | 304.2 (M - 1) | (CDCl3, 300 MHz) δ: 8.85-8.75 (br, 1H), 7.95-7.85 (d, J = 8.4 Hz, 1H), 7.24 (s, 1H), 6.20-6.15 (m, 1H), 2.28 (s, 3H), HPLC-MS(m/e): 304.2 (M - 1). |
| 10 | S00108 | 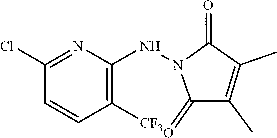 [SMILES: CC1=C(C)C(=O)N(Nc2nc(Cl)ccc2C(F)(F)F)C1=O] | 319.7 (M + 1) | (CDCl3, 300 MHz) δ: 7.80-7.70 (dd, J - 0.6, 7.8 Hz, 1H), 6.95-6.85 (dd, J = 0.6, 7.8 Hz, 1H), 6.82 (s, 1H), 2.07 (s, 6H), HPLC-MS (m/e); 318.0 (M - 1, negative mode) |
| 11 | S00451 | 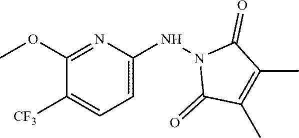 | 314.2 (M - 1) | (CDCl3, 300 MHz) δ: 7.63 (d, J = 8.4 Hz, 1H), 6.82 (s, 1H), 6.28 (d, J = 8.4 Hz, 1H), 3.61 (s, 3H), 2.05 (s, 6H). HPLC-MS (m/e): 314.2 (M - 1) |
| 12 | S00145 | 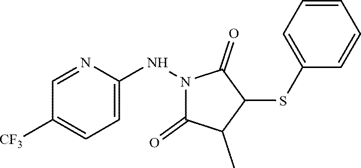 [SMILES: CC1C(=O)N(Nc2ccc(C(F)(F)F)cn2)C(=O)C1Sc1ccccc1] Mixture of isomers | 382.1 (M +1) | (CDCl3, 300 MHz) δ: 8.30 (d, J = 16.6 Hz, 1H), 7.75-7.65 (m, 1H), 7.60 - 7.50 (m, 2H), 7.50-7.30 (m, 3H), 6.80-6.60 (br, 1H), 6.60-6.45 (dd, J = 8.0, 18.4 Hz, 1H), 3.70 (d, J = 5.6 Hz, 0.5H), 3.20-2.95 (t, J = 18.4, 46.4 Hz, 1H), 2.95-2.90 (t, J = 5.6, 7.6 Hz, 0.5H), 1.72 (s, 1.5H), 1.55-1.45 (d, J = 7.2 Hz, 1.5H). HPLC-MS (m/e): 382.1 (M ++ 1) |
| 13 | S00110 | 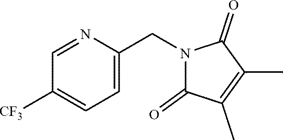 [SMILES: CC1=C(C)C(=O)N(Cc2ccc(C(F)(F)F)cn2)C1=O] | | (CDCl3, 300 MHz) δ: 8.79 (s, 1H), 7.857.89 (dd, J = 2.2, 8.3 Hz, 1H), 7.33-7.36 d, J = 8.0 Hz, 1H), 4.87 (s, 2H), 2.01 (s, 6H) |
| 14 | S00362 | 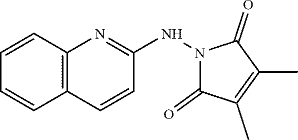 [SMILES: CC1=C(C)C(=O)N(Nc2ccc3ccccc3n2)C1=O] | 268.2 (M + 1) | (CDCl3, 300 MHz) δ: 7.91 (d, J = 8.4 Hz, 1H), 7.69-7.62 (m, 2H), 7.58-7.55 (m, 1H), 7.34-7.29 (m, 1H), 6.79 (d, J = 8.7 Hz, 1H), 2.08 (s, 6H) |
| 15 | S00622 | 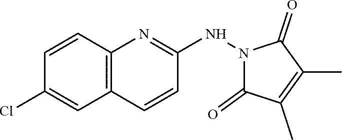 [SMILES: CC1=C(C)C(=O)N(Nc2ccc3cc(Cl)ccc3n2)C1=O] | 302.15 (M + 1) | (CDCl3, 300 MHz) δ: 7.88 (d, J = 8.7 Hz, 1H), 7.62 (m, 2H), 7.51 (d, J = 2.4 Hz, 1H), 6.84 (d, J = 9 Hz, 1H),5.38 (m,1H), 2.09 (s, 6H) |
| 16 | S00585 | 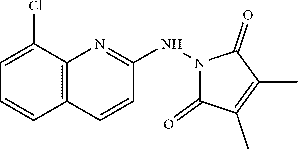 [SMILES: CC1=C(C)C(=O)N(Nc2ccc3cccc(Cl)c3n2)C1=O] | 302.13 (M + 1) | (CDCl3, 300 MHz) δ: 7.90 (d, J = 6.9 Hz, 1H). 7.64-7.66 (dd, J = 0.8, 5.4 Hz, 1H), 7.53-7.56 (dd, J = 0.8, 6.3 Hz. 1H), 7.19-7.23 (dd, J = 5.7, 6 Hz, 1H), 6.86 (d, J = 6.6 Hz, 1H), 2.10 (s, 6H) |
| 17 | S00295 | 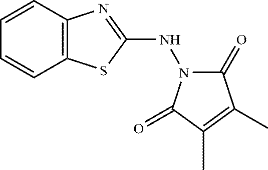 [SMILES: CC1=C(C)C(=O)N(Nc2nc3ccccc3s2)C1=O] | | (CDCl3, 300 MHz) δ: 7.51-7.55 (m, 2H), 7.29-7.35 (m,1H), 7.12-7.17 (m, 1H), 2.07 (s,6H) |
| 18 | S00454 | 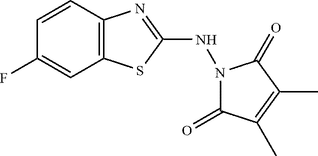 [SMILES: CC1=C(C)C(=O)N(Nc2nc3ccc(F)cc3s2)C1=O] | 292.2 (M + 1) | (CDCl3, 300 MHz) δ: 7.50 (dd, J = 4.8, 9.0 Hz, 1H), 7.28 (m,1H), 7.05 (1d, J = 2.1, 9.0, 17.7 Hz, 1H), 2.02 (s, 6H) |
| 19 | S00590 | 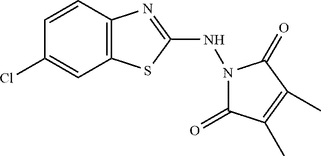 [SMILES: CC1=C(C)C(=O)N(Nc2nc3ccc(Cl)cc3s2)C1=O] | 307.8 (M + 1) | (CDCl3, 300 MHz) δ: 7.53 (d, J = 1.2 Hz, 1H), 7.48 (d, J = 6.3 Hz, 1H), 7.30 (d, J = 1.5 Hz, 1H), 2.07 (s, 6H) |
| 20 | S00756 | 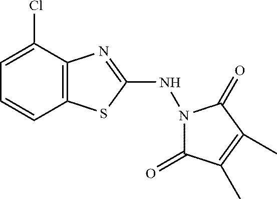 [SMILES: CC1=C(C)C(=O)N(Nc2nc3c(Cl)cccc3s2)C1=O] | | (CDCl3, 300 MHz) δ: 2.02 (s, 6H), 7.07 (d, J = 12 Hz, 1H), 7.32 (d, J = 6.9 Hz, 1H), 7.46 (d, J = 6.9 Hz, 1H) |
| 21 | S00319 | 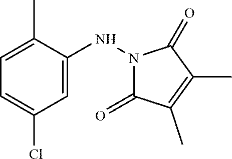 [SMILES: CC1=C(C)C(=O)N(Nc2cc(Cl)ccc2C)C1=O] | | (CDCl3, 300 MHz) δ: 7.05-6.95 (d, J = 7.8 Hz, 1H), 6.90-6.80 (dd, J = 2J, 7.8 Hz, 1H), 6.50-6.45 (d, J = 2.1 Hz, 1H), 5.77 (s, 1H), 2.25 (s, 3H), 2.04 (s, 6H) |
| 22 | S00512 | 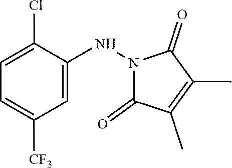 [SMILES: CC1=C(C)C(=O)N(Nc2cc(C(F)(F)F)ccc2Cl)C1=O] | 297.1 (M - 1) | (CDCl3, 300 MHz) δ. 7.18 (d, J = 8.1 Hz, 1H), 7.10 (d, J = 7.8 Hz, 1H), 6.70 (s, 1H), 5.87 (s, 1H), 2.35 (s, 3H), 2.07 (s, 6H) |
| 23 | S00623 | 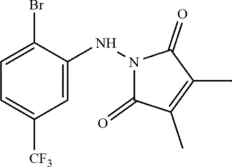 [SMILES: CC1=C(C)C(=O)N(Nc2cc(C(F)(F)F)ccc2Br)C1=O] | | (CDCl3, 300 MHz) δ: 7.60 (dd, J = 0.3. 6.3 Hz, 1H), 7.04-7.07 (m, 1H), 6.75 (d, J = 1.8 Hz, 1H), 6.44 (s, 1H), 2.08 (s, 6H) |
| 24 | S00649 | 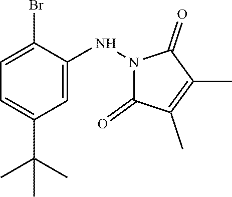 [SMILES: CC1=C(C)C(=O)N(Nc2cc(C(C)(C)C)ccc2Br)C1=O] | 350.9 (M + 1) | (CDCl3, 300 MHz) δ: 7.36 (d, J = 8.4 Hz, 1H), 6.85-6.82 (dd, J = 2.4, 8.4 Hz, 1H), 6.54 (d, J = 2.4 Hz, 1H), 6.32 (s, 1H), 2.06 (s, 6H), 1.20 (s, 9H) |
| 25 | S00305 | 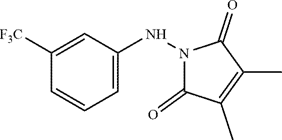 [SMILES: CC1=C(C)C(=O)N(Nc2cccc(C(F)(F)F)c2)C1=O] | | (CDC13,300 MHz) δ; 7.34 (t, J=8.1 Hz 1H), 7.20 (d, J = 8.1 Hz, 1 H), 6.95 (s, IH), 6.90 (dd, 1 Hz 1H) J = 2A 8.1 HZ 1H) , H), 2.06 (s, 6H) 6.02 (s..1H), , |
| 26 | S00515 | 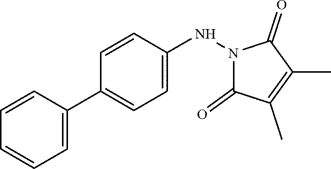 [SMILES: CC1=C(C)C(=O)N(Nc2ccc(-c3ccccc3)cc2)C1=O] | 292.9 (M + 1) | (CDC13, 300 MHz) ô: 7, 52-737 (m, 6H), 7.39 ( 7.39 (d, J = 7.2 Hz, 1H), 6.83-6.80 ( dd,J=2.1, 6.83 -6.80 (dd, J = 2.1, 6.6 HZ, 2H), 5.98 (s, , 1H), 2.05 (s, 6H). HPLC-MS (m/e): 292.9 (M + 1). |
| 27 | S00406 | 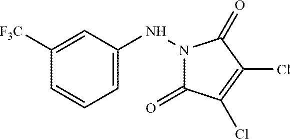 [SMILES: O=C1C(Cl)=C(Cl)C(=O)N1Nc1cccc(C(F)(F)F)c1] | | ( CDC13, 300 MHz) δ: 7.30 (m, 1H), 7.26 (d, J = 9.3 Hz, 1H), 7.01(s, IH), 6.94 ( d J =8.1 Hz, 1H), 6.16 (s. 1H) |
| 28 | S00294 | 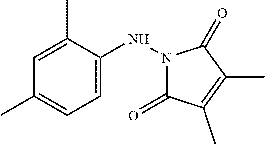 | | (CDC13, 300 MHz) δ: 6.91 (s, 1H), 6.87 (d, J = 7.8 Hz, IH), 6.44 (d, J = 7.8 Hz, 1H), 5.75 (s_{.} 1H), 2.30 (s, 3H), 2.23 (s, 3H), 2.03 (s, 6H) |
| 29 | S00499 | 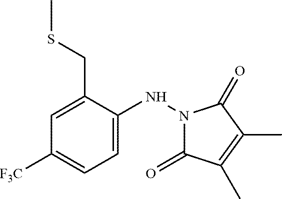 [SMILES: CSCc1cc(C(F)(F)F)ccc1NN1C(=O)C(C)=C(C)C1=O] | | (CDC13, 300 MHz) δ 7.60 (s, 1 H), 7.40 (d, J = 8.4 Hz, 1H), 7.34 (s, 1H), 6.61 (d,J = 8.4 Hz, 1H), 3.88 (s, 2H), 2.03 (s, 6H), 1.59 (s, 3H) |
| 30 | S00699 | 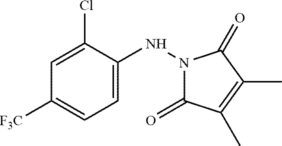 [SMILES: CC1=C(C)C(=O)N(Nc2ccc(C(F)(F)F)cc2Cl)C1=O] | | (CDC13, 300 MHz) δ 2.05 (s, 6H), 6.50 (s, 1H), 6.62 (d, J = 8.7 Hz, 1H), 7.36 (d, J = 8.4, 1H), 7.59 (s, 1H) |
| 31 | S00624 | 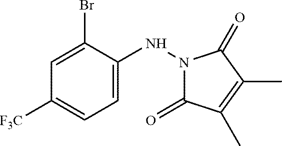 [SMILES: CC1=C(C)C(=O)N(Nc2ccc(C(F)(F)F)cc2Br)C1=O] | | (CDCl3, 300 MHz) δ: 7.75 (d, J = 1.2 Hz, 1H), 7.39-7.42 (m, 1H), 6.59 (d, J = 8.7 Hz, 1 H), 6.50 (s, 1H), 2.06 (s, 6H) |
| 32 | S00627 | 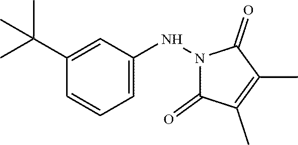 [SMILES: CC1=C(C)C(=O)N(Nc2cccc(C(C)(C)C)c2)C1=O] | 271.1 (M - 1) | (CDCl3, 300 MHz) δ: 7.16-7.11 (t, J = 8.1 Hz, 1H), 6.95 (d, J = 7.8 Hz, 1H), 6.84-6.82 (t, J = 7.8 Hz, 1H), 6.45 (d, J = 8.1 Hz, 1H), 5.90 (s, 1H), 2.04 (s, 6H),1.27 (s, 9H) |
| 33 | S00452 | 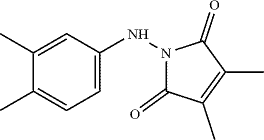 | 245.0 (M + 1) | (CDCl3, 300 MHz) δ: 6.95 (d, J = 8.4 Hz. 1H), 6.56 (d, J = 2.4 Hz, 1H), 6.52-6.48(dd, J = 2.4, 7.8 Hz, 1H), 5.81 (s, 1H), 2.18 (s, 3H)2.15 (s, 3H ), 2.03 (s, 6H) |
| 34 | S00697 | 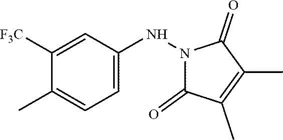 | | (CDCl3, 300 MHz) δ: 7.12 (d. J = 8.1 Hz, 1H), 6.99 (d, J = 2.4 Hz, 1H), 6.79 (d, J = 8.4 Hz, 1H), 5.93 (s, 1H), 2.37 (s, 3H), 2.05 (s, 6H) |
| 35 | S00405 | 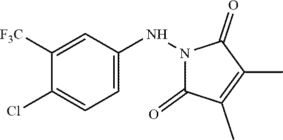 [SMILES: CC1=C(C)C(=O)N(Nc2ccc(Cl)c(C(F)(F)F)c2)C1=O] | | (CDCl3, 300 MHz) δ: 7.33(m, 1H), 7.04 (m, 1H)6.82 (m, 1H), 6.01 (s, 1H), 2.05 ( s, 6H) |
| 36 | S00516 | 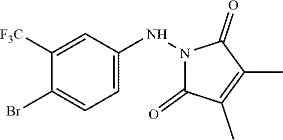 [SMILES: CC1=C(C)C(=O)N(Nc2ccc(Br)c(C(F)(F)F)c2)C1=O] | | (CDCl3, 300 MHz) δ: 7.50 (d, J =9.0 Hz, 1H), 7.05 (d, J = 3.0 Hz, 1H), 6.76-6.72 (dd, J = 2.7, 8.4 Hz, 1H), 6.10 (s, 1H), 2.05 (s, 6H) |
| 37 | S00479 | 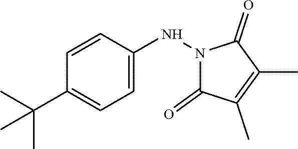 [SMILES: CC1=C(C)C(=O)N(Nc2ccc(C(C)(C)C)cc2)C1=O] | 273 (M + 1) | (CDCl3, 300 MHz) δ: 7.24 (s 1H), 7.21 (s, 1H), 6.70 (s, 1H), 6.67 (s, 1H), 5.87 (s, 1H), 2.03 (s, 6H), 1.25 (s, 9H) |
| 38 | S00456 | 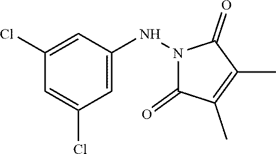 [SMILES: CC1=C(C)C(=O)N(Nc2cc(Cl)cc(Cl)c2)C1=O] | | (CDCl3, 300 MHz) δ: 6.90 (s, 1H), 6.59 (s, 2H) 6.05 (s, 1H), 2.05 (s, 6H) |
| 39 | S00587 | 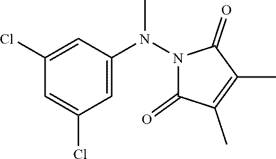 [SMILES: CC1=C(C)C(=O)N(N(C)c2cc(Cl)cc(Cl)c2)C1=O] | 296.9 (M - 1) | (CDCl3 , 300 MHz) δ: 6.86-6.85 (t, J = 1.5 Hz, 1H), 6.55 (d, J = 1.2 Hz, 2H), 3.24 (s, 3H), 2.03 (s, 6h) |
| 40 | S00474 | 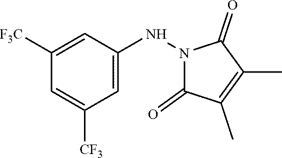 [SMILES: CC1=C(C)C(=O)N(Nc2cc(C(F)(F)F)cc(C(F)(F)F)c2)C1=O] | | (CDCl3, 300 MHz) δ: 7.42 (s, 1H), 7.11 (s, 2H), 6.24 (s, 1H), 2.08 (s, 6H) |
| 41 | S00475 | 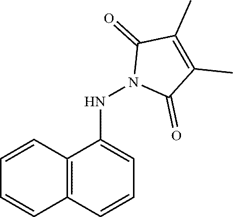 [SMILES: CC1=C(C)C(=O)N(Nc2cccc3ccccc23)C1=O] | 267.5 (M + 1) | (CDCl3, 300 MHz) δ: 7.96-7.99 (t, J = 4.5, 5.1 Hz, 1H), 7.82-7.85 (m, 1H), 7.46-7.52 (m, 3H), 7.27-7.32 (t, J = 7.8, 8.1 Hz, 1H), 6.62 (d, J = 7.8 Hz, 1H), 6.57 (s, 1H), 2.07 (s, 6H) |
| 42 | S00739 | 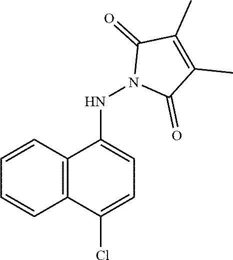 [SMILES: CC1=C(C)C(=O)N(Nc2ccc(Cl)c3ccccc23)C1=O] | | (CDCl3, 300 MHz) δ: 8.24-8.27 (dd, J =0.6, 8.1 Hz, 1H), 7.97-8.00 (d, J = 8.4 Hz, 1H), 7.52-7.64 (m, 2H), 7.35-7.38 (d, J = 8.4 Hz, 1H). 6.60 (s, 1H), 6.54-6.56 (d, J = 8.4 Hz, 1H), 2.07 (s, 6H) |
| 43 | S00651 | 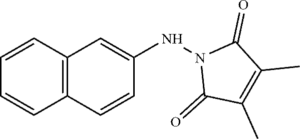 [SMILES: CC1=C(C)C(=O)N(Nc2ccc3ccccc3c2)C1=O] | | {CDCl3 , 300 MHz) δ: 7.73 (d, J = 8.7 Hz, 1H), 7.63 (d, J = 8.1 Hz, 1H). 7.28-7.42 (m, 2H), 7.02-7.07 (dd, J = 2.1, 8.7 Hz, 1H), 6.98 (s, 1H), 6.07 (s, 1H), 2.07 (s, 6H) |
| 44 | S00698 | 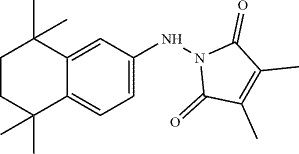 [SMILES: CC1=C(C)C(=O)N(Nc2ccc3c(c2)C(C)(C)CCC3(C)C)C1=O] | 326.9 (M + 1) | (CDCl3, 300 MHz) δ: 7.13(d, J = 8.4 Hz, 1H), 6.73 (s, 1 H), 6.48 (d, J = 7.8 Hz, 1H), 5.82 (s, 1H), 2.03 (s, 6H), 1.63 (s, 4H), 1.20- 1.24 (m, 12H) |
| 45 | S00663 | 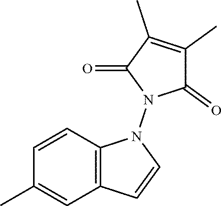 [SMILES: CC1=C(C)C(=O)N(n2ccc3cc(C)ccc32)C1=O] | | (CDCl3, 300 MHz) δ: 9.96 (s, 1H), 6.84 (d, J = 7.5 Hz, 1H), 6.33 (d, J = 8.1 Hz, 1H), 3.80 {m, 2H), 3.09 (m, 2H), 2.25 (s, 2H), 2.02 (s, 6H) |
| 46 | S00662 | 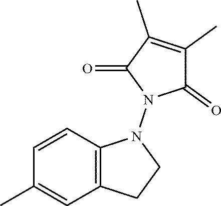 [SMILES: CC1=C(C)C(=O)N(N2CCc3cc(C)ccc32)C1=O] | | (CDCl3, 300 MHz) δ: 7.42 (d, J = 0.9 Hz, 1H, 7.04 (d, J = 8.4 Hz, 1H), 6.95 (d, J= 14.7 Hz, 2H), 6.56 (d, J = 4.2 Hz, 1H), 2.43 (s, 3H), 2.12 (s, 6H) |
| 47 | S00412 | 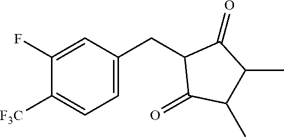 [SMILES: CC1C(=O)C(Cc2ccc(C(F)(F)F)c(F)c2)C(=O)C1C] | | (CDCl3, 300 MHz) δ: 7.51-7.56 (m, 1H), 7.14-7.21 (m, 2H), 4.66 (s, 2H), 2.00 (s, 6H) |
| 48 | S00513 | 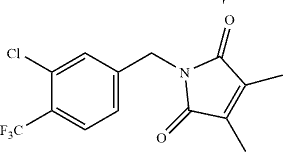 | | (CDCl3,300 MHz) δ: 7.60 (d,J = 8.1 Hz, 1H), 7.46 (s, 1H), 7.30 (d, J = 8.1 Hz, 1H, 4.64 (s, 2H), 1.98 (s, 6H) |
| 49 | S00201 | 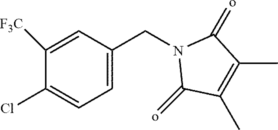 | | (CDCl3, 300 MHz) δ: 7.65 (s, 1H), 7.45 (d, J = 3 Hz, 2H), 4.64 (s, 2H), 1.97 (s, 6H) |
| 50 | S00088 | 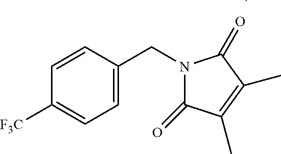 | | (CDCl3, 300 MHz) δ: 7.50 (d, J = 8.1 Hz, 2H), 7.40 (d, J = 8.7 Hz. 2H), 4.69 (s, 2H), 1.97 (s, 6H) |
| 51 | S00408 | 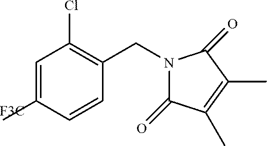 | | (CDCl3, 300 MHz) δ: 7.63 (s, 1H), 7.44-7.46 (d, J = 8.0 Hz, 1H)i, 7.26-7.27 (d, J = 6.2 Hz, 2H) 4.82 (s, 2H), 2.01 (s, 6H) |
| 52 | S00543 | 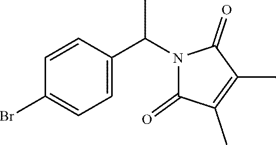 [SMILES: CC1=C(C)C(=O)N(C(C)c2ccc(Br)cc2)C1=O] | | (CDCl3, 300 MHz) δ: 7.43 (m, 2H), 7.30 (m, 2H), 5.25 (m, 1H), 1.92 (s, 6H), 1.77 (d, J = 5.4 Hz, 3H) |
| 53 | S00628 | 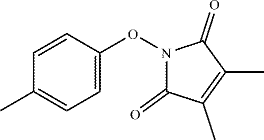 | 230.0 (M - 1) | (CDCl3, 300 MHz) δ: 7.09 (d, J = 8.4 Hz, 2H), 7.00 (d, J = 9.0 Hz, 2H) 2.30 (s, 3H), 2.02 (s, 6H), HPLC-MS (m/e): 230.0 (M - 1). |
| 54 | S00409 | 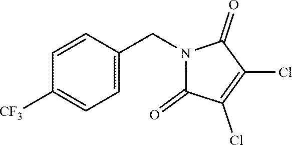 [SMILES: O=C1C(Cl)=C(Cl)C(=O)N1Cc1ccc(C(F)(F)F)cc1] | 324.1 (M + 1) | (CDCl3,300 MHz) δ: 7.60 (d, J = 8.1 Hz, 2H), 7.48 (d, J = 8.4 Hz, 2H), 4.78 (s, 2H) |
| 55 | S00410 | 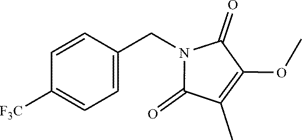 [SMILES: COC1=C(C)C(=O)N(Cc2ccc(C(F)(F)F)cc2)C1=O] | 299.3 (M + 1) | (CDCl3,300 MHz) δ: 7.56 (d, J = 8.1 Hz, 2H), 7.44 (d, J = 8.1 Hz, 2H), 4.67 (s, 2H). 4.16 (s, 3H), 1.98 (s, 3H) |

Table 2 below presents structures, IUPAC name, ID number (“SCID”), mass, and ¹H NMR values for representative compounds.

TABLE 2

REPRESENTATIVE COMPOUNDS Physicochemical Characters

| | | | | |
|---|---|---|---|---|
| SCID | Structure | IUPAC Name | MS (m/e) | 1H NMR |
| S01860 | 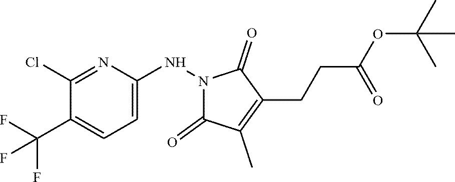 | tert-butyl 3-(1-{[6-chloro-5-(trifluoromethyl) (2-pyridyl)] amino }-4-methyl-2 .5-dio xoazolin-3-yl)propanoate | 432.2 (M⁻ - 1) | (CDCl₃, 300 MHz) δ: 7.79 (d, J =8.5 Hz, 1H), 6.77 (s, 1H), 6.50 (d, J = 8.5 Hz, 1H), 2.75 (t, J= 7.4 Hz, 2H), 2.58 (t, J = 7.0 Hz, 2H), 2.11 (s, 3H), 1.43 (s, 9H), |
| S01861 | 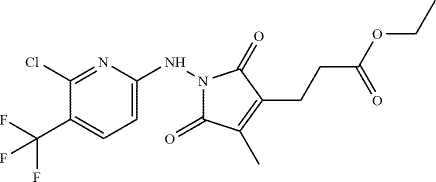 | ethyl 3-(1-{[6- chloro-5-(trifluorou methyl)(2-pyridyl)] amino }-4-methyl-2.5-dioxoazolin- 3-yl)-propanpoate | 404.1 (M⁻ - 1) | (CDCl₃, 300 MHz) δ: 7.79 (d, J = 8.4 Hz, 1H), 6.88 (s, 1H), 6.50 (d, J = 8.4 Hz, 1H), 4.14 (q, J = 7.2 Hz, 2H), 2.79-2.65 (m, 4H), 2.12 (s, 3H), 1.26 (t, J = 7.1 Hz, 3H). |
| S01078 | 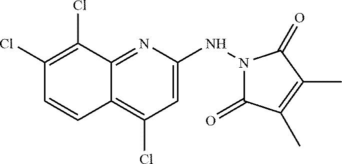 | 3,4-dimethyl-1-[(4,7,8-trichloro (2-quinolyl)) amino\|azoline-2.5 - dione | 370.2 (M⁺ + 1) | (CDCl₃, 300 MHz) δ: 7.88-7.84 (d, J = 8.7 Hz, 1H), 7.45-7.41 (f, J = 9.3 Hz, 1H), 6.94 (s, 1H), 2.10 (s, 6H) |
| S01247 | 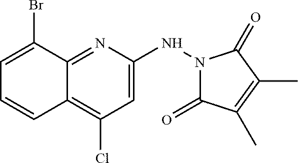 | 1-[(8-bromo-4-chloro(2-quinolyl))amino]-3,4-dimethylazoline-2,5-dione | 380.1 (M⁺ + 1) | (CDCl₃, 300 MHz) δ: 8.00-7.96 (dd, J = 8.4 Hz, 1.2 Hz, 1H), 7.92-7.89 (dd,J = 7.8 Hz,1.2 Hz, 1H), 7.23-7.20 (d, J = 7.8 Hz, 1H), 7.07 (br, 1H), 6.96 (s, 1H), 2.08 (s, 6H) |
| S01589 | 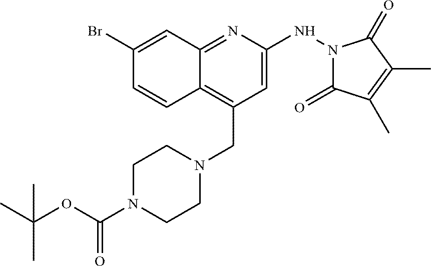 | lert-butyl 4- ({2-[(3,4-dimethyl-2,5-dioxoazolinyl) amino]-7-bromo-4-quinolyl}methyl)-piperazine-Carboxylate | 544.3 (M⁻ - 1) | (CDCl₃, 300 MHz) δ: 7.86-7.83 (d, J =8.4 Hz, 1H), 7.83 (s, 1H), 7.40-7.37 (dd, J = 8.7 Hz, 2.1 Hz, 1H), 7.00-6.80 (br, 1H), 6.85 (s, 1H), 3.65 (s, 2H), 3.41-3.38 (m, 4H), 2.40-2.33 (m, 4H), 2.09 (s, 6H), 1.46 (s, 9H) |
| S01648 | 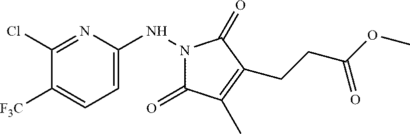 | methyl 3-(1-{[6-chloro-5-(trifluoro-methyl)(2-pyridyl)]amino}-4-methyl-2,5-dioxoazolin-3-yl)propanoate | 390 (M⁻ - 1) | (CDCl₃, 300 MHz) δ: 7.78 (d, J = 8.8 Hz, 1H), 7.11 (s, 1H), 6.50 (d, J = 8.5 Hz, 1H),2.81-2.66 (m, 4H), 2.11 (s, 3H) |
| S01796 | 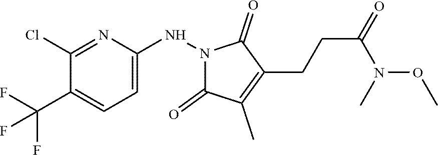 | dioxoazolin-3-yl)-N-methoxy-N*methylpropananide | 419.2 (M⁻ - 1) | (CDCl₃, 300 MHz) δ: 7.76 (d, J = 8.4 Hz, 1H), 7.27 (s, 1H), 6.51 (d, J = 8.5 Hz, 1H),3.67 (s, 3H), 3.17 (s, 3H), 2.81 (s, 4H), 2.11 (s, 3H) |
| S01879 | 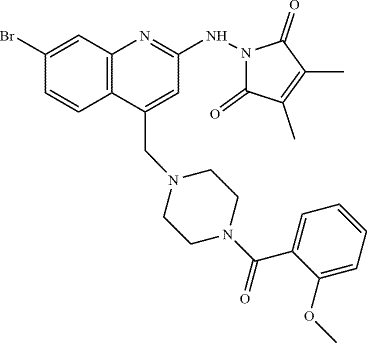 | 1-{[7-bromo-4-({4-[(2-methoxyphenyl)carbonyl] piperazinyl} methyl)(2-quinolyl)] amino}-3,4-dimethylazoline-2,5-dione | 578 (M⁺ + 1) | (CDCl₃, 300 MHz) δ: 7.89-7.83 (m, 2H), 7.41-7.31 (m, 2H), 7.25-7.22 (m, 1H), 7.01-6.98 (m, 1H), 6.96-9.87 (m, 2H), 3.86-3.72 (m, 7H), 3.26-3.20 (m, 2H), 2.63-2.52 (m, 2H), 2.42-2.31 (m, 2H), 2.10 (s, 6H) |
| S01981 | 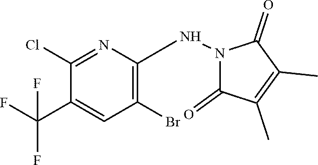 | 1-{[3-bromo-6-chloro-5-(influoro-methyl)(2-pyridyl)] amino}-3,4-dimethylazoline-2,5-dione | 396 (M⁻ - 1) | (CDCl₃, 300 MHz) δ: 7.97 (s, 1H), 7.07 (s, 1H), 2.07 (s, 6H) |
| S00109 | 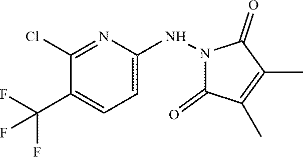 | 1-{[6-chloro-3-(trifluoromethyl) (2-pyridyl)] amino}-3,4-dimethylazoline-2,5-dione | 318.0 (M- - 1) | (CDCl₃, 300 MHz) δ: 7.80-7.70 (d, J = 8.7 Hz, 1H), 7.10 (s, 1H), 6.55-6.45 (d,8.7 Hz, 1H), 2.07 (s, 6H) |
| S00170 | 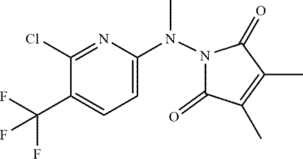 | 1-{[6-chloro-5-{trifluoromethyl) (2-pyridyl)] methylamino}-3,4-dimethylazoline-2,5-dione | | (CDCl₃, 300 MHz) δ: 7.70 (d, J = 8.4 Hz, 1H), 6.40 (d, 8.7 Hz, 1H), 3.44 (s, 3H), 2.08 (s, 6H) |
| S01007 | 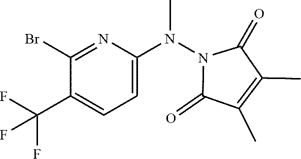 | 1-{[6-bromo-5-(trifluoromethyl) (2-pyridyl)] methylamino}-3,4-dimethylazoline-2,5-diome | 375.9 (M- - 1) | (CDCl₃, 300 MHz) δ: 7.70-7.67 (d, J = 8.7 Hz, 1H), 6.48-6.45 (d, J = 8.7 Hz, 1H), 3.44 (s, 3H), 2.06 (s, 6H) |
| S01554 | 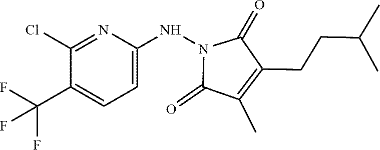 | 1-{[6-chloro-5-(trifluoromethyl) (2-pyridyl)]amino}-4-methyl-3-(3-methylbutyl) azoline-2,5-dione | 374 (M- - 1) | (CDCl₃, 300 MHz) δ: 7.81-7.77 (d, J = 8.7 Hz, 1H), 6.73 (s, 1H), 6.51-6.47 (d, J = 8.7 Hz, 1H), 2.50-2.44 (m, 2H), 2.07 (s, 3H), 1.50-1.42 (m, 3H), 0.97-0.94 (d, J = 6.6 Hz, 6H) |
| S01599 | 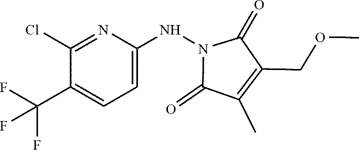 | 1-{[6-chloro-5-trifluoromethyl) (2-pyridyl)]amino}-3-(methoxymethyl)-4-methylazoline-2,5-dione | 348 (M- - 1) | (CDCl₃, 300 MHz) δ: 7.80-7.77 (d, J = 8.4 Hz, 1H), 6.92 (s, 1H), 6.53-6.50 (d, J = 8.7 Hz, 1H), 4.38 (s, 2H), 3.44 (s, 3H), 2.20 (s, 3H) |
| S01455 | 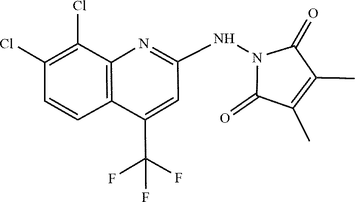 | 1-{(7,8-dichloro-4-(trifluoromethyl) (2-quinolyl)] amino}-3,4-dimethylazoline-2,5-dione | 404 (M⁺ + 1) | (CDCl₃, 300 MHz) δ: 7.78-7.72 (m, 1H), 7.47-7.44 (d, J = 9.4 Hz, 1H), 7.35 (br, 1H), 7.16 (s, 1H), 2.12 (s, 6H) |
| S01711 | 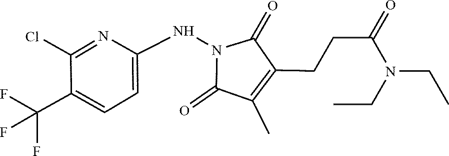 | 3-(1-{[6-chloro-5-(trifluoromethyl) (2-pyridyl)]amino}-4-methyl-2,5-dioxoazoline-3-yl)-N,N-diethylpropanamide | 431 (M- - 1) | (CDCl₃, 300 MHz) δ: 7.77 (d, J = 8.7 Hz 1H), 7.13 (s, 1H), 6.52 (d, J = 8.4 Hz 1H), 3.40-3.26 (m, 4H), 2.84-2.67 (m, 4H), 2.13 (s, 3H), 1.18-1.08 (m, 6H) |
| S01712 | 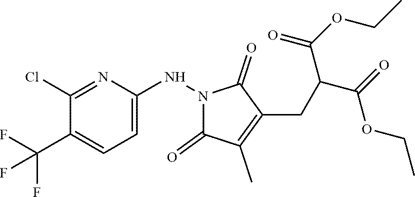 | diethyl 2-[((1-{[6-chloro-5-(trifluoro-methyl)(2-pyridyl)] amino}-4-methyl-2,5-dioxoazolin-3-yl)methyl] propane-1,3-dioate | 476 (M- - 1) | (CDCl₃, 300 MHz) δ: 7.79 (d, J = 8.6 Hz 1H), 6.94 (s, 1H), 6.50 (d, J = 8.2 Hz, 1H), 4.25-4.16 (m, 4H), 3.86 (t, J = 7.9 Hz, 1H), 3.05 (d, 7.9 Hz, 2H), 2.11 (s, 3H), 1.27 (t, J = 7.1 Hz, 6H). |
| S01758 | 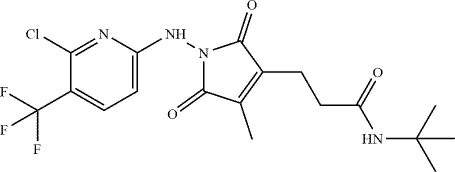 | N-(tert-butyl)-3-(1-{[6-chloro-5-(trifluoromethyl) (2-pyridyl)] amino}-4-methyl-2,5-dioxoazolin-3-yl)propanamide | 431.3 (M- - 1) | (CDCl₃, 300 MHz) δ: 7.79 (d, J = 8.6 Hz, 1H), 7.12 (s, 1H), 6.53 (d, J = 8.6 Hz, 1H), 5.33 (s, 1H), 2.79 (t, J = 7.2 Hz, 2H), 2.43 (t, J = 7.3 Hz, 2H), 2.10 (s, 3H), 1.32 (s, 9H). |
| S01925 | 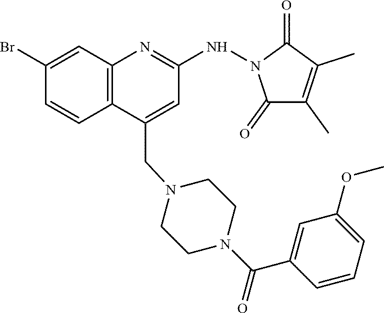 | 1-{[7-bromo-4-{[4-methoxy-phenyl)carbonyl] piperazinyl] methyl)(2-quinolyl)] amino}-3,4-dimethylazoline-2,5-dione | 576.3 (M- - 1) | (CDCl₃, 300 MH_{z}) δ: 7.87-7.83 (m,2H), 7.41-7.37 (dd, J -1.2 Hz, 1.4 Hz, 1H), 7.33-7.27 (m, 1H), 6.96-6.93 (m, 3H) 6.85 (s, 1H), 3.90-3.60 (br, 2H), 3.82 (s, 3H), 3.69 (s, 2H), 3.42 (br, 2H), 2.54 (br, 2H), 2.41 (br, 2H), 2.09 (s, 6H) |
| S00994 | 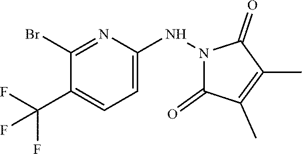 | 1-{[6-bromo-5-(trifluoromethyl) (2-pyridyl)amino]-3,4-dimethylazoline-2,5-dione | 362.0 (M- - 1) | (CDCl₃, 300 MHz) δ: 7.76-7.73 (d, J -8.4 Hz, 1H), 6.77 (br, 1H), 6.53-6.50 (d, J - 8.7 Hz, 1H), 2.08 (s, 6H) |
| S01005 | 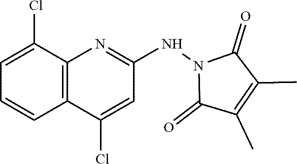 | 1-[(4,8-dichloro (2-quinolyl)) amino]-3,4-dimethylazoline-2,5-dione | ( 336.4 (M⁺ + 1) | (CDCl₃, 300 MHz) δ: 7.95:-7.91 (dd, J = 8.4 Hz, 1.5 Hz, 1H), 7.73-7.69 (dd, J = 7.8 Hz., 1.5 Hz, 1H), 7.33-7.29 (d, J = 8.1 Hz, 1H), 6.94 (s, 1H), 2.11 (s, 6H) |
| S01266 | 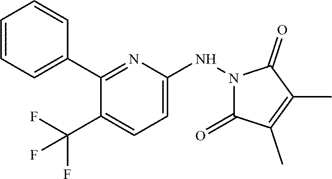 | 3,4-dimethyl-1-{[6-phenyl-5-(trifluoromethyl) (2-pyridyl)amino}azoline-2,5-dione | 360.2 (M⁻ - 1) | (CDCl₃ 300 MHz) δ: 7.86-7.82 (d, J = 9.0 Hz, 1H), 7.45-7.35 (m, 5H), 6.48-6.44 (d, J = 9.0 Hz, 1H), 2.02 (s, 6H) |
| S01470 | 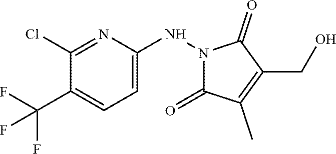 | 1-{[6-chloro-5-(trifluoromethyl) (2-pyridyl)]amino}-3-(hydroxy-methyl)-4-methylazoline-2,5-dione | 336 (M⁺ + 1) | (CDCl₃, 300 MHz) δ: 7.93-7.90 (d, J = 8.5 Hz, 1H), 7.69 (s, 1H), 6.28-6.25, (d, J = 8.5 Hz, 1H), 3.69 (s, 2H), 2.90-2.70 (br, 1H), 2.14 (s, 3H) |
| S01473 | 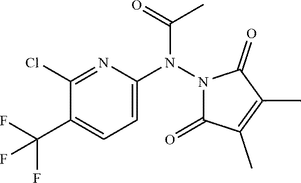 | N-(3,4-dimethyl-2,5-dioxoazolinyl)-N-(6-chloro-5-(trifluoromethyl) (2-pyridyl)}acetamide | 360.0 (M⁻ - 1) | (CDCl₃, 300 MHz) δ: 8.28-8.25 (m, 1H), 7.99-7.97 (d, J = 6.6 Hz, 1H), 2.28 2.11 (s, 6H) |
| S01878 | 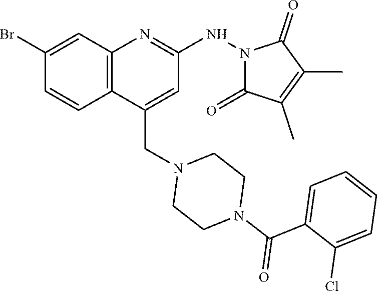 | 1-{(7-bromo-4-({4- [(2-chlorophenyl) carbonyl]piperazinyl] methyl) (2-quinolyl)]amino]-3,4- dimethylazoline-2,5-dione | 582 (M⁺ + 1) | (CDCl₃, 300 MHz) δ: 7.87-7.83 (m, 2H, 7.41-7.26 (m. 5H), 6.85 (s, 1H), 3.84-3.80 (m, 2H). 3.71 (s, 2H), 3.26-3.18 (m, 2H), 2.61-2.57 (m, 2H), 2.47-2.44 (m, 1H), 2.37-2.34 (m, 1H), 2.10 (s, 6H) |
| S01883 | 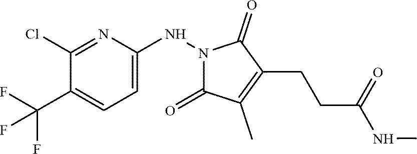 | 3-(1-([6-chloro-5- (trifluoromethyl) (2-pyridyl)]amino}-4-methyl-2, 5-dioxoazolin-3-yl)-N-methylpropanamide | 398.1 (M⁻ - 1) | (CDCl₃, 300 MHz) 7.80-7.77 8.4 Hz, 1H), 7.08 (s, 1H), 6 56-6.53(d, J = 8.4 Hz, 1H), 5.60-5.50 (br, 1H), 2.90-2.75 (m, 5H), 2.55- 2.50 (t,J = 7.2 Hz, 2H), 2.10 (s, 3H) |
| S00585 | 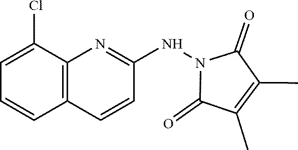 | 1-[(8-chloro{2-quinolyl))amino]-3,4-dimethylazoline-2,5-dione | 302.1 (M⁺ + 1) | (CDCl₃, 300 MHz) δ: 7.92-7.89 (d, J = 7.66-7.63 (dd, J = 8.4 Hz, 1.2 Hz, 1H), 7.56-7.53 (dd, J = 6.3 Hz, 1.2 Hz, 1H), 7.24-7.19 (m, 1H), 6.87-6.84 (d, J = 6.6 Hz, 1H), 2.10 (s, 6H) |
| S00832 | 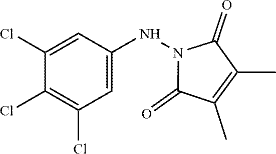 | 3,4-dimethyl-1-[(3,4,5-trichlorophenyl)amino] azoline-2,5-dione | No Mass | (CDCl₃, 300 MHz) δ: 6.75 (s, 2H), 6.04 (s, 1H), 2.06 (s, 6H) |
| S00873 | 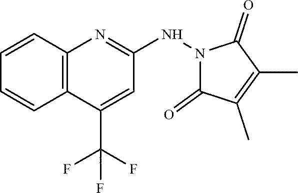 | 3,4-dimethyl-1-{[4-(trifluoromethyl) (2-quinolyl)]amino] azoline-2,5-dione | 336.0 (M⁺ + 1) | (CDCl₃, 300 MHz) δ: 7.97-7.73 (d,J = 8.7 Hz. 1H), 7.78-7.74 (m, 1H), 7.67-7.61 (m, 1H), 7.47-7.41 (m, 1H), 7.13 (s, 1H), 6.88 (s, 1H), 2.11 (s, 6H) |
| S01311 | 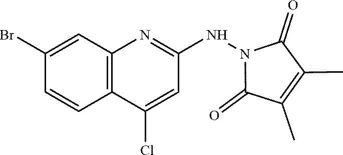 | 1-[(7-bromo-4-chloro(2-quinolyl)) amino]-3,4-)) dimethylazoline-2,5-dione | 380.2 (M⁺ + 1) | (CDCl_{3,} 300 MHz) δ: 7.89-7.84 (m, 2H), 7.50-7.48 (m, 1H), 6.90 (s, 1H), 2.11 (s, 6H) |
| S01313 | 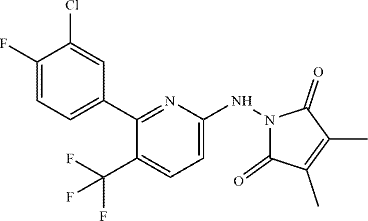 | 1-{[6-(3-chlom-4- fluorophenyl )-5-(trifluoromethyl) (2-pyridyl)] amino]-3,4-dimethylazoline-2 ,S- dione ’ | 414.0 (M⁺ + 1) | (CDCl₃, 300 MHz) δ: 7.89-7.85 (d, J = 8.4 Hz, 1H), 7.51-7.47 (dd, J=7.5 Hz, 2.1 Hz, 1H), 7.40-7.35 (br, 1H), 7.18-7.12 (m,1Hi), 6.82 (s, 1H), 6.62-6.58 (d, J = 8.7 Hz, 1H), 2.05 (s, 6H) |
| S01457 | 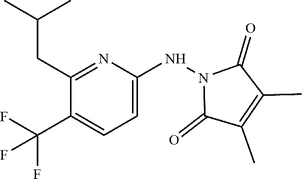 | 3,4-dimethyl-1-{ [6-(2-methyl-(trifluorimethyl)(2-pyridyl)]amino} azoline-2.5-dione - | 340.3 (M⁺ + 1) | (CDCl₃, 300 MHz) δ: 7.70-7.67 (d,J-6.3 Hz, 1H), 6.65 (br, 1H), 6.47-6.44 (d,J- 6.6 Hz, 1H), 2.61 (d, J = 5.4 Hz, 2H), 2.07 (s, 6H), 0.85 (s, 3H), 0.84 (s, 3H) |
| S01737 | 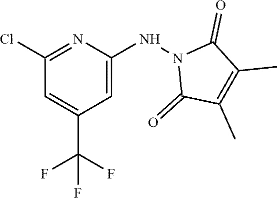 | 1 -{[6-chloro-4-(trifluoromethyl)(2-pyridyl)]amino}, 3,4-dimethyl azoline-2.5 -dione | 320 (M⁺ + 1) | (CDCl₃, 300 MHz) b: 7.03 (s, 1H), 6.93 (s, 1H), 6.62 (s, 1H), 2.07 (s, 6H) |
| S01865 | 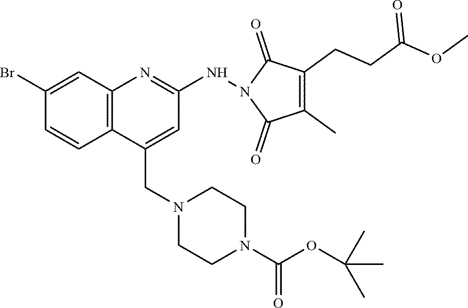 | methyl 3-(1-{[4-({4-[(tert-butyl) oxycaibonyl) piperazinyl) methyl)-7-bromo(2-quinolyl)] amino] -4-methyl-2,5-dioxoazolin-3-yl)propanoate | 616 (M⁺ + 1) | (CDCl₃, 300 MHz) ô: 7.86-7.79 2H), 7.40-7.37 (d*d,J₁ -8.7 Hz, J₂₋2.1 Hz, 1H), 6.85 (s, 1H), 3.72 (s, 3H), 3.65 (s, 2H), 3.42-3.39 (m, 4H), 2.83-2.81 (t, 2H), 2.74-2.72 (t, 2H), 2.40 (m, 4H), 2.14 (s, 3H), 1.46 (s, 9H) |
| S01880 | 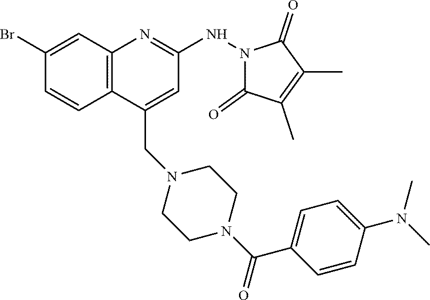 | 1-({4-[(4-([4-(dimethylamino ) phenyl] carbonyl)piperasziayl) methyl]-7-bromo(2-quinolyl)} amino)-3,4-dimethylaxoline-2.53-dione | 591 (M⁺ + 1) | (CDCl_{3,} 300 MHz) ô: 7.89-7.84 (m,2H), 2-7 7.42 -7.33 (m, 3H), 6.88 (s, 1H), 6.67-6.64 (d*d, J₁ -7,2 Hz, J₂ -2.1 Hz, 2H), 3.74 (s, 2H), 3.63 (m, 4H), 2.99 (s, 6H), 2.50 (m, 4H), 2.10 (s, 6H) |
| S01098 | 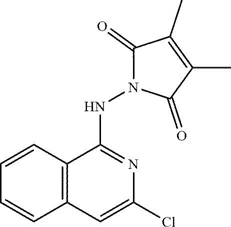 | 1-\|(3-chlomisoquinoly] amino\|-3,4-dimethylazoline-2.5-dione | 302.2 (M⁺ + 1) | (CDCl_{3,}300 MHz) ô: 7.78-7.74 (m, 2H), 7.56-7.47 (m, 2H), 7.38-7.32 (m, 1H), 7.04 (s, 1H), 2.10 (s,6H) |
| S01553 | 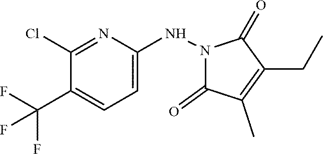 | 1-([6chloro-5-(trifluoromethyl()2-pyridyl)]amino}-3-ethyl-4-methylazoline-2.5-diose | 3321) | (CDCl_{3,} 300 MHz) ô: 7.79-7.76 (d, J = 8.4 Hz, 1H), 6.95 (s, 1H), 6.50-6.47 (d, J = 8.4 Hz, 1H), 2.54-2.46 (m, 2H), 2.07 (s, 3H), 1.27-1.17(m,3H) |
| S01734 | 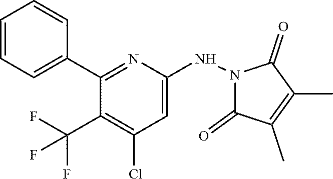 | 1-{[4-chloro-6-phenyl-5-(trifluoromethyl) (2-pyridyl)] amino}-3.4-dimethylazo line-2.5-dione | -396.3 (M⁺ + 1) | (CDCl_{3,} 300 MHz) ô: 7. 52 (br, 1H), 7.37-7.33 (m, 5H), 6.49 (s, 1H), 2.02 (s, 6H) |
| S01864 | 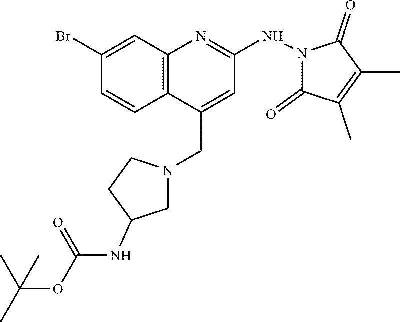 | N-[1-({2-[(3,4-dimwrhyl-2.5-dioxoalinyl)amino]-7-bromo(4-qainolyl)}methyl) pymolidin-3-yl] (tert-butoxy) carboxamide | 542.2 (M- - 1) | (CDCl₃,30O MHz) ô: 7.82- 7.70 (m, 3H), 7.40-7.35 (dd, J ₋ 9.0 Hz, 2.1 Hz, 1H), 6.86 (s, 1H), 4.90-4.80 (br, 0.5H), 4.20-4.10 (br, 0.5H), 3.8O (s, 2H), 2.90-2.80 (m, 1H), 2.70-2.55 (m, 2H), 2.40-2.20 (m,2H), 2.00 (s, 6H), 1.70-1.50 (m, 2H), 1.43 (s,9H) |
| S01877 | 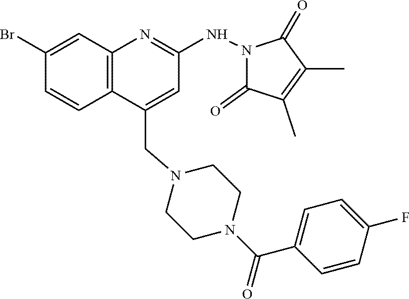 | 1-{[7-bromo-4-({4-\|(4-fluompheftyl) carbonyl]piperazi nyl ) methyl)(2-quinolyl)]amino}-3.4-dimethylazoline-2.5-dione | 566 (M⁺ + 1) | (CDCl_{3,} 300 MHz) ô: 7.86-7.83 (m, 2H ), 7.43-7.37 (m, 3H), 7.11 -7.06 (m, 2H) 6.82 (s, 1H), 3.77-3.44 (m,6H), 2.52-2.11 (m, 4H), 2.10 (s, 6H) |
| S01475 | 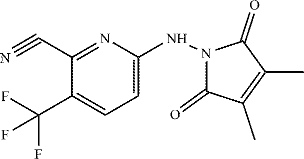 | 6-[(3.4-dimethyl-2.5-dioxoazolinyl) amino]-3-(trifluoromethyl) pyridine-2-carbonitrile | 309.2 (M- - 1) | (CDCl₃, 300 MHz) ô: 7.83-7.81 (d, J = 6.6 Hz, 1H), 7.31 (s, 1H), 6.86-6.84 (d, J = 6.6 Hz, 1H), 2.08 (s, 6H) |
| S00186 | 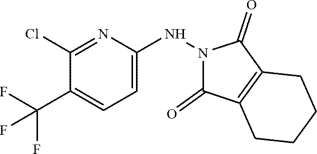 | 2-{[6-chloro-5-(trifluoromethyl)-2-pyridyl]amino}-4,5,6,7-tetrahydroisoindole-1 ,3-dione | | (CDCl₃, 300 MHz) ô: 7.80-7.70 (d, J = 8.7 Hz, 1H), 6.55-6.45 (d, J = 8.7 Hz, 1 H), 2.50-2.30 (m, 4H), 1.90-1.75 (m, 4H) |
| S00516 | 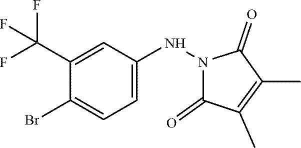 | -1- ([4-bromo-3-(trifluoromethyl) phenyl]amino)-3.4-dimethylazoline-2.5-dione | 360.9 (M⁻ - 1) | (CDCl₃, 300 MHz) ô: 7.54-7.50 (d, J= 9.0 Hz, 1H), 7.07-7.05(d, J = 3.0 Hz, 1H), 6.76-6.72 (dd, J - 8.7 Hz, 2.7 Hz. 1H), 6.10 (s, 1H), 2.08 (s, 6H) |
| S00738 | 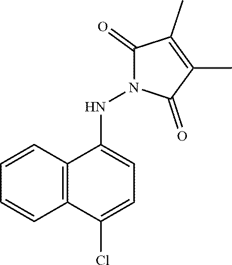 | 1-[(4-chloronaphthyl) | No Mass | (C»CI s. 3<X) MHz) δ 8.27-8.23 (dd, J= 8.7 Hz, 1.5 Hz, 1H), 8.01-7.97. (d,J J = 8.7 Hz, 1H), 7.65-7.52 , ( 1 H (m, 1H), 7 38-7.35 (d,J J - 8.1 Hz, 1H), 6.60 (s, 1H). 6.57.. 6.53 (d. , J - 8.4 Hz, 1H), 2.09 (s, 6H) |
| S00935 | 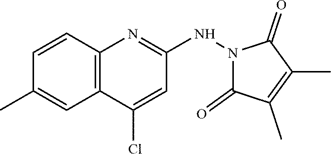 | m5 amino]-3,4 dimethyzoline-. | 315.9 (M⁺ + 1) | (CDCl₃3.3l3lCDD MHz) ft: 7.79 (s, 1H)_{.} 7.61-7.51 (d, J-8.4 (d, 1H),7.45-7.42 (d, 8.7 Hz, 111), 6.86 (s, iFH. 2,49 (s, 3H), ₂₉₈ (i>.6H) |
| S00942 | 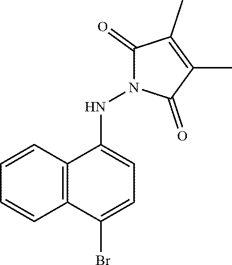 | 1-[(4-bromonspkyl( | 342.9 (M- - 1) | (CDC1, 300 MHz) õ: 8.25-8.21 (d,J J = 8.1 Hz, 1H). 7.99-7.95 (d, J = 8.4 Hz, IH), 7.65-7.52 (m, 3H), 6.56 (s, 1H), 6.52-6.49.6 (d,J-8 Hz, 2.8(s,6H) |
| S01037 | 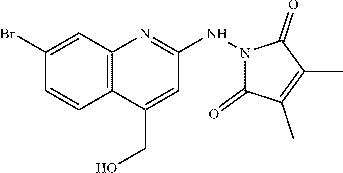 | 1-{[7-bromo-4-(hydroxymethyl) 2-quinolyl)] , amino}--dimethylazoline-2,5-dione | 376.1 (M⁺ + 1) | (CDCl₃, 300 MHz) δ: 7.85-7.84 (d, J = 1.8 Hz, 1H), 7.52-7.49 (d, J = 8.7 Hz, 1H), 7.40-7.36 (dd, J= 8.7 Hz, 1.8 Hz, 1H), 6.99 (s, 1H), 4.99 (s, 2H), 2.11 (s, 1H), 2.10 (s, 6H) |
| S01047 | 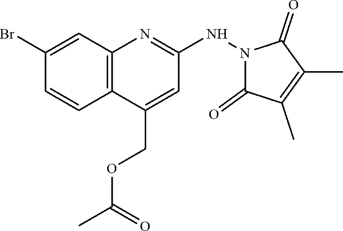 | {2-[(3,4-dimethyl-2,5-dioxoazolinyl) amino]-7-bromo-4-quinolyl} methyl acetate | 418.0 (M⁺ + 1) | CDCl₃, 300 MHz) δ: 7.86 - 7.85 (d, J = 1.8 Hz, 1H), 7.58-7.54 (d, J = 8.7 Hz, 1H), 7.45 -7.41 (dd, J = 9.0 Hz, 2.1 Hz, 1H), 6.85 (s, 1H), 5.27 (s, 2H), 2.13 (s, 3H), 2.02 (s, 6H) |
| S01191 | 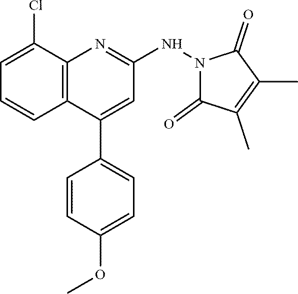 | 1-{[S-chloro-4-(4-methoxyphenyl) (2-quinolyl)] amino}-3,4-dimethylazoline-2,5-dione | 408.2 (M⁺ + 1) | (CDCl₃, 300 MHz) δ: 7.67-7.60 (m, 2H), 7.37-7.32 (m, 2H), 7.18-7.13 (m, 1H), 7.05-7.01 (m, 2H), 6.84 (br, 1H), 6.78 (s, 1H), 3.88 (s, 1H), 2.10 (s, 6H) |
| S01207 | 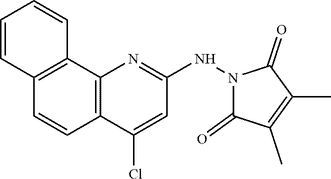 | 1 [4-chlorobenzo[h] quinolin-2-yl)\ ¡unitw¡-3,4-dimerhyiazo line-2,$-diow | ( 352.2 (M⁺ + 1) | (cm j, 300 MHz) δ: 8,81-8.7 id, J= 8.7 Hz, 00000000000 7.96 (d. J =8.7 Hz, 1H) 7.87-7.83 (d, 9.0 fₕ,IH), 7,33- 7.99 (d, J = 9.0 Hz, lH),7.67–7.55 (m, 2H), 7.00 (s, 1H), 6.84 (br, , 1H), 2.02 _{IS}, 6H) |
| S01268 | 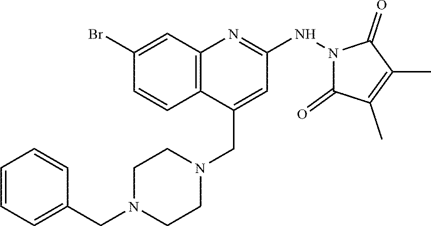 | .1 - [(7-bromo-4-{(4- benzylpiperazinyl}] methyl}(2-, i^{:} quinolyl))amino]-3,4-dimethylazoline-2.5-dione | ( 534.3 (M⁺ + 1) | (CDC1₃, 300 MHz) δ 7.88-7.82 (m, 2H), 7.40-7.25 m, 6H), 6.89 (s, 1H}, 3.73 (s, 2H). 3.51 (s, 2H), 2.60-.2.40)),^{,} (m, 8H), 2.09 (s, 6H) |
| S01371 | 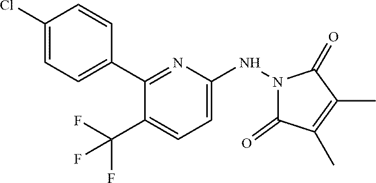 | 1-{[6-(4-chlorophenyl)-5-{trifluoromethyl)-(2-pyridyl)]amino ]-3 .4-dimethylazol ine-2.5-dione | 394.4 (M- - 1) | (CDC1 300 MHz) 6: 7.85-7.81 (d, J = 8.7 Hz. IH). 7.57 b 7H) (br, 1H), 7,38-7.31 (m, 4H), 6.47-6,44 (d, J = 8.4 Hz, IH), 2.04 ), (s. 6H) |
| S01393 | 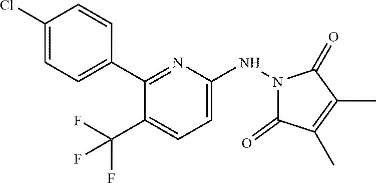 | 3.4-dimethyl∼1∼[{6∼ (4-methylphenyl)∼ 5-(trifluoromethyl) (2 - pyridyl) \| amino } azoline∼2,5∼dione | 374.3 (M- - 1) | (CDC1₃, 300 MHz) ð: 7.87-7.84 (d,J J= 9.0 Hz, 1H), 7.36-7.33 (d. J= 8.1 Hz 2H),7.21 7.18(d,J» 8.1 Hz, 2H). 6.81 (s. 1 H), 6.54-6.51 (d. J = 8.7 Hz, 1 H). 2.39 (s, 3H). 2.04 (s_{,} 6H) |
| S01474 | 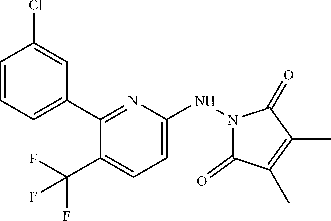 | l-f [O-f3-chloropheny l)-5-trifluoromethyl)-2-pyridyl)] amino}₋3,4- dimethy lazol line-2.5-dione | 394 (M- - 1) | (CDCl₃, 300 MHz) 6: 7.87-7.84 (d, J= 6.6 Hz, 1H), 7.41-7,31 (m,4H), 7.21 (br, 1H), 6.56-6.54 (d, J = 6.6 Hz, 1H), 2.04 (s, 6H) |
| S01600 | 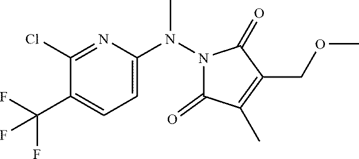 | 1-{[6-chloro∼5∼ (trifluoromethy l)-(2-pyridyl)] 2 methylamino]∼3∼ (methoxymethyl)∼ 4-methylazoline-2,5-dione | (M³⁶²-⁻⁻1) | (CDC1₃,_{.} 300 MHz) 6: 7.71-7.74 (d, J= 8.6 Hz, 1H), 6.47∼ 6.44 (d, J = 8.4 Hz. 1H), 4.37 (s, 2H ), 3.45 (s, 3H), 3.44 (s. 3H), 2.20 (s, 3H) |
| S01683 | 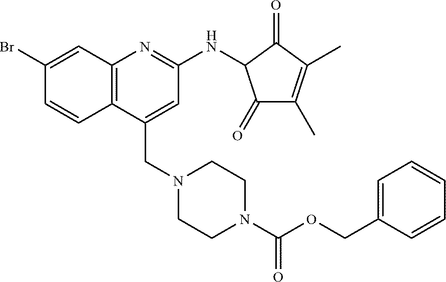 | phenylmethyl 4-({2-[(3,4-dimethyl-2,5-dioxoazolinyl) amino]-7-bromo-4∼quinolyl }methyl) piperazinecarboxylate ) | 578 (M⁺ + 1) | (CDCl_{3,} 300 MHz) 6; 7.84 (m, 2H), 7.41 (m, 1H), 7.37-7.30 (m, 5H), 6.86 (s, 1H). 6.76 (br, 1H), 5.14(s, < 2H). 3.70 (s, b 2H), 3. 50 (m, 4H), 2.44 (m, 4H) 2.09 (s, 6H) |
| S01688 | 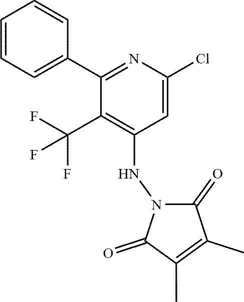 | 1-{[6-chloro-2-phenyl-3-(trifluoromethyl)(4-pyridyl)] amino)-3.4--dimethylazoline-2,5-dione | 394.3 (M⁻ - 1) | (CDCl₃, 300 MHz) δ: 7.39-7.35 (m, 5H), 7.26 (s, 1H), 6.55 (d, J = 6.0 Hz, 1H), 2.03 (s, 6H) |
| S01691 | 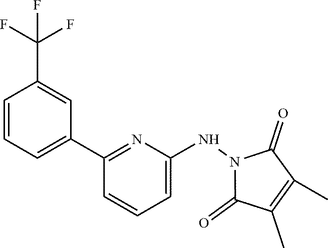 | 3,4-dimethyl-1-({6-[3-(trifluoromethyl)phenyl] (2-pyridyl)}amino) azoline-2,5-dione | 362.3 (M⁺ + 1) | (CDCl₃, 300 MHz) δ: 8.13 (br, 1H), 8.00-7.97 (d,J = 7.8 Hz, 1H), 7.65-7.58 (m, 2H), 7.52-7.47 (t, 1H), 7.33 -7.31 (d, J = 7.5 Hz, 1H), 6.65-6.63 (t, 2H). 2.07 (s, 6H) |
| S01699 | 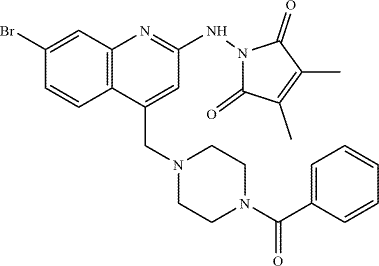 | 1-[(7-bromo-4-{[4-(phenylcarbonyl) piperazinyl)methyl} (2-quinolyl)) amino]-3,4-dimethylazoline-2,5-dione | 548 (M⁺ + 1) | (CDCl₃, 300 MHz) δ: 7.88-7.84 (m, 2H), 7.41-7.38 (m, 6H), 6.86 (s, 1H), 3.79-3.73 (m, 4H), 3.42 (m, 2H), 2.54 (m,4H), 2.09 (s, 6H) |
| S01759 | 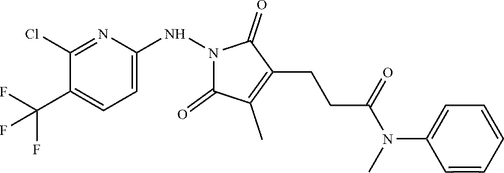 | 3-(1- {[6-chloro-5-(trifluoromethyl) (2-pyridyl)]amino}-4-methyl-2.5-dioxoazolin-3-yl)-N-methyl-N-phenylpropanamide | 465.3 (M- - 1) | (CDCl₃, 300 MHz) δ: 7.94 (s, 1H), 7.73 (d, J = 8.6 Hz, 1H), 7.43-7.33 (m, 3H), 7.19-7.16 (m, 2H), 6.51 (d, J = 8.5 Hz, 1H}, 3.24 (s, 3H), 2.70 (i, j = 7.1 Hz, 2H), 2.41 (t, J = 7.2 Hz, 2H), 2.06 s, 3H). |
| S01762 | 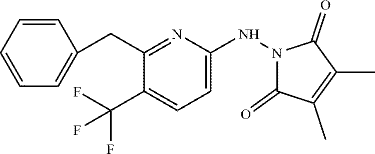 | 3,4-dimethyl-1-{[6-benzyl-5-(trifluoromethyl) (2-pyridyl)]amino) zoline-2,5-dione | 374.3 (M- - 1) | (CDCl₃, 300 MHz) δ 7.67-7.63 (d, J = 8.4 Hz, 1H), 7.22-7,12 (m, 5H), 6.84 (s, 1H), 6.45-6.41 (d, J = 8.7 Hz, 1H), 4.08 (s, 2H), 2.00 (s, 6H ) |
| S01800 | 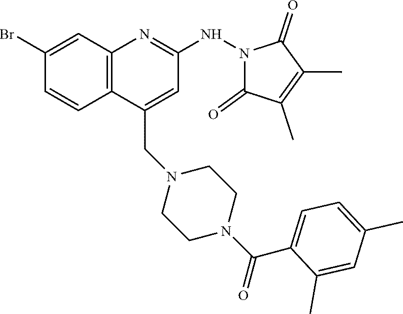 | 1-{[4-({4-[(2,4-dimethylphenyl) carbonyl]piperazinyl methyl)-7-bromo(2-quinolyl) lamino}-3,4-dimethylazoline-2,5-dione | 576 (M⁺ + 1) | (CDCl₃, 300 MHz) δ: 7.87- 7.83 (m, 2H), 7.41 - 7.37 (d*d, J₁ = 8.7 Hz, J₂ = 2.1 Hz, 1H), 7.05-7.00 (m, 3H), 6.85 (s, 1H), 3.81 (m, 2H), 3.72 (s, 2H), 3.22 (m, 2H), 2.58 - 2.55 (m, 2H) 2.36 -2.33 (m, 2H), 2.31 (s, 3H), 2.27 (s, m), 2.10 (s, 6H) |
| S01801 | 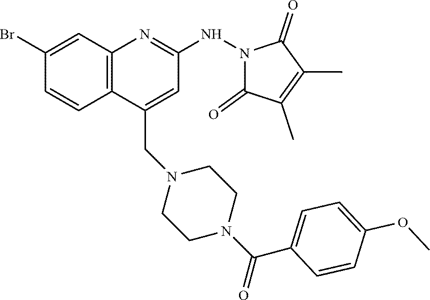 | 1-{[7-bromo-4-({4-[(4-methoxyphenyl)carbonyl) piperazinyl}methyl) (2-quinolyl)] amino)-3,4-dimethylaxoline-2,5-dione | 578 (M⁺ + 1) | (CDCl₃, 300 MHz) δ: 7.90-7.82 (m, 2H), 7.41-7.36 (m, 3H), 6.92-6.88 (m, 3H), 3.83 (s, 3H), 3.78 (s, 2H), 3.62 (m, 4H), 2.51 (m, 4H), 2.10 (s, 6H) |
| S01820 | 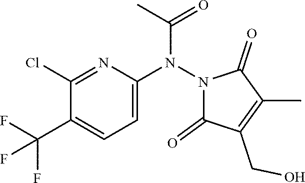 | N-[6-chloro-5-(trifluoromethyl) (2-pyridyl)]-N-[4-(hydroxymethyl)-3-methyl-2,5-dioxoazolinyl] acetamide | 376 (M- - 1) | (CDCl₃, 300 MHz) δ: 8.27-8.26 (m, 1H), 8.02-7.99 (r, 1H) 4.69 -4.67 (d, J = 5.1 Hz, 2H), 5.1 Hz, 2H), 2.31 (s, 3H), 2.28 (s, 3H) |
| S01822 | 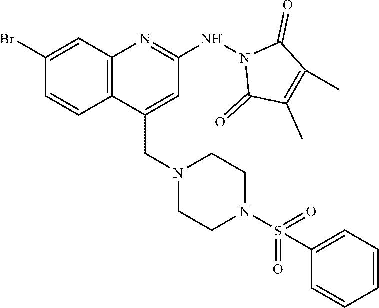 | 1-[(7-bromo-4-{[4-(phenylsulfonyl) piperazinyl]methyl} (2-quinolyl)) amino]-3,4-dimethylazoline-2,5-dione | 584 (M⁺ + 1) | (CDCl₃, 300 MHz) δ: 7.80 - 7.71 (m, 4H), 7.62-7.52 (m, 3H), 7.34-7.31 (d″d, J₁ = 8.7 Hz, J₂ = 2.1 Hz, 1H), 6.75 (s, 1H), 3.65 (s, 2H), 3.01 (m, 4H), 2.56-2.53 (m, 4H), 2.07 (s, 6H) |
| S00871 | 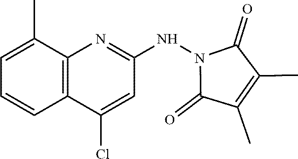 | 1-[(4-choloro-8-methyl(2-quinolyl))amino]-3,4-dimethylazoline-2,5-dione | 316 (M⁺ + 1) | (CDCl₃, 300 MHz) δ: 7.91-7.88 (d, J = 8.4 Hz, 1H), 7.47-7.44 (d, J = 6.6 Hz, 1H), 7.32-7.28 (d,J = 8.4 Hz, 1H), 6 95 (s, 1H). 6.78 (br, 1H), 2.42 (s, 3H), 2.10 (s, 6H) |
| S01862 | 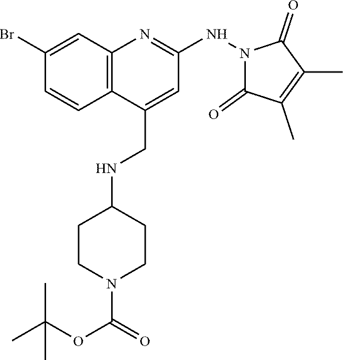 | tert-butyl 4-({2-[(3,4-dimethyl-2,5-dioxoazoli imyl)amino}-7-bromo-4-quinolyl}methyl) amino] piperidinecarboxylate | 556.2 (M- - 1) | (CDCl₃, 300 MHz) δ: 7.81-7.80 (d, J = 1.8 Hz, 1H), 7.60-7.55 (d, J = 9.0 Hz, 1H), 7.40- 7.35 (dd. J = 8.7 Hz 2.1 Hz, 1H), 6.85 (s, 1H), 4.10-3.90 (m, 2H), 3.76 (s, 2H), 2.90-2.80 (m, 2H), 2.70-2.55 (m, 1H), 2.09 (s, 6H), 1.90- 2H), 1.80 (m, 2H), 1.46 (s, 9H), 1.40-1,30 (m, 2H) |
| S01928 | 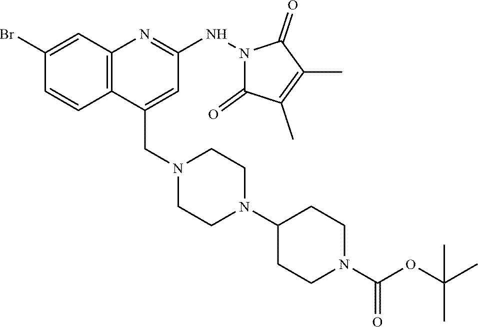 | tert-butyl 4-[4-((2-[(3,4-dimethyl-2,5-dioxoazolinyl)amino]-7-bromo-4-quimolyl)methyl) piperizinyl] piperidinecarboxylate | 627 (M⁺ + 1) | (CDCl₃, 300 MHz)δ: 7.88-7.83 (m, 2H), 7.40-7.36 (m, 1H), 6,87 (s, 1H), 4.15-4.08 (m, 1H), 3.68 (s, 2H), 2.73-2.65 (m, 1H), 2.54-2.52 (m 8H), 2.36 (m, 2H). 2.06 (s, 6H), 1.81-1.77 (m, 2H), 1.44 (s, 9H), 1,42-1.35 (m, 2H) |
| S01929 | 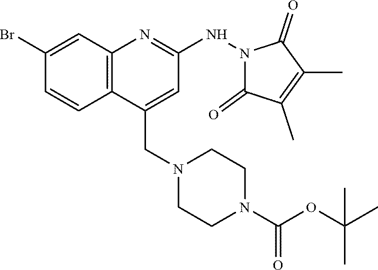 | 1-[(4-{(4-(3,3-dimethylbutanoyl) piperazimyl}methyl}-7-bromo(2-quinolyl))amino]-3,4-dimethylazoline-2,5-dione | 542 (M⁺ + 1) | (CDCl₃, 300 MHz) δ: 7.89 -7.84 (m,2H), 7.42-7.38(m, 1H), 6.87 (s, 1H), 3.72 (s, 2H), 3.67-3.63 (m, 2H), 3.49-3.46 (m, 2H), 2.50-2.41 (m, 4H), 2.25 (s,2H), 2.10 (s, 6H), 7.65 (s, 9H) |

Table 3 presents structures, IUPAC name, ID number (“SCID”), mass, and 1H NMR values for further representative compounds.

TABLE 3

REPRESENT ATIVE COMPOUNDS

| SCID | Structure | IUPAC Name | MS (m/e) | ¹H NMR |
|---|---|---|---|---|
| S03518 | 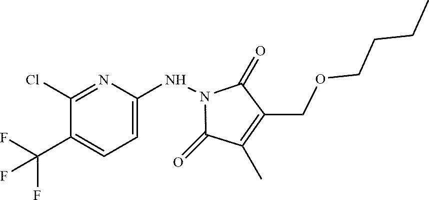 [SMILES: CCCCOCC1=C(C)C(=O)N(Nc2ccc(C(F)(F)F)c(Cl)n2)C1=O] | 3-(Butoxymethyl)-1-{[6-chloro-5-(trifluoromethyl) (2-pyridyl)] amino]-4-methylazoline-2,5-dione | 390.2 (M- - 1) | (CDCl₃, 300 MHz) δ: 0.90-0.95 (t, J = 7.2 Hz, 3H), 1.35-1.43 (m, 2H), 1.54-1.63 (m, 2H), 2.20 (s, 3H), 3.50-3.55 (t, J = 6.6 Hz, 2H),4.41 (s, 2H), 6.49-6.52 (d, J = 8.4 Hz, 1H). 6.88 (s, 1H), 7.77-7.79 (d, J = 8.4 Hz, 1H) |
| S02225 | 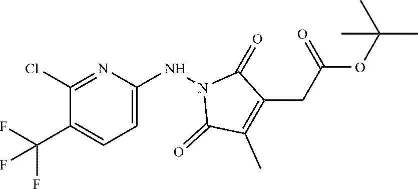 [SMILES: CC1=C(CC(=O)OC(C)(C)C)C(=O)N(Nc2ccc(C(F)(F)F)c(Cl)n2)C1=O] | tert-butyl 2-(1-{[6-chloro-5-(trifluoromethyl) (2-pyridyl)] amino}-4-methyl-2,5-dioxoazolin-3-yl)acetat | 418.0 (M- - 1) | (CDCl₃, 300 MHz) δ: 7.79-7.76 (d, J = 8.4 Hz, 1H), 7.04 (s, 1H), 6.51-6.48 (d, J = 8.7 Hz, 1H), 3.45 (s, 2H), 2.11 (s, 3H), 1.25 (s, 9H) |
| S02264 | 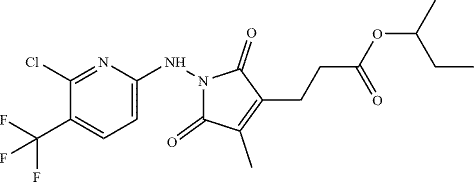 [SMILES: CCC(C)OC(=O)CCC1=C(C)C(=O)N(Nc2ccc(C(F)(F)F)c(Cl)n2)C1=O] | 4-methylphenyl 3-(1-{[6-chloro-5-(trifluoromethyl)(2-pyridyl)]amino}-4-methyl-2.5-dioxoazolin-3-yl)propanoate | 433.9 (M⁺ + 1) | (CDCl₃, 300 MHz) δ; 7.78-7.75 (d, J = 8.7 Hz, 1H), 7.08 (s, 1H), 6.50-6.47 (d, J = 8.4 Hz, 1H). 4.85-4.83 (m, 1H), 2.79-2.66 (m, 4H), 1.59-1.52 (m, 2H), 1.21-1.18 (d, J = 6.3 Hz, 3H), 0.90-0.86 (t, 3H) |
| S02366 | 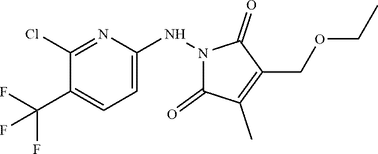 [SMILES: CCOCC1=C(C)C(=O)N(Nc2ccc(C(F)(F)F)c(Cl)n2)C1=O] | 1-{[6-Chloro-5-(trifluoromethyl) (2-pyridyl)]amino}-3-(ethoxymethyl)-4-methylazoline-2,5-dione | 364.0 (M⁺ + 1) | (CDCl₃, 300 MHz) δ: 1.23-1.28 (t, J = 6.9 Hz, 3H), 2.21 (t, J = 1.2 Hz, 3H), 3.56-3.63 (q, J = 6.9 Hz, 2H), 4.41 (q, J = 1.2 Hz, 2H), 5.48-6.51 (d, J = 8.7 Hz, 1H), 7.0 (s, 1H), 7.75-7.78 (d, J = 8.4 Hz, 1H) |
| S03405 | 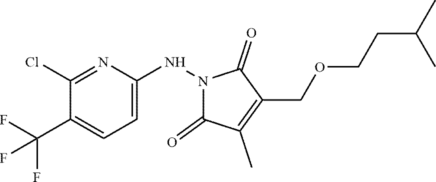 [SMILES: CC1=C(COCCC(C)C)C(=O)N(Nc2ccc(C(F)(F)F)c(Cl)n2)C1=O] | 1-{[6-Chloro-S-(trifluoromethyl)-(2-pyridyl)]amino}-4-methyl-3-[(3-methylbutoxy) methyl] azoline-2,5-dione | 404.2 (M⁻ - 1) | (CDCl₃, 300 MHz) δ: 0.87-0.95 (m, 6H), 1.47-1.54 (m, 2H), 1.66-1.75 (m, 111),2.19 (s, 3H), 3.53-3.57 (t, J = 7.2 Hz, 2H), 4.41 (s, 2H), 6.49-6.52 (d, J = 8.4 Hz. 1H), 6.90 (s, 1H), 7.77-7.80 (d, J = 8.4 Hz, 1H) |
| S03448 | 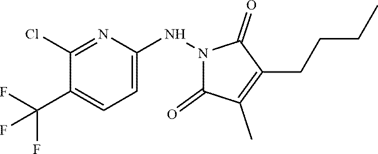 [SMILES: CCCCC1=C(C)C(=O)N(Nc2ccc(C(F)(F)F)c(Cl)n2)C1=O] | 3-butyl-1-{(6-chloro-5-(trifluoromethyl) (2-pyridyl)]amino}-4-methylazoline-2,5-dione | 360.0 (M⁺ + 1) | (CDCl₃, 300 MHz) δ: 7.78-7.74 (d, J = 9.0 Hz, 1H), 7.05 (s, 1H), 6.50-6.46 (d, J = 8.7 Hz, 1H), 2.51-2.45 (t, J = 7.5 Hz, 2H), 2.07 (s, 3H), 1.60-1.52 (m, 2H), 1.42-1.34 (m, 2H), 0.97-0.92 (t, J = 7.2 Hz, 3H) |
| S03456 | 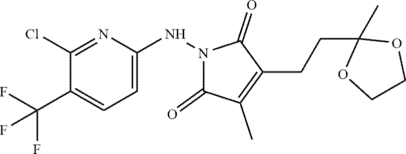 [SMILES: CC1=C(CCC2(C)OCCO2)C(=O)N(Nc2ccc(C(F)(F)F)c(Cl)n2)C1=O] | 1-{[6-Chloro-5-(trifluoromethyl) (2-pyridyl)]amino}-4-methyl-3-[2-(2-methyl(1,3-dioxolan-2-yl)) ethyl]azoline-2,5-dione | 418.2 (M⁻ - 1) | (CDCl₃, 300 MHz) δ: 1.35 (s, 3H), 1.98-2.03 (t, J = 7.2 Hz, 2H), 2.07 (s, 3H), 2.56-2.61 (t, J = 7.2 Hz, 2H), 3.88-4.00 (m, 4H), 6.47-6.50 (d, J = 8.7 Hz, 1H), 6.78 (s, 1H), 7.76-7.79 (d, J = 8.7 Hz, 1H) |
| S03552 | 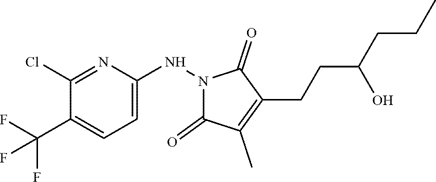 [SMILES: CCCC(O)CCC1=C(C)C(=O)N(Nc2ccc(C(F)(F)F)c(Cl)n2)C1=O] | 1-{[6-chloro-5-(trifluoromethyl)(2-pyridyl)]amino}-4-(3-hydroxyhexyl)-3-methylazoline-2,5-dione | 404.2 (M⁻ - 1) | (CDCl₃, 300 MHz) δ: 7.77-7.74 (d, J = 8.7 Hz, 1H), 7.21 (br, 1H), 6.51-6.48 (d, J = 8.7 Hz, 1H ), 3.61-3.59 (m, 1H), 2.65-2.60 (t, 2H), 2.09 (s, 3H), 1.77-1.62 (m, 3H), 1.47-1.25 (m, 4H), 0.94-0.90 (m, 3H) |
| S03742 | 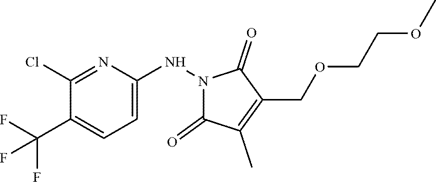 [SMILES: COCCOCC1=C(C)C(=O)N(Nc2ccc(C(F)(F)F)c(Cl)n2)C1=O] | 1-{[6-Chloro-5-(trifluoromethyl)(2-pyridyl)]amino}-3-[(2-methoxy ethoxy)methyl]-4-methylazoline-2,5-dione | 392.0 (M⁻ - 1) | (CDCl₃, 300 MHz) δ: 7.77-7.74 (d, J = 8.4 Hz, 1H), 7.20 (s, 1H), 6.52-6.49 (d, J = 8.4 Hz, 1H), 4.48 (s, 1H), 3.72-3.69 (m, 2H), 3.60-3.56 (m, 2H), 3.39 (s, 3H), 2.20 (s, 3H) |
| S03745 | 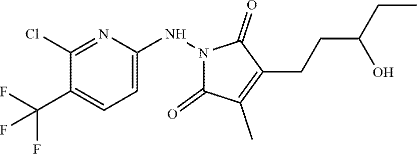 [SMILES: CCC(O)CCC1=C(C)C(=O)N(Nc2ccc(C(F)(F)F)c(Cl)n2)C1=O] | 1-{ [6-chloro-5-(trifluoromethyl)(2-pyridyl)]amino}-4-(3-hyroxy-pentyl)-3-methylazoline-2,5-dione | 390.0 (M⁻ - 1) | (CDCl₃, 300 MHz) δ: 7.76-7.73 (d, J = 8.7 Hz, 1H), 7.36 (br, 1H), 6.51-6.48 (d, J = 8.7 Hz, 1H), 3.52-3.51 (m, 1H), 2.64-2.59 (t, 2H), 2.09 (s, 3H), 1.83-1.62 (m, 2H), 1.55-1.42 (m, 2H), 0.94-0.90 (m, 3H) |
| S03747 | 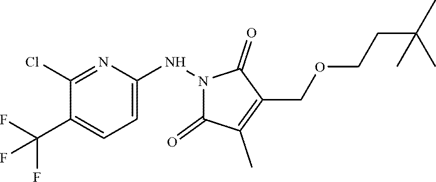 [SMILES: CC1=C(COCCC(C)(C)C)C(=O)N(Nc2ccc(C(F)(F)F)c(Cl)n2)C1=O] | 3-[(3,3-Dimethylbutoxy) methyl]-1-{[6-chloro-5-(trifluoromethyl) (2-pyridyl)]amino}-4-methylazoline-2,5-dione | 418.1 (M⁻ - 1) | (CDCl₃, 300 MHz) δ: 7.77-7.80 (d, J = 8.7 Hz, 1H), 6.77 (s, 1H), 6.49-6.52 (d, J = 8.4 Hz, 1H), 4.40 (s, 2H), 3.55-3.60 (t, J = 7.5 Hz, 2H), 2.20 (s, 3H), 1.53-1.58 (t, J = 6.9 Hz, 2H), 0.92-0.96 (s, 9H). |
| S03873 | 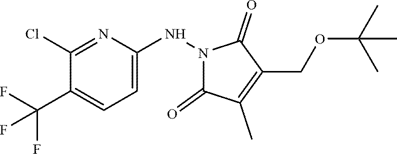 [SMILES: CC1=C(COC(C)(C)C)C(=O)N(Nc2ccc(C(F)(F)F)c(Cl)n2)C1=O] | 4-{(tert-Butoxy) methyl]-1-{[6-chloro-5-(trifluoromethyl) (2-pyridyl)] amino}-3-methylazoline-2,5-dione | 390.2 (M⁻ - 1) | (CDCl₃, 300 MHz) δ: 7.80-7.77 (d, J = 8.4 Hz, 1H), 6.85 (br, 1H), 6.51-6.49 (d, J = 8.4 Hz, 1H), 4.37 (s, 2H), 2.21 (s, 3H), 1.28 (s, 9H) |
| S03955 | 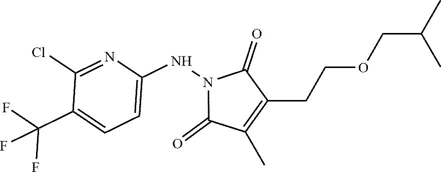 [SMILES: CC1=C(CCOCC(C)C)C(=O)N(Nc2ccc(C(F)(F)F)c(Cl)n2)C1=O] | 1-{[6-Chloro-5-(trifluoromethyl) (2-pyridyl)]amino}-4-methyl-3-[2-(2-methylpropoxy) ethyl]azoline-2,5-dione | 404.1 (M⁻ - 1) | (CDCl₃, 300 MHz) δ: 0.86-0.88 (d, J = 8.4 Hz, 6H), 1.79-1.83 (m, 1H), 2.21 (s, 3H), 2.72-2.76 (t, J = 6.6 Hz, 2H), 3.17-3.19 (d, J = 6.6 Hz, 2H), 3.60-3.64 (t, J = 6.6 Hz, 2H), 6.45-6.48 (d, J = 8.7 Hz, 1H), 7.03 (s, 1H), 7.75-7.78 (d, J = 8.4 Hz, 1H) |
| S03956 | 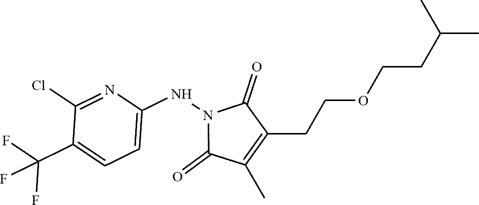 [SMILES: CC1=C(CCOCCC(C)C)C(=O)N(Nc2ccc(C(F)(F)F)c(Cl)n2)C1=O] | 1-{[6-Chloro-5-(trifluoromethyl) (2-pyridyl)]amino}-4-methyl-3-[2-(3-methylbutoxy) ethyl]azoline-2,5-dione | 418.3 (M⁻ - 1) | CDCl₃, 300 MHz) δ: 0.85-0.92 (d, J = 6.6 Hz, 6H), 1.39-1.46 (m, 2H), 1.60-1.69 (m, 1H), 2.09 (s, 3H), 2.72-2.76 (t, J = 6.6 Hz, 2H), 3.41-3.46 (d, J = 6.6 Hz, 2H), 3.60-3.64 (t, J = 6.6 Hz, 2H), 6.45-6,47 (d, J = 8.4 Hz, 1H), 7.38 (s, 1H), 7.73-7.76 (d, J = 8.4 Hz, 1H) |
| S03960 | 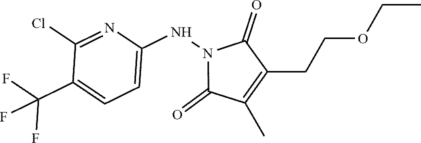 [SMILES: CCOCCC1=C(C)C(=O)N(Nc2ccc(C(F)(F)F)c(Cl)n2)C1=O] | 1-{[6-Chloro-5-(trifluoromethyl) (2-pyridyl)] amino}-3{2-ethoxyethyl)-4-methylazoline-2,5-dione | 376.2 (M- -1) | (CDCl₃,300 MHz) δ: 7.76-7.73 (d, J = 8.4 Hz. 1H), 7^{.}41 (s, 1H ), 6.48-6.45 (d, J = 8.4 Hz, 1H), 3.65-3.61 (t, 2H), 3.52-3.45 (q, 2H), 2.76-2.72 (t, 2H), 2.09 (s, 3H), 1.19-1.14 (t, 3H) |
| S03962 | 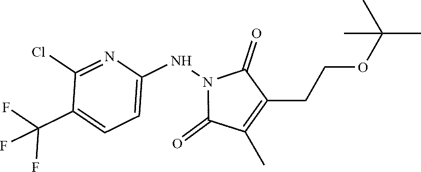 [SMILES: CC1=C(CCOC(C)(C)C)C(=O)N(Nc2ccc(C(F)(F)F)c(Cl)n2)C1=O] | 3-[(2,2-Dimmethyl-propoxy)methyl]-1-{[6-chloro-5-(trifluoromethy) (2-pyridyl)]amino}-4-methylazoline-2,5-dione | 404.2 (M- -1) | (CDCl₃, 300 MHz) δ: 7.80-7.77 (d, J = 8.7 Hz, 1H), 6.83 (s, 1H), 6.53-6.50 (d, J = 8.7 Hz, 1H), 4.43 (s, 2H), 3.17 (s, 2H), 2.22 (s,3H), 0.94 (s, 9H) |
| S03963 | 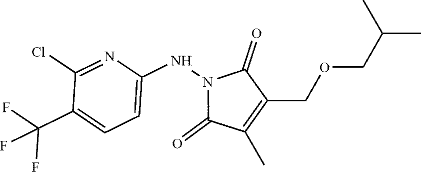 [SMILES: CC1=C(COCC(C)C)C(=O)N(Nc2ccc(C(F)(F)F)c(Cl)n2)C1=O] | 1-{[6-Chloro-5-(trifluoromethyl)(2-pyridyl)]amino}-4-methyl-3-[(2-methylpropoxy) methyl)azoline-2,5-dione | 390.2 (M- -1) | (CDCl₃, 300 MHz) δ: 7.78-7.75 (d, J = 8.7 Hz, 1H), 7.12 (s, 1H), 6.51-6.48 (d, J = 8.4 Hz, 1H), 4.4: (s, 2H), 3.30-3.28 (d, J = 6.6 Hz, 2H), 2.21 (s, 3H), 1.95-1.86 (m, 1H), 0.94-0.91 (d, J = 6.6 Hz, 6H) |
| S03964 | 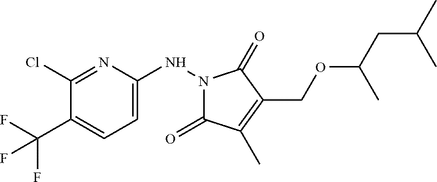 [SMILES: CC1=C(COC(C)CC(C)C)C(=O)N(Nc2ccc(C(F)(F)F)c(Cl)n2)C1=O] | 4.[(1.3-Dimethyl-butoxy)methyl]-1-{[6-chloro-5-(trifluoromethyl) (2.pyridyl)]amino}-3-methyl azoline-2,5-dione | 418.1 (M- - 1) | (CDCl₃, 300 MHz) δ: 7.77-7.74 (d, J∞ 8.4 Hz, 1H), 7.15 (br, 1H), 6.50-6-47 (d. J = 8.4 Hz, 1H), 4.51-4.30 {m, 2H), 3.64-3.57 (m, 1H), 2.20 (s, 3H), 1.76-1.69 (m, 1H), 1.56-1.49 (m, 1H), 1.27-1.23 (m, 1H), 1.21-1.18 (m, 3H), 0.92-0.88 (m, 6H) |
| S04034 | 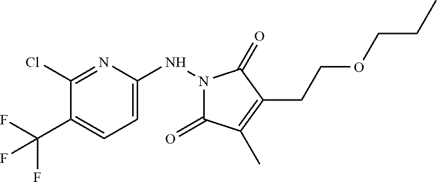 [SMILES: CCCOCCC1=C(C)C(=O)N(Nc2ccc(C(F)(F)F)c(Cl)n2)C1=O] | 1-{[6-chloro-5-(trifluoromethyl) (2-pyridyl)]amino}-3-methyl-4-(2-propoxyethyl) azoline-2.5-dione | 390.3 (M- -1) | (CDCl₃, 300 MHz) δ: 7.73 (s, 1H), 7.70 (br, 1H),6.47-6.44 (d, J = 8.7 Hz, 1H), 3.64-3.60 (t, 2H), 3.39-3.50 (t, 2H), 2.76-2.72 (t, 2H), 2.09 (s, 3H), 1.61-1.49 (m, 2H), 0.91-0.86 (t, 3H |

### Example 6: Synthesis of Representative Compounds

The following examples are intended to serve as illustrations, and all compounds herein could be synthesized using methods similar to those described in these examples.

General Procedure for the Synthesis of Substituted 2-pyridylhydrazines.

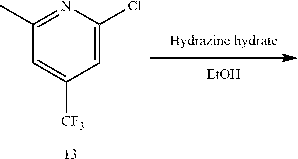

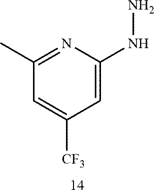

The general procedure for the synthesis of substituted 2-pyridylhydrazines is represented here in the synthesis of 4-(trifluoromethyl)-6-methyl-2-pyridylhydrazine (intermediate 14). One equivalent of 2-chloro-4-(trifluoromethyl)-6-methylpyridine (intermediate 13) and 1.5 equivalent of hydrazine hydrate were mixed in ethanol. The solution turned yellow after being stirred for several minutes. The reaction mixture was refluxed until TLC analysis showed no starting material left. The solvent was then removed under vacuum, and the resulting slurry was extracted with ether three times. The combined ether solution was dried over anhydrous MgSO₄ and evaporated to afford the crude product, which was then re-crystallized from ethanol to give intermediate compound 14.

### A. Synthesis of S00069

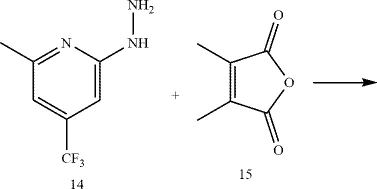

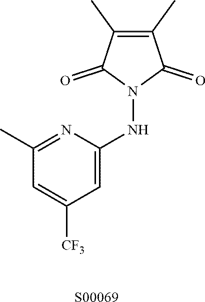 [SMILES: CC1=C(C)C(=O)N(Nc2cc(C(F)(F)F)cc(C)n2)C1=O ; "S00069"]

The anhydride 15 (1 eq.) was added to a solution of the hydrazine 14 (1.0 mmol) in chloroform and stirred under reflux for 4 hr. The reaction was determined to be completed by TLC (petroleum ether:ethyl acetate=3:1). The solvent was evaporated and the residue was purified by flash chromatography (petroleum ether:ethyl acetate=2:1) to give the product S00069.

### B. Synthesis of S00084

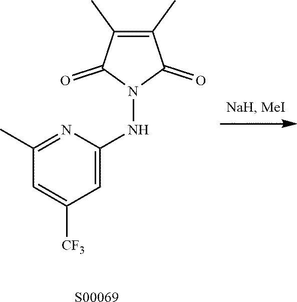

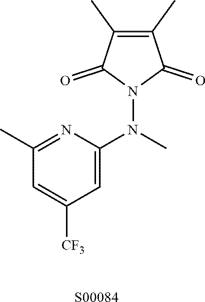 [SMILES: CC1=C(C)C(=O)N(N(C)c2cc(C(F)(F)F)cc(C)n2)C1=O ; "S00084"]

To a solution of S00069 (35 mg, 0.117 mmol) in THF (6 mL) at 0° C. was added NaH (60% in mineral oil, 8 mg, 0.12 mmol). The mixture was stirred for 30 min, and then MeI (20 mg) was added. The reaction mixture was stirred for 2 h at room temperature, and then poured into the saturated aqueous NH₄Cl. This was extracted with CHCl₃. The organic layer was dried over anhydrous Na₂SO₄. The solvent was removed and the residue was purified by preparative TLC (5:1 petroleum ether/diethyl ether) to afford S00084 (3 mg).

### C. Synthesis of S00109

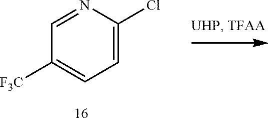

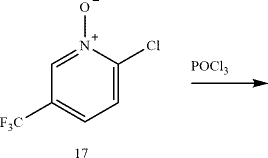

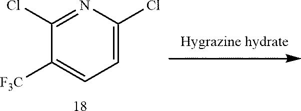

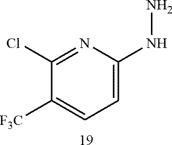

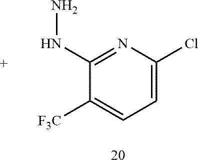

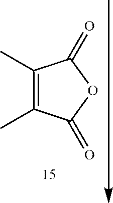

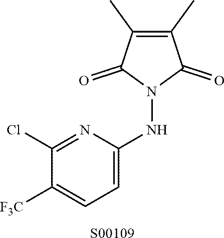 [SMILES: CC1=C(C)C(=O)N(Nc2ccc(C(F)(F)F)c(Cl)n2)C1=O ; "S00109"]

### Step 1: Synthesis of 2-Chloro-5-Trifluoromethyl-Pyridine-N-Oxide (17)

2-Chloro-5-trifluoromethyl-pyridine (16, 10 mmol) was dissolved in CH₂Cl₂ (20 mL) and UHP (Urea-hydrogen peroxide addition compound, 21 mmol) was added. The mixture was cooled to 0° C., trifluoroacetic anhydride (20 mmol) was then slowly added to the reaction mixture. It was allowed to warm to room temperature and stirred until the reaction was completed judged by TLC. The reaction was quenched with aqueous Na₂SO₃, stirred for4 h, washed with saturated aqueous NaHCO₃, and dried over anhydrous MgSO₄. Column chromatography afforded 1.8 g of intermediate compound 17 as oil.

### Step 2: Synthesis of 2,6-Dichloro-5-Trifluoromethyl-Pyridine (18)

2-Chloro-5-trifluoromethyl-pyridine-N-oxide (17, 4 mmol) was dissolved in freshly distilled POCl₃ (4.5 mL). The reaction mixture was heated to 80° C. for 17 h. After cooling to room temperature, the solvent was removed under reduced pressure. Ice was added, and the mixture was allowed to stand for 4 h. The mixture was partition between CH₂Cl₂ (50 mL) and saturated aqueous NaHCO₃. Column chromatography afforded intermediate compound 18 as yellow oil (yield: 50%).

### Step 3: Synthesis of 6-Chloro-5-Trifluoromethyl-2-Pyridylhydrazine (19)

To the solution of 2,6-dichloro-5-trifluoromethyl-pyridine (18, 2 g, 9.26 mmol) in ethanol (30 mL) was added hydrazine hydrate (2.9 g, 46 mmol). The reaction mixture was stirred for 4 h at room temperature, then concentrated to remove the solvent, and added ethyl acetate, washed with water. The organic layer was dried over anhydrous Na₂SO₄. Column chromatography (Silica, petroleum ether/ethyl acetate=4/1^{~}3/1) afforded intermediate compound 19 as white solid (yield: 56%) and another isomer 20 (yield: 18%).

### Step 4: Synthesis of S00109

2,3-Dimethylmaleic anhydride (15, 0.126 g, 1.0 mmol) was added to a solution of 6-chloro-5-trifluoromethyl-2-pyridylhydrazine (19, 0.211 g, 1.0 mmol) in5 ml of chloroform and the mixture was refluxed for 4 hours. The solvent was removed and the residue was purified by flash chromatography (5:1 to 2:1 petroleum ether/ethyl acetate) to give S00109 (0.21 g).

### D. Synthesis of S00170

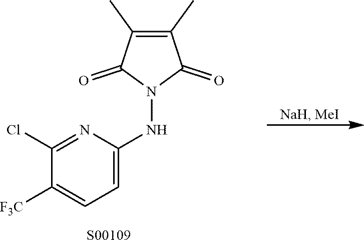

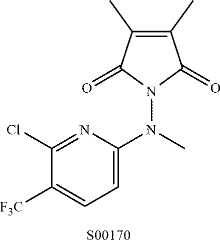 [SMILES: CC1=C(C)C(=O)N(N(C)c2ccc(C(F)(F)F)c(Cl)n2)C1=O ; "S00170"]

Compound S00109 (40 mg, 0.125 mmol) and NaH (60% in mineral oil, 7 mg, 0.188 mmol) were suspended in 2 ml of anhydrous THF and the mixture was stirred at 0° C. for 30 min. Methyl iodide (21 mg, 0.150 mmol) was added slowly to the solution at the same temperature and the mixture was then warmed to 25^{~}30° C. and stirred for overnight. The solvent was evaporated, and acetic acid was added to make the solution at pH=4. This was extracted with chloroform three times, and the combined organic phase was washed with 1N HCl, and then saturated aqueous NaHCO₃. It was then dried over anhydrous Na₂SO₄. The solvent was removed and the residue was purified by preparative TLC (4:1 petroleum ether/diethyl ether) to give compound S00170 (4.2 mg).

### E. Synthesis of S00585

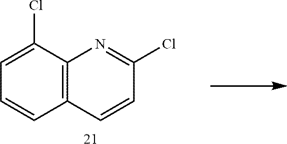

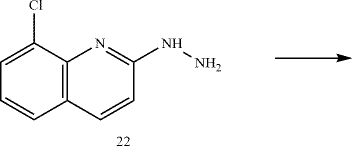

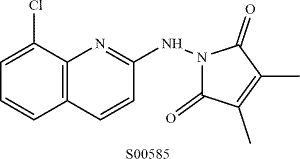

Intermediate Compound 21 was converted to Intermediate Compound 22. Intermediate Compound 22 was converted to Compound S00585.

General Procedure for the Synthesis of Substituted Phenylhydrazines.

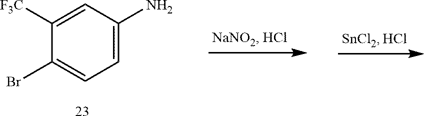

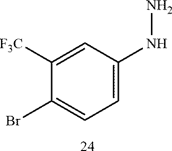

The general procedure for the synthesis of substituted phenylhydrazines is represented here in the synthesis of 3-(trifluoromethyl)-4-bromophenylhydrazine (intermediate 24). The corresponding benzylamine 23 (0.08 mol) was added to conc. HCl (40 mL). The mixture was cooled to -5° C. by ice and salt with stirring. Then sodium nitrite (5.52 g, 0.08 mol) dissolved in water (20 mL) was added. Stirring was continued for 1 h, and stannous chloride (30 g) in conc. HCl (30 mL) was added slowly over a period of two hours, while keeping the temperature below 0° C. The mixture was stirred for another hour after the addition and filtered. The filtered solid was treated with dilute aqueous sodium hydroxide and the then extracted with ether. The ether layer was washed with water, dried over anhydrous Na₂SO₄. The solvent was removed and the residue was crystallized from hexane to give the intermediate Compound 24.

### F. Synthesis of S00516

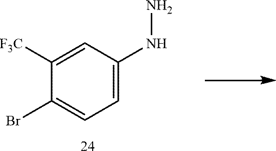

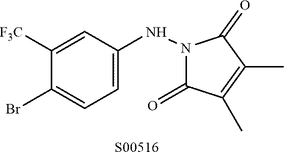

Compound S00516 was synthesized from the corresponding hydrazine 24.

### G. Synthesis of S00756

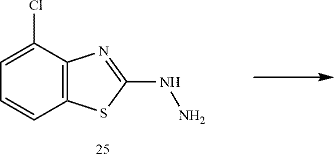

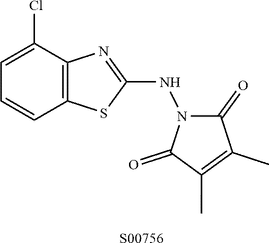 [SMILES: CC1=C(C)C(=O)N(Nc2nc3c(Cl)cccc3s2)C1=O ; "S00756"]

Intermediate Compound 25 was synthesized according to literature procedure (Eur. J. Med. Chem. Chim. Ther. 1997, 32(5), 397-408). It was converted to Compound S00756.

### H. Synthesis of S00513

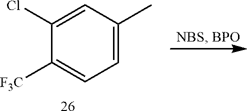

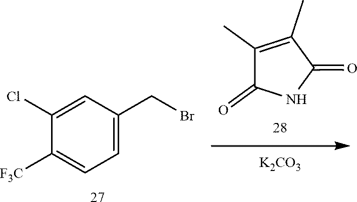

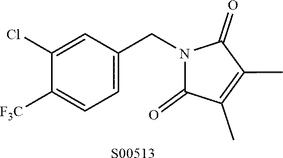

### Step 1: 3-Chloro-4-Trifluoromethylbenzylbromide (27)

A mixture of 2-chloro-4-methyl-1-trifluoromethylbenzene (Compound 26) (0.20 g, 1 mmol), N-bromosuccinimide (0.17 g, 1 mmol) and benzoyl peroxide (7.4 mg, 0.03 mmol) in carbon tetrachloride (2 mL) was heated to reflux for 2 hours. Another portion of benzoyl peroxide (20 mg, 0.08 mmol) was added. The mixture was heated to reflux for another 0.5 hours. The reaction mixture was further stirred at room temperature for 16 hours. The solid was removed by filtration. The solvent was removed under reduced pressure. The crude product was purified by flash chromatography on silica, using petroleum ether as eluent, to give 0.22 g (80%) of intermediate Compound 27.

### Step 2: Compound S00513

To a solution of 3,4-dimethylmaleimide (Compound 28) (43 mg, 0.34 mmol) in 1.3 mL of acetone was added anhydrous potassium carbonate (50 mg, 0.37 mmol) and intermediate Compound 27 (100 mg, 0.37 mmol). The reaction mixture was stirred at room temperature overnight. Water was added and the mixture was extracted with ethyl acetate. The organic extract was washed with brine, dried (Na₂SO₄), and concentrated under reduced pressure. The crude product was purified by chromatography on silica, using petroleum ether/ethyl acetate (10:1) as eluent, to give 70 mg (60%) of Compound S00513.

### I. Synthesis of S00628

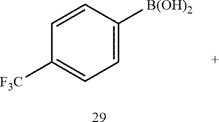

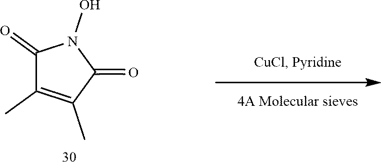

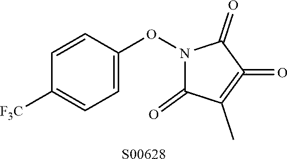

Into a solution of 1-hydroxy-3,4-dimethylazoline-2,5-dione (Compound 30) (56 mg, 0.39 mmol, 1 equiv) in 1,2-dichloroethane (2.5 mL), CuCl (39 mg, 0.39 mmol, 1 equiv), freshly activated 4 Å molecular sieves (~100 mg), and 4-trifluoromethylphenylboronic acid (Compound 29) (150 mg, 0.78 mmol, 2 equiv) were added, followed by pyridine (34 mg, 0.43 mmol, 1.1 equiv). The resulting light brown suspension was stirred for 16 h. The reaction mixture was filtered. Chromatography of the filtrate (petroleum ether/ethyl acetate=7:1) afforded Compound S00628 as a white solid (65 mg, 59%).

### Example 7: Synthetic Procedures

All compounds listed in Tables 1, 2, and 3 were synthesized using methods identical to or similar to those described in the examples below.

General Procedure for the Synthesis from Halide-Substituted Pyridine Analogs to Target Compounds.

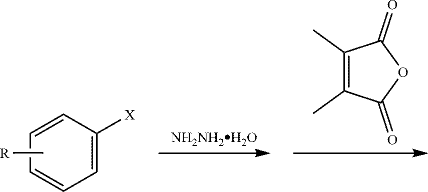

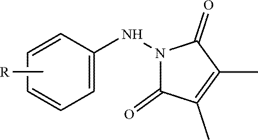

Starting material dissolved in ethanol and hydrazine hydrate (10.0 eq) was added to form a mixture, the mixture was stirred at 50-60° C. (oil temperature) for several hours (completion was checked by TLC), the solvent was evaporated, water was added and the resulting mixture was extracted with ethyl acetate, dried and concentrated to form a crude preparation that was used without further purification for the next step. The crude preparation was dissolved in chloroform (or toluene, acetic acid, or another suitable solvent), anhydride was added (1.0 eq), the mixture was heated at 50-60° C. (oil temperature) for several hours (completion checked by TLC), the solvent was evaporated, and the preparation was purified by Prep-TLC to provide the desired compound.

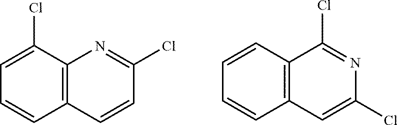 [SMILES: Clc1ccc2cccc(Cl)c2n1 ; Clc1cc2ccccc2c(Cl)n1]

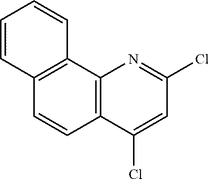 [SMILES: Clc1cc(Cl)c2ccc3ccccc3c2n1]

The starting materials were commercially available, so the synthetic route of compounds S00585, S01098, S01207 was similar to general procedure.

### A. Compound S00109

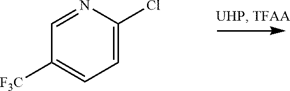

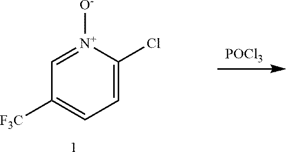

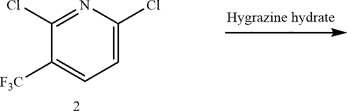

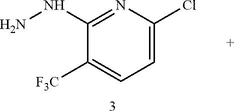

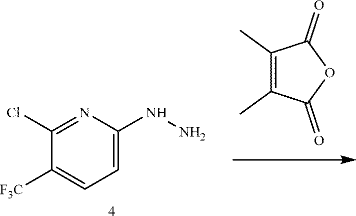

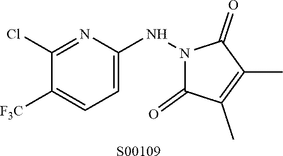 [SMILES: CC1=C(C)C(=O)N(Nc2ccc(C(F)(F)F)c(Cl)n2)C1=O ; "S00109"]

### B. Intermediate 1

2-Chloro-5-trifluoromethyl-pyridine (10 mmol) was dissolved in CH₂Cl₂ (20 mL) and UHP (Urea-hydrogen peroxide addition compound, 21 mmol) was added. The mixture was cooled to 0° C., trifluoroacetic anhydride (20 mmol) was then slowly added to the reaction mixture. It was allowed to warm to room temperature and stirred until the reaction was completed monitored by TLC. The reaction was quenched with aqueous Na₂S₂O₃, stirred for 4 h, washed with saturated aqueous NaHCO₃, and dried over anhydrous MgSO₄. Column chromatography afforded 1.8 g of intermediate compound 1 as oil.

### C. Intermediate 2

Intermediate compound 1 (4 mmol) was dissolved in freshly distilled POCl₃ (4.5 mL). The reaction mixture was heated to 80° C. for 17 h. After cooling to room temperature, the solvent was removed under reduced pressure. Ice was added, and the mixture was allowed to stand for 4 h. The mixture was partition between CH₂Cl₂ (50 mL) and saturated aqueous NaHCO₃. Column chromatography afforded intermediate compound 2 as yellow oil (yield: 50%).

### D. Intermediate 4

To the solution of intermediate compound 2 (2 g, 9.26 mmol) in ethanol (30 mL) was added hydrazine hydrate (2.9 g, 46 mmol). The reaction mixture was stirred for 4 h at room temperature, then concentrated to remove the solvent, and added ethyl acetate, washed with water. The organic layer was dried over anhydrous Na₂SO₄. Column chromatography (Silica, petroleum ether/ethyl acetate=4/1∼3/1) afforded intermediate compound 4 as white solid (yield: 56%) and another isomer 3 (yield: 18%).

### E. Compound S00109

The synthetic procedure was similar to general procedure.

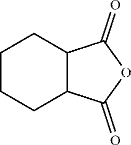 [SMILES: O=C1OC(=O)C2CCCCC12]

### F. Compound S00186

The starting material (anhydride) was commercially available, so the synthetic route of compounds S00186 was similar to general procedure (anhydride react with intermediate 4).

### G. Compound S00994

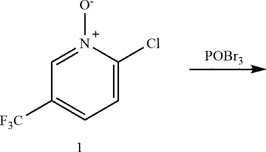

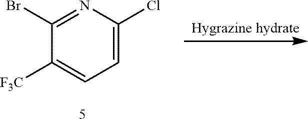

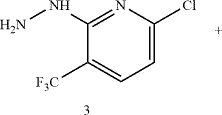

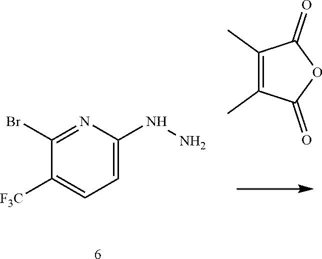

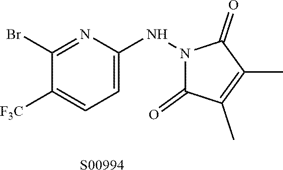

### H. Intermediate 5

Intermediate compound 1 (4 mmol) was dissolved in freshly distilled POBr₃ (4.5 mL). The reaction mixture was heated to 80° C. for 17 h. After cooling to room temperature, the solvent was removed under reduced pressure. Ice was added, and the mixture was allowed to stand for 4 h. The mixture was partitioned between CH₂Cl₂ (50 mL) and saturated aqueous NaHCO₃. Column chromatography afforded intermediate compound 5 as yellow oil (yield: 50%).

### I. Intermediate 6

Hydrazine hydrate was added to the solution of intermediate compound 5 in ethanol. The reaction mixture was stirred for 4 h at room temperature, then concentrated to remove the solvent, ethyl acetate was added, and the mixture was washed with water. The organic layer was dried over anhydrous Na₂SO₄. Column chromatography (Silica, petroleum ether/ethyl acetate=4/1^{~}3/1) afforded intermediate compound 6 as a white solid.

### J. Compound S00994

The synthetic procedure was similar to general procedure.

### K. Compound S01860

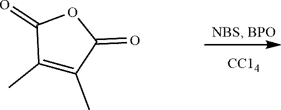

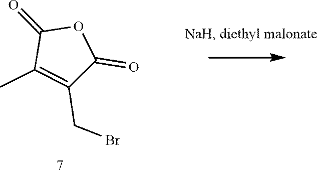

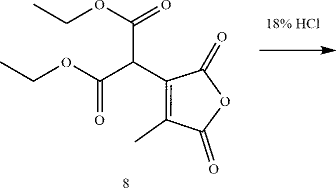

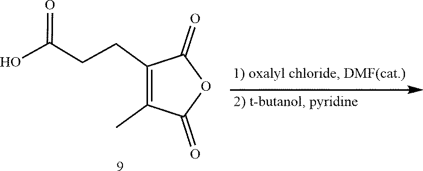

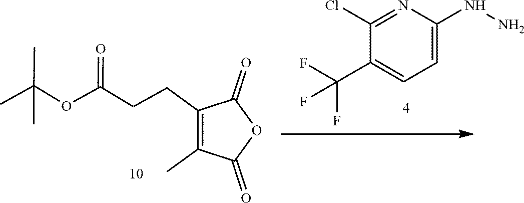

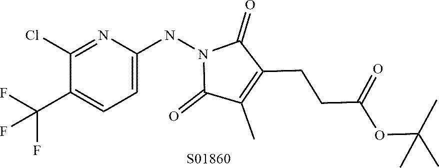

### L. Intermediate 7

A solution of starting material (5.0 g, 0.040 mol), NBS (10.6 g, 0.059 mol) BPO (296 mg) in 300 ml CCl₄ was stirred under reflux for 5 hrs. The reaction mixture was then cooled to room temperature, and another portion of BPO (296 mg) was added, and the reaction was stirred under reflux for another 5 hrs. The reaction mixtures was then held at room temperature overnight. Then it was filtered and the residue was washed by CCl₄ for three (3) times, and the combined organic layer was washed by water and brine, then dried and concentrated and purified by column chromatography (PE:EA=4:1) to give crude product that was then was purified by distillation. The second fraction obtained at 128° C.^{~}135° C. (3 mmHg) was intermediate 7.

### M. Intermediate 8

To the slurry of sodium hydride (60 mg, 1.5 mmol) in benzene (5 mL), diethylmalonate (320 mg, 2.0 mmol) was added dropwise at room temperature. The reaction mixture was stirred for 5 min, then a solution of intermediate 7 (210 mg, 1.0 mmol) in benzene (5 mL) was added. The mixture was stirred at room temperature for another 8 h. Then the mixture was acidified with diluted HCl and extracted with EtOAc (2×15 mL). The combined organic layers were washed with water, brine and dried over anhydrous Na₂SO₄. Concentration of the organic layers in vacuo followed by silica gel column chromatographic purification of the residue (petroleum ether:EtOAc=4:1) furnished the product (intermediate 8) as a thick oil. Yield was 200 mg, (74.0%).

### N. Intermediate 9

A solution of intermediate compound 8 (80 mg, 0.3 mmol) in diluted hydrochloride (2 mL, 18%) was refluxed with stirring for 12 h. The reaction mixture was cooled to room temperature, and saturated by adding solid sodium chloride. The filtered aqueous layer was extracted with EtOAc, dried over anhydrous Na₂SO₄ and concentrated to furnish pure acid (intermediate 9). Yield was 50 mg (90.6%).

### O. Intermediate 10

To a stirred solution of intermediate 9 (0.46 g, 2.5 mmol) and two drops of DMF in DCM (10 ml) was added oxalyl chloride (0.48 g, 3.75 mmol) dropwise. The mixture stirred at room temperature (oil temperature 20-30° C.) for two hours, then the solvent was evaporated. The residue and tert-butanol (0.22 g, 3 mmol) were dissolved in 10 ml of DCM, pyridine (0.3 g, 3.75 mmol) was added to this solution dropwise at room temperature. The resulting mixture stirred at room temperature for an hour. Added sat. NH₄Cl to quench the reaction, adjusted pH to 2 with 1 N HCl and extracted with ethyl acetate, the combined organic layer dried over Na₂SO₄, filtered and evaporated. The residue purified by flash chromatography to give intermediate 10 as white solid (0.42 g, 70%).

### P. Compound S01860

Intermediates 10 (119 mg, 0.45 mmol) and 4 (95 mg, 0.49 mmol) were added to 5 ml of DCM and refluxed overnight, then the solvent was evaporated and the residue purified by Prep-TLC to give the product (S01860). (Yield=150 mg, 77%)

### Q. Compound S01861

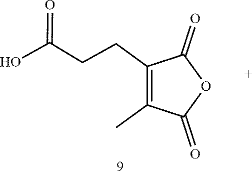

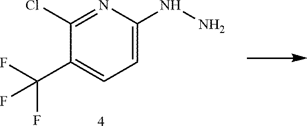

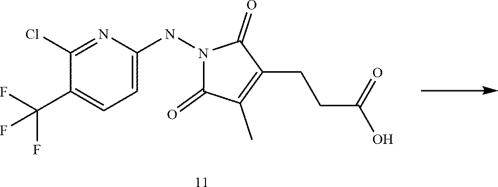

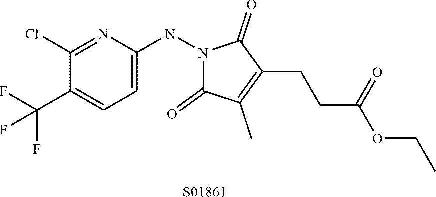

### R. Intermediate 11

Intermediates 9 (1.0 g, 5.43 mmol) and 4 (1.15 g, 5.43 mmol) were dissolved in 20 ml of chloroform and refluxed for 48 h, then evaporated the solvent and the residue recrystallized to give intermediate 11 (1.4 g, 68.2%).

### S. Compound S01861

Intermediate 11 (15 mg, 0.04 mmol), EDCI (45 mg, 0.24 mmol), Et₃ N (1 drop) and ethanol (1 mL) was stirred at room temperature for about 3 h. Then the solvent was removed under vacuum. The product (S01861) was separated by Prep-TLC. Yield was 12 mg (76.7%).

### T. Compounds S01648, S01796, S01711, S01758, S01883, and S01759

The synthetic route of compounds S01648, S01796, S01711, S01758, S01883, and S01759 was similar to S01861 (i.e., intermediate 11 coupled to different chemicals).

### U. Compound S01589

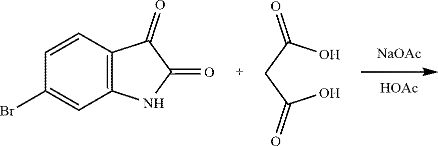

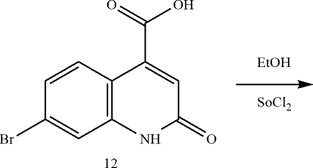

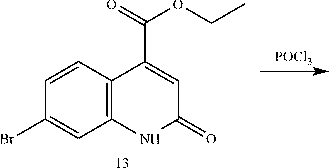

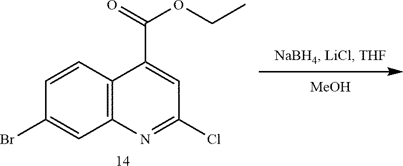

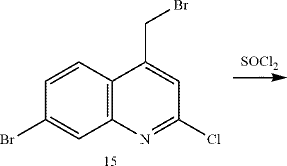

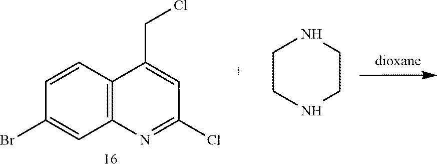

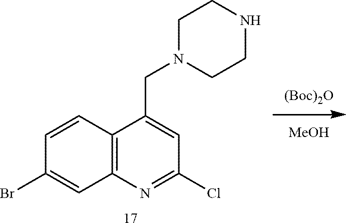

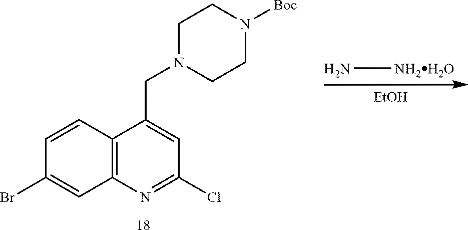

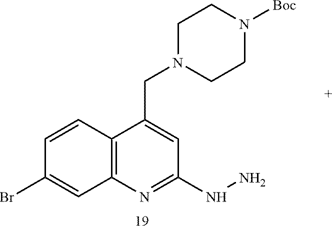

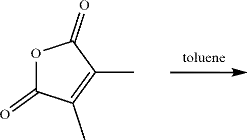

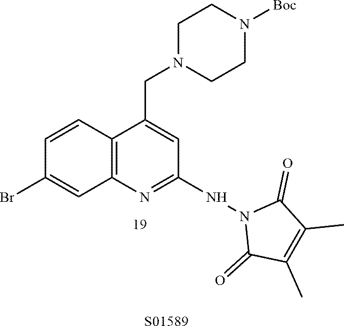

### V. Intermediate 12

The mixture of starting material (6.5 g, 28.7 mmol), malonic acid (3.3 g, 31.7 mmol), HOAc (60 ml), NaOAc (2.95 g, 36 mmol) were stirred at RT. After 6-7 hrs, NaOAc (2.95 g, 36 mmol) was added additional, then refluxed overnight. After cooling, the mixture was filtered and the filtrate was washed with water and ethyl acetate, then dried under reduced pressure. 5 g thin brown solid was collected to afford intermediate 12 (yield=65.4%).

### W. Intermediate 13

Four (4) ml of SOCl₂ was added dropwise to a suspension of intermediate compound 12 and EtOH, in an ice bath, and the mixture was stirred for 30 min at room temperature, then refluxed for 6 hrs. After cooling, the mixture was filtered and washed with chilled EtOH, and dried in vacuo to obtain 5.25 g pale grey powder as intermediate 13 (yield=95%)

### X. Intermediate 14

A mixture of intermediate compound 13 and POCl₃ (15 ml) was stirred at room temperature for 15 min, then refluxed for 2 hrs. The mixture was concentrated in vacuo. The residue was quenched with cooled water and extracted with ethyl acetate, washed with saturated NaHCO₃ and brine, dried over MgSO₄, concentrated and 4.68 g thin brown solid was collected as intermediate 14.

### Y. Intermediate 15

To a solution of THF and MeOH, intermediate compound 14 (4.68 g, 14.9 mmol) and LiCl was added with ice-salt bath, NaBH₄ was added by portions. After addition, the reaction mixture was stirred at room temperature, checked by TLC, concentrated in vacuo, and dilute HCl was added slowly to the residue over an ice bath until the mixture reached pH 7. The mixture was then extracted with ethyl acetate and washed with saturated NaHCO₃, NH₄Cl, NaCl solutions (in sequence), dried over MgSO₄, concentrated, and 4.15 g thin brown solid was collected as intermediate 15.

### Z. Intermediate 16

Intermediate Compound 15 (4.15 g) was dissolved in SOCl₂ and refluxed overnight. The solvent was evaporated, water was added to the residue, the mixture was extracted with ethyl acetate, the combined organic layer was dried over anhydrous Na₂SO₄, the solvent was evaporated, and 4.0 g intermediate compound 16 was collected.

### AA. Intermediate 17

Intermediate Compound 16 (200 mg, 0.69 mmol) was dissolved in dioxane, and anhydrous piperazine (177 mg, 2.05 mmol) was added, and the mixture was stirred overnight. The mixture was filtered, the filtrate was concentrated in vacuo, and 250 mg crude product was collected as intermediate 17.

### BB. Intermediate 18

A solution of di-tert-butyl dicarbonate (0.246 g, 1.13 mmol) in MeOH was added dropwise to intermediate compound 17 (0.35 g, 1.03 mmol) in MeOH at room temperature. The reaction mixture was stirred overnight at room temperature. The solvent was evaporated, the residue was extracted in the usual manner as described above, and the extract was purified by chromatography column (EA:PE=1:10). The product (intermediate 18) was obtained as a white solid.

### CC. Compound S01589

The synthetic procedure from intermediate 18 to compound S01589 was similar to general procedure described herein.

### DD. Compounds S01037 and S01047

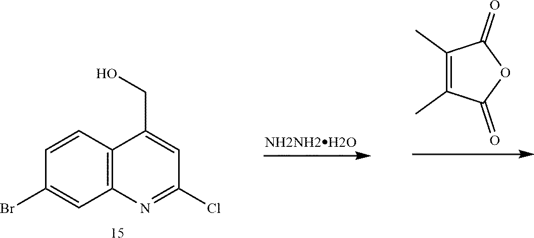

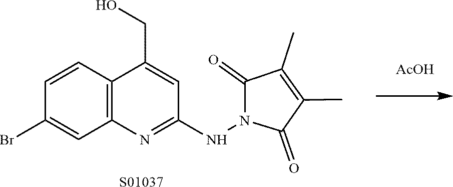

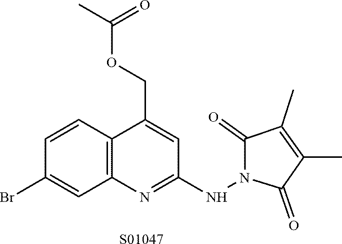

### EE. Compound S01037

The synthetic procedure from intermediate 15 to compound S01037 is similar to general procedure.

### FF. Compound S01047

Starting material (0.145 g, 0.54 mmol) was dissolved in 5 ml of acetic acid and the mixture was heated with refluxing for 1 h, then evaporated and purified by Prep-TLC (petroleum ether:ethyl acetate=1:1) to give the product (S01047).

### GG. Compound S01879

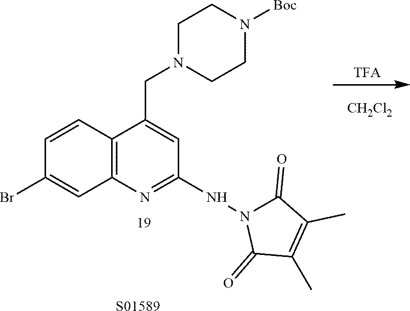

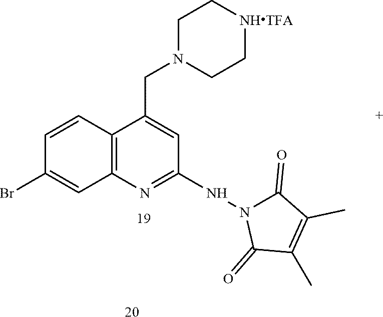

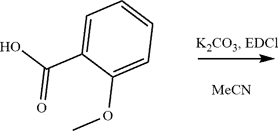

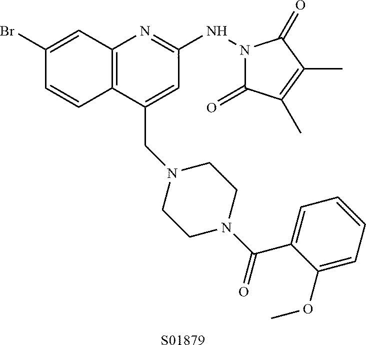

### HH. Intermediate 20

Starting material (50 mg, 0.09 mmol) was dissolved in 5 ml of CH₂Cl₂ and TFA (5 ml) was added dropwise to the stirred mixture over an ice bath. The resulting mixture was stirred for 1 h at room temperature and checked by TLC. The solvent was evaporated to give the product (intermediate 20) as yellow solid which was used without further purification. (40 mg).

### II. Compound S01879

Intermediate Compound 20 was dissolved in MeCN, and K₂CO₃ (3 eq) was added, after which the mixture was stirred for about 30 min, and benzoic acid (1 eq) and EDCI (2 eq) was added and the mixture was stirred overnight, then concentrated and worked up in the usual manner described above. The final preparation was purified by Prep plate TLC and product (S01879) was obtained as a thin yellow solid.

### JJ. Compounds S01925, S01878, S01877, S01699, S01800, S01801, S01822, S01880, S01683, S01928, S01929

The synthetic route of compounds S01925, S01878, S01877, S01699, S01800, S01801, S01822, S01880, S01683, S01928, S01929 was similar to S01879 (intermediate 20 coupled with different chemicals).

### KK. Compound S01981

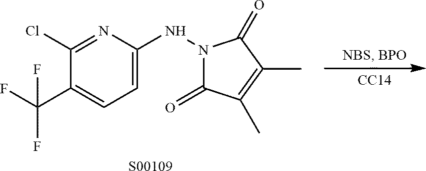

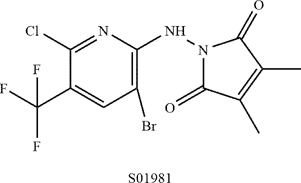

Starting material (100 mg, 0.314 mmol) was dissolved in CCl₄, NBS (112 mg, 0.629 mmol) and BPO (1.5 mg, 0.0062 mmol) were added, and the mixture was refluxed for about 4 hrs. The reaction mixture was quenched with water, extracted with ethyl acetate, the organic layer washed with brine, dried over MgSO₄ and concentrated in vacuo, then purified by prep plate to obtain product (S01981).

### LL. Compound S00170

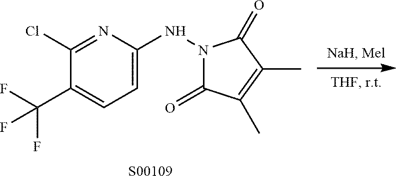

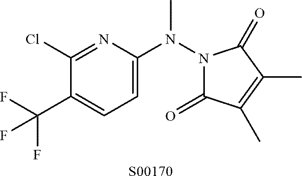 [SMILES: CC1=C(C)C(=O)N(N(C)c2ccc(C(F)(F)F)c(Cl)n2)C1=O ; "S00170"]

NaH (8 mg, 0.12 mmol) was added dropwise to a solution of hydrazine (35 mg, 0.117 mmol) in THF (6 mL), at 0° C. The mixture was stirred for 30 min, then added MeI (20 mg). The reaction mixture was stirred for 2 h at room temperature, then poured into the Sat. NH₄Cl aq.; extracted with CHCl₃. The organic layer was dried over Na₂SO₄, then chromatography (PE/AE, 5/1) to obtain the product S00170 (3 mg).

### MM. Compounds S01007 and S01473

The synthetic route of compounds S01007 and S01473 was similar to S00170.

### NN. Compound S01470

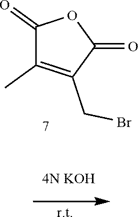

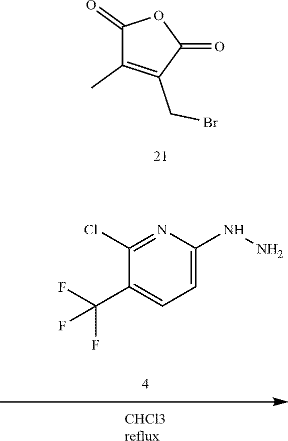

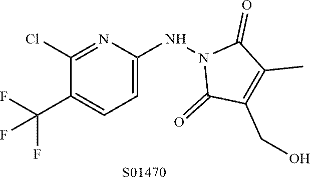

### OO. Intermediate 21

Intermediate Compound 2 (1 g, 4.9 mmol) was added to an ice cold solution of 4 N aq. KOH (5 ml), and the mixture was stirred at room temperature for 5 hrs. The mixture was slowly acidified with 6 N H₂SO₄ (5 ml), then saturated with solid NaCl and stirred at room temperature for 30 min. The aqueous layer was extracted with ethyl acetate and the organic layer was washed with brine and dried. The organic layer was concentrated in vacuo and the concentrate was applied to silica gel (PE:EA=1:1) to furnish 355 mg of product (intermediate 21).

### PP. Compound S01470

The synthetic procedure was similar to general procedure.

### QQ. Compounds S01599 and S01600

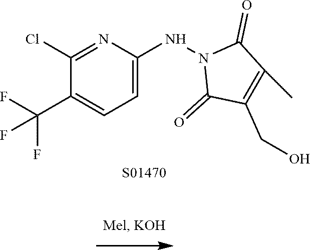

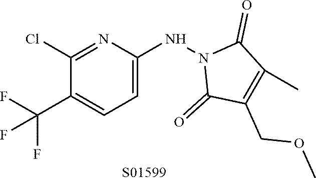

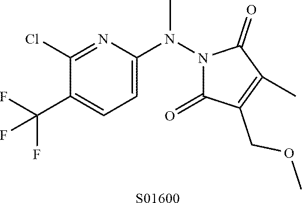

Starting material (80 mg, 0.24 mmol), MeI (40 uL, 0.64 mmol), and KOH (30 mg, 0.54 mmol) in DMSO (5 mL) was stirred at room temperature for 1 h, then diluted with EtOAc, washed with water, brine, dried over anhydrous Na₂SO₄. The solvent was removed in vacuum and the residue was purified by Prep-TLC to obtain the two target compounds (S01599 and S01600).

### RR. Compound S01712

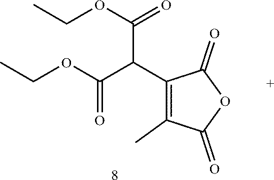

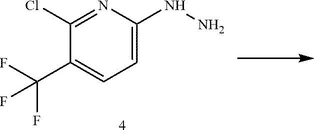

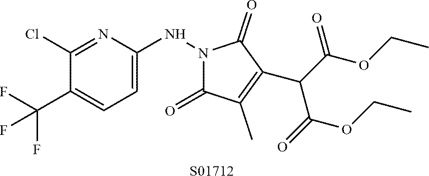

The synthetic procedure was similar to the general procedure.

### SS. Compound S01266

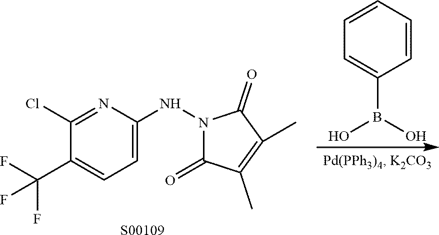

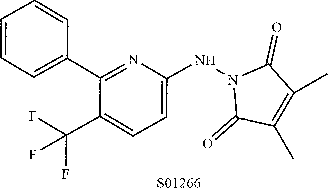

Pd(PPh₃)₄ (16 mg) was added to a mixture of starting material (50 mg, 0.14 mmol), benzeneboronic acid (19 mg, 0.15 mmol), potassium carbonate (59 mg, 0.43 mmol) in 10 ml of toluene under a nitrogen atmosphere. The resulting mixture was refluxed for 16 h, after which the solvent was evaporated and the residue purified by preparative TLC to give 4 mg of product (S01266).

### TT. Compounds S01313, S01457, S01691, S01371, S01393, S01474

The synthetic route of compounds S01313, S01457, S01691, S01371, S01393, S01474 was similar to S01266.

### UU. Compound S01737

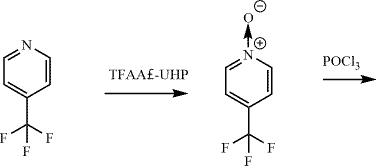

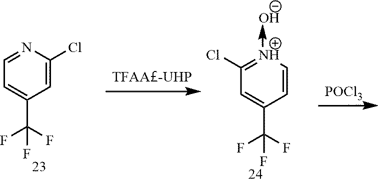

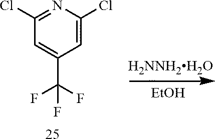

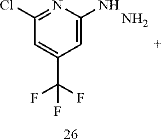

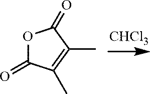

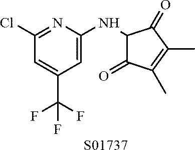

### VV. Intermediate 22

The pyridine (500 mg, 3.4 mmol) was dissolved in CH₂Cl₂ and UHP (700 mg, 7.4 mmol) was added, which was cooled to 0° C., TFAA (1.43 g, 6.8 mmol) was then slowly added to the reaction mixture. After TLC indicated starting material was consumed, work up as usual manner to afford 420 mg of target compound (intermediate 22).

### WW. Intermediate 23

Intermediate Compound 22 (420 mg, 2.57 mmol) was dissolved in POCl₃ (3 ml), then heated at 90° C. overnight. The reaction mixture was quenched to water carefully, extracted by CH₂Cl₂, washed with brine and dried over MgSO4, concentrated in vacuo. Purified by chromatography column (CH₂Cl₂:PE=1:3) then obtained 300 mg target compound (intermediate 23).

### XX. Intermediate 24

The reaction and work-up procedure was the same as for intermediate 22, and 170 mg target compound (intermediate 24) was obtained.

### YY. Intermediate 25

The reaction and work-up procedure was the same as for intermediate 23, and 120 mg target compound (intermediate 25) was obtained.

### ZZ. Compound S01737

The synthetic procedure from intermediate 25 to target compound (S01737) was similar to the general procedure.

### AAA. Compound S01865

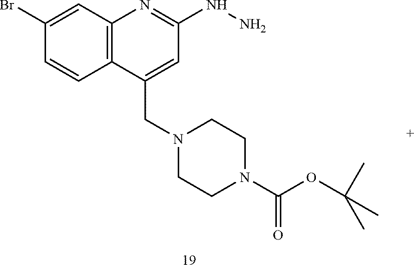

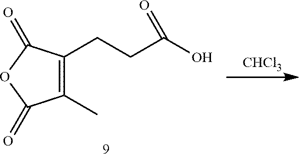

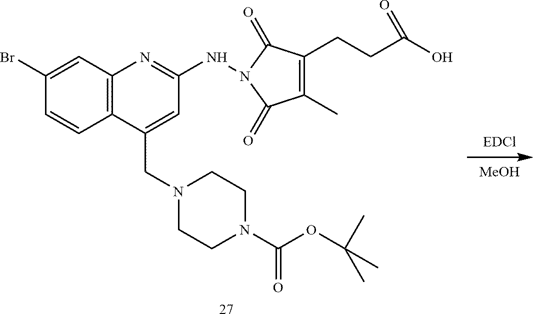

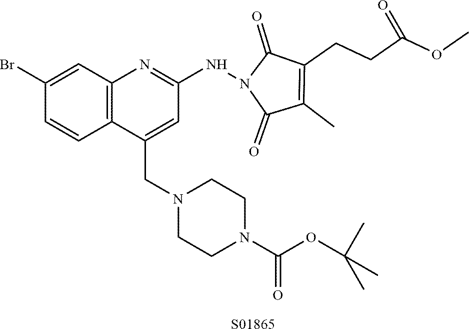

### BBB. Intermediate 27

Two starting materials were dissolved in CHCl₃ and refluxed overnight, then concentrated and purified by chromatography column (EA:PE=1:1). The product (intermediate 27) was obtained as a light yellow solid.

### CCC. Compound S01865

Intermediate Compound 27 was dissolved in anhydrous MeOH, EDCI was added, then stirred overnight. Concentrated in vacuo, work up as usual manner and purified by Prep-TLC to obtain the final product (S01865) as a light yellow solid.

### DDD. Compounds S01734 and S01688

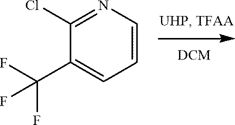

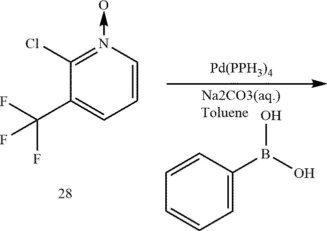

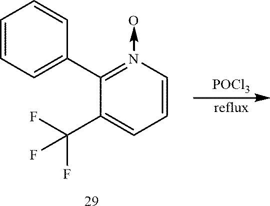

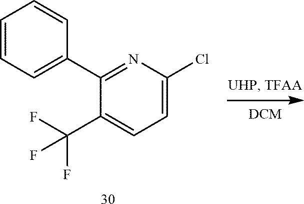

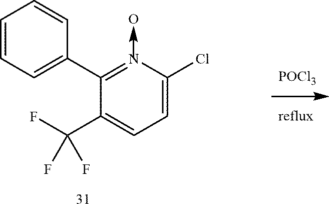

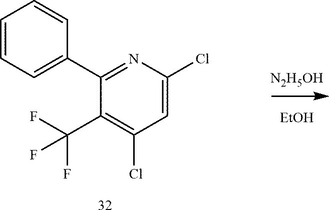

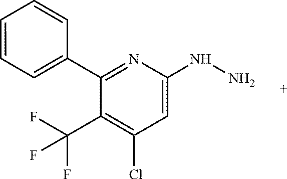

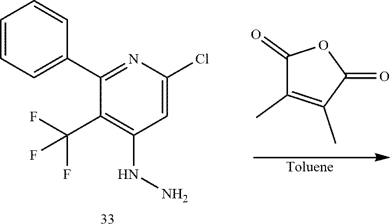

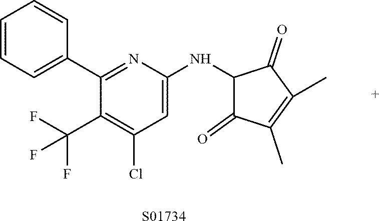

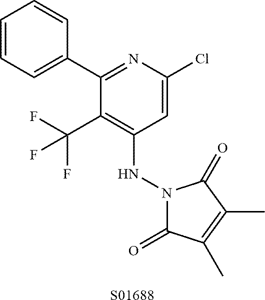

### EEE. Intermediate 28

To a solution of starting material (9.26 g, 0.05 mol), UHP (9.9 g, 0.105 mol) was added. With ice-bath, TFAA (21 g, 0.100 mol) was added dropwise. After addition, the reaction was maintained at room temperature, for 4 hrs. Neutralize the reaction with Na₂CO₃ (aq.) and the mixture was extracted with DCM for 3 times. The organic layer was collected, dried and concentrated and purified by flash chromatography (PE:EA=3:1) to give the pure product (intermediate 28) 8.1 g.

### FFF. Intermediate 29

A solution of intermediate compound 28 (0.8 g, 4.07 mmol) and in 2 ml Na₂CO₃ (aq. 2N) and 3 ml toluene was stirred under an atmosphere of N₂ and at room temperature. Then Pd(PPh₃)₄ was added. The mixture was stirred under reflux at an atmosphere of N₂ for 3 hrs. Then the solvent was removed under vacuum. The residue was treated with water and extracted with EA, and the organic phase was collected, dried and concentrated to be purified by recrystallization to give. 0.75 g of pale yellow powder as intermediate 29.

### GGG. Intermediate 31

A solution of intermediate compound 29 (0.75 g, 3.15 mmol) in 5 ml POCl₃ and the mixture was stirred under reflux for 5 hrs. Then the reaction mixture was poured into ice and the aqueous layer was extracted with ethyl acetate for 3 times. Then the organic phase was collected, washed with Na₂CO₃ aqueous solution and then dried, concentrated and to be purified by column chromatography to afford 800 mg intermediate 30, which was dissolved in 5 ml DCM and UHP, followed by TFAA was added to the above mixture under ice-bath. Then the reaction mixture was stirred at room temperature, overnight. Then neutralized the reaction mixture by Na₂CO₃ aqueous solution and the aqueous phase was extracted by DCM for 3 times. The organic phase was collected, dried, concentrated and purified by column chromatography (PE:EA=5:1) to give 350 mg of pure intermediate compound 31.

### HHH. Intermediate 32

A solution of intermediate compound 31 (350 mg, 1.28 mmol) in 5 ml POCl₃ was stirred under reflux for 4 hrs. Then the mixture was poured into ice water and extracted with ethyl acetate. The organic layer was washed with Na₂CO₃ aqueous solution and dried, concentrated and purified to give 180 mg pure intermediate compound 32.

### III. Compounds S01734 and S01688

The synthetic procedure from intermediate 32 to the target compounds (S01734 and S01688) was similar to the general procedure.

### JJJ. Compound S01864

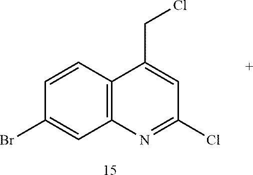

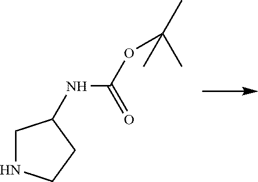

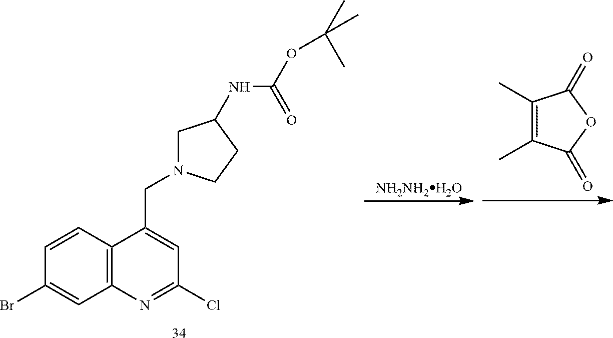

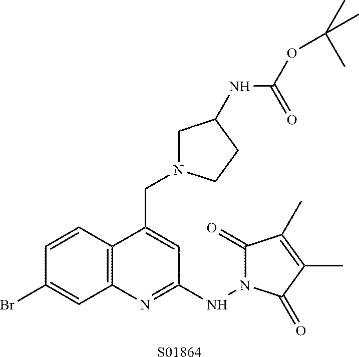

### KKK. Intermediate 34

Starting material and intermediate 15 were dissolved in acetonitrile and stirred at room temperature overnight, then filtered and the solvent was evaporated. The residue was purified by preparative TLC to afford the product (intermediate 34).

### LLL. Compound S01864

The synthetic procedure from intermediate 34 to the target compound (S01864) is similar to the general procedure.

### MMM. Compounds S01268 and S01862

The synthetic route for compounds S01268 and S01862 was similar to that for compound S01864.

### NNN. Compound S01475

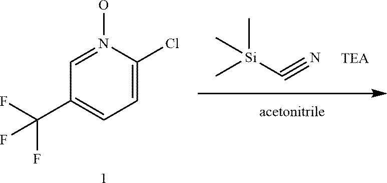

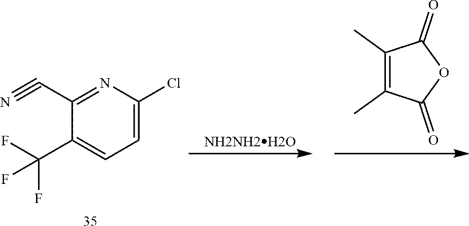

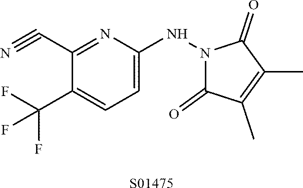

### OOO.Intermediate 35

Trimethylsilyl cyanide (7.44 g, 75 mmol, 10 ml) was added to a stirred solution of intermediate 35 (5.92 g, 30 mmol) and TEA (4.55 g, 45 mmol, 6.3 ml) in 25 ml of acetonitrile at room temperature. The mixture was then heated to 110° C. (oil bath temperature) for 12 h, cooled down to room temperature, and the solvent was evaporated. DCM and saturate NaHCO₃ (aq.) were added and the layers were separated. The organic layer was dried over anhydrous Na₂SO₄ and evaporated. The residue was washed with ether and filtered, then evaporated to give the crude product as a black oil that was then purified by flash chromatography to give the product (intermediate 35) as a yellow oil. Yield was 4.4 g (71%).

### PPP. Compound S01475

The synthetic procedure from intermediate 35 to Compound S01475 was similar to the general procedure.

### QQQ. Compound S01762

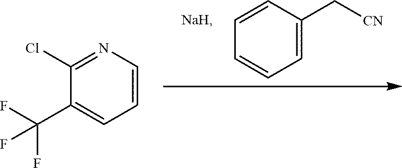

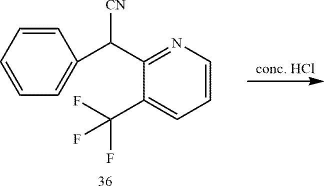

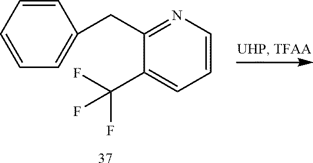

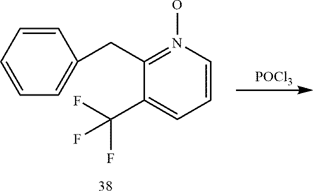

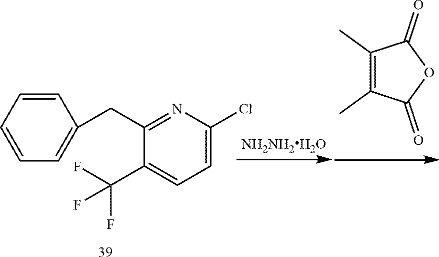

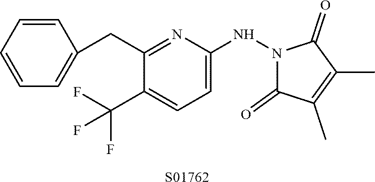

### RRR. Intermediate 36

Sodium hydride (0.264 g, 6.6 mmol, 60%) was added to a stirred solution of benzyl cyanide (0.645 g, 5.5 mmol) in 10 ml of DMF at room temperature. Starting material (1.0 g, 5.5 mmol) was added to the mixture after 30 min and the resulting mixture was stirred at room temperature for 2 h. Brine was added to quench the reaction and the mixture was extracted with ethyl acetate. The combined organic layer was dried over anhydrous sodium sulfate and evaporated. The residue was purified by flash chromatography (eluted with petroleum ether:ethyl acetate=8:1 to 5:1) to give 0.475 g of product (intermediate 36) (yield=33%).

### SSS. Intermediate 37

Intermediate 36 (0.15 g, 0.57 mmol) mixed with 5 ml of concentrated HCl and refluxed overnight. Then the mixture cooled to room temperature, 15 ml of water was added, the pH was adjusted to 8-9 with sodium carbonate, and the mixture was extracted with ethyl acetate (10 ml). The combined organic layer was dried over anhydrous sodium sulfate and evaporated to give 0.14 g grams of product (intermediate 37) (yield=100%), which was used without further purification for the next step.

### TTT. Intermediate 38

Intermediate 37 (0.14 g, 0.57 mmol) was dissolved in 5 ml of DCM, then UHP (0.17 g, 1.77 mmol) was added and after that, TFAA (0.36 g, 1.71 mmol, 0.24 ml) was added dropwise with ice-bath cooling. The mixture was then warmed to room temperature and stirred overnight at the same temperature. Five (5) ml of water was added and the mixture was neutralized with sodium carbonate to pH 8-9 and then extracted with DCM. The combined organic layer was dried over anhydrous sodium sulfate and evaporated to give 0.14 g of the crude product (intermediate 38) (yield=95%), which was used without further purification for next step.

### UUU. Intermediate 39

Intermediate 38 (0.14 g, 0.55 mmol) was dissolved in 5 ml of POCl₃ and the mixture was heated to 80-90° C. for 2 h. The mixture was then cooled to room temperature, poured into ice-water, and extracted with ethyl acetate. The combined organic layer was washed with sat. NaHCO₃, dried over Na₂SO₄ and evaporated. The residue was purified by preparative TLC to afford 0.13 g of product (intermediate 39) (yield=87%).

### VVV. Compound S01762

The synthetic procedure from intermediate 39 to compound S01762 was similar to the general procedure.

### WWW. Compound S01820

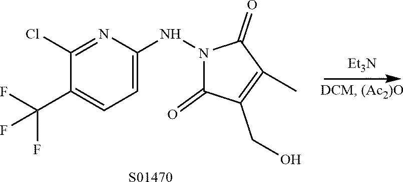

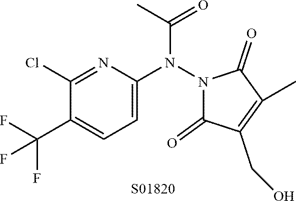

A solution of S01470 in 3 ml DCM was stirred at room temperature and Et₃ N was added. Then Ac₂O was added under ice-bath. The reaction mixture was warmed to room temperature and stirred overnight. The reaction was then quenched and worked-up in the usual manner as described above. The residue was purified by prep-TLC (PE:EA=3:1) to furnish pure compound S01820.

### XXX. Compound S00935

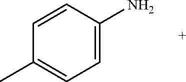

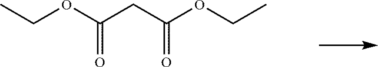

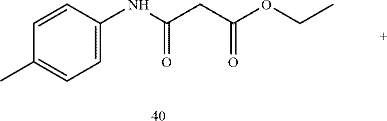

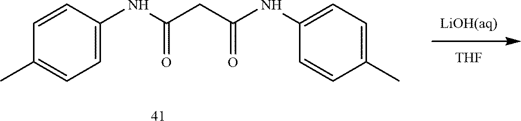

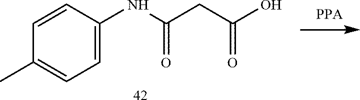

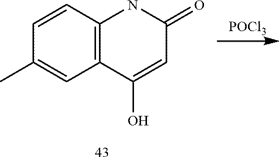

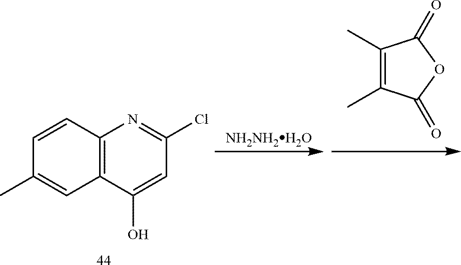

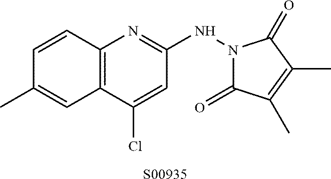

### YYY. Intermediate 40

Starting material (9.08 g, 84.4 mmol) was added to 19.2 ml of diethyl malonate, the mixture was heated to 150° C. (oil bath temperature) for 6 h, evaporated, filtered and washed with ethyl acetate to give 3.7 g of white solid, it was intermediate 41 (check it by LC-MS), the filtrate was evaporated, the residue cooled to afford second batch solid, washed with a solution of Petroleum ether:Ethyl acetate equal to 5:1, check it by LC-MS, it was intermediate 40 (5.42 g).

### ZZZ. Intermediate 42

To a stirred solution of intermediate 40 (5.42 g, 24.5 mmol) in THF, 60 ml of 2 N LiOH was added and the resulting mixture was stirred at room temperature for 3 h. The solvent was evaporated, the residue washed with ethyl acetate, filtered, and the cake was added to 10 ml of concentrated HCl and stirred for 30 min, then the cake was filtered and dried to give 3.1 g of product (intermediate 42).

### AAAA. Intermediate 43

Intermediate 42 (3.1 g, 16 mmol) was added to 20 ml of PPA and the mixture was heated to 150° C. for 4 h. The reaction mixture was poured into ice-water with stirring, then filtered, and the cake was washed with water and dried to give 2.92 g of product (intermediate 43).

### BBBB. Intermediate 44

Intermediate 43 (0.47 g, 2.7 mmol) was added to 10 ml of POCl₃, and the mixture was heated with refluxing for 5 h. The resulting mixture was cooled to room temperature and poured into ice-water, then extracted with ethyl acetate. The combined organic layer was dried over Na₂SO₄, and evaporated to give the crude product (intermediate 44) (0.45 g), which was used without further purification.

### CCCC. Compound S00935

From intermediate 43 to compound S00935, the synthetic procedure was similar to the general procedure.

### Compounds S00871, S01005, S01078, S01247, and S01311

The synthetic route of compounds S00871, S01005, S01078, S01247, and S01311 is similar to compound S00935.

### DDDD. Compound S00516

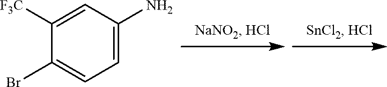

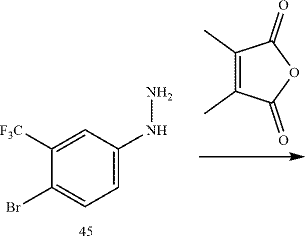

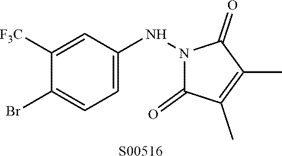

### EEEE. Intermediate 45

Starting material (0.08 mol) was added to conc. HCl (40 mL). The mixture was cooled to -5° C. by ice and salt with stirring. Then sodium nitrite (5.52 g, 0.08 mol) dissolved in water (20 mL) was added. Stirring was continued for 1 h, and stannous chloride (30 g) in conc. HCl (30 mL) was added slowly over a period of two hours, while keeping the temperature below 0° C. The mixture was stirred for another hour after the addition and filtered. The filtered solid was treated with dilute aqueous sodium hydroxide and the then extracted with ether. The ether layer was washed with water, dried over anhydrous Na₂SO₄. The solvent was removed and the residue was crystallized from hexane to give the intermediate Compound 45.

### FFFF. Compound S00516

The synthetic procedure is similar to general procedure.

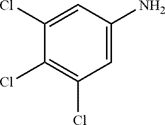

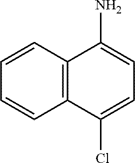 [SMILES: Nc1ccc(Cl)c2ccccc12]

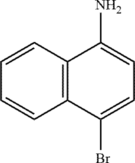 [SMILES: Nc1ccc(Br)c2ccccc12]

### GGGG. Compounds S00738, S00832, and S00942

The starting materials are commercially available, so the synthetic route of compounds S00738, S00832, S00942 was similar to S00516.

### HHHH. Compound S01191

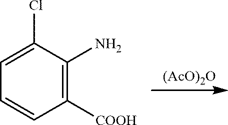 [SMILES: "(AcO)2O" ; Nc1c(Cl)cccc1C(=O)O]

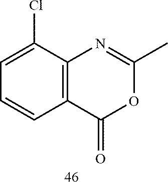

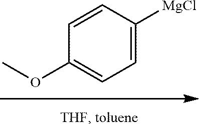

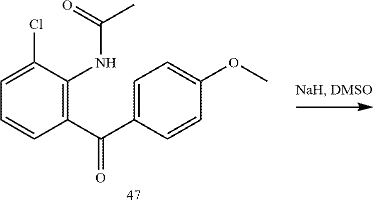

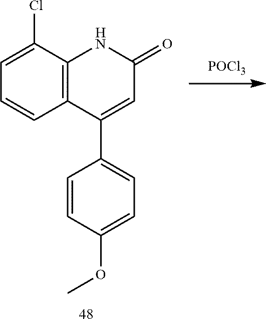

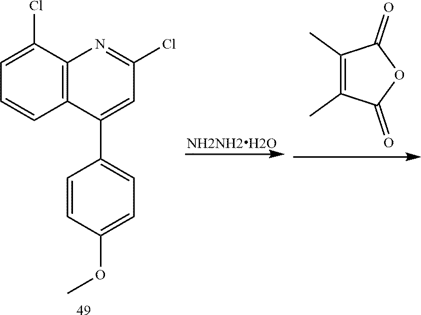

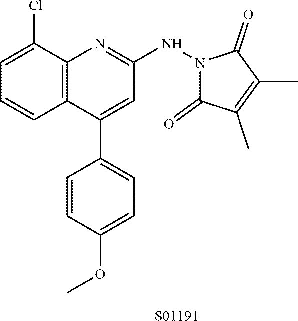

### IIII. Intermediate 46

A mixture of 2-amino-3-chlorobenzoic acid (500 mg, 2.91 mmol) and acetic anhydride (1.2 mL) was heated with refluxing for 1 hour, and excess acetic anhydride was removed under vacuum. The residue was cooled and treated with diethyl ether to give a bulk precipitate, which was filtered off, washed with cold ether and dried to give 550 mg of the desired product (intermediate 46) as a pale yellow solid (yield=97%).

### JJJJ. Intermediate 47

Into a three-necked flask, which had been oven dried and flushed with N₂, was added a small amount of I₂ to a mixture of magnesium (59 mg, 2.47 mmol) in 0.5 mL of dry THF. When the reaction mixture became colorless, a solution of 4-bromoanisole (440 mg, 2.35 mmol) in 1.5 mL of dry THF was added to the mixture. The reaction mixture was stirred at room temperature until Mg was eliminated.

The Grignard reagent from 4-bromoanisole in 2 mL of THF was treated with intermediate compound 46 (460 mg, 2.35 mmol) in 4.5 mL dry toluene at 0° C. for 1 hour and at 30° C. for an additional 1 hour. The solution was carefully acidified with dilute sulphuric acid, and washed with aqueous NaHCO₃ and water. The organic layer was dried over anhydrous Na₂SO₄ and evaporated to give an oil. The residue was purified by silica gel chromatography (petroleum ether/ethyl acetate=4:1) to give 450 mg of the desired product (intermediate 47) as pale brown solid (yield=63%).

### KKKK. Intermediate 48

A mixture of intermediate compound 47 (400 mg, 1.32 mmol), NaH (60% in oil, 316 mg, 13.20 mmol) in 1 mL of DMSO was heated at 60-70° C. overnight. The reaction mixture was poured into ice-water and extracted with ethyl acetate, then washed with water and brine. The organic layer was dried over anhydrous Na₂SO₄ and evaporated to dryness. The residue was recrystallized from ethanol to give 80 mg of the desired intermediate compound 48 as a brown solid (yield=21%).

### LLLL. Compound S01191

The synthetic procedure from intermediate 48 to the target compound S01191 was similar to the general procedure.

### MMMM. Compound S01553

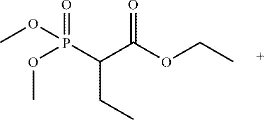

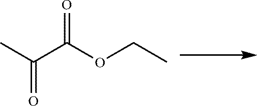

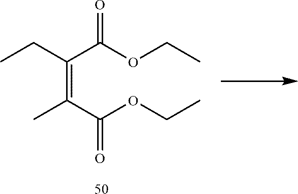

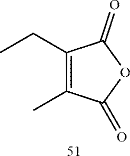

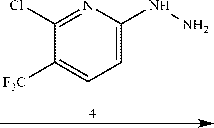

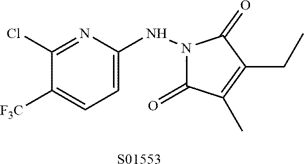

### NNNN. Intermediate 50

A solution of ethyl 2-(dimethoxyphosphoryl)butanonate (1.0 g, 4.0 mmol) in 1,2-dimethoxyethane (5 mL) was added to a stirred slurry of sodium hydride in 1,2-dimethoxyethane (10 mL). When evolution of hydrogen ceased, ethyl pyruvate (480 mg, 4.1 mmol) in 1,2-dimethoxyethane (5 mL) was added to solution. The mixture was stirred at 50° C. overnight. Then the solution was diluted with EtOAc (100 mL), washed with water and brine, and dried over anhydrous Na₂SO₄. The solvent was removed in vacuo, and the residue was purified by chromatography to give the product (intermediate 50). Yield was 710 mg (87.2%)

### OOOO. Intermediate 51

A solution of diethyl 2-ethyl-3-methylmaleate (75 mg, 0.35 mmol) in ethanol (0.8 mL) was added dropwise to aqueous NaOH (2 M, 0.4 mL) dropwise. The mixture was stirred at room temperature for 30 min, then diluted with water (10 mL) and washed with ether (5 mL). The aqueous layer was acidified with 5% aq. HCl, then extracted with EtOAc. The organic layer was washed with brine and dried over anhydrous Na₂SO₄. The solvent was removed in vacuo, and the residue was purified by chromatography on a silica gel column. Yield of intermediate 51 was 41 mg (83.7%)

### PPPP. Compound S01553

The synthetic procedure from intermediate 51 to compound S01553 was similar to the general procedure.

### QQQQ. Compound S01554

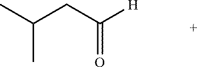

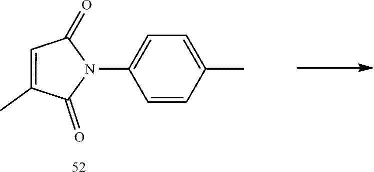

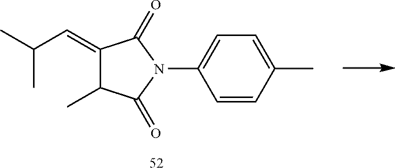

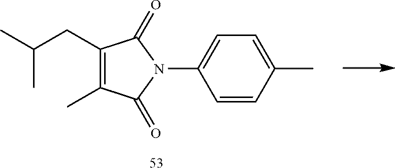

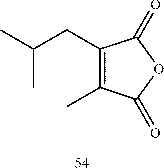

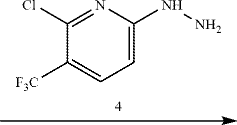

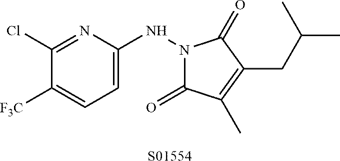

### RRRR. Intermediate 52

A mixture of citraconimide (200 mg, 1.0 mmol) and PPh₃ (320 mg, 1.2 mmol) in glacial AcOH (7 mL) was stirred at room temperature for 1 hour. Isovaleraldehyde (160 µl, 1.5 mmol), was added and the reaction mixture was refluxed with stirring for 24 hours. HOAc was distilled off in vacuo, the residue was dissolved in EtOAc (30 mL), and the organic layer was washed with H₂O, brine and dried over anhydrous NaSO₄. The solvent was removed in vacuo and the residue was purified by chromatography on a silica gel column. Yield of intermediate 52: (90 mg, 35.0%)

### SSSS. Intermediate 53

To a stirred solution of intermediate 52 (90 mg) in THF (2 mL) was added Et₃ N (0.4 mL). The reaction mixture was refluxed for 48 hours, and then was concentrated in vacuo. The residue was dissolved in EtOAc and the organic layer washed with water, brine and dried over anhydrous Na₂SO₄. The solvent was removed in vacuo and the residue was purified by chromatography on a silica gel column. Yield of intermediate 53: (85 mg, 94.4%).

### TTTT. Intermediate 54

To the solution of 53 (50 mg, 0.19 mmol) in THF (0.3 mL) and MeOH (0.6 mL) was added aq. KOH (1 mL, 30%) and the reaction mixture was refluxed for 12 hours with stirring. Then the reaction mixture was concentrated in vacuo, the obtained residue was acidified with dilute aq. HCl and extracted with EtOAc (20 mL). The organic layer was washed with water, brine and dried over anhydrous Na₂SO₄. The solvent was removed in vacuo and the residue was purified by chromatography on a silica gel column. Yield of intermediate 54: (26 mg, 81.3%).

### UUUU. Compound S01554

The synthetic procedure from intermediate 54 to compound S01554 was similar to the general procedure.

### VVVV. Compound S00873

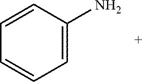

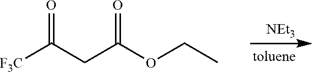

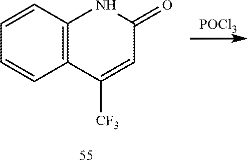

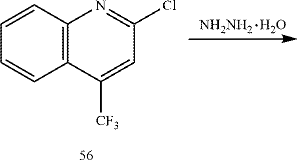

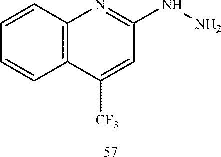

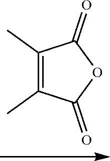

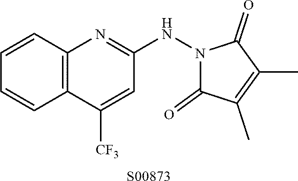

### WWWW. Intermediate 55

To a solution of the ester (5.46 mmol) and triethylamine (101 g, 10.86 mmol) in toluene (5 mL) was added a solution of aniline (6.52 mmol) in toluene (2 mL) at room temperature. The reaction mixture was refluxed until the reaction was complete. After workup, intermediate compound 55 was obtained, which was pure enough to be used in the next step.

### XXXX. Intermediate 57

The mixture of intermediate 55 and POC1₃ (5 mL) was refluxed for 5 h, and then poured into the ice water. The ether extract was washed with brine and dried over anhydrous Na₂SO₄, and then concentrated to afford the intermediate compound 56, which was directly used in next step.

The mixture of intermediate 56 and hydrazine hydrate in 5 mL of ethanol was refluxed for several hours until the starting material disappeared. After workup, intermediate compound 57 was obtained.

### YYYY. Compound S00873

The synthetic procedure from intermediate 57 to compound S00873 was similar to the general procedure.

### ZZZZ. Compound S01455

The synthetic route of compound S01455 is similar to compound S00873.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/-5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

### REFERENCES

Wang J, Barve M, Chiorean EG, et al. Interim Results from a Phase 1 Trial of SL-801, a Novel XPO1 Inhibitor, in Patients with Advanced Solid Tumors. Annals of Oncology (2019) 30 (suppl_5): v159-v193.

Zhou Y, Hou Y, Shen J, et al. Network-based drug repurposing for novel coronavirus 2019-nCoV/SARS-CoV-2. Cell Discov. 2020 Mar 16;6:14.

Mathew C and Ghildyal R. CRM1 Inhibitors for Antiviral Therapy. Front Microbiol. 2017 Jun 28;8:1171.

Freundt EC, Yu L, Park E, et al. Molecular Determinants for Subcellular Localization of the Severe Acute Respiratory Syndrome Coronavirus Open Reading Frame 3b Protein. J Virol. 2009 Jul;83(13):6631-40.

Sharma K, Akerström S, Sharma AK, et al. SARS-CoV 9b Protein Diffuses into Nucleus, Undergoes Active Crm1 Mediated Nucleocytoplasmic Export and Triggers Apoptosis When Retained in the Nucleus. PLoS One. 2011; 6(5): e19436.

Gordon DE, Jang GM, Bouhaddou M, et al. A SARS-CoV-2 Protein Interaction Map Reveals Targets for Drug Repurposing. Nature. 2020 Apr 30.

Funk C, Raschbichler V, Lieber D, et al. Traffic. 2019 Feb; 20(2):152-167.

Sanchez V, Mahr JA, Orazio NI, et al. J Virol. 2007 Nov; 81(21):11730-6.

Liu Y, Zhang Z, Zhao X, et al. Virus Res. 2012 Jun; 166(1-2):31-42.

Boyle SM, Ruvolo V, Gupta AK, et al. J Virol. 1999 Aug; 73(8):6872-81.

Rawlinson SM, Pryor MJ, Wright PJ, et al. J Biol Chem. 2009 Jun 5; 284(23):15589-97.

Oh W, Yang MR, Lee EW, et al. J Biol Chem. 2006 Oct 6; 281(40):30166-74.

Forgues M, Marrogi AJ, Spillare EA, et al. J Biol Chem. 2001 Jun 22; 276(25):22797-803.

Atasheva S, Fish A, Fornerod M, et al. J Virol. 2010 May; 84(9):4158-71.

Thomas S, Rai J, John L, et al. Virol J. 2013 Aug 28; 10:269.

Blachon S, Bellanger S, Demeret C, et al. J Biol Chem. 2005 Oct 28; 280(43):36088-98.

Ghildyal R, Ho A, Dias M, et al. J Virol.2009 Jun; 83(11):5353-62.

Schmid M, Gonzalez RA, Dobner T. J Virol.2012 Feb; 86(4):2282-92.

Wang YE, Park A, Lake M, et al. PLoS Pathog. 2010 Nov 11;6(11):e1001186.

What is claimed is:

1. A method of treating a viral infection in a subject comprising administering to the subject an effective amount of a compound having the formula of Structure (II): 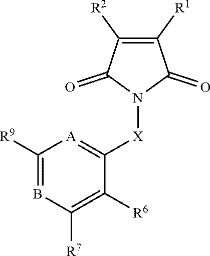
wherein:
R¹ and R² are independently selected from alkyl, substituted alkyl, and optionally substituted alkoxy, wherein at least one of R¹ and R² is methyl;
X is NR³;
R³ is H, alkyl, or acyl;
A is N or CH;
B is CR⁸;
R⁶ is selected from H, alkyl, substituted alkyl, and halogen;
R⁷ is selected from H, alkyl, substituted alkyl, halogen, optionally substituted aryl, optionally substituted heteroaryl, optionally substituted alkoxy, optionally substituted aryloxy, cyano, optionally substituted alkylthio, optionally substituted alkylsufinyl, optionally substituted alkylsulfonyl, optionally substituted arylthio, optionally substituted acyl, optionally substituted amino, carboxyl, optionally substituted alkoxycarbonyl, and optionally substituted carbamoyl, wherein R⁶ and R⁷ optionally form a fused aryl group when each of R⁶ and R⁷ is alkyl;
R⁸ is selected from H, alkyl, substituted alkyl, and halogen; and
R⁹ is selected from H, alkyl, substituted alkyl, halogen, optionally substituted aryl, and cyano, wherein R⁸ and R⁹ optionally form one or more optionally substituted fused aryl groups when each of R⁸ and R⁹ is alkyl or substituted alkyl;
wherein at least one of R⁶, R⁷, R⁸, and R⁹ is halogen selected from Br and Cl, or alkyl substituted with one or more halogen groups selected from Br, Cl, and F;
or a salt thereof.

2. The method of claim 1, wherein the compound has the formula of Structure (IV): 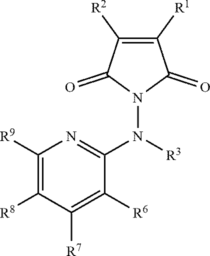
.

3. The method of claim 1 or 2, wherein one of R¹ and R² is methyl, and the other of R¹ and R² is alkyl or alkyl substituted with alkoxy, hydroxy, carboxy, or alkoxycarbonyl.

4. The method of any one of claims 1-3, wherein R³ is H or alkyl.

5. The method of any one of claims 1-4, wherein R³ is H or methyl.

6. The method of any one of claims 1-5, wherein R⁶ is H, R⁷ is H, R⁸ is halogen or alkyl substituted with one or more halogen groups, and R⁹ is halogen.

7. The method of any one of claims 1-6, wherein one of R¹ and R² is methyl, and the other of R¹ and R² is alkyl or alkyl substituted with alkoxy, hydroxy, carboxy, or alkoxycarbonyl, R⁶ is H, R⁷ is H, R⁸ is CF₃, and R⁹ is Cl.

8. The method of any one of claims 1-7, wherein the compound is 3-[(3,3-dimethylbutoxy)methyl]-1-{[6-chloro-5-(trifluoromethyl)(2-pyridyl)]amino}-4-methylazoline-2,5-dione (S03747), or a salt thereof, having the following structure: 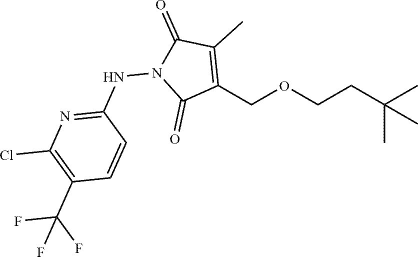 [SMILES: CC1=C(COCCC(C)(C)C)C(=O)N(Nc2ccc(C(F)(F)F)c(Cl)n2)C1=O]
.

9. The method of any one of claims 1-8, wherein the compound has a selectivity index of greater than 1 in animal cells.

10. The method of any one of claims 1-8, wherein the compound has a selectivity index of between 1 and 1000 in animal cells.

11. The method of any one of claims 1-10, wherein the compound is administered as a pharmaceutical composition comprising the compound and a pharmaceutically acceptable excipient.

12. The method of any one of claims 1-11, wherein the compound binds XPO1.

13. The method of any one of claims 1-12, wherein the compound binds to Cys528 of XPO1.

14. The method of claim 12 or 13, wherein the binding is reversible.

15. The method of any one of claims 1-14, wherein contacting a cell with the compound increases nuclear retention of a viral protein.

16. The method of any one of claims 1-15, wherein contacting a cell with the compound increases nuclear retention of a viral ribonucleoprotein (vRNP).

17. The method of any one of claims 1-16, wherein contacting a cell with the compound blocks nuclear export of a vRNP or viral protein.

18. The method of any one of claims 1-17, wherein the viral infection is caused by a virus belonging to the Togaviridae, Arenaviridae, Poxviridae, Toroviridae, Paramyxoviridae, Herpesviridae, Retroviridae, Coronaviridae, Flaviviridae, Bunyaviridae, Pneumoviridae, Filoviridae, Adenoviridae, Papovaviridiae, Hepadnaviridae, or Orthomyxoviridae family.

19. The method of any one of claims 1-18, wherein the viral infection is caused by dengue virus (DENV), respiratory syncytial virus (RSV), Venezuelan equine encephalitis virus (VEEV), influenza virus, human immunodeficiency virus (HIV), herpes simplex virus (HSV), cytomegalovirus (CMV), Ebola virus, rubulavirus, Nipah virus, Hepatitis B virus, BK virus, JC virus, papillomavirus, adenovirus-5, cowpox virus, measles virus, varicella-zoster virus, Epstein-Barr virus, Kaposi’s sarcoma associated herpesvirus, West Nile virus, Chikungunya virus (CHIKV), or coronavirus.

20. The method of any one of claims 1-19, wherein the viral infection is an influenza infection.

21. The method of claim 20, wherein the influenza infection is an influenza A, influenza B, or influenza C infection.

22. The method of claim 21, wherein the influenza A infection comprises infection by H1N1, H1N2, H3N2, H5N1, or H7N9 subtypes of influenza.

23. The method of any one of claims 1-19, wherein the viral infection is a Coronavirus infection.

24. The method of claim 23, wherein the Coronavirus infection comprises infection by SARS-CoV2.

25. The method of claim 23 or 24, wherein the Coronavirus infection causes COVID-19.

26. The method of any one of claims 1-19, wherein the viral infection is a human immunodeficiency virus (HIV) infection.

27. The method of any one of claims 1-19, wherein the viral infection is a Nipah virus infection.

28. The method of any one of claims 1-27, wherein the method of treating a viral infection comprises reducing the duration of infection.

29. The method of any one of claims 1-28, wherein the method of treating a viral infection comprises reducing the symptoms of infection.

30. The method of any one of claims 1-29, wherein the method of treating a viral infection comprises reducing the severity of the infection.

31. The method of any one of claims 1-30, wherein the method of treating a viral infection comprises reducing viral infectivity.

32. The method of any one of claims 1-31, wherein the method of treating a viral infection comprises reducing viral replication.

33. The method of any one of claims 1-32, wherein the method of treating a viral infection comprises reducing viral shedding.

34. The method of any one of claims 1-33, wherein the subject is a human patient.

35. The method of any one of claims 1-33, wherein the subject is a cell and the method is an in vitro method.

36. The method of any one of claims 1-33, wherein the subject is a human patient’s cell and the method is an ex vivo method.

37. A compound as recited in any one of claims 1-36 for use in the manufacture of a medicament for treating a viral infection.

38. The use of a compound as recited in any one of claims 1-36 for treating a viral infection.
